(19) United States
(12) Patent Application Publication
(10) Pub. No.: US 2026/0232578 A1
(43) Pub. Date: Aug. 13, 2026

(54) COMPOSITIONS AND METHODS FOR TRANSMUCOSAL ABSORPTION

(71) Applicant: Tonix Pharma Limited, Dublin (IE)

(72) Inventors: Seth Lederman, Austin, TX (US); Giorgio Reiner, Como (IT); Harry G. Brittain, Milford, NJ (US)

(21) Appl. No.: 19/409,704

(22) Filed: Dec. 4, 2025

Related U.S. Application Data
(62) Division of application No. 13/918,692, filed on Jun. 14, 2013, now abandoned.
(60) Provisional application No. 61/660,593, filed on Jun. 15, 2012.
(60) Provisional application No. 61/667,774, filed on Jul. 3, 2012.
(60) Provisional application No. 61/725,402, filed on Nov. 12, 2012.
(60) Provisional application No. 61/792,900, filed on Mar. 15, 2013.

(51) Int. Cl. A61K 9/00; A61K 9/20; A61K 31/135

(52) U.S. Cl. CPC A61K 9/006; A61K 9/2009; A61K 31/135; A61K 9/2018; A61K 9/2059

(57) ABSTRACT

The invention provides compositions and methods for administering compounds for transmucosal absorption. The compositions and methods have a number of surprising pharmacokinetic benefits over oral administration of a compound.

This application is a divisional of U.S. patent application Ser. No. 13/918,692, filed Jun. 14, 2013, which claims priority from U.S. Provisional Application 61/660,593, filed Jun. 15, 2012, U.S. Provisional Application 61/667,774, filed Jul. 3, 2012, U.S. Provisional Application 61/725,402, filed Nov. 12, 2012, and U.S. Provisional Application 61/792,900, filed Mar. 15, 2013. The disclosures of each of those applications are hereby incorporated by reference in their entireties.

### BACKGROUND OF THE INVENTION

Cyclobenzaprine, or 3-(5H-dibenzo[a,d]cyclohepten-5-ylidene)-N,N-dimethyl-1-propanamine, was first approved by the U.S. Food and Drug Administration in 1977 for the treatment of acute muscle spasms of local origin. (Katz, W., et al., Clinical Therapeutics 10:216-228 (1988)). Amitriptyline, or 3-(10,11-dihydro-5H-dibenzo [a,d]cycloheptene-5-ylidene)-N,N-dimethyl-1-propanamine, was first approved by the U.S. Food and Drug Administration for the treatment of depression.

Subsequent studies have shown cyclobenzaprine to also be effective in the treatment of fibromyalgia syndrome, post-traumatic stress disorder (PTSD), traumatic brain injury (TBI), generalized anxiety disorder and depression. Furthermore, the utility of cyclobenzaprine as an agent for improving the quality of sleep, as a sleep deepener, or for treating sleep disturbances has been investigated. However, while FDA-approved therapeutics address pain and mood, but there are currently no FDA-approved treatments that address the disturbed sleep and fatigue associated with fibromyalgia syndrome. Treatment with cyclobenzaprine may be particularly useful in treating sleep disturbances caused by, exacerbated by, or associated with fibromyalgia syndrome, prolonged fatigue, chronic fatigue, chronic fatigue syndrome, a sleep disorder, a psychogenic pain disorder, chronic pain syndrome (type II), the administration of a drug, autoimmune disease, stress or anxiety, or for treating an illness caused by or exacerbated by sleep disturbances, and symptoms of such illness. See, for example, U.S. Pat. Nos. 6,395,788 and 6,358,944, incorporated herein by reference.

Despite the broad therapeutic usefulness of cyclobenzaprine, cyclobenzaprine is absorbed slowly into the blood stream after oral administration and should be taken approximately one to two hours before an effect is desired. If the effect is desired sooner, the patient must wait for the effect to occur, which is not desirable for use as a sleep aid and is not desirable for a patient with symptoms of muscle spasms seeking relief. In part because oral cyclobenzaprine has a slow onset of action, in desperation fibromyalgia patients sometimes try to manage the non-restorative sleep associated with fibromyalgia through the use of prescription sedatives or hypnotics, which are not effective for treating the sleep quality problems associated with fibromyalgia and can be addictive. Despite the broad therapeutic usefulness of cyclobenzaprine, cyclobenzaprine often causes fatigue, somnolence, a groggy feeling, or cognitive impairment in individuals, which is not desirable during normal periods of wakefulness. Cyclobenzaprine also is only recommended for short-term usage, as it has not been shown to provide benefits during long-term administration. In part because cyclobenzaprine is not a chronic treatment, in desperation fibromyalgia patients sometimes try to manage the pain associated with fibromyalgia through the use of opiate analgesics, which are not effective for treating fibromyalgia pain and can be addictive. Studies with cyclobenzaprine in various formulations, have led to the conclusion that the slow and delayed absorption of cyclobenzaprine after oral administration leads to undesirable characteristics for a bedtime medication designed to target the sleeping brain in daily therapy. Thus, improved cyclobenzaprine formulations are desirable.

### SUMMARY OF THE INVENTION

In some embodiments, the invention provides a composition comprising cyclobenzaprine, wherein the composition is suitable for transmucosal absorption. In some embodiments, the invention provides a composition comprising cyclobenzaprine and a basifying agent, wherein the composition is suitable for transmucosal absorption.

In some embodiments, the invention provides a composition comprising amitriptyline, wherein the composition is suitable for transmucosal absorption. In some embodiments, the invention provides a composition comprising amitriptyline and a basifying agent, wherein the composition is suitable for transmucosal absorption.

In certain embodiments, the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate and trisodium citrate.

In certain embodiments, the transmucosal absorption is oral absorption. In certain embodiments, the composition is suitable for sublingual administration. In further embodiments, the composition is in a form selected from the group consisting of a sublingual tablet, a sublingual film, a sublingual powder, and a sublingual spray solution.

In certain embodiments, the composition is suitable for buccal administration. In further embodiments, the composition is in a form selected from the group consisting of a buccal tablet, a lozenge, a buccal powder, and a buccal spray solution.

In certain embodiments, the transmucosal absorption is intranasal absorption. In further embodiments, the composition is in a form of a nasal spray solution. In certain embodiments, the transmucosal absorption is pulmonary absorption. In further embodiments, the composition is in a form selected from the group consisting of an aerosolized composition and an inhalable dry powder.

In some embodiments, a composition is characterized in that, when administered by transmucosal absorption, the cyclobenzaprine or amitriptyline has a dnC* of greater than or equal to 50±25%×10⁻⁹ mL⁻¹ 10 minutes after administration. In some embodiments, a composition is characterized in that, when administered by transmucosal absorption, the cyclobenzaprine or amitriptyline has a dnC* of greater than or equal to 125±25%×10⁻⁹ mL⁻¹ 15 minutes after administration. In some embodiments, a composition is characterized in that, when administered by transmucosal absorption, the cyclobenzaprine or amitriptyline has a dnC* of greater than or equal to 150±25%×10⁻⁹ mL⁻¹ 20 minutes after administration. In some embodiments, a composition is characterized in that, when administered by transmucosal absorption, the cyclobenzaprine or amitriptyline has a dnC* of greater than or equal to 300±25%×10⁻⁹ mL⁻¹ 30 minutes after administration. In some embodiments, a composition is characterized in that, when administered by transmucosal absorption, the cyclobenzaprine or amitriptyline has a dnC* of greater than or equal to 450±25%×10⁻⁹ mL⁻¹ 45 minutes after administration. In some embodiments, a composition is characterized in that, when administered by transmucosal absorption, the cyclobenzaprine or amitriptyline has a dnC* of greater than or equal to 600±25%×10⁻⁹ mL⁻¹ 1 hour after administration. In some embodiments, a composition is characterized in that, when administered by transmucosal absorption, the cyclobenzaprine or amitriptyline has a dnC* of greater than or equal to 700±25%×10⁻⁹ mL⁻¹ 2 hours after administration. In some embodiments, a composition is characterized in that, when administered by transmucosal absorption, the cyclobenzaprine or amitriptyline has a dnC* of greater than or equal to 750±25%×10⁻⁹ mL^{-1 2.5} (150 min) hours after administration. In some embodiments, a composition is characterized in that, when administered by transmucosal absorption, the cyclobenzaprine or amitriptyline has a dnC* of greater than or equal to 850±25%×10⁻⁹ mL⁻¹ 3 hours after administration. In some embodiments, a composition is characterized in that, when administered by transmucosal absorption, the cyclobenzaprine or amitriptyline has a dnC* of greater than or equal to 900±25%×10⁻⁹ mL⁻¹ 3.3 hours (200 min) after administration. In some embodiments, a composition is characterized in that, when administered by transmucosal absorption, the cyclobenzaprine or amitriptyline has a dnC* of greater than or equal to 950±25%×10⁻⁹ mL⁻¹ 3.7 (220 min) hours after administration. In some embodiments, a composition is characterized in that, when administered by transmucosal absorption, the cyclobenzaprine or amitriptyline has a dnC* of greater than or equal to 1000±25%×10⁻⁹ mL⁻¹ 4 hours after administration. In some embodiments, a composition is characterized in that, when administered by transmucosal absorption, the cyclobenzaprine or amitriptyline has a dnC* of greater than or equal to 1000±25%×10⁻⁹ mL⁻¹ 4.33 (260 min) hours after administration. In some embodiments, a composition is characterized in that, when administered by transmucosal absorption, the cyclobenzaprine or amitriptyline has a dnC* of greater than or equal to 1050±25%×10⁻⁹ mL⁻¹ 4.67 hours (280 min) after administration. In some embodiments, a composition is characterized in that, when administered by transmucosal absorption, the cyclobenzaprine or amitriptyline has a dnC* of greater than or equal to 1000±25%×10⁻⁹ mL⁻¹ 5 hours after administration. In some embodiments, a composition is characterized in that, when administered by transmucosal absorption, the cyclobenzaprine or amitriptyline has a dnC* of greater than or equal to 1000±25%×10⁻⁹ mL⁻¹ 5.5 hours after administration. In some embodiments, a composition is characterized in that, when administered by transmucosal absorption, the cyclobenzaprine or amitriptyline has a dnC* of greater than or equal to 900±25%×10⁻⁹ mL⁻¹ 6 hours after administration. In some embodiments, a composition is characterized in that, when administered by transmucosal absorption, the cyclobenzaprine or amitriptyline has a dnC* of greater than or equal to 700±25%×10⁻⁹ mL⁻¹ 8 hours after administration. In some embodiments, a composition is characterized in that, when administered by transmucosal absorption, the cyclobenzaprine or amitriptyline has a dnC* of less than or equal to 650±25%×10⁻⁹ mL⁻¹ 10 hours after administration. In some embodiments, a composition is characterized in that, when administered by transmucosal absorption, the cyclobenzaprine or amitriptyline has a dnC* of less than or equal to 500±25%×10⁻⁹ mL⁻¹ 12 hours after administration. In some embodiments, a composition is characterized in that, when administered by transmucosal absorption, the cyclobenzaprine or amitriptyline has a dnC* of less than or equal to 500±25%×10⁻⁹ mL⁻¹ 14 hours after administration. In some embodiments, a composition is characterized in that, when administered by transmucosal absorption, the cyclobenzaprine or amitriptyline has a dnC* of less than or equal to 350±25%×10⁻⁹ mL⁻¹ 16 hours after administration. In some embodiments, a composition is characterized in that, when administered by transmucosal absorption, the cyclobenzaprine or amitriptyline has a dnC* of less than or equal to 350±25%×10⁻⁹ mL⁻¹ 18 hours after administration. In some embodiments, a composition is characterized in that, when administered by transmucosal absorption, the cyclobenzaprine or amitriptyline has a dnC* of less than or equal to 300±25%×10⁻⁹ mL⁻¹ 20 hours after administration. In some embodiments, a composition is characterized in that, when administered by transmucosal absorption, the cyclobenzaprine or amitriptyline has a dnC* of less than or equal to 300±25%×10⁻⁹ mL⁻¹ 22 hours after administration. In some embodiments, a composition is characterized in that, when administered by transmucosal absorption, the cyclobenzaprine or amitriptyline has a dnC* of less than or equal to 300±25%×10⁻⁹ mL⁻¹ 24 hours after administration. In some embodiments, a composition is characterized in that, when administered by transmucosal absorption, the cyclobenzaprine or amitriptyline has a dnC* of less than or equal to 200±25%×10⁻⁹ mL⁻¹ 36 hours after administration. In some embodiments, a composition is characterized in that, when administered by transmucosal absorption, the cyclobenzaprine or amitriptyline has a dnC* of less than or equal to 150±25%×10⁻⁹ mL⁻¹ 48 hours after administration. In some embodiments, a composition is characterized in that, when administered by transmucosal absorption, the cyclobenzaprine or amitriptyline has a dnC* of less than or equal to 100±25%×10⁻⁹ mL⁻¹ 72 hours after administration. In some embodiments, a composition is characterized in that, when administered by transmucosal absorption, the cyclobenzaprine or amitriptyline has a dnC* of greater than or equal to 2.0±25%×10⁻⁹ mL⁻¹ 30 minutes after administration. In some embodiments, a composition is characterized in that, when administered by transmucosal absorption, the cyclobenzaprine or amitriptyline has a dnC* of greater than or equal to 2.0±25%×10⁻⁹ mL⁻¹ 45 minutes after administration. In some embodiments, a composition is characterized in that, when administered by transmucosal absorption, the cyclobenzaprine or amitriptyline has a dnC* of greater than or equal to 2.0±25%×10⁻⁹ mL⁻¹ 1 hour after administration. In some embodiments, a composition is characterized in that, when administered by transmucosal absorption, the cyclobenzaprine or amitriptyline has a dnC* of greater than or equal to 2.0±25%×10⁻⁹ mL⁻¹ 2 hours after administration. In some embodiments, a composition is characterized in that, when administered by transmucosal absorption, the cyclobenzaprine or amitriptyline has a dnC* of greater than or equal to 2.0±25%×10⁻⁹ mL⁻¹ 3 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 8 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 10 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 12 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 14 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 16 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 18 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 20 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 22 hours after administration. In some embodiments, a composition is characterized in that, when administered by transmucosal absorption, the cyclobenzaprine or amitriptyline has a dnC* of less than or equal to 5.0±25%×10⁻⁷ mL⁻¹ 24 hours after administration.

In some embodiments, a composition is characterized in that, when administered by transmucosal absorption, the cyclobenzaprine or amitriptyline has a dnAUC_{0-8 h} of greater than or equal to 5±25%×10⁻⁶ mL⁻¹ hr. In some embodiments, a composition is characterized in that, when administered by transmucosal absorption, the cyclobenzaprine or amitriptyline has a dnAUC_{0-∞h} of greater than or equal to 20±25%×10⁻⁶ mL⁻¹ hr. In some embodiments, a composition is characterized in that, when administered by transmucosal absorption, the cyclobenzaprine or amitriptyline has a dnCₘₐₓ* of greater than or equal to 1.0±25%×10⁻⁶ mL⁻¹. In some embodiments, a composition is characterized in that, when administered by transmucosal absorption, the cyclobenzaprine or amitriptyline has a partial AUC₀₋₂₀min of greater than or equal to 37±25% ng hr L⁻¹, an AUC₀₋₃₀min of greater than or equal to 128±25% ng hr L⁻¹, an AUC₀₋₄₅min of greater than or equal to 333±25% ng hr L⁻¹, an AUC_{0-1 h} of greater than or equal to 614±25% ng hr L⁻¹, an AUC_{0-2 h} of greater than or equal to 2098±25% ng hr L⁻¹, an AUC_{0-2.5 h} of greater than or equal to 2955±25% ng hr L⁻¹, an AUC_{0-3 h} of greater than or equal to 3931±25% ng hr L⁻¹. In some embodiments, a composition is characterized in that, when administered by transmucosal absorption, the cyclobenzaprine or amitriptyline has a partial AUC₀₋₂₀min of greater than or equal to 23±25% ng hr L⁻¹, an AUC₀₋₃₀min of greater than or equal to 86±25% ng hr L⁻¹, an AUC₀₋₄₅min of greater than or equal to 223±25% ng hr L⁻¹, an AUC_{0-1 h} of greater than or equal to 405±25% ng hr L⁻¹, an AUC_{0-2 h} of greater than or equal to 1478±25% ng hr L⁻¹, an AUC_{0-2.5 h} of greater than or equal to 2167±25% ng hr L⁻¹. In some embodiments, the dnAUC₀₋₂₀min is about 0.02±25%×10⁻⁶ hr mL⁻¹, the dnAUC₀₋₃₀min is about 0.05±25%×10⁻⁶ hr mL⁻¹, the dnAUC₀₋₄₅min is about 0.15±25%×10⁻⁶ hr mL⁻¹, the dnAUC_{0-1 h} is about 0.25±25%×10⁻⁶ hr mL⁻¹, the dnAUC_{0-2 h} is about 0.9±25%×10⁻⁶ hr mL⁻¹, the dnAUC_{0-2.5 h} is about 1.2±25%×10⁻⁶ hr mL⁻¹, the dnAUC_{0-3 h} is about 1.5±25%×10⁻⁶ hr mL⁻¹, the dnAUC_{3.3h} is about 1.8±25%×10⁻⁶ hr mL⁻¹, dnAUC_{0-3.7 h} is about 2.3±25%×10⁻⁶ hr mL⁻¹, the dnAUC_{0-4 h} is about 2.6±25%×10⁻⁶ hr mL⁻¹, the dnAUC_{0.4.3h} is about 3.0±25%×10⁻⁶ hr mL⁻¹, the dnAUC_{0-4.7 h} is about 3.3±25%×10⁻⁶ hr mL⁻¹, the dnAUC_{0-5 h} is about 3.7±25%×10⁻⁶ hr mL⁻¹, the dnAUC_{0-5.5 h} is about 4.2±25%×10⁻⁶ hr mL⁻¹, the dnAUC_{0-6 h} is about 4.7±25%×10⁻⁶ hr mL⁻¹, the dnAUC_{0-8 h} is 6.30±25%×10⁻⁶ hr mL⁻¹, the dnAUC_{0-12 h} is about 20±25%×10⁻⁶ hr mL⁻¹, and the dnAUC_{0-∞h} is about 25±25%× 10⁻⁶ hr mL⁻¹.

In some embodiments, a composition is characterized in that, when administered by transmucosal absorption, the cyclobenzaprine or amitriptyline has a dbmnAUC₀₋₂₀min of greater than or equal to 1.0±25%×10⁻⁶ kg hr mL⁻¹. In some embodiments, a composition is characterized in that, when administered by transmucosal absorption, the cyclobenzaprine or amitriptyline has a dbmnAUC₀₋₃₀min of greater than or equal to 3.5±25%×10⁻⁶ kg hr mL⁻¹. In some embodiments, a composition is characterized in that, when administered by transmucosal absorption, the cyclobenzaprine or amitriptyline has a dbmnAUC₀₋₄₅min of greater than or equal to 10±25%×10⁻⁶ kg hr mL⁻¹. In some embodiments, a composition is characterized in that, when administered by transmucosal absorption, the cyclobenzaprine or amitriptyline has a dbmnAUC_{0-1 h} of greater than or equal to 18±25%×10⁻⁶ kg hr mL⁻¹. In some embodiments, a composition is characterized in that, when administered by transmucosal absorption, the cyclobenzaprine or amitriptyline has a dbmnAUC_{0-2 h} of greater than or equal to 60±25%×10⁻⁶ kg hr mL⁻¹. In some embodiments, a composition is characterized in that, when administered by transmucosal absorption, the cyclobenzaprine or amitriptyline has a dbmnAUC_{0-2.5 h} of greater than or equal to 85±25%×10⁻⁶ kg hr mL⁻¹. In some embodiments, a composition is characterized in that, when administered by transmucosal absorption, the cyclobenzaprine or amitriptyline has a dbmnAUC_{0-3 h} of greater than or equal to 115±25%×10⁻⁶ kg hr mL⁻¹. In some embodiments, a composition is characterized in that, when administered by transmucosal absorption, the cyclobenzaprine or amitriptyline has a dbmnAUC_{3.3h} of greater than or equal to 135±25%×10⁻⁶ kg hr mL⁻¹. In some embodiments, a composition is characterized in that, when administered by transmucosal absorption, the cyclobenzaprine or amitriptyline has adbmnAUC_{0-3.7 h} of greater than or equal to 160±25%×10⁻⁶ kg hr mL⁻¹. In some embodiments, a composition is characterized in that, when administered by transmucosal absorption, the cyclobenzaprine or amitriptyline has a dbmnAUC_{0-4 h} of greater than or equal to 180±25%×10⁻⁶ kg hr mL⁻¹. In some embodiments, a composition is characterized in that, when administered by transmucosal absorption, the cyclobenzaprine or amitriptyline has a dbmnAUC_{0-4.3 h} of greater than or equal to 210±25%×10⁻⁶ kg hr mL⁻¹. In some embodiments, a composition is characterized in that, when administered by transmucosal absorption, the cyclobenzaprine or amitriptyline has a dbmnAUC_{0-4.7 h} of greater than or equal to 230±25%×10⁻⁶ kg hr mL⁻¹. In some embodiments, a composition is characterized in that, when administered by transmucosal absorption, the cyclobenzaprine or amitriptyline has a dbmnAUC_{0-5 h} of greater than or equal to 250±25%×10⁻⁶ kg hr mL⁻¹. In some embodiments, a composition is characterized in that, when administered by transmucosal absorption, the cyclobenzaprine or amitriptyline has a dbmnAUC_{0-5.5 h} of greater than or equal to 290±25%×10⁻⁶ kg hr mL⁻¹. In some embodiments, a composition is characterized in that, when administered by transmucosal absorption, the cyclobenzaprine or amitriptyline has a dbmnAUC_{0-6 h} of greater than or equal to 330±25%×10⁻⁶ kg hr mL⁻¹. In some embodiments, a composition is characterized in that, when administered by transmucosal absorption, the cyclobenzaprine or amitriptyline has a dbmnAUC_{0-8 h} is 440±25%×10⁻⁶ kg hr mL⁻¹. In some embodiments, a composition is characterized in that, when administered by transmucosal absorption, the cyclobenzaprine or amitriptyline has a dbmnAUC_{0-12 h} of greater than or equal to 1500±25%×10⁻⁶ kg hr mL⁻¹. In some embodiments, a composition is characterized in that, when administered by transmucosal absorption, the cyclobenzaprine or amitriptyline has a dbmnAUC_{0-inf} of greater than or equal to 1800±25%×10⁻⁶ kg hr mL⁻¹.

In some embodiments, a composition is characterized in that, when administered by transmucosal absorption, the cyclobenzaprine or amitriptyline has a plasma concentration of 50% or less of the Cₘₐₓ 8 hours after administration. In some embodiments, a composition is characterized in that, when administered by transmucosal absorption, the cyclobenzaprine or amitriptyline has a plasma concentration of 50% or less of the Cₘₐₓ 12 hours after administration.

In some embodiments, cyclobenzaprine is present in a composition of the invention in an amount from 0.1 mg to 10 mg, for example, from 0.1 mg to 5 mg. In certain embodiments, the cyclobenzaprine is present in an amount of about 2.4 mg, less than about 2.4 mg, about 4.8 mg, or less than about 4.8 mg. In certain embodiments, the cyclobenzaprine is present in an amount of about 2.8 mg, less than about 2.8 mg, about 5.6 mg, or less than about 5.6 mg. In certain embodiments, the cyclobenzaprine is present in an amount of about 4.5 mg, less than about 5 mg, about 9 mg, or less than about 10 mg.

In some embodiments, a composition is characterized in that, when administered by transmucosal absorption, the composition affords a Cₘₐₓ of cyclobenzaprine greater than or equal to 10 ng/mL. In some embodiments, a composition is characterized in that, when administered by transmucosal absorption, the composition affords a Cₘₐₓ of cyclobenzaprine greater than or equal to 15 ng/mL, greater than or equal to 20 ng/mL, greater than or equal to 25 ng/mL or greater than or equal to 30 ng/mL. In some embodiments, a composition is characterized in that, when administered by transmucosal absorption, the composition affords a Cₘₐₓ of cyclobenzaprine greater than or equal to 2.5 ng/mL, greater than or equal to 3 ng/mL, greater than or equal to 4 ng/mL, greater than or equal to 10 ng/mL. In some embodiments, a composition is characterized in that, when administered by transmucosal absorption, the composition affords a Cₘₐₓ of cyclobenzaprine greater than or equal to 2.74 ng/mL, greater than or equal to 3.20 ng/mL, greater than or equal to 5.13 ng/mL or greater than or equal to 10.27 ng/mL.

In some embodiments, a composition is characterized in that, when administered by transmucosal absorption, the composition affords a Cₘₐₓ of cyclobenzaprine greater than or equal to 10 ng/mL, greater than or equal to 15 ng/mL, greater than or equal to 20 ng/mL, greater than or equal to 25 ng/mL, or greater than or equal to 30 ng/mL above the baseline level of cyclobenzaprine in the individual immediately prior to administration. In some embodiments relating to repeated, repetitive, daily and chronic dosing, a composition is characterized in that, when administered by transmucosal absorption, the composition affords a Cₘₐₓ of cyclobenzaprine greater than or equal to 10 ng/mL, greater than or equal to 15 ng/mL, greater than or equal to 20 ng/mL, greater than or equal to 25 ng/mL, or greater than or equal to 30 ng/mL above the baseline level of cyclobenzaprine in the individual immediately prior to administration. In some embodiments, a composition is characterized in that, when administered by transmucosal absorption, the composition affords a Cₘₐₓ of cyclobenzaprine greater than or equal to 2.74 ng/ml, greater than or equal to 3.20 ng/ml, greater than or equal to 5.13 ng/ml, or greater than or equal to 10.27 ng/ml above the baseline level of cyclobenzaprine in the individual immediately prior to administration. In some embodiments relating to repeated, repetitive, daily and chronic dosing, a composition is characterized in that, when administered by transmucosal absorption, the composition affords a Cₘₐₓ of cyclobenzaprine greater than or equal to 10 ng/ml, greater than or equal to 15 ng/ml, greater than or equal to 20 ng/ml, greater than or equal to 25 ng/ml, or greater than or equal to 30 ng/ml above the baseline level of cyclobenzaprine in the individual immediately prior to administration.

In some embodiments, a composition is characterized in that, when administered by transmucosal absorption, the composition affords a tₘₐₓ of cyclobenzaprine of less than 4.70 hours.

In some embodiments, a composition is characterized in that, when administered by transmucosal absorption, the composition affords a plasma level of cyclobenzaprine that decreases by at least 50%, by at least 60%, by at least 70%, by at least 80% the Cₘₐₓ by 8 hours after administration.

In some embodiments, the invention provides a method for treating a disease or condition in an individual in need thereof comprising administering a composition as described herein by transmucosal absorption. An exemplary disease or condition is post-traumatic stress disorder (PTSD). In further embodiments, administration of the composition treats the development of PTSD following a traumatic event, the initiation of PTSD following a traumatic event, the consolidation of PTSD following a traumatic event, or the perpetuation of PTSD following a traumatic event. In certain embodiments, the disease or condition is selected from the group consisting of fibromyalgia, depression, traumatic brain injury, sleep disorder, non-restorative sleep, chronic pain, muscle spasm, acute pain, and anxiety disorder.

In some embodiments, the basifying agent useful in methods of the invention is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, tripotassium citrate, dipotassium citrate, trisodium citrate and disodium citrate.

In some embodiments, the oral absorption in a method of the invention is sublingual absorption. In certain embodiments, the composition is in a form selected from the group consisting of a sublingual tablet, a sublingual film, a sublingual powder, and a sublingual spray solution.

In some embodiments, the oral absorption in a method of the invention is buccal absorption. In certain embodiments, the composition is selected from the group consisting of a buccal tablet, a lozenge, a buccal powder, and a buccal spray solution.

In some embodiments, the transmucosal absorption useful in a method of the invention is intranasal absorption. In certain embodiments, the composition is in a form of a nasal spray solution.

In some embodiments, the transmucosal absorption useful in a method of the invention is pulmonary absorption. In certain embodiments, the composition is in a form selected from the group consisting of an aerosolized composition and an inhalable dry powder.

In some embodiments, the invention provides a method wherein the cyclobenzaprine or amitriptyline has a dnC* of greater than or equal to 8.0±25%×10⁻⁷ mL⁻¹ 15 minutes after administration, greater than or equal to 1.0±25%×10⁻⁶ mL⁻¹ 30 minutes after administration, greater than or equal to 1.0±25%×10⁻⁶ mL⁻¹ 45 minutes after administration, greater than or equal to 1.0±25%×10⁻⁶ mL⁻¹ 1 hour after administration, greater than or equal to 1.0±25%×10⁻⁶ mL⁻¹ 2 hours after administration, or greater than or equal to 1.0×mL⁻¹ 3 hours after administration. In some embodiments, the invention provides a method wherein the cyclobenzaprine or amitriptyline has a dnC* of less than or equal to 5.0±25%×10⁻⁷ mL⁻¹ 8 hours after administration. In some embodiments, the invention provides a method wherein the cyclobenzaprine or amitriptyline has a dnC* of less than or equal to 5.0±25%×10⁻⁷ mL⁻¹ 10 hours after administration. In some embodiments, the invention provides a method wherein the cyclobenzaprine or amitriptyline has a dnC* of less than or equal to 5.0±25%×10⁻⁷ mL⁻¹ 12 hours after administration. In some embodiments, the invention provides a method wherein the cyclobenzaprine or amitriptyline has a dnC* of less than or equal to 5.0±25%×10⁻⁷ mL⁻¹ 14 hours after administration. In some embodiments, the invention provides a method wherein the cyclobenzaprine or amitriptyline has a dnC* of less than or equal to 5.0±25%×10⁻⁷ mL⁻¹ 16 hours after administration. In some embodiments, the invention provides a method wherein the cyclobenzaprine or amitriptyline has a dnC* of less than or equal to 5.0±25%×10⁻⁷ mL⁻¹ 18 hours after administration. In some embodiments, the invention provides a method wherein the cyclobenzaprine or amitriptyline has a dnC* of less than or equal to 5.0±25%×10⁻⁷ mL⁻¹ 20 hours after administration. In some embodiments, the invention provides a method wherein the cyclobenzaprine or amitriptyline has a dnC* of less than or equal to 5.0±25%×10⁻⁷ mL⁻¹ 22 hours after administration. In some embodiments, the invention provides a method wherein the cyclobenzaprine or amitriptyline has a dnC* of less than or equal to 5.0±25%×10⁻⁷ mL⁻¹ 24 hours after administration. In some embodiments, the invention provides a method wherein the cyclobenzaprine or amitriptyline has a dnC* of greater than or equal to 50±25%×10⁻⁹ mL⁻¹ 10 minutes after administration. In some embodiments, the invention provides a method wherein the cyclobenzaprine or amitriptyline has a dnC* of greater than or equal to 150±25%×10⁻⁹ mL⁻¹ 20 minutes after administration. In some embodiments, the invention provides a method wherein the cyclobenzaprine or amitriptyline has a dnC* of greater than or equal to 300±25%×10⁻⁹ mL⁻¹ 30 minutes after administration. In some embodiments, the invention provides a method wherein the cyclobenzaprine or amitriptyline has a dnC* of greater than or equal to 450±25%×10⁻⁹ mL⁻¹ 45 minutes after administration. In some embodiments, the invention provides a method wherein the cyclobenzaprine or amitriptyline has a dnC* of greater than or equal to 600±25%×10⁻⁹ mL⁻¹ 1 hour after administration. In some embodiments, the invention provides a method wherein the cyclobenzaprine or amitriptyline has a dnC* of greater than or equal to 700±25%×10⁻⁹ mL^{-1 2} hours after administration. In some embodiments, the invention provides a method wherein the cyclobenzaprine or amitriptyline has a dnC* of greater than or equal to 750±25%×10⁻⁹ mL⁻¹ 2.5 hours after administration. In some embodiments, the invention provides a method wherein the cyclobenzaprine or amitriptyline has a dnC* of greater than or equal to 850±25%×10⁻⁹ mL⁻¹ 3 hours after administration. In some embodiments, the invention provides a method wherein the cyclobenzaprine or amitriptyline has a dnC* of greater than or equal to 900±25%×10⁻⁹ mL⁻¹ 3.3 hours after administration. In some embodiments, the invention provides a method wherein the cyclobenzaprine or amitriptyline has a dnC* of greater than or equal to 950±25%×10⁻⁹ mL⁻¹ 3.7 hours after administration. In some embodiments, the invention provides a method wherein the cyclobenzaprine or amitriptyline has a dnC* of greater than or equal to 1000±25%×10⁻⁹ mL⁻¹ 4 hours after administration. In some embodiments, the invention provides a method wherein the cyclobenzaprine or amitriptyline has a dnC* of greater than or equal to 1050±25%×10⁻⁹ mL⁻¹ 4.33 hours after administration. In some embodiments, the invention provides a method wherein the cyclobenzaprine or amitriptyline has a dnC* of less than or equal to 1050±25%×10⁻⁹ mL⁻¹ 4.67 hours after administration. In some embodiments, the invention provides a method wherein the cyclobenzaprine or amitriptyline has a dnC* of less than or equal to 1000±25%×10⁻⁹ mL⁻¹ 5 hours after administration. In some embodiments, the invention provides a method wherein the cyclobenzaprine or amitriptyline has a dnC* of less than or equal to 1000±25%×10⁻⁹ mL⁻¹ 5.5 hours after administration. In some embodiments, the invention provides a method wherein the cyclobenzaprine or amitriptyline has a dnC* of less than or equal to 900±25%×10⁻⁹ mL^{-1 6} hours after administration. In some embodiments, the invention provides a method wherein the cyclobenzaprine or amitriptyline has a dnC* of less than or equal to 700±25%×10⁻⁹ mL⁻¹ 8 hours after administration. In some embodiments, the invention provides a method wherein the cyclobenzaprine or amitriptyline has a dnC* of less than or equal to 500±25%×10⁻⁹ mL⁻¹ 12 hours after administration. In some embodiments, the invention provides a method wherein the cyclobenzaprine or amitriptyline has a dnC* of less than or equal to 350±25%×10⁻⁹ mL⁻¹ 16 hours after administration. In some embodiments, the invention provides a method wherein the cyclobenzaprine or amitriptyline has a dnC* of less than or equal to 300±25%×10⁻⁹ mL⁻¹ 24 hours after administration. In some embodiments, the invention provides a method wherein the cyclobenzaprine or amitriptyline has a dnC* of less than or equal to 180±25%×10⁻⁹ mL⁻¹ 36 hours after administration. In some embodiments, the invention provides a method wherein the cyclobenzaprine or amitriptyline has a dnC* of less than or equal to 140±25%×10⁻⁹ mL⁻¹ 48 hours after administration. In some embodiments, the invention provides a method wherein the cyclobenzaprine or amitriptyline has a dnC* of less than or equal to 90±25%×10⁻⁹ mL⁻¹ 72 hours after administration.

In some embodiments, the invention provides a method wherein the cyclobenzaprine or amitriptyline has a dnAUC_{0-8 h} of greater than or equal to 5 mL⁻¹ hr. In some embodiments, the cyclobenzaprine or amitriptyline has a dnAUC_{0-∞h} of greater than or equal to 20 mL⁻¹ hr. In some embodiments, the invention provides a method wherein the cyclobenzaprine or amitriptyline has a dnCₘₐₓ* of greater than or equal to 1.0±25%×10⁻⁶ mL⁻¹. In some embodiments, the invention provides a method wherein the cyclobenzaprine or amitriptyline has a dnAUC_{0-8 h} of greater than or equal to 6.3±25%×10⁻⁶ hr mL⁻¹. In some embodiments, the cyclobenzaprine or amitriptyline has a dnAUC_{0-∞h} of greater than or equal to 25±25%×10⁻⁶ hr mL⁻¹. In some embodiments, the invention provides a method wherein the cyclobenzaprine or amitriptyline has a dnCₘₐₓ* of greater than or equal to 1.1±25%×10⁻⁶ mL⁻¹.

In some embodiments, the invention provides a method wherein the cyclobenzaprine or amitriptyline has a plasma concentration of 50% or less of the Cₘₐₓ 4 hours after administration, 50% or less of the Cₘₐₓ 6 hours after administration, 50% or less of the Cₘₐₓ 8 hours after administration, or 50% or less of the Cₘₐₓ 12 hours after administration.

In some embodiments, the invention provides a method wherein the cyclobenzaprine is present in the composition in an amount from 0.1 mg to 10 mg. In certain embodiments, the cyclobenzaprine is present in the composition in an amount from 0.1 mg to 5 mg, for example, in an amount of about 2.4 mg, in an amount of less than about 2.4 mg, in an amount of about 4.8 mg, or in an amount of less than about 4.8 mg or in an amount of about 2.8 mg, in an amount of less than about 2.8 mg, in an amount of about 5.6 mg, or in an amount of less than about 5.6 mg, in an amount of about 9.0 mg, in an amount of less than about 10 mg

In some embodiments, the invention provides a method wherein a composition affords a Cₘₐₓ of cyclobenzaprine greater than or equal to 10 ng/mL, greater than or equal to 15 ng/mL, greater than or equal to 20 ng/mL, greater than or equal to 25 ng/mL, greater than or equal to 30 ng/mL, greater than or equal to 40 ng/mL, greater than or equal to 50 ng/mL, greater than or equal to 60 ng/mL, greater than or equal to 70 ng/mL, greater than or equal to 80 ng/mL, greater than or equal to 90 ng/mL, greater than or equal to 100 ng/mL, greater than or equal to 120 ng/mL, greater than or equal to 140 ng/mL, greater than or equal to 150 ng/mL, greater than or equal to 160 ng/mL, greater than or equal to 170 ng/mL, greater than or equal to 180 ng/mL, greater than or equal to 190 ng/mL, greater than or equal to 200 ng/mL, greater than or equal to 220 ng/mL, greater than or equal to 240 ng/mL, greater than or equal to 260 ng/mL, or greater than or equal to 280 ng/mL. In some embodiments, the invention provides a method wherein a composition affords a Cₘₐₓ of cyclobenzaprine greater than or equal to 2.74 ng/ml, greater than or equal to 3.20 ng/ml, greater than or equal to 5.13 ng/ml, greater than or equal to 10.27 ng/ml, greater than or equal to 2 ng/ml, greater than or equal to 3 ng/ml.

In some embodiments, the invention provides a method wherein a composition affords a Cₘₐₓ of cyclobenzaprine greater than or equal to 10 ng/mL above the baseline level, greater than or equal to 15 ng/mL above the baseline level, greater than or equal to 20 ng/mL above the baseline level, greater than or equal to 25 ng/mL above the baseline level, greater than or equal to 30 ng/mL above the baseline level, greater than or equal to 40 ng/mL above the baseline level, greater than or equal to 50 ng/mL above the baseline level, greater than or equal to 60 ng/mL above the baseline level, greater than or equal to 70 ng/mL above the baseline level, greater than or equal to 80 ng/mL above the baseline level, greater than or equal to 90 ng/mL above the baseline level, greater than or equal to 100 ng/mL above the baseline level, greater than or equal to 120 ng/mL above the baseline level, greater than or equal to 140 ng/mL above the baseline level, greater than or equal to 150 ng/mL above the baseline level, greater than or equal to 160 ng/mL above the baseline level, greater than or equal to 170 ng/mL above the baseline level, greater than or equal to 180 ng/mL above the baseline level, greater than or equal to 190 ng/mL above the baseline level, greater than or equal to 200 ng/mL above the baseline level, greater than or equal to 220 ng/mL above the baseline level, greater than or equal to 240 ng/mL above the baseline level, greater than or equal to 260 ng/mL above the baseline level, or greater than or equal to 280 ng/mL above the baseline level of cyclobenzaprine in the individual immediately prior to administration. In some embodiments relating to repeated, repetitive, daily and chronic dosing, the invention provides a method wherein a composition affords a Cₘₐₓ of cyclobenzaprine greater than or equal to 10 ng/mL above the baseline level, greater than or equal to 15 ng/mL above the baseline level, greater than or equal to 20 ng/mL above the baseline level, greater than or equal to 25 ng/mL above the baseline level, greater than or equal to 30 ng/mL above the baseline level, greater than or equal to 40 ng/mL above the baseline level, greater than or equal to 50 ng/mL above the baseline level, greater than or equal to 60 ng/mL above the baseline level, greater than or equal to 70 ng/mL above the baseline level, greater than or equal to 80 ng/mL above the baseline level, greater than or equal to 90 ng/mL above the baseline level, greater than or equal to 100 ng/mL above the baseline level, greater than or equal to 120 ng/mL above the baseline level, greater than or equal to 140 ng/mL above the baseline level, greater than or equal to 150 ng/mL above the baseline level, greater than or equal to 160 ng/mL above the baseline level, greater than or equal to 170 ng/mL above the baseline level, greater than or equal to 180 ng/mL above the baseline level, greater than or equal to 190 ng/mL above the baseline level, greater than or equal to 200 ng/mL above the baseline level, greater than or equal to 220 ng/mL above the baseline level, greater than or equal to 240 ng/mL above the baseline level, greater than or equal to 260 ng/mL above the baseline level, or greater than or equal to 280 ng/mL above the baseline level, of cyclobenzaprine in the individual immediately prior to administration. In some embodiments, the invention provides a method wherein a composition affords a Cₘₐₓ of cyclobenzaprine greater than or equal to 2.74 ng/ml above the baseline level, greater than or equal to 3.20 ng/ml above the baseline level, greater than or equal to 5.13 ng/ml above the baseline level, greater than or equal to 10.27 ng/ml above the baseline level, greater than or equal to 2 ng/ml above the baseline level, greater than or equal to 3 ng/ml above the baseline level, 10 ng/ml, greater than or equal to 15 ng/ml above the baseline level, greater than or equal to 20 ng/ml above the baseline level, greater than or equal to 25 ng/ml above the baseline level, greater than or equal to 30 ng/ml above the baseline level, greater than or equal to 40 ng/ml above the baseline level.

In some embodiments, the invention provides a method wherein a composition affords a tₘₐₓ of cyclobenzaprine of less than 4 hours, less than 3 hours, less than 2 hours, less than 1 hour, less than 45 minutes, less than 30 minutes, less than 15 minutes, less than 10 minutes, or less than 5 minutes.

In some embodiments, the invention provides a method wherein the composition affords a plasma level of cyclobenzaprine that decreases by at least 50% of the Cₘₐₓ by 8 hours after administration, by at least 60% of the Cₘₐₓ by 8 hours after administration, by at least 70% of the Cₘₐₓ by 8 hours after administration, by at least 80% of the Cₘₐₓ by 8 hours after administration, by at least 90% of the Cₘₐₓ by 8 hours after administration, or by at least 95% of the Cₘₐₓ by 8 hours after administration.

In some embodiments, the invention provides a composition comprising cyclobenzaprine for transmucosal administration comprising from about 2 to about 20 mg of cyclobenzaprine or a salt thereof, the formulation affording a Cₘₐₓ of cyclobenzaprine from about 20 to about 200 ng/mL from about 0.05 to about 2.5 hours after administration, and a minimum cyclobenzaprine plasma concentration from about 1 to about 5 ng/mL from about 22 to about 26 hours after administration, wherein the composition is administered for four days or more of daily administration. In some embodiments, the invention provides a composition comprising cyclobenzaprine for transmucosal administration comprising from about 2 to about 20 mg of cyclobenzaprine or a salt thereof, the formulation affording a Cₘₐₓ of cyclobenzaprine from about 1.0 ng/ml to about 30.0 ng/ml from about 2 to about 5.0 hours after administration, and a minimum plasma concentration from about 1 to about 5 ng/ml from about 22 to about 26 hours after administration, wherein the composition is administered for four days or more of daily administration, and wherein the composition is administered within two hours of sleep·ng/ml.

In some embodiments, the invention provides a composition comprising cyclobenzaprine for transmucosal administration comprising from about 2 to about 20 mg of cyclobenzaprine or a salt thereof, the formulation affording a dnCₘᵢₙ₍₂₄₎* of cyclobenzaprine from about 100±25%×10⁻⁹ mL⁻¹ to about 1000±25%×10-9 mL⁻¹ about 22 to about 26 hours after administration, wherein the composition is administered for four days or more of daily administration, and wherein the composition is administered within two hours of sleep. In some embodiments, the invention provides a composition comprising cyclobenzaprine for transmucosal administration comprising from about 2 to about 20 mg of cyclobenzaprine or a salt thereof, the formulation affording a dnCₘᵢₙ₍₂₄₎* of cyclobenzaprine less then or equal to 300±25%×10⁻⁹ mL⁻¹ (calculated using the mean Cₘᵢₙ₍₂₄₎ of 706.55 pg/mL at 24 hours and the dnCₘᵢₙ₍₂₄₎* was ((706.55 pg/mL)/(2.4 mg))=294.40 pg/(mg mL), or 300±25%×10⁻⁹ mL⁻¹) at 24 hours or between 22 to about 26 hours after administration, wherein the composition is administered for four days or more of daily administration, and wherein the composition is administered within two hours of sleep.

In some embodiments, the invention provides a method for reducing the symptoms of fibromyalgia in a human patient, comprising administering a transmucosal dosage formulation comprising from about 2 to about 20 mg of cyclobenzaprine or a salt thereof, said formulation affording a Cₘₐₓ of cyclobenzaprine from about 20 to about 200 ng/mL from about 0.05 to about 2.5 hours after administration, and a minimum plasma concentration from about 1 to about 5 ng/mL from about 22 to about 26 hours after administration, wherein the composition is administered for four days or more of daily administration, and wherein the composition is administered within two hours of sleep.

In some embodiments, the invention provides a method for reducing the symptoms of fibromyalgia in a human patient, comprising administering a transmucosal dosage formulation comprising of about 2.4 mg of cyclobenzaprine or a salt thereof, said formulation affording in a single dose study, a Cₘₐₓ of cyclobenzaprine of about 2.74 pg mL⁻¹ at about 4.70 hours after administration, and a minimum plasma concentration of about 706.55 ng mL⁻¹ at about 24 hours after administration, wherein the composition is administered for four days or more of daily administration, and wherein the composition is administered within two hours of sleep.

In some embodiments, the invention provides a method for reducing the symptoms of PTSD in a human patient, comprising administering a transmucosal dosage formulation comprising from about 2 to about 20 mg of cyclobenzaprine or a salt thereof, said formulation affording a Cₘₐₓ of cyclobenzaprine from about 1.0 ng/ml to about 30.0 ng/ml from about 2 to about 5.0 hours after administration, and a minimum plasma concentration from about 1 to about 5 ng/ml from about 22 to about 26 hours after administration, wherein the composition is administered for four days or more of daily administration, and wherein the composition is administered within two hours of sleep.

In some embodiments, the invention provides a method for reducing the symptoms of muscle spasm and acute painful musculoskeletal conditions, including local pain and restriction of movement, in a human patient, comprising administering a transmucosal dosage formulation comprising from about 2 to about 20 mg of cyclobenzaprine or a salt thereof, said formulation affording a Cₘₐₓ of cyclobenzaprine from about 1.0 ng/ml to about 30.0 ng/ml from about 2 to about 5.0 hours after administration.

In some embodiments, the invention provides a composition comprising amitriptyline for transmucosal administration comprising from about 2 to about 25 mg of amitriptyline or a salt thereof, said formulation affording a Cₘₐₓ of amitriptyline from about 20 to about 200 ng/mL from about 0.05 to about 2.5 hours after administration, and a minimum amitriptyline plasma concentration from about 1 to about 5 ng/mL from about 22 to about 26 hours after administration, wherein the composition is administered for four days or more of daily administration. In some embodiments, the invention provides a composition comprising amitriptyline for transmucosal administration comprising from about 2 to about 25 mg of amitriptyline or a salt thereof, said formulation affording a Cₘₐₓ of amitriptyline from about 20 to about 200 ng/mL from about 0.05 to about 5 hours after administration, and a minimum amitriptyline plasma concentration from about 1 to about 5 ng/mL from about 22 to about 26 hours after administration, wherein the composition is administered for four days or more of daily administration.

In some embodiments, the invention provides a method for reducing the symptoms of fibromyalgia in a human patient, comprising administering a transmucosal dosage formulation comprising from about 2 to about 25 mg of amitriptyline or a salt thereof, said formulation affording a Cₘₐₓ of amitriptyline from about 20 to about 200 ng/mL from about 0.05 to about 2.5 hours after administration, and a minimum plasma concentration from about 1 to about 5 ng/mL from about 22 to about 26 hours after administration, wherein the composition is administered for four days or more of daily administration, and wherein the composition is administered within two hours of sleep. In some embodiments, the invention provides a method for reducing the symptoms of fibromyalgia in a human patient, comprising administering a transmucosal dosage formulation comprising from about 7.5 to about 50 mg of amitriptyline or a salt thereof, said formulation affording a Cₘₐₓ of amitriptyline from about 3 to about 90 ng/ml from about 2 to about 5 hours after administration, and a minimum amitriptyline plasma concentration from about 3 to about 15 ng/ml from about 22 to about 26 hours after administration, wherein the composition is administered for four days or more of daily administration.

Some embodiments of the invention are:

1. A composition comprising cyclobenzaprine, wherein the composition is suitable for transmucosal absorption.

2. A composition comprising cyclobenzaprine and a basifying agent, wherein the composition is suitable for transmucosal absorption.

3. A composition comprising amitriptyline, wherein the composition is suitable for transmucosal absorption.

4. A composition comprising amitriptyline and a basifying agent, wherein the composition is suitable for transmucosal absorption.

5. The composition of any one of embodiments 2 and 4, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

6. The composition of any one of embodiments 1-5, wherein the transmucosal absorption is oral absorption.

7. The composition of embodiment 6, wherein the composition is suitable for sublingual administration.

8. The composition of embodiment 7, wherein the composition is in a form selected from the group consisting of a sublingual tablet, a sublingual film, a sublingual powder, and a sublingual spray solution.

9. The composition of embodiment 6, wherein the composition is suitable for buccal administration.

10. The composition of embodiment 9, wherein the composition is in a form selected from the group consisting of a buccal tablet, a lozenge, a buccal powder, and a buccal spray solution.

11. The composition of any one of embodiments 1-5, wherein the transmucosal absorption is intranasal absorption.

12. The composition of embodiment 11, wherein the composition is in a form of a nasal spray solution.

13. The composition of any one of embodiments 1-5, wherein the transmucosal absorption is pulmonary absorption.

14. The composition of embodiment 13, wherein the composition is in a form selected from the group consisting of an aerosolized composition and an inhalable dry powder.

15. The composition of any one of embodiments 1-14, characterized in that, when administered by transmucosal absorption, the cyclobenzaprine or amitriptyline has a dnC* of greater than or equal to 0.1±25%×10⁻⁷ mL⁻¹ 15 minutes after administration.

16. The composition of any one of embodiments 1-15, characterized in that, when administered by transmucosal absorption, the cyclobenzaprine or amitriptyline has a dnC* of greater than or equal to 0.5±25%×10⁻⁷ mL⁻¹ 30 minutes after administration.

17. The composition of any one of embodiments 1-16, characterized in that, when administered by transmucosal absorption, the cyclobenzaprine or amitriptyline has a dnC* of greater than or equal to 1.5±25%×10⁻⁷ mL⁻¹ 45 minutes after administration.

18. The composition of any one of embodiments 1-17, characterized in that, when administered by transmucosal absorption, the cyclobenzaprine or amitriptyline has a dnC* of greater than or equal to 4.0±25%×10⁻⁷ mL⁻¹ 1 hour after administration.

19. The composition of any one of embodiments 1-18, characterized in that, when administered by transmucosal absorption, the cyclobenzaprine or amitriptyline has a dnC* of greater than or equal to 5.2±25%×10⁻⁷ mL⁻¹ 2 hours after administration.

20. The composition of any one of embodiments 1-19, characterized in that, when administered by transmucosal absorption, the cyclobenzaprine or amitriptyline has a dnC* of greater than or equal to 9.0±25%×10⁻⁶ mL⁻¹ 3 hours after administration.

21. The composition of any one of embodiments 1-20, characterized in that, when administered by transmucosal absorption, the cyclobenzaprine or amitriptyline has a dnC* of less than or equal to 5.0±25%×10⁻⁷ mL⁻¹ 10 hours after administration.

22. The composition of any one of embodiments 1-21, characterized in that, when administered by transmucosal absorption, the cyclobenzaprine or amitriptyline has a dnC* of less than or equal to 5.0±25%×10⁻⁷ mL⁻¹ 12 hours after administration.

23. The composition of any one of embodiments 1-22, characterized in that, when administered by transmucosal absorption, the cyclobenzaprine or amitriptyline has a dnC* of less than or equal to 5.0±25%×10⁻⁷ mL⁻¹ 14 hours after administration.

24. The composition of any one of embodiments 1-23, characterized in that, when administered by transmucosal absorption, the cyclobenzaprine or amitriptyline has a dnC* of less than or equal to 5.0±25%×10⁻⁷ mL⁻¹ 16 hours after administration.

25. The composition of any one of embodiments 1-24, characterized in that, when administered by transmucosal absorption, the cyclobenzaprine or amitriptyline has a dnC* of less than or equal to 5.0±25%×10⁻⁷ mL⁻¹ 18 hours after administration.

26. The composition of any one of embodiments 1-25, characterized in that, when administered by transmucosal absorption, the cyclobenzaprine or amitriptyline has a dnAUC₀₋₂₀min of greater than or equal to 0.02±25%×10⁻⁶ hr mL⁻¹, a dnAUC₀₋₃₀min of greater than or equal to 0.05±25%×10⁻⁶ hr mL⁻¹, a dnAUC₀₋₄₅min of greater than or equal to 0.14±25%×10⁻⁶ hr mL⁻¹, a dnAUC_{0-1 h} of greater than or equal to 0.26±25%×10⁻⁶ hr mL⁻¹, a dnAUC_{0-2 h} of greater than or equal to 0.87±25%×10⁻⁶ hr mL⁻¹, or a dnAUC_{0-2.5 h} of greater than or equal to 1.23±25%×10⁻⁶ hr mL⁻¹.

27. The composition of any one of embodiments 1-26, characterized in that, when administered by transmucosal absorption, the cyclobenzaprine or amitriptyline has a dnAUC_{0-∞h} of greater than or equal to 20 mL⁻¹ hr⁻¹.

28. The composition of any one of embodiments 1-27, characterized in that, when administered by transmucosal absorption, the cyclobenzaprine or amitriptyline has a dnCₘₐₓ* of greater than or equal to 1.0±25%×10⁻⁶ mL⁻¹.

29. The composition of any one of embodiments 1-28, characterized in that, when administered by transmucosal absorption, the cyclobenzaprine or amitriptyline has a plasma concentration of 50% or less of the Cₘₐₓ 4 hours after administration.

30. The composition of any one of embodiments 1-29, characterized in that, when administered by transmucosal absorption, the cyclobenzaprine or amitriptyline has a plasma concentration of 50% or less of the Cₘₐₓ 6 hours after administration.

31. The composition of any one of embodiments 1-30, characterized in that, when administered by transmucosal absorption, the cyclobenzaprine or amitriptyline has a plasma concentration of 50% or less of the Cₘₐₓ 8 hours after administration.

32. The composition of any one of embodiments 1-31, characterized in that, when administered by transmucosal absorption, the cyclobenzaprine or amitriptyline has a plasma concentration of 50% or less of the Cₘₐₓ 12 hours after administration.

33. The composition of any one of embodiments 1-32, wherein the cyclobenzaprine is present in an amount from 0.1 mg to 10 mg.

34. The composition of embodiment 33, wherein the cyclobenzaprine is present in an amount from 0.1 mg to 5 mg.

35. The composition of embodiment 34, wherein the cyclobenzaprine is present in an amount of about 2.4 mg.

36. The composition of embodiment 34, wherein the cyclobenzaprine is present in an amount of less than about 2.4 mg.

37. The composition of embodiment 34, wherein the cyclobenzaprine is present in an amount of about 4.8 mg.

38. The composition of embodiment 34, wherein the cyclobenzaprine is present in an amount of less than about 4.8 mg.

39. The composition of any one of embodiments 1-38, characterized in that, when administered by transmucosal absorption, the composition affords a Cₘₐₓ of cyclobenzaprine greater than or equal to 10 ng/mL.

40. The composition of embodiment 39, characterized in that, when administered by transmucosal absorption, the composition affords a Cₘₐₓ of cyclobenzaprine greater than or equal to 15 ng/mL.

41. The composition of embodiment 40, characterized in that, when administered by transmucosal absorption, the composition affords a Cₘₐₓ of cyclobenzaprine greater than or equal to 20 ng/mL.

42. The composition of embodiment 41, characterized in that, when administered by transmucosal absorption, the composition affords a Cₘₐₓ of cyclobenzaprine greater than or equal to 25 ng/mL.

43. The composition of embodiment 42, characterized in that, when administered by transmucosal absorption, the composition affords a Cₘₐₓ of cyclobenzaprine greater than or equal to 30 ng/mL.

44. The composition of any one of embodiments 1-43, characterized in that, when administered by transmucosal absorption, the composition affords a Cₘₐₓ of cyclobenzaprine greater than or equal to 10 ng/mL above the baseline level of cyclobenzaprine in the individual immediately prior to administration.

45. The composition of embodiment 44, characterized in that, when administered by transmucosal absorption, the composition affords a Cₘₐₓ of cyclobenzaprine greater than or equal to 15 ng/mL above the baseline level of cyclobenzaprine in the individual immediately prior to administration.

46. The composition of embodiment 45, characterized in that, when administered by transmucosal absorption, the composition affords a Cₘₐₓ of cyclobenzaprine greater than or equal to 20 ng/mL above the baseline level of cyclobenzaprine in the individual immediately prior to administration.

47. The composition of embodiment 46, characterized in that, when administered by transmucosal absorption, the composition affords a Cₘₐₓ of cyclobenzaprine greater than or equal to 25 ng/mL above the baseline level of cyclobenzaprine in the individual immediately prior to administration.

48. The composition of embodiment 47, characterized in that, when administered by transmucosal absorption, the composition affords a Cₘₐₓ of cyclobenzaprine greater than or equal to 30 ng/mL above the baseline level of cyclobenzaprine in the individual immediately prior to administration.

49. The composition of any one of embodiments 1-48, characterized in that, when administered by transmucosal absorption, the composition affords a tₘₐₓ of cyclobenzaprine of less than 4 hours.

50. The composition of embodiment 49, characterized in that, when administered by transmucosal absorption, the composition affords a tₘₐₓ of cyclobenzaprine of less than 3 hours.

51. The composition of embodiment 50, characterized in that, when administered by transmucosal absorption, the composition affords a tₘₐₓ of cyclobenzaprine of less than 2 hours.

52. The composition of embodiment 51, characterized in that, when administered by transmucosal absorption, the composition affords a tₘₐₓ of cyclobenzaprine of less than 1 hour.

53. The composition of embodiment 52, characterized in that, when administered by transmucosal absorption, the composition affords a tₘₐₓ of cyclobenzaprine of less than 45 minutes.

54. The composition of embodiment 53, characterized in that, when administered by transmucosal absorption, the composition affords a tₘₐₓ of cyclobenzaprine of less than 30 minutes.

55. The composition of embodiment 54, characterized in that, when administered by transmucosal absorption, the composition affords a tₘₐₓ of cyclobenzaprine of less than 15 minutes.

56. The composition of any one of embodiments 1-55, characterized in that, when administered by transmucosal absorption, the composition affords a plasma level of cyclobenzaprine that decreases by at least 50% of the Cₘₐₓ by 8 hours after administration.

57. The composition of embodiment 56, characterized in that, when administered by transmucosal absorption, the composition affords a plasma level of cyclobenzaprine that decreases by at least 60% of the Cₘₐₓ by 8 hours after administration.

58. The composition of embodiment 57, characterized in that, when administered by transmucosal absorption, the composition affords a plasma level of cyclobenzaprine that decreases by at least 70% of the Cₘₐₓ by 8 hours after administration.

59. The composition of embodiment 58, characterized in that, when administered by transmucosal absorption, the composition affords a plasma level of cyclobenzaprine that decreases by at least 80% of the Cₘₐₓ by 8 hours after administration.

60. The composition of embodiment 59, characterized in that, when administered by transmucosal absorption, the composition affords a plasma level of cyclobenzaprine that decreases by at least 90% of the Cₘₐₓ by 8 hours after administration.

61. The composition of embodiment 60, characterized in that, when administered by transmucosal absorption, the composition affords a plasma level of cyclobenzaprine that decreases by at least 95% of the Cₘₐₓ by 8 hours after administration.

62. A method for treating a disease or condition in an individual in need thereof comprising administering a composition of any one of embodiments 1-61 by transmucosal absorption.

63. The method of embodiment 62, wherein the disease or condition is post-traumatic stress disorder (PTSD).

64. The method of embodiment 63, wherein administration of the composition treats the development of PTSD following a traumatic event.

65. The method of embodiment 63, wherein administration of the composition treats the initiation of PTSD following a traumatic event.

66. The method of embodiment 63, wherein administration of the composition treats the consolidation of PTSD following a traumatic event.

67. The method of embodiment 63, wherein administration of the composition treats the perpetuation of PTSD following a traumatic event.

68. The method of embodiment 62, wherein the disease or condition is selected from the group consisting of fibromyalgia, depression, traumatic brain injury, sleep disorder, non-restorative sleep, chronic pain, muscle spasm, and anxiety disorder.

69. The method of any one of embodiments 62-68, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

70. The method of any one of embodiments 62-69, wherein the oral absorption is sublingual absorption.

71. The method of embodiment 70, wherein the composition is in a form selected from the group consisting of a sublingual tablet, a sublingual film, a sublingual powder, and a sublingual spray solution.

72. The method of any one of embodiments 62-69, wherein the oral absorption is buccal absorption.

73. The method of embodiment 72, wherein the composition is selected from the group consisting of a buccal tablet, a lozenge, a buccal powder, and a buccal spray solution.

74. The method of any one of embodiments 62-73, wherein the transmucosal absorption is intranasal absorption.

75. The method of embodiment 74, wherein the composition is in a form of a nasal spray solution.

76. The method of any one of embodiments 62-73, wherein the transmucosal absorption is pulmonary absorption.

77. The method of embodiment 76, wherein the composition is in a form selected from the group consisting of an aerosolized composition and an inhalable dry powder.

78. The method of any one of embodiments 62-77, wherein the cyclobenzaprine or amitriptyline has a dnC* of greater than or equal to 1.0±25%×10⁻⁷ mL⁻¹ 20 minutes after administration.

79. The method of any one of embodiments 62-78, wherein the cyclobenzaprine or amitriptyline has a dnC* of greater than or equal to 2.5±25%×10⁻⁷ mL⁻¹ 30 minutes after administration.

80. The method of any one of embodiments 62-79, wherein the cyclobenzaprine or amitriptyline has a dnC* of greater than or equal to 3.0±25%×10⁻⁷ mL⁻¹ 45 minutes after administration.

81. The method of any one of embodiments 62-80, wherein the cyclobenzaprine or amitriptyline has a dnC* of greater than or equal to 4.2±25%×10⁻⁷ mL⁻¹ 1 hour after administration.

82. The method of any one of embodiments 62-81, wherein the cyclobenzaprine or amitriptyline has a dnC* of greater than or equal to 6.0±25%×10⁻⁷ mL⁻¹ 2 hours after administration.

83. The method of any one of embodiments 62-82, wherein the cyclobenzaprine or amitriptyline has a dnC* of greater than or equal to 7.0±25%×10⁻⁷ mL⁻¹ 3 hours after administration.

84. The method of any one of embodiments 62-83, wherein the cyclobenzaprine or amitriptyline has a dnC* of greater than or equal to 8.0±25%×10⁻⁷ mL⁻¹ 3.3 hours after administration.

85. The method of any one of embodiments 62-84, wherein the cyclobenzaprine or amitriptyline has a dnC* of less than or equal to 5.0±25%×10⁻⁷ mL⁻¹ 12 hours after administration.

86. The method of any one of embodiments 62-85, wherein the cyclobenzaprine or amitriptyline has a dnC* of less than or equal to 5.0±25%×10⁻⁷ mL⁻¹ 14 hours after administration.

87. The method of any one of embodiments 62-86, wherein the cyclobenzaprine or amitriptyline has a dnC* of less than or equal to 5.0±25%×10⁻⁷ mL⁻¹ 16 hours after administration.

88. The method of any one of embodiments 62-87, wherein the cyclobenzaprine or amitriptyline has a dnC* of less than or equal to 5.0±25%×10⁻⁷ mL⁻¹ 18 hours after administration.

89. The method of any one of embodiments 62-88, wherein the cyclobenzaprine or amitriptyline has a dnAUC₀₋₂₀min of greater than or equal to 0.02±25%×10⁻⁶ hr mL⁻¹, a dnAUC₀₋₃₀min of greater than or equal to 0.05±25%×10⁻⁶ hr mL⁻¹, a dnAUC₀₋₄₅min of greater than or equal to 0.14±25%×10⁻⁶ hr mL⁻¹, a dnAUC_{0-1 h} of greater than or equal to 0.26±25%×10⁻⁶ hr mL⁻¹, a dnAUC_{0-2 h} of greater than or equal to 0.87±25%×10⁻⁶ hr mL⁻¹, or a dnAUC_{0-2.5 h} of greater than or equal to 1.23±25%×10⁻⁶ hr mL⁻¹¹.

90. The method of any one of embodiments 62-89, wherein the cyclobenzaprine or amitriptyline has a dnAUC_{0-∞n} of greater than or equal to 20 mL⁻¹ hr⁻¹.

91. The method of any one of embodiments 62-90, wherein the cyclobenzaprine or amitriptyline has a dnCₘₐₓ* of greater than or equal to 1.0±25%×10⁻⁶ mL⁻¹.

92. The method of any one of embodiments 62-91, wherein the cyclobenzaprine or amitriptyline has a plasma concentration of 50% or less of the Cₘₐₓ 4 hours after administration.

93. The method of any one of embodiments 62-92, wherein the cyclobenzaprine or amitriptyline has a plasma concentration of 50% or less of the Cₘₐₓ 6 hours after administration.

94. The method of any one of embodiments 62-93, wherein the cyclobenzaprine or amitriptyline has a plasma concentration of 50% or less of the Cₘₐₓ 8 hours after administration.

95. The method of any one of embodiments 62-94, wherein the cyclobenzaprine or amitriptyline has a plasma concentration of 50% or less of the Cₘₐₓ 12 hours after administration.

96. The method of any one of embodiments 62-95, wherein the cyclobenzaprine is present in the composition in an amount from 0.1 mg to 10 mg.

97. The method of embodiment 96, wherein the cyclobenzaprine is present in the composition in an amount from 0.1 mg to 5 mg.

98. The method of embodiment 97, wherein the cyclobenzaprine is present in the composition in an amount of about 2.4 mg.

99. The method of embodiment 98, wherein the cyclobenzaprine is present in an amount of less than about 2.4 mg.

100. The method of embodiment 99, wherein the cyclobenzaprine is present in an amount of about 4.8 mg.

101. The method of embodiment 100, wherein the cyclobenzaprine is present in an amount of less than about 4.8 mg.

102. The method of any one of embodiments 62-101, wherein the composition affords a Cₘₐₓ of cyclobenzaprine greater than or equal to 10 ng/mL.

103. The method of embodiment 102, wherein the composition affords a Cₘₐₓ of cyclobenzaprine greater than or equal to 15 ng/mL.

104. The method of embodiment 103, wherein the composition affords a Cₘₐₓ of cyclobenzaprine greater than or equal to 20 ng/mL.

105. The method of embodiment 104, wherein the composition affords a Cₘₐₓ of cyclobenzaprine greater than or equal to 25 ng/mL.

106. The method of embodiment 105, wherein the composition affords a Cₘₐₓ of cyclobenzaprine greater than or equal to 30 ng/mL.

107. The method of any one of embodiments 62-106, wherein the composition affords a Cₘₐₓ of cyclobenzaprine greater than or equal to 10 ng/mL above the baseline level of cyclobenzaprine in the individual immediately prior to administration.

108. The method of embodiment 107, wherein the composition affords a Cₘₐₓ of cyclobenzaprine greater than or equal to 15 ng/mL above the baseline level of cyclobenzaprine in the individual immediately prior to administration.

109. The method of embodiment 108, wherein the composition affords a Cₘₐₓ of cyclobenzaprine greater than or equal to 20 ng/mL above the baseline level of cyclobenzaprine in the individual immediately prior to administration.

110. The method of embodiment 109, wherein the composition affords a Cₘₐₓ of cyclobenzaprine greater than or equal to 25 ng/mL above the baseline level of cyclobenzaprine in the individual immediately prior to administration.

111. The method of embodiment 110, wherein the composition affords a Cₘₐₓ of cyclobenzaprine greater than or equal to 30 ng/mL above the baseline level of cyclobenzaprine in the individual immediately prior to administration.

112. The method of any one of embodiments 62-111, wherein the composition affords a tₘₐₓ of cyclobenzaprine of less than 4 hours.

113. The method of embodiment 112, wherein the composition affords a tₘₐₓ of cyclobenzaprine of less than 3 hours.

114. The method of embodiment 113, wherein the composition affords a tₘₐₓ of cyclobenzaprine of less than 2 hours.

115. The method of embodiment 114, wherein the composition affords a tₘₐₓ of cyclobenzaprine of less than 1 hour.

116. The method of embodiment 115, wherein the composition affords a tₘₐₓ of cyclobenzaprine of less than 45 minutes.

117. The method of embodiment 116, wherein the composition affords a tₘₐₓ of cyclobenzaprine of less than 30 minutes.

118. The method of embodiment 117, wherein the composition affords a tₘₐₓ of cyclobenzaprine of less than 15 minutes.

119. The method of any one of embodiments 62-118, wherein the composition affords a plasma level of cyclobenzaprine that decreases by at least 50% of the Cₘₐₓ by 8 hours after administration.

120. The method of embodiment 119, wherein the composition affords a plasma level of cyclobenzaprine that decreases by at least 60% of the Cₘₐₓ by 8 hours after administration.

121. The method of embodiment 120, wherein the composition affords a plasma level of cyclobenzaprine that decreases by at least 70% of the Cₘₐₓ by 8 hours after administration.

122. The method of embodiment 121, wherein the composition affords a plasma level of cyclobenzaprine that decreases by at least 80% of the Cₘₐₓ by 8 hours after administration.

123. The method of embodiment 122, wherein the composition affords a plasma level of cyclobenzaprine that decreases by at least 90% of the Cₘₐₓ by 8 hours after administration.

124. The method of embodiment 123, wherein the composition affords a plasma level of cyclobenzaprine that decreases by at least 95% of the Cₘₐₓ by 8 hours after administration.

125. A composition comprising cyclobenzaprine for transmucosal administration comprising from about 2 to about 20 mg of cyclobenzaprine or a salt thereof, said formulation affording a Cₘₐₓ of cyclobenzaprine from about 20 to about 200 ng/mL from about 0.0.05 to about 2.5 hours after administration, and a minimum cyclobenzaprine plasma concentration from about 1 to about 5 ng/mL from about 22 to about 26 hours after administration, wherein the composition is administered for four days or more of daily administration.

126. A method for reducing the symptoms of fibromyalgia in a human patient, comprising administering a transmucosal dosage formulation comprising from about 2 to about 20 mg of cyclobenzaprine or a salt thereof, said formulation affording a Cₘₐₓ of cyclobenzaprine from about 20 to about 200 ng/mL from about 0.05 to about 2.5 hours after administration, and a minimum plasma concentration from about 1 to about 5 ng/mL from about 22 to about 26 hours after administration, wherein the composition is administered for four days or more of daily administration, and wherein the composition is administered within two hours of sleep.

127. A composition comprising amitriptyline for transmucosal administration comprising from about 2 to about 25 mg of amitriptyline or a salt thereof, said formulation affording a Cₘₐₓ of amitriptyline from about 20 to about 200 ng/mL from about 0.05 to about 2.5 hours after administration, and a minimum amitriptyline plasma concentration from about 1 to about 5 ng/mL from about 22 to about 26 hours after administration, wherein the composition is administered for four days or more of daily administration.

128. A method for reducing the symptoms of fibromyalgia in a human patient, comprising administering a transmucosal dosage formulation comprising from about 2 to about 25 mg of amitriptyline or a salt thereof, said formulation affording a Cₘₐₓ of amitriptyline from about 20 to about 200 ng/mL from about 0.05 to about 2.5 hours after administration, and a minimum plasma concentration from about 1 to about 5 ng/mL from about 22 to about 26 hours after administration, wherein the composition is administered for four days or more of daily administration, and wherein the composition is administered within two hours of sleep.

129. The composition of any one of embodiments 1-14, characterized in that, when administered by transmucosal absorption, the cyclobenzaprine or amitriptyline has a dnC* of greater than or equal to 1.0±25%×10⁻⁷ mL⁻¹ 20 minutes after administration.

130. The composition of any one of embodiments 1-14, characterized in that, when administered by transmucosal absorption, the cyclobenzaprine or amitriptyline has a dnC* of greater than or equal to 157.60×10⁻⁹ mL⁻¹ 20 minutes after administration.

131. The composition of any one of embodiments 1-14, characterized in that, when administered by transmucosal absorption, the cyclobenzaprine or amitriptyline has a dnC* of 2.5±25%×10⁻⁷³⁰ minutes after administration.

132. The composition of any one of embodiments 1-14, characterized in that, when administered by transmucosal absorption, the cyclobenzaprine or amitriptyline has a dnC* of greater than or equal to 3.0±25%×10⁻⁷ mL⁻¹ 45 minutes after administration.

133. The composition of any one of embodiments 1-14, characterized in that, when administered by transmucosal absorption, the cyclobenzaprine or amitriptyline has a dnC* of greater than or equal to 4.2±25%×10⁻⁷ mL⁻¹ 60 minutes after administration.

134. The composition of any one of embodiments 1-14, characterized in that, when administered by transmucosal absorption, the cyclobenzaprine or amitriptyline has a dnC* of greater than or equal to 6.0±25%×10⁻⁷ mL⁻¹ 2 hours after administration.

135. The composition of any one of embodiments 1-14, characterized in that, when administered by transmucosal absorption, the cyclobenzaprine or amitriptyline has a dnC* of greater than or equal to 6.5±25%×10⁻⁷ mL⁻¹ 2.5 hours after administration.

136. The composition of any one of embodiments 1-14, characterized in that, when administered by transmucosal absorption, the cyclobenzaprine or amitriptyline has a dnC* of greater than or equal to 7.0±25%×10⁻⁷ mL⁻¹ 3 hours after administration.

137. The composition of any one of embodiments 1-32, wherein the cyclobenzaprine is present in an amount of 2.8 mg.

138. The composition of any one of embodiments 1-32, wherein the cyclobenzaprine is present in an amount of 5.6 mg.

139. The composition of any one of embodiments 1-32, wherein the cyclobenzaprine is present in an amount of less than about 9 mg.

140. The method of any one of embodiments 62-77, wherein the cyclobenzaprine or amitriptyline has a dnC* of greater than or equal to 1.0±25%×10⁻⁷ mL⁻¹ 20 minutes after administration.

141. The method of any one of embodiments 62-77, wherein the cyclobenzaprine or amitriptyline has a dnC* of greater than or equal to 2.5±25%×10⁻⁷ 30 minutes after administration.

142. The method of any one of embodiments 62-77, wherein the cyclobenzaprine or amitriptyline has a dnC* of greater than or equal to 3.0±25%×10⁻⁷ 45 minutes after administration.

143. The method of any one of embodiments 62-77, wherein the cyclobenzaprine or amitriptyline has a dnC* of greater than or equal to 4.2±25%×10⁻⁷ 1 hour after administration.

144. The method of any one of embodiments 62-77, wherein the cyclobenzaprine or amitriptyline has a dnC* of greater than or equal to 6.0±25%×10⁻⁷ mL⁻¹ 2 hours after administration.

145. The method of any one of embodiments 62-77, wherein the cyclobenzaprine or amitriptyline has a dnC* of greater than or equal to 6.5±25%×10⁻⁷ mL⁻¹ 2.5 hours after administration.

146. The method of any one of embodiments 62-77, wherein the cyclobenzaprine or amitriptyline has a dnC* of greater than or equal to 727.67±25%×10⁻⁹ mL⁻¹ 2.5 hours after administration.

147. The method of any one of embodiments 62-77, wherein the cyclobenzaprine or amitriptyline has a dnC* of greater than or equal to 7.0±25%×10⁻⁷ mL⁻¹ 3 hours after administration.

148. The composition of any one of embodiments 1-38, characterized in that, when administered by transmucosal absorption, the composition affords a Cₘₐₓ of cyclobenzaprine greater than or equal to 2 ng/mL.

149. The composition of any one of embodiments 1-43, characterized in that, when administered by transmucosal absorption, the composition affords a Cₘₐₓ of cyclobenzaprine greater than or equal to 2 ng/mL above the baseline level of cyclobenzaprine in the individual immediately prior to administration.

### BRIEF DESCRIPTION OF THE DRAWINGS

FIG. 1a depicts a linear plot of mean plasma cyclobenzaprine concentration±standard deviation and mean plasma norcyclobenzaprine concentration±standard deviation in 10 healthy human subjects after oral (PO) treatment with 5 mg of cyclobenzaprine HCl immediate release tablets under fasting conditions.

FIG. 1b depicts a log plot of mean plasma cyclobenzaprine concentration±standard deviation and mean plasma norcyclobenzaprine concentration±standard deviation in 10 healthy human subjects after PO treatment with 5 mg of cyclobenzaprine HCl immediate release tablets under fasting conditions.

FIG. 2 depicts a linear plot of mean cyclobenzaprine concentration standard deviation in female beagle dog plasma over time. Cyclobenzaprine was administered orally (PO) by nasogastric (NG) tube into the stomach, sublingually, or intravenously (IV).

FIG. 3 depicts a semilogarithmic plot of mean cyclobenzaprine concentration±standard deviation in female beagle dog plasma over time. Cyclobenzaprine was administered orally (PO) by NG tube, sublingually, or intravenously.

FIG. 4 depicts mean cyclobenzaprine concentration time profiles after IV administration of cyclobenzaprine HCl±standard deviation in female beagle dog plasma, comparing the mean of dogs treated with and without pre-anesthesia with propofol (IV Investigation) with the mean of dogs from the IV data in FIG. 2.

FIG. 5 depicts mean cyclobenzaprine concentration time profiles after sublingual administration of cyclobenzaprine HCl solution±standard deviation in female beagle dog plasma, comparing the mean of dogs treated with and without pre-anesthesia with propofol (Sublingual Investigation) with the mean of dogs from the sublingual data in FIG. 2.

FIG. 6 depicts a linear plot of mean cyclobenzaprine concentration standard deviation in female beagle dog plasma over time. Cyclobenzaprine was administered sublingually in tablets that either contained or lacked the basifying agent K₂HPO₄.

FIG. 7 depicts a log plot of mean cyclobenzaprine concentration standard deviation in female beagle dog plasma over time. Cyclobenzaprine was administered sublingually in tablets with and without the basifying agent, K₂HPO₄.

FIG. 8 depicts a chart showing daily assessments performed over the course of a study of cyclobenzaprine HCl administration to humans.

FIG. 9 depicts a chart showing hourly assessments performed during a study of cyclobenzaprine HCl 5 mg immediate release tablets after PO administration to humans.

FIG. 10 depicts a chart showing cyclobenzaprine plasma concentration-time profiles from 0 to 1 hr after administration of sublingual (SL, Subject 7 only), oral (PO, group mean), and intravenous (IV, group mean) doses of cyclobenzaprine.

FIG. 11 depicts a chart showing cyclobenzaprine plasma concentration-time profiles from 0 to 24 hrs after administration of sublingual (Subject 7 only), oral (group mean), and intravenous (group mean) doses of cyclobenzaprine.

FIG. 12 depicts a chart showing mean cyclobenzaprine plasma concentration-time profiles from 0 to 24 hrs after administration of sublingual cyclobenzaprine at pH 7.1 (mean for all subjects except Subjects 7 and 10), pH 3.5 (mean for all subjects except Subject 4), and oral (group mean) cyclobenzaprine.

FIG. 13 depicts a chart showing norcyclobenzaprine plasma concentration-time profiles from 0 to 24 hrs after administration of sublingual (Subject 7 only), oral (group mean), and intravenous (group mean) doses of cyclobenzaprine.

FIG. 14a depicts a chart showing cyclobenzaprine plasma concentration from 0 to 2 hrs after administration of 2.4 mg of sublingual cyclobenzaprine and 5 mg of oral cyclobenzaprine. FIG. 14b depicts a chart showing cyclobenzaprine plasma concentration from 0 to 2 hrs after administration of 4.8 mg of sublingual cyclobenzaprine and 5 mg of oral cyclobenzaprine.

FIG. 15 depicts a chart showing cyclobenzaprine plasma concentration from 0 to 8 hrs after administration of 4.8 mg of sublingual cyclobenzaprine and 5 mg of oral cyclobenzaprine.

FIG. 16 depicts a chart showing cyclobenzaprine plasma concentration from 0 to 8 hrs after administration of 2.4 and 4.8 mg of sublingual cyclobenzaprine.

FIG. 17a depicts a chart showing cyclobenzaprine plasma concentration from 0 to 2 hrs after administration of 2.4 mg of sublingual cyclobenzaprine with and without phosphate. FIG. 17b depicts a chart showing cyclobenzaprine plasma concentration from 0 to 8 hrs after administration of 2.4 mg of sublingual cyclobenzaprine with and without phosphate.

FIG. 18a depicts equilibrium binding studies of cyclobenzaprine (circles) and norcyclobenzaprine (triangles) on serotonin (5-HT_{1A}) receptor expressed in the central nervous system of humans. FIG. 18b depicts equilibrium binding studies of cyclobenzaprine (circles) and norcyclobenzaprine (triangles) on serotonin (5-HT_{2A}) receptor expressed in the central nervous system of humans. FIG. 18c depicts equilibrium binding studies of cyclobenzaprine (circles) and norcyclobenzaprine (triangles) on serotonin (5-HT_{2B}) receptor expressed in the central nervous system of humans. FIG. 18d depicts equilibrium binding studies of cyclobenzaprine (circles) and norcyclobenzaprine (triangles) on serotonin (5-HT_{2C}) receptor expressed in the central nervous system of humans. FIG. 18e depicts equilibrium binding studies of cyclobenzaprine (circles) and norcyclobenzaprine (triangles) on histamine H₁ (H₁) receptor expressed in the central nervous system of humans. FIG. 18f depicts equilibrium binding studies of cyclobenzaprine (circles) and norcyclobenzaprine (triangles) on adrenergic α_{1A} (α_{1A}) receptor expressed in the central nervous system of humans. FIG. 18g depicts equilibrium binding studies of cyclobenzaprine (circles) and norcyclobenzaprine (triangles) on muscarinic M₁ (M₁) receptor expressed in the central nervous system of humans. FIG. 18h depicts equilibrium binding studies of cyclobenzaprine (circles) and norcyclobenzaprine (triangles) on dopamine D₁ (D₁) receptor expressed in the central nervous system of humans.

FIG. 19 depicts equilibrium binding studies of cyclobenzaprine (circles) and norcyclobenzaprine (triangles) on the norepinephrine (NE), 5-HT, and dopamine (D) transporters expressed in the central nervous system of humans.

FIG. 20a depicts G-protein-dependent signal transduction studies of cyclobenzaprine (circles) and norcyclobenzaprine (triangles) on serotonin (5-HT_{1A}) receptor expressed in the central nervous system of humans. FIG. 20b depicts G-protein-dependent signal transduction studies of cyclobenzaprine (circles) and norcyclobenzaprine (triangles) on serotonin (5-HT_{2A}) receptor expressed in the central nervous system of humans. FIG. 20c depicts G-protein-dependent signal transduction studies of cyclobenzaprine (circles) and norcyclobenzaprine (triangles) on serotonin (5-HT_{2B}) receptor expressed in the central nervous system of humans. FIG. 20d depicts G-protein-dependent signal transduction studies of cyclobenzaprine (circles) and norcyclobenzaprine (triangles) on serotonin (5-HT_{2C}) receptor expressed in the central nervous system of humans. FIG. 20e depicts G-protein-dependent signal transduction studies of cyclobenzaprine (circles) and norcyclobenzaprine (triangles) on histamine H₁ (H₁) receptor expressed in the central nervous system of humans. FIG. 20f depicts G-protein-dependent signal transduction studies of cyclobenzaprine (circles) and norcyclobenzaprine (triangles) on adrenergic α_{1A} (α_{1A}) receptor expressed in the central nervous system of humans. FIG. 20g depicts G-protein-dependent signal transduction studies of cyclobenzaprine (circles) and norcyclobenzaprine (triangles) on muscarinic M₁ (M₁) receptor expressed in the central nervous system of humans. FIG. 20h depicts G-protein-dependent signal transduction studies of cyclobenzaprine (circles) and norcyclobenzaprine (triangles) on dopamine Di (Di) receptor expressed in the central nervous system of humans.

FIG. 21a depicts G-protein-independent signal transduction studies of cyclobenzaprine (circles) and norcyclobenzaprine (triangles) on histamine H₁ (H₁) receptor expressed in the central nervous system of humans. FIG. 21b depicts G-protein-independent signal transduction studies of cyclobenzaprine (circles) and norcyclobenzaprine (triangles) on adrenergic ais (ais) receptor expressed in the central nervous system of humans. FIG. 21c depicts G-protein-independent signal transduction studies of cyclobenzaprine (circles) and norcyclobenzaprine (triangles) on muscarinic M₁ (M₁) receptor expressed in the central nervous system of humans. FIG. 21d depicts G-protein-independent signal transduction studies of cyclobenzaprine (circles) and norcyclobenzaprine (triangles) on serotonin (5-HT_{2A}) receptor expressed in the central nervous system of humans.

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well known and commonly used in the art.

The methods and techniques of the present invention are generally performed, unless otherwise indicated, according to conventional methods well known in the art and as described in various general and more specific references that are cited and discussed throughout this specification.

Chemistry terms used herein are used according to conventional usage in the art, as exemplified by “The McGraw-Hill Dictionary of Chemical Terms”, Parker S., Ed., McGraw-Hill, San Francisco, C. A. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

A “patient”, “subject”, or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results include, but are not limited to, alleviation or amelioration of one or more symptoms associated with a disease or condition described herein.

“Administering” or “administration of” a substance, a compound or an agent to a subject can be carried out using one of a variety of methods known to those skilled in the art. For example, a compound or an agent can be administered sublingually or intranasally, by inhalation into the lung or rectally. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

The invention provides compositions and methods for administering compounds for transmucosal absorption. The compositions and methods have a number of surprising pharmacokinetic benefits in comparison to the oral administration of a compound, which results predominantly in the absorption of compounds in the stomach, small intestine and colon.

Each embodiment described herein may be used individually or in combination with any other embodiment described herein.

### Compounds

The compounds useful in embodiments of the present invention include cyclobenzaprine and amitriptyline. In some embodiments, the compounds are micronized. In alternative embodiments, the compounds are not micronized. In some embodiments, the compounds may be present in one or more crystal isoforms.

As used herein, “cyclobenzaprine” includes cyclobenzaprine and pharmaceutically acceptable salts of cyclobenzaprine (e.g., cyclobenzaprine HCl). In some embodiments, cyclobenzaprine may be modified by the covalent addition of lysine or by binding to albumin.

As used herein, “amitriptyline” includes amitriptyline and pharmaceutically acceptable salts of amitriptyline (e.g., amitriptyline HCl). In some embodiments, amitriptyline may be modified by the covalent addition of lysine or by binding to albumin.

### Doses

A “therapeutically effective amount” of a drug or agent is an amount of a drug or an agent that, when administered to a subject will have the intended therapeutic effect, e.g. reducing the symptoms of fibromyalgia or post-traumatic stress disorder (PTSD) or treating the development of fibromyalgia or post-traumatic stress disorder (PTSD). The full therapeutic effect does not necessarily occur by administration of one dose, and may occur only after administration of a series of doses. Generally, cyclobenzaprine therapy can be carried out indefinitely to alleviate the symptoms of interest and frequency of dosage may be changed to be taken as needed. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled worker can readily determine the effective amount for a given situation by routine experimentation. Generally, a therapeutically effective amount of cyclobenzaprine or amitriptyline administered to a subject is between 0.1 mg and 20.0 mg, between 0.1 mg and 5.0 mg, between 0.1 mg and 4.0 mg, or between 0.1 and 3.0 mg, or between 1 and 50 mg or between 1 and 75 mg. In some embodiments, a therapeutically effective amount is about 0.1 mg, 0.5 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 6.0 mg, 6.5 mg, 7.0 mg, 7.5 mg, 8.0 mg, 8.5 mg, 9.0 mg, 9.5 mg, 10.0 mg, 11.0 mg, 12.0 mg, 13.0 mg, 14.0 mg, 15.0 mg, 16.0 mg, 17.0 mg, 18.0 mg, 19.0 mg, or 20.0 mg. In some embodiments, a therapeutically effective amount is about 21.0 mg, 22.0 mg, 23.0 mg, 24.0 mg, 25.0 mg, 26.0 mg, 27.0 mg, 28.0 mg, 28.0 mg, 29.0 mg, 30.0 mg, 31.0 mg, 32.0 mg, 33.0 mg, 34.0 mg, 35.0 mg, 36.0 mg, 37.0 mg, 38.0 mg, 39.0 mg, 40.0 mg, 41.0 mg, 42.0 mg, 43.0 mg, 44.0 mg, 45.0 mg, 46.0 mg, 47.0 mg, 48.0 mg, 49.0 mg, or 50.0 mg. In some embodiments, amtriptyline is present in a composition of the invention in an amount from 1 mg to 25 mg, for example, from 1 mg to 10 mg. In certain embodiments, amitriptyline is present in an amount of about 8 mg, less than about 16 mg, about 16 mg, or less than about 24 mg.

### Administration

Appropriate methods of administering a substance, a compound or an agent to a subject will depend, for example, on the age of the subject, whether the subject is active or inactive at the time of administering, whether the subject is experiencing symptoms of a disease or condition at the time of administering, the extent of the symptoms, and the chemical and biological properties of the compound or agent (e.g. solubility, digestibility, bioavailability, stability and toxicity). In some embodiments, the compound is administered for transmucosal absorption. Absorption properties of compounds of the invention through transmucosal delivery cannot be predicted without experimentation. The suitability of compounds of the invention for transmucosal absorption is a surprising feature. Transmucosal absorption can occur through any mucosa. Exemplary mucosa include oral mucosa (e.g., buccal mucosa and sublingual mucosa), nasal mucosa, rectal mucosa, and pulmonary mucosa. In some embodiments, a composition is suitable for transmucosal absorption. In some embodiments, a composition is formulated for transmucosal absorption.

Methods of administering compositions for transmucosal absorption are well known in the art. For example, a composition may be administered for buccal absorption through buccal tablets, lozenges, buccal powders, and buccal spray solutions. A composition may be administered for sublingual absorption through sublingual tablets, sublingual films, liquids, sublingual powders, and sublingual spray solutions. A composition may be administered for intranasal absorption through nasal sprays. A composition may be administered for pulmonary absorption through aerosolized compositions and inhalable dried powders. When administered via sprays or aerosolized compositions, a composition may be prepared with saline as a solution, employ benzyl alcohol or other suitable preservatives, or include absorption promoters to enhance bioavailability, fluorocarbons, and/or other solubilizing or dispersing agents.

Doses and dosing regimens can be determined by one of skill in the art according to the needs of a subject to be treated. The skilled worker may take into consideration factors such as the age or weight of the subject, the severity of the disease or condition being treated, and the response of the subject to treatment. A composition of the invention can be administered, for example, as needed or on a daily basis. In some embodiments, a composition can be administered immediately prior to sleep or several hours before sleep. Administration prior to sleep may be beneficial by providing the therapeutic effect before the onset of the symptoms of the disease or condition being treated. Dosing may take place over varying time periods. For example, a dosing regimen may last for 1 week, 2 weeks, 3 weeks, 4 weeks, 5 weeks, 6 weeks, 7 weeks, 8 weeks, 9 weeks, 10 weeks, 11 weeks, 12 weeks, or longer. In some embodiments, a dosing regimen will last 1 month, 2 months, 3 months, 4 months, 5 months, 6 months, 7 months, 8 months, 9 months, 10 months, 11 months, 12 months, or longer.

### Therapeutic Uses

The compounds of the invention may be employed for treating or preventing the development of fibromyalgia syndrome, also known as fibrositis (see, e.g., Moldofsky et al., J Rheumatol 38(12):2653-2663 (2011) and Thomas, J Rheumatol 38(12):2499-2500 (2011)). Fibromyalgia is a chronic, non-inflammatory rheumatic disorder. The American College of Rheumatology (ACR) published a classification criteria for fibromyalgia in 1990 (Wolfe, F., et al., Arthritis and Rheumatism 33:160-172 (1990)). Subsequently, a modification to the ACR criteria been published (Wolfe et al., J Rheumatol 38(6):1113-22 (2011)). Diagnostic criteria have also been published by an international network of working groups called, “Outcome Measures in Rheumatology” clinical trials or OMERACT (Mease P, et al. J Rheumatol. 2009; 36(10):2318-29). Fibromyalgia is traditionally characterized by stiffness or diffuse pain, aches, muscle soreness, sleep disturbances or fatigue. The pain is generally widespread and generally localized at specific “tender points,” which may bring on widespread pain and muscle spasm when touched. Other symptoms include mental and emotional disturbances such as poor concentration and irritability, neuropsychiatric symptoms such as depression and anxiety, joint swelling, headache, numbness. Fibromyalgia is associated with nonrefreshing sleep, tiredness, sleepiness, reflux, mental fog and cognitive impairments including difficulty multi-tasking. Fibromyalgia also is often comorbid with sleep disorders, fatigue, non-restorative sleep, anxiety, and depression. The compositions and methods of the invention can be used to treat any one of the above-identified conditions, and any combination thereof.

Some practitioners further classify fibromyalgia into two categories—primary or secondary-concomitant fibromyalgia. Generally, primary fibromyalgia syndrome can be considered fibromyalgia occurring in the absence of another significant condition whereas secondary-concomitant fibromyalgia can be considered fibromyalgia occurring in the presence of another significant medical disorder, which may have been caused by or is merely associated with the patient's fibromyalgia. Secondary or concomitant fibromyalgia can include fibromyalgia in patients with classical or definite rheumatoid arthritis, osteoarthritis of the knee or hand, low back pain syndromes, cervical pain syndromes, cancer pain syndromes, temporomandibular joint disorders, migraine headaches, menopause, post-traumatic stress disorder and interstitial cystitis or painful bladder syndrome (or combinations thereof).

The compounds of the invention also may be employed for treating or preventing the development (either the initiation, consolidation or perpetuation) of a PTSD symptom following a traumatic event. A traumatic event is defined as a direct personal experience that involves actual or threatened death or serious injury, or other threat to one's physical integrity, or witnessing an event that involves death, injury, or a threat to the physical integrity of another person; or learning about unexpected or violent death, serious harm, or threat of death or injury experienced by a family member or other close associate. Traumatic events that are experienced directly include, but are not limited to, military combat, violent personal assault (sexual assault, physical attack, robbery, mugging), being kidnapped, being taken hostage, terrorist attack, torture, incarceration as a prisoner of war or in a concentration camp, natural or manmade disasters, severe automobile accidents, or being diagnosed with a life-threatening illness. For children, sexually traumatic events may include developmentally inappropriate sexual experiences without threatened or actual violence or injury. Witnessed events include, but are not limited to, observing the serious injury or unnatural death of another person due to violent assault, accident, war, or disaster or unexpectedly witnessing a dead body or body parts. Events experienced by others that are learned about may include, but are not limited to, violent personal assault, serious accident, or serious injury experienced by a family member or a close friend, learning about the sudden, unexpected death of a family member or a close friend, or learning that one's child has a life-threatening disease. The disorder may be especially severe or long lasting when the stressor is of human design (e.g., torture or rape). The initiation of a PTSD symptom typically occurs immediately following the traumatic event, during which the symptoms of PTSD appear and become increasingly severe. One theory of how PTSD develops is that there is a type of “learning” or reinforcement process during which the memories of the trauma are engrained in the mind. As these memories become more fixed (a process called consolidation), symptoms such as flashbacks and nightmares grow in severity and frequency. Interventions during this critical time may prevent some patients from developing full-blown PTSD. The consolidation of a PTSD symptom typically occurs during the weeks and months following a traumatic event. A person's memories of that event become consolidated into highly vivid and concrete memories that are re-experienced with increasing frequency either as flashbacks or nightmares. During this time, hyperarousal symptoms and avoidant behavior can become increasingly severe and disabling. The perpetuation of a PTSD symptom occurs once traumatic memories are consolidated, and the re-experienced symptoms (flashbacks and nightmares) and hyperarousal symptoms become persistent and remain at a level that is functionally disabling to the patient.

The compositions and methods of the invention may be used to treat different phases of PTSD development at various time intervals after a traumatic event. For example, treating the initiation phase of PTSD may require the administration of a composition of the invention soon after the traumatic event, for example within the first week, within the second week, within the third week, or within the fourth week or later. By contrast, when treating the consolidation phase of PTSD, the skilled worker may be able to administer a composition of the invention later after the traumatic event and later during the development of the symptoms, for example, within the first month, within the second month, or within the third month or later. The perpetuation phase of PTSD may be treated with a composition of the invention administered 3 months or longer after the traumatic event, for example within the third month, within the fourth month, within the fifth month, or later. As a result of treatment at the initiation, consolidation, or perpetuation phase, PTSD symptoms will be ameliorated or be eliminated.

The compositions and methods of the invention also can be used to treat traumatic brain injury (TBI). TBI is associated with sleep disorders, sleep disturbances, fatigue, non-restorative sleep, anxiety, and depression. The compositions and methods of the invention also can be used to treat any of the above conditions, in combination with or independently of treating TBI.

The compositions and methods of the invention also can be used to chronic traumatic encephalopathy (CTE). CTE is associated with sleep disorders, sleep disturbances, fatigue, non-restorative sleep, anxiety, and depression. The compositions and methods of the invention also can be used to treat any of the above conditions, in combination with or independently of treating CTE.

The compositions and methods of the invention may be used to treat sleep disorders or sleep disturbances. A “sleep disorder” may be any one of four major categories of sleep dysfunction (DSM-IV, pp. 551-607; see also The International Classification of Sleep Disorders: (ICSD) Diagnostic and Coding Manual, 1990, American Sleep Disorders Association). One category, primary sleep disorders, comprises sleep disorders that do not result from another mental disorder, a substance, or a general medical condition. They include without limitation primary insomnia, primary hypersomnia, narcolepsy, circadian rhythm sleep disorder, nightmare disorder, sleep terror disorder, sleepwalking disorder, REM sleep behavior disorder, sleep paralysis, day/night reversal and other related disorders; substance-induced sleep disorders; and sleep disorders due to a general medical condition. Primary insomnia non-restorative sleep is described by the DSM-IV-TR as a type of primary insomnia wherein the predominant problem is waking up feeling unrefreshed or nonrefreshed. A second category comprises those sleep disorders attributable to substances, including medications and drugs of abuse. A third category comprises sleep disturbances arising from the effects of a general medical condition on the sleep/wake system. A fourth category of sleep disorders comprises those resulting from an identifiable mental disorder such as a mood or anxiety disorder. A fifth category of sleep disorders comprises those described as non-restorative sleep. One definition of non-restorative sleep is in the DSM-IV-TR as a type of primary insomnia (A1.3) wherein the predominant problem is waking up feeling unrefreshed or nonrefreshed. Symptoms of each category of sleep disorder are known in the art. A “sleep disturbance” may be an impairment in refreshing sleep. Such a clinical diagnosis may be made based on a patient's self described feeling of fatigue upon waking or the patient's report of poor quality sleep. Such impediments to good quality sleep may be described as shallow sleep or frequent awakenings which may be associated with an increase in the Cyclic Alternating Pattern (CAP) A2 or A3 rate or cycle duration or an increase in the normalized CAP A2+A3 which is determined by CAP (A2+A3)/CAP (A1+A2+A3) in non-REM sleep (see, e.g., Moldofsky et al., J Rheumatol 38(12):2653-2663 (2011) and Thomas, J Rheumatol 38(12):2499-2500 (2011)), alpha rhythm contamination in non-REM sleep, or absence of delta waves during deeper physically restorative sleep. Such “sleep disturbances” may or may not rise to the level of a “sleep disorder” as defined in the DSM-IV, although they may share one or more symptom in common. Symptoms of sleep disturbances are known in the art. Among the known symptoms are groggy or spacey feelings, tiredness, feelings of being run down, and having difficulty concentrating during waking hours. Among the sleep-related conditions that may be treated with the methods and compositions of the invention are dyssomnias (e.g., intrinsic sleep disorders such as sleep state misperception, psychophysiological insomnia, idiopathic insomnia, obstructive sleep apnea syndrome, central sleep apnea syndrome, central alveolar hypoventilation syndrome, restless leg syndrome, and periodic limb movement disorder; extrinsic sleep disorders such as environmental sleep disorder, adjustment sleep disorder, limit-setting sleep disorder, stimulant-dependent sleep disorder, alcohol-dependent sleep disorder, toxin-induced sleep disorder, sleep onset association disorder, hypnotic dependent sleep disorder, inadequate sleep hygiene, altitude insomnia, insufficient sleep syndrome, nocturnal eating syndrome, and nocturnal drinking syndrome; and circadian rhythm sleep disorders such as jet lag syndrome, delayed sleep phase syndrome, advanced sleep phase syndrome, shift work sleep disorder, non-24 hour sleep-wake disorder, and irregular sleep-wake patterns), parasomnias (e.g., arousal disorders such as sleepwalking, confusional arousals, and sleep terrors and sleep-wake transition disorders such as rhythmic movement disorder, sleep talking and sleep starts, and nocturnal leg cramps), and sleep disorders associated with medical or psychiatric conditions or disorders.

### Basifying Agents

The compositions of the invention may include a basifying agent in addition to a compound useful in the compositions of the invention. As used herein, a “basifying agent” refers to an agent (e.g., a substance that increases the local pH of the liquid near a mucosal surface including potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, disodium citrate, trisodium citrate, borate, hydroxide, silicate, nitrate, dissolved ammonia, the conjugate bases of some organic acids (including bicarbonate), and sulfide) that raises the pH of a solution containing a compound (e.g., cyclobenzaprine HCl or amitriptyline HCl) useful in the compositions and methods of the invention. The solution of interest is the layer of aqueous material overlying a mucous membrane. Therefore the basifying agent is sometimes an ingredient (and excipient) in a tablet, and the basifying agent exerts its effects during the time the tablet is being dispersed in the mucous material, while parts of the formulation are dissolving in the mucous material and for a period of time after the tablet is dissolved in the mucous material. Surprisingly, the addition of a basifying agent to a composition of the invention improves the pharmacokinetic properties of the composition. This is exemplified by cyclobenzaprine HCl as one particular compound useful in the methods and compositions of the invention. A basifying agent with particular effects on cyclobenzaprine HCl is dipotassium hydrogen phosphate (K₂HPO₄). Another basifying agent with particular effects on cyclobenzaprine HCl is potassium dihydrogen phosphate (KH₂PO₄). Another basifying agent with particular effects on cyclobenzaprine HCl is disodium hydrogen phosphate (Na₂HPO₄). Another basifying agent with particular effects on cyclobenzaprine HCl is tripotassium citrate. Another basifying agent with particular effects on cyclobenzaprine HCl is trisodium citrate. In some embodiments, amitriptyline HCl is a particular compound useful in the methods and compositions of the invention. A basifying agent with particular effects on amitriptyline HCl is K₂HPO₄. Another basifying agent with particular effects on amitriptyline HCl is Na₂HPO₄. Another basifying agent with particular effects on amitriptyline HCl is KH₂PO₄. Another basifying agent with particular effects on amitriptyline HCl is tripotassium citrate. Another basifying agent with particular effects on amitriptyline HCl is trisodium citrate.

Cyclobenzaprine HCl has an acid dissociation constant (or pKa) for the amine group of approximately 8.5 at 25° C., indicating that at pH 8.5, the compound is 50% ionized or protonated (and 50% un-ionized or free base) (M. L. Cotton, G. R. B. Down, Anal. Profiles Drug Subs. 17, 41-72 (1988)). The pH of an aqueous solution of cyclobenzaprine HCl from 10 gm/100 mL (0.32 molar) to 30 gm/100 mL (0.96 molar) is between approximately 3.1 and 3.3, thereby providing a condition wherein nearly all the cyclobenzaprine is ionized and soluble. The skilled worker, with this knowledge in hand, would therefore look to maintain cyclobenzaprine at a low pH, maximizing its solubility. However, ionized cyclobenzaprine may not have optimal absorption by and across mucosal surfaces because of its charge. This problem is solved by combining the cyclobenzaprine with a basifying agent. Indeed, we have discovered that combining cyclobenzaprine HCl with a basifying agent such as dipotassium hydrogen phosphate (K₂HPO₄) or potassium dihydrogen phosphate (KH₂PO₄) improves the pharmacokinetic properties of a composition comprising cyclobenzaprine for transmucosal absorption. In experiments with oral and intravenous solutions containing cyclobenzaprine and a basifying agent, the pH was adjusted to about pH 7.1 to pH 7.4. In experiments with tablet formulations containing cyclobenzaprine and a basifying agent, the addition of the basifying agent results in a higher pH when the tablet is dissolved in water. Combining cyclobenzaprine with a basifying agent also enhances the uptake of cyclobenzaprine by transmucosal absorption. The effects of a basifying agent like K₂HPO₄ or KH₂PO₄ on the transmucosal pharmacokinetic properties of a composition comprising cyclobenzaprine are remarkable because cyclobenzaprine HCl in a solution at 2.5 mg/ml at 7.4 containing K₂HPO₄ or KH₂PO₄ appears to be close to its saturation point where cyclobenzaprine falls out of solution, or becomes insoluble, presumably because the concentration of cyclobenzaprine free base increases relative to ionized cyclobenzaprine. Without wishing to be bound by theory, it is possible that the basifying agent increases the pH of the microenvironment local to the mucosal membrane and brings more of the cyclobenzaprine into an un-ionized or free-base state at the mucosal surface, which helps drive cyclobenzaprine across mucosa and into the bloodstream, thereby offsetting any decrease in the solubility of cyclobenzaprine resulting from the basifying agent action in the solution near the mucous membrane. Without wishing to be bound by theory, the basifying agent may create a transition state involving hydration of the free base, such that the free base is formed in situ near the mucosal surface and crosses the mucosal membrane.

A basifying agent useful in the compositions and methods of the invention may be any agent that increases the pH of a solution containing a compound useful in the methods and compositions of the invention. Exemplary basifying agents include potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), sodium carbonate, sodium bicarbonate, calcium carbonate, calcium bicarbonate, TRIS buffer, potassium carbonate, potassium bicarbonate, potassium acetate, sodium acetate, potassium citrate and sodium citrate. In some embodiments, a composition of the invention has a molar ratio of a compound (e.g., cyclobenzaprine or amitriptyline) to a basifying agent of 1:1, 1:2, 1:3, 1:4, 1:5 1:6, 1.7, 1.8, 1.9 or 2.0. In some embodiments, the ratio of cyclobenzaprine HCl (2.4 mg, MW 275.387) to K₂HPO₄ (1.05 mg, MW 174.2) is 0.69.

### Cyclobenzaprine and Amitriptyline Metabolism

Cyclobenzaprine rapidly distributes out of the vasculature after intravenous (IV) bolus administration in humans (Hucker et al., J Clin Pharmacol 17:719-727 (1977), Hucker et al., Drug Metab Dispos 6:659-672 (1978), Till at al., Annu Rev Pharmacol Toxicol 40:581-616 (2000), and Winchell et al., J Clin Pharmacol 42:61-69 (2002)). The amount of cyclobenzaprine in plasma within 3 to 30 minutes was less than 5% of the theoretical initial concentration (Cᵢₙᵢₜ) of each infused dose, and a 3 minute time point resulted in a relatively higher Cᵢₙᵢₜ. Together, this information shows that cyclobenzaprine is rapidly partitioned out of plasma. Since greater than 95% of injected cyclobenzaprine is cleared from plasma before the first time point of any of the four studies listed above, they are not informative about the half-life of the first phase of IV cyclobenzaprine distributing out of plasma, beyond establishing that an upper limit for the half-life of the first phase is clearly less than 3-5 minutes.

Amitriptyline is structurally related to cyclobenzaprine and differs from cyclobenzaprine chemically by lacking the C10-C11 double-bond in the central cycloheptyl ring. However, changes in chemical structure can affect drug actions on normal or pathological tissues as well as absorption, disposition, metabolism and excretion. Amitriptyline and its desmethyl metabolite nortriptyline are the active pharmaceutical ingredients of the tricyclic anti-depressants (TCAs) Elavil® and Pamelor®, respectively. Amitriptyline, like cyclobenzaprine, is rapidly partitioned out of plasma. The pharmacokinetics of IV administration of cyclobenzaprine or amitriptyline or IV administration of amitriptyline can be described by a two-compartment model containing a plasma “central” compartment and a “peripheral” compartment.

Neither cyclobenzaprine nor amitriptyline is an effective long-term treatment for fibromyalgia in any formulation that has been tested, for example currently available formulations were not effective over six months of treatment (Carette, S. Arthritis Rheum. 1994 January; 37(1):32-40). Cyclobenzaprine is not an effective treatment for fibromyalgia in a twelve week study (Bennett et al., Arthritis Rheum. 31: 1535-1542 (1988)). In general, cyclobenzaprine is not recommended for long-term use. We hypothesized that the side effects of fatigue, somnolence and grogginess have overwhelmed the treatment effects from cyclobenzaprine or amitriptyline, but it was not known how to shorten the plasma half-life of either cyclobenzaprine or amitriptyline. In addition, we have shown for the first time that the ineffectiveness of cyclobenzaprine may be due to the metabolism of cyclobenzaprine by the liver. Whereas amitriptyline was known to be metabolized to nortriptyline, and the delayed plasma half life of nortriptyline has been reported (Bhatt, Biomed Chromatogr 24(11):1247-54 (2010)), the manner in which nortriptyline accumulation may decrease efficacy of bedtime amitriptyline as a chronic treatment was not understood.

Cyclobenzaprine is extensively metabolized and in humans, excreted predominantly by the kidney as the N+-glucuronide conjugate. Glucuronidation of an aliphatic tertiary amine group in a molecule results in a quaternary ammonium-linked glucuronide metabolite (i.e. N+-glucuronide) (Hucker et al., Drug Metab Dispos 6:659-672 (1978), Hawes, Drug Metab Dispos 26:830-837 (1998)). Amitriptyline has been studied in more detail and more recently than cyclobenzaprine, and the enzyme UDP-glucuronysl-transferase (UGT) UGT2B10 was found to be the high-affinity component (Zhou et al., Drug Metab Dispos 38:863-870 (2010)), while UGT1A4 is the low-affinity enzyme for glucuronidation in human liver microsomes (HLM) (Breyer-Pfaff et al., Drug Metab Dispos 25:340-345 (1997), Nakajima et al., Drug Metab Dispos 30:636-642 (2002)). It is likely that cyclobenzaprine is similarly metabolized to cyclobenzaprine-N+-glucuronide by UGT2B10 and, to a lesser extent, by UGT1A4.

Cyclobenzaprine also is N-demethylated to 3-(5H-dibenzo[a,d]cyclohepten-5-ylidene)-N-methyl-1-propanamine (norcyclobenzaprine), predominantly by hepatic enzymes P450 3A4 and 1A2 (Wong et al., J Anal Toxicol 19:218-224 (1995)). Amitriptyline is similarly subject to P450 mediated N-demethylation to nortriptyline. Nortriptyline represents a significant percentage of the plasma amitriptyline plus nortriptyline content in subjects who ingest amitriptyline either as a single dose or chronically, whereas norcyclobenzaprine had not previously been measured in human plasma except in cases of overdose (Hucker et al., Drug Metab Dispos 6:659-672 (1978), Wong et al., J Anal Toxicol 19:218-224 (1995)).

Previously, a number of studies had been performed and published showing cyclobenzaprine, amitriptyline and nortriptyline binding to various receptors in the central nervous system and peripheral tissues. We undertook a systematic analysis of the binding affinities of these molecules (cyclobenzaprine, amitriptyline and nortriptyline) and of norcyclobenzaprine (which had never been studied before to our knowledge) and determined the Kᵢ of these molecules on a variety of receptors. Cyclobenzaprine, norcyclobenzaprine, amitriptyline and nortriptyline binding to receptors was studied by methods described in: adrenergic alpha-2A (Langin et al., Eur J Pharmacol 167: 95-104 (1989), -2B, and -2C receptors (Devedjian et al., Eur J Pharmacol 252: 43-49 (1994)), the histamine H1 receptor (Smit et al., Brit. J. Pharmacol 117: 1071-1080 (1996)), the muscarinic M1 and M2 receptors (Dorje et al., J Pharmacol Exp Ther 256: 727-733 (1991)), and the 5-HT1A (Mulheron et al., J Biol Chem 269: 12954-12962 (1994)), 5-HT2A (Bonhaus et al., Brit J Pharmacol 115: 622-628 (1995)) and 5-HT2C receptors (Stam et al., Eur J Pharmacol 269: 339-348 (1994)). The binding constants Kᵢ are listed in Table 1, below. In vitro, cyclobenzaprine and norcyclobenzaprine exhibited high affinity binding (Kᵢ) to receptors: 5HT2a (5.2 and 13 nM) and 5HT2c (5.2 and 43 nM, respectively), alpha-adrenergic alpha-1A (5.6 and 34 nM), alpha-2B (Kᵢ=21 and 150 nM) and alpha-2C (Kᵢ=21 and 48 nM,); H1 (1.3 nM and 5.6 nM); and M1 (7.9 nM and 30 nM). Like cyclobenzaprine, norcyclobenzaprine is a functional antagonist at 5HT2a (IC₅₀=92 nM) by Ca⁺ mobilization. Cyclobenzaprine is also an antagonist on 5HT2b (IC₅₀=100 nM). Cyclobenzaprine and norcyclobenzaprine are functional antagonists on 5HT2c (IC₅₀=0.44 μM and 1.22 μM) and on alpha-2A (IC₅₀=4.3 μM and 6.4 μM). By contrast, both cyclobenzaprine and norcyclobenzaprine are functional agonists on 5HT1a (EC₅₀=5.3 μM and 3.2 μM). Cyclobenzaprine's antagonist activity on 5HT2b is consistent with the lack of any association with heart valve pathology. Antagonists of 5HT2a and H-1 are known to have effects on sleep and sleep maintenance. Adrenergic antagonists may have effects on autonomic dysfunction.

Without wishing to be bound by theory, we hypothesize that the primary activity of cyclobenzaprine that relates to effects on fibromyalgia, PTSD, TBI and sleep disturbances is the binding to 5-HT2a. Plasma cyclobenzaprine and norcyclobenzaprine were measured over 168 hr in ten healthy, fasting subjects who received 5 mg oral (PO) immediate release cyclobenzaprine HCl. The oral bioavailability of cyclobenzaprine was similar to published results (Cₘₐₓ=4.12 ng mL⁻¹, tₘₐₓ=3.5 h, AUC_{0-∞}=103.1 ng hr mL⁻¹), but plasma norcyclobenzaprine was unexpectedly high and persistent (Cₘₐₓ=1.27 ng mL⁻¹, tₘₐₓ=24.0 h, AUC_{0-∞}=169.5 ng hr mL⁻¹). We have calculated for the first time the half-life of norcyclobenzaprine in human plasma after oral ingestion of a 5 mg immediate release tablet of cyclobenzaprine HCl (72.8 hours), which is significantly longer than the half-life of cyclobenzaprine (31.0 hours) in the same study.

Without wishing to be bound by theory, we hypothesize that the accumulation of norcyclobenzaprine interferes with the efficacy of cyclobenzaprine treatment over extended periods of time for bedtime or once daily use in which the goal of therapy is to have cyclobenzaprine levels change dynamically over the course of the day. The accumulation of biologically active norcyclobenzaprine may affect responses to cyclobenzaprine therapy in a chronic bedtime dosing regimen. Without wishing to be bound by theory, we hypothesize the chronic occupancy of 5-HT2A and other receptors leads to adaptation by a variety of mechanisms which control plasticity of neuronal signaling and responsiveness. We found that norcyclobenzaprine has 13.2 nM Kᵢ for 5-HT2a and cyclobenzaprine has 5.1 nM Kᵢ for 5HT2A, which indicates that norcyclobenzaprine competes with the binding of cyclobenzaprine to 5-HT2A. Similarly, nortriptyline has 16 nM K, for 5-HT2A, and amitriptyline has 2.5 nM K, for 5-HT2A, which indicates that nortriptyline competes with the binding of amitriptyline to 5-HT2A. Although cyclobenzaprine and amitriptyline are more avid 5-HT2A binders than norcyclobenzaprine and nortriptyline, the concentrations of norcyclobenzaprine and nortriptyline become significant because of their longer half lives and accumulation, particularly with repeated dosing on a daily dosing schedule. By administering cyclobenzaprine or amitriptyline for transmucosal absorption, the cyclobenzaprine or amitriptyline avoids first pass metabolism in the liver, thereby reducing or eliminating the formation of norcyclobenzaprine or nortriptyline, respectively, by p450 metabolism in the gut and liver. Thus, cyclobenzaprine and amitriptyline can be effectively administered over longer treatment regimens than currently possible without accumulation of the demethylated metabolite. Without wishing to be bound by theory, we hypothesize the long half lives of norcyclobenzaprine and notriptyline are due to the instability of norcyclobenzaprine-N+-glucuronide and notriptyline (which we have observed in attempting to synthesize norcyclobenzaprine-N+-glucuronide) and possibly the inability of human enzymes, including UDP-glucuronysl-transferase (UGT) UGT2B10 and UGT1A4 to form the-N+-glucuronide metabolites which can be exerted in the kidney. Because norcyclobenzaprine had not been measured in plasma of animals after therapeutic dosing and norcyclobenzaprine's long half-life was not previously known, and because norcyclobenzaprine was not known to bind to 5-HT2A or to other receptors in the CNS and peripheral tissues, the benefit of transmucosal dosing on decreasing norcyclobenzaprine and increasing the therapeutic potential of cyclobenzaprine were surprising and novel. By contrast, the long half life of nortriptyline was known after either ingestion of amitriptyline or nortriptyline and the long half life of nortriptyline in plasma and at the site of action is believed to be an advantage in the treatment of depression and major depressive disorder.

TABLE 1
Binding affinities of cyclobenzaprine, norcyclobenzaprine, amitriptyline
and nortriptyline for various receptors
Ki (nM)

| | Alpha2A | Alpha2B | Alpha2C | H1 | | M1 | M2 | 5-HT1A | 5-HT2A | 5-HT2C |
|---|---|---|---|---|---|---|---|---|---|---|
| Cyclo | 360 | 21 | 25 | 1.3 | | 7.9 | 250 | 1100 | 5.2 | 43 |
| Norcyclo | 1800 | 150 | 48 | 5.6 | | 30 | 76 | 76 | 13 | 43 |
| Amitrip | 250 | 3.6 | 14 | 0.6 | | 4.3 | 61 | 220 | 2.5 | 28 |
| Nortrip | 1000 | 23 | 15 | 2.8 | | 24 | 140 | 100 | 16 | 16 |
| | Alpha1A | Alpha1B | D1 | D2S | D3 | D4.4 | D5 | 5-HT5A | 5-HT6 | 5-HT7 |
| Cyclo | 5.6 | 9.1 | 12 | 120 | 34 | 180 | 60 | 730 | 480 | 67 |
| Norcyclo | 34 | 11 | 57 | 410 | 98 | 250 | 280 | 1600 | 1400 | 140 |

### Pharmacokinetic Properties

Transmucosal absorption of a compound useful in the compositions and methods of the invention has a number of beneficial effects on the pharmacokinetic properties of the compound in addition to the benefit of avoiding the production of norcyclobenzaprine. Transmucosal delivery allows a compound of the invention to be absorbed more rapidly than if administered orally, resulting in a shorter time to therapeutic concentrations of cyclobenzaprine or amitriptyline in the plasma. In some embodiments, the compositions of the invention afford therapeutic concentrations of cyclobenzaprine or amitriptyline in the plasma at less than 3.3 hours, less than 3 hours, less than 2.5 hours, less than 2 hours, less than 1 hour, less than 45 minutes, less than 30 minutes, or less than 20 minutes. In some embodiments, the compositions of the invention afford increased concentrations of cyclobenzaprine or amitriptyline in the plasma, relative to oral doses, at time less than or equal to 3.3 hours, less than or equal to 3 hours, less than or equal to 2.5 hours, less than or equal to 2 hours, less than or equal to 1 hour, less than or equal to 45 minutes, less than or equal to 30 minutes, or less than or equal to 20 minutes. In some embodiments, the compositions of the invention afford increased AUCs of cyclobenzaprine or amitriptyline in the plasma, relative to oral doses, at times 0 to 3.3 hours, 0 to 3 hours, 0 to 2.5 hours, 0 to 2 hours, 0 to 1 hour, 0 to 45 minutes, 0 to 30 minutes, or 0 to 20 minutes. In some embodiments, the compositions of the invention afford increased dose-normalized concentrations (dnC*) of cyclobenzaprine or amitriptyline in the plasma, relative to oral doses, at time less than or equal to 3.3 hours, less than or equal to 3 hours, less than or equal to 2.5 hours, less than or equal to 2 hours, less than or equal to 1 hour, less than or equal to 45 minutes, less than or equal to 30 minutes, or less than or equal to 20 minutes. In some embodiments, the compositions of the invention afford increased dose normalized AUCs (dnAUC*) of cyclobenzaprine or amitriptyline in the plasma, relative to oral doses, at times 0 to 3.3 hours, 0 to 3 hours, 0 to 2.5 hours, 0 to 2 hours, 0 to 1 hour, 0 to 45 minutes, 0 to 30 minutes, or 0 to 20 minutes. Transmucosal delivery allows a compound of the invention to be absorbed more rapidly than if administered orally, resulting in a shorter time to maximum concentration, or tₘₐₓ. In some embodiments, the compositions of the invention afford a tₘₐₓ of cyclobenzaprine or amitriptyline of less than 5 hours, less than 4 hours, less than 3.5 hours, less than 3 hours, less than 2.5 hours, less than 2 hours, less than 1.5 hours, less than 1 hour, less than 45 minutes, less than 30 minutes, less than 15 minutes, less than 10 minutes, or less than 5 minutes. In some embodiments, the compositions of the invention afford a tₘₐₓ of cyclobenzaprine or amitriptyline of about 5 hours, about 4 hours, about 3 hours, about 2.5 hours, about 2 hours, about 1.5 hours, about 1 hour, about 45 minutes, about 30 minutes, about 15 minutes, about 10 minutes, or about 5 minutes.

Transmucosal absorption also produces higher plasma concentrations of a compound as compared to oral administration. A plasma concentration may be an individual plasma concentration or a mean plasma concentration when observing multiple individuals. The higher plasma concentrations produced by transmucosal absorption may be determined by measuring the plasma concentration of the compound being administered or by calculating the ratio of the plasma concentration and the dose administered, which is the dose-normalized plasma concentration (C) or dnC*, measured in mL⁻¹. The dnC* is calculated by determining the ratio of plasma level to dose administered. For example, if 2.4 mg of cyclobenzaprine or amitriptyline is administered and the plasma level is 2.4 ng/mL at 3 hours, the dnC* at 3 hours is ((2.4 ng/mL)/(2.4 mg))=1.0×10⁻⁶ mL⁻¹. The dnC* can be measured either at fixed time points or at a variable time point, e.g., the time point corresponding to Cₘₐₓ. The dose normalized concentration of cyclobenzaprine dnC* of cyclobenzaprine in plasma after ingestion of 5 mg immediate release cyclobenzaprine was: at 20 min was 0.00; at 30 min was 1.95×10⁻⁹ mL⁻¹; at 45 min was 19.31×10⁻⁹ mL⁻¹; at 1 hour was 50.00×10⁻⁹ mL⁻¹; at 2 hour was 378.65×10⁻⁹ mL⁻¹; at 2.5 hours (150 min) was 510.94×10⁻⁹ mL⁻¹; at 3 hours was 625.29×10⁻⁹ mL⁻¹×10⁻⁹ mL⁻¹; at 3.3 hours (200 min) was 698.49×10⁻⁹ mL⁻¹; at 3.67 hours (220 min) was 818.31×10⁻⁹ mL⁻¹; at 4 hours was 848.33×10⁻⁹ mL⁻¹; at 4.33 hours (260 min) was 968.09×10⁻⁹ mL⁻¹; at 4.67 hours (280 min) was 933.95×10⁻⁹ mL⁻¹; at 5 hours was 932.86×10⁻⁹ mL⁻¹; at 5.5 hours (330 min) was 920.94×10⁻⁹ mL⁻¹; at 6 hours was 953.40×10⁻⁹ mL⁻¹; at 8 hours was 801.23×10⁻⁹ mL⁻¹; at 12 hours was 516.73×10⁻⁹ mL⁻¹; at 16 hours was 347.39×10⁻⁹ mL⁻¹; at 24 hours was 320.44×10⁻⁹ mL⁻¹; at 36 hours was 233.66×10⁻⁹ mL⁻¹; at; at 48 hours was 199.41×10⁻⁹ mL⁻¹; and at 72 hours was 136.80. The dose normalized concentration of cyclobenzaprine dnC* of cyclobenzaprine in plasma after ingestion of 2.4 mg sublingual cyclobenzaprine with phosphate was: at 20 min was 157.60×10⁻⁹ mL⁻¹; at 30 min was 301.60×10⁻⁹ mL⁻¹; at 45 min was 432.58×10⁻⁹ mL⁻¹; at 1 hour was 598.85×10⁻⁹ mL⁻¹; at 2 hour was 683.58×10⁻⁹ mL⁻¹; at 2.5 hours (150 min) was 727.67×10⁻⁹ mL⁻¹; at 3 hours was 840.33×10⁻⁹ mL⁻¹×10⁻⁹ mL⁻¹; at 3.3 hours (200 min) was 923.58×10⁻⁹ mL⁻¹; at 3.67 hours (220 min) was 952.71×10⁻⁹ mL⁻¹; at 4 hours was 1012.35×10⁻⁹ mL⁻¹; at 4.33 hours (260 min) was 1030.10×10⁻⁹ mL⁻¹; at 4.67 hours (280 min) was 1038.58×10⁻⁹ mL⁻¹; at 5 hours was 990.90×10⁻⁹ mL⁻¹; at 5.5 hours (330 min) was 1046.42×10⁻⁹ mL⁻¹; at 6 hours was 911.07×10⁻⁹ mL⁻¹; at 8 hours was 696.33×10⁻⁹ mL⁻¹; at 12 hours was 504.90×10⁻⁹ mL⁻¹; at 16 hours was 354.04×10⁻⁹ mL⁻¹; at 24 hours was 294.40×10⁻⁹ mL⁻¹; at 36 hours was 184.19×10⁻⁹ mL⁻¹; at; at 48 hours was 143.37×10⁻⁹ mL⁻¹; and at 72 hours was 88.23. For example, dnC* can be measured 5 minutes after administration, 10 minutes after administration, 15 minutes after administration, 20 minutes after administration, 30 minutes after administration, 45 minutes after administration, 1 hour after administration, 2 hours after administration, 3 hours after administration, 4 hours after administration, 5 hours after administration, 6 hours after administration, 7 hours after administration, 8 hours after administration, 9 hours after administration, 10 hours after administration, 11 hours after administration, or 12 hours after administration. For example, a dnC* value may be about, or greater than about, 8.0±25%×10⁻⁷ mL⁻¹, 0.001±25%×10⁻⁶ mL⁻¹, 0.01±25%×10⁻⁶ mL⁻¹, 0.05±25%×10⁻⁶ mL⁻¹, 0.1±25%×10⁻⁶ mL⁻¹, 0.5±25%×10⁻⁶ mL⁻¹, 1.0±25%×10⁻⁶ mL⁻¹, 5.0±25%×10⁻⁶ mL⁻¹, 10.0±25%×10⁻⁶ mL⁻¹, 50.0±25%×10⁻⁶ mL⁻¹, or 100.0±25%×10⁻⁶ mL⁻¹, 125.0±25%×10⁻⁶ mL⁻¹, 150.0±25%×10⁻⁶ mL⁻¹, 175.0±25%×10⁻⁶ mL⁻¹, 200.0±25%×10⁻⁶ mL⁻¹, 300.0±25%×10⁻⁶ mL⁻¹, 400.0±25%×10⁻⁶ mL⁻¹, 500.0±25%×10⁻⁶ mL⁻¹, 600.0±25%×10⁻⁶ mL⁻¹ or 700.0±25%×10⁻⁶ mL⁻¹. For example, a dnC* value may be about, or greater than or equal to 50±25%×10⁻⁹ mL⁻¹ 10 minutes after administration, greater than or equal to 125±25%×10⁻⁹ mL⁻¹ 15 minutes after administration, greater than or equal to 150±25%×10⁻⁹ mL⁻¹ 20 minutes after administration, greater than or equal to 300±25%×10⁻⁹ mL⁻¹ 30 minutes after administration, greater than or equal to 450±25%×10⁻⁹ mL⁻¹ 45 minutes after administration, greater than or equal to 600±25%×10⁻⁹ mL⁻¹ 1 hour after administration, greater than or equal to 700±25%×10⁻⁹ mL⁻¹ 2 hours after administration, greater than or equal to 750±25%×10⁻⁹ mL⁻¹ 2.5 hours after administration, greater than or equal to 850±25%×10⁻⁹ mL⁻¹ 3 hours after administration, greater than or equal to 900±25%×10⁻⁹ mL⁻¹ 3.3 hours after administration, greater than or equal to 950±25%×10⁻⁹ mL⁻¹ 3.7 hours after administration, greater than or equal to 1000±25%×10⁻⁹ mL⁻¹ 4 hours after administration, greater than or equal to 1050±25%×10⁻⁹ mL⁻¹ 4.33 hours after administration, greater than or equal to 1050±25%×10⁻⁹ mL⁻¹ 4.67 hours after administration, less than or equal to 1000±25%×10⁻⁹ mL⁻¹ 5 hours after administration, less than or equal to 1000±25%×10⁻⁹ mL⁻¹ 5.5 hours after administration, less than or equal to 900±25%×10⁻⁹ mL⁻¹ 6 hours after administration, less than or equal to 700±25%×10⁻⁹ mL⁻¹ 8 hours after administration, less than or equal to 650±25%×10⁻⁹ mL⁻¹ 10 hours after administration, less than or equal to 500±25%×10⁻⁹ mL⁻¹ 12 hours after administration, less than or equal to 400±25%×10⁻⁹ mL⁻¹ 14 hours after administration, less than or equal to 350±25%×10⁻⁹ mL⁻¹ 16 hours after administration, less than or equal to 340±25%×10⁻⁹ mL⁻¹ 18 hours after administration, less than or equal to 320±25%×10⁻⁹ mL⁻¹ 20 hours after administration, less than or equal to 310±25%×10⁻⁹ mL⁻¹ 22 hours after administration, less than or equal to 300±25%×10⁻⁹ mL⁻¹ 24 hours after administration, less than or equal to 180±25%×10⁻⁹ mL⁻¹ 36 hours after administration, less than or equal to 140±25%×10⁻⁹ mL⁻¹ 48 hours after administration, or less than or equal to 90±25%×10⁻⁹ mL⁻¹ 72 hours after administration. In some embodiments, dnC* can be measured 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, or 36 hours after administration. For example, a dnC* value may be about, or less than about, 1.0±25%×10⁻⁹ mL⁻¹, 1.0±25%×10⁻⁸ mL⁻¹, 0.7±25%×10⁻⁷ mL⁻¹, 1.0±25%×10⁻⁷ mL⁻¹, 2.0±25%×10⁻⁷ mL⁻¹, 3.0±25%×10⁻⁷ mL⁻¹, 4.0±25%×10⁻⁷ mL⁻¹, 5.0±25%×10⁻⁷ mL⁻¹, 1.0±25%×10⁻⁶ mL⁻¹, or 5.0±25%×10⁻⁶ mL⁻¹ In some embodiments, the dnC* value can relate to single dosing. In some embodiments, the dnC* value can relate to a multi-dose regimen (e.g., repeated daily administration). In some embodiments, the plasma concentration used to calculate the dnC* may be adjusted to reflect a baseline plasma concentration (e.g., a baseline plasma level because of repeated daily administration). Cₘₐₓ is defined as the peak plasma concentration of a compound of the invention after administration. If a dnC* value is calculated at the time point corresponding to Cₘₐₓ, the value may alternatively be referred to as a dose normalized Cₘₐₓ or dnCₘₐₓ*. In some embodiments, a dnCₘₐₓ* is greater than or equal to 1.0±25%×10⁻⁶ mL⁻¹, greater than or equal to 1.5±25%×10⁻⁶ mL⁻¹, greater than or equal to 2.0±25%×10⁻⁶ mL⁻¹, greater than or equal to 2.5±25%×10⁻⁶ mL⁻¹, greater than or equal to 3.0±25%×10⁻⁶ mL⁻¹, greater than or equal to 3.5±25%×10⁻⁶ mL⁻¹, greater than or equal to 4.0±25%×10⁻⁶ mL⁻¹, greater than or equal to 4.5±25%×10⁻⁶ mL⁻¹, greater than or equal to 5.0±25%×10⁻⁶ mL⁻¹.

Transmucosal absorption also produces higher plasma concentrations of a compound as compared to oral administration. A plasma concentration may be an individual plasma concentration or a mean plasma concentration when observing multiple individuals. The higher plasma concentrations produced by transmucosal absorption may be determined by measuring the plasma concentration of the compound being administered or by calculating the ratio of the plasma concentration*body mass product and the dose administered, which is the dose- and body mass-normalized plasma concentration (C) or dbmnC*, measured in kg mL⁻¹. The dbmnC* is calculated by determining the ratio of plasma level times body mass product to dose administered. For example, if 2.4 mg of cyclobenzaprine or amitriptyline is administered to a 70 kg animal and the plasma level is 4.8 ng/mL at 15 minutes, the dbmnC* at 15 minutes is ((4.8 ng/mL)×(70 kg)/(2.4 mg))=dbmnC_{(0.25h)}*=140.0×10⁻⁶ kg mL⁻¹. The dbmnC* can be measured either at fixed time points or at a variable time point, e.g., the time point corresponding to Cₘₐₓ. For example, dbmnC* can be measured 5 minutes after administration, 10 minutes after administration, 15 minutes after administration, 30 minutes after administration, 45 minutes after administration, 1 hour after administration, 2 hours after administration, 3 hours after administration, 4 hours after administration, 5 hours after administration, 6 hours after administration, 7 hours after administration, 8 hours after administration, 9 hours after administration, 10 hours after administration, 11 hours after administration, or 12 hours after administration. For example, a dbmnC* value may be about, or greater than about, 80.0±25%×10⁻⁷ kg mL⁻¹, 0.01±25%×10⁻⁶ kg mL⁻¹, 0.1±25%×10⁻⁶ kg mL⁻¹, 0.5±25%×10⁻⁶ kg mL⁻¹, 1.0±25%×10⁻⁶ kg mL⁻¹, 5.0±25%×10⁻⁶ kg mL⁻¹, 10.0±25%×10⁻⁶ kg mL⁻¹, 50.0±25%×10⁻⁶ kg mL⁻¹, 100.0±25%×10⁻⁶ kg mL⁻¹, 500.0±25%×10⁻⁶ kg mL⁻¹, or 1000.0±25%×10⁻⁶ kg mL⁻¹, 1250.0±25%×10⁻⁶ kg mL⁻¹, 1500.0±25%×10⁻⁶ kg mL⁻¹, 1750.0±25%×10⁻⁶ kg mL⁻¹, 2000.0±25%×10⁻⁶ kg mL⁻¹, 3000.0±25%×10⁻⁶ kg mL⁻¹, 4000.0±25%×10⁻⁶ kg mL⁻¹, 5000.0±25%×10⁻⁶ kg mL⁻¹, 6000.0±25%×10⁻⁶ kg mL⁻¹ or 7000.0±25%×10⁻⁶ kg mL⁻¹. In some embodiments, dbmnC* can be measured 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, or 36 hours after administration. For example, a dbmnC* value may be about, or less than about, 1.0±25%×10⁻⁹ mL⁻¹, 1.0±25%×10⁻⁸ mL⁻¹, 0.7±25%×10⁻⁷ mL⁻¹, 1.0±25%×10⁻⁷ mL⁻¹, 2.0±25%×10⁻⁷ mL⁻¹, 3.0±25%×10⁻⁷ mL⁻¹, 4.0±25%×10⁻⁷ mL⁻¹, 5.0±25%×10⁻⁷ mL⁻¹, 1.0±25%×10⁻⁶ mL⁻¹, or 5.0±25%×10⁻⁶ mL⁻¹. In some embodiments, the dbmnC* value can relate to single dosing. In some embodiments, the dbmnC* value can relate to a multi-dose regimen (e.g., repeated daily administration). In some embodiments, the plasma concentration used to calculate the dbmnC* may be adjusted to reflect a baseline plasma concentration (e.g., a baseline plasma level because of repeated daily administration). Cₘₐₓ is defined as the peak plasma concentration of a compound of the invention after administration. If a dbmnC* value is calculated at the time point corresponding to Cₘₐₓ, the value may alternatively be referred to as a dose and body mass normalized Cₘₐₓ or dbmnCₘₐₓ*. In some embodiments, a dbmnCₘₐₓ* is greater than or equal to 10.0±25%×10⁻⁶ kg mL⁻¹, greater than or equal to 15±25%×10⁻⁶ kg mL⁻¹, greater than or equal to 20±25%×10⁻⁶ kg mL⁻¹, greater than or equal to 25±25%×10⁻⁶ kg mL⁻¹, greater than or equal to 30±25%×10⁻⁶ kg mL⁻¹, greater than or equal to 35±25%×10⁻⁶ kg mL⁻¹, greater than or equal to 40±25%×10⁻⁶ kg mL⁻¹, greater than or equal to 45±25%×10⁻⁶ kg mL⁻¹, greater than or equal to 50±25%×10⁻⁶ kg mL⁻¹. In some embodiments, a dbmnCₘₐₓ* is greater than or equal to 100.0±25%×10⁻⁶ kg mL⁻¹, greater than or equal to 150±25%×10⁻⁶ kg mL⁻¹, greater than or equal to 200±25%×10⁻⁶ kg mL⁻¹, greater than or equal to 250±25%×10⁻⁶ kg kg mL⁻¹, greater than or equal to 300±25%×10⁻⁶ mL⁻¹, greater than or equal to 350±25%×10⁻⁶ kg mL⁻¹, greater than or equal to 400±25%×10⁻⁶ kg mL⁻¹, greater than or equal to 450±25%×10⁻⁶ kg mL⁻¹, greater than or equal to 500±25%×10⁻⁶ kg mL⁻¹.

As described above, Cₘₐₓ is defined as the peak plasma concentration of a compound of the invention after administration. By administering a composition of the invention for transmucosal absorption, it is possible to obtain higher Cₘₐₓ values than if the composition were administered orally. In some embodiments, a composition affords a Cₘₐₓ of a compound greater than or equal to 10 ng/mL, greater than or equal to 11 ng/mL, greater than or equal to 12 ng/mL, greater than or equal to 13 ng/mL, greater than or equal to 14 ng/mL, greater than or equal to 15 ng/mL, greater than or equal to 16 ng/mL, greater than or equal to 17 ng/mL, greater than or equal to 18 ng/mL, greater than or equal to 19 ng/mL, greater than or equal to 20 ng/mL, greater than or equal to 21 ng/mL, greater than or equal to 22 ng/mL, greater than or equal to 23 ng/mL, greater than or equal to 24 ng/mL, greater than or equal to 25 ng/mL, greater than or equal to 26 ng/mL, greater than or equal to 27 ng/mL, greater than or equal to 28 ng/mL, greater than or equal to 29 ng/mL, greater than or equal to 30 ng/mL, greater than or equal to 31 ng/mL, greater than or equal to 32 ng/mL, greater than or equal to 33 ng/mL, greater than or equal to 34 ng/mL, greater than or equal to 35 ng/mL, greater than or equal to 36 ng/mL, greater than or equal to 37 ng/mL, greater than or equal to 38 ng/mL, greater than or equal to 39 ng/mL, greater than or equal to 40 ng/mL, greater than or equal to 50 ng/mL, greater than or equal to 60 ng/mL, greater than or equal to 70 ng/mL, greater than or equal to 80 ng/mL, greater than or equal to 90 ng/mL, greater than or equal to 100 ng/mL, greater than or equal to 120 ng/mL, greater than or equal to 140 ng/mL, greater than or equal to 160 ng/mL, greater than or equal to 180 ng/mL, or greater than or equal to 200 ng/mL.

A Cₘₐₓ may be measured either after administration of a first dose or after the administration of any dose of a composition of the invention. However, because the compositions and methods of the invention may be used to prolong a therapeutic regimen, plasma levels of a compound useful in the compositions and methods may not return to 0 between dosing (i.e., there may be a baseline level of a compound in circulation). Accordingly, a composition may afford a Cₘₐₓ that can be compared to a baseline level of the compound rather than taken as an absolute numerical value compared to a starting plasma concentration of 0 ng/mL. In some embodiments, a composition affords a Cₘₐₓ of a compound greater than or equal to 10 ng/mL, greater than or equal to 11 ng/mL, greater than or equal to 12 ng/mL, greater than or equal to 13 ng/mL, greater than or equal to 14 ng/mL, greater than or equal to 15 ng/mL, greater than or equal to 16 ng/mL, greater than or equal to 17 ng/mL, greater than or equal to 18 ng/mL, greater than or equal to 19 ng/mL, greater than or equal to 20 ng/mL, greater than or equal to 21 ng/mL, greater than or equal to 22 ng/mL, greater than or equal to 23 ng/mL, greater than or equal to 24 ng/mL, greater than or equal to 25 ng/mL, greater than or equal to 26 ng/mL, greater than or equal to 27 ng/mL, greater than or equal to 28 ng/mL, greater than or equal to 29 ng/mL, greater than or equal to 30 ng/mL, greater than or equal to 31 ng/mL, greater than or equal to 32 ng/mL, greater than or equal to 33 ng/mL, greater than or equal to 34 ng/mL, greater than or equal to 35 ng/mL, greater than or equal to 36 ng/mL, greater than or equal to 37 ng/mL, greater than or equal to 38 ng/mL, greater than or equal to 39 ng/mL, greater than or equal to 40 ng/mL, greater than or equal to 50 ng/mL, greater than or equal to 60 ng/mL, greater than or equal to 70 ng/mL, greater than or equal to 80 ng/mL, greater than or equal to 90 ng/mL, greater than or equal to 100 ng/mL, greater than or equal to 120 ng/mL, greater than or equal to 140 ng/mL, greater than or equal to 160 ng/mL, greater than or equal to 180 ng/mL, or greater than or equal to 200 ng/mL above a baseline level (e.g., plasma concentration) of the compound as measured immediately prior to a second administration. As used herein, “immediately prior to administration” means within 1 hour, 45 minutes, 30 minutes, 15 minutes, 10 minutes, 5 minutes, or 1 minute of administration.

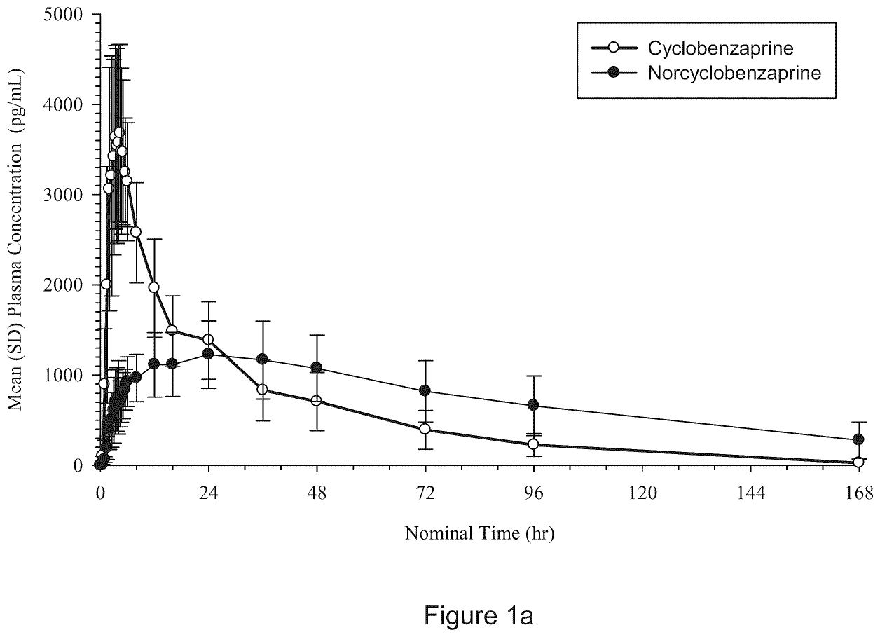

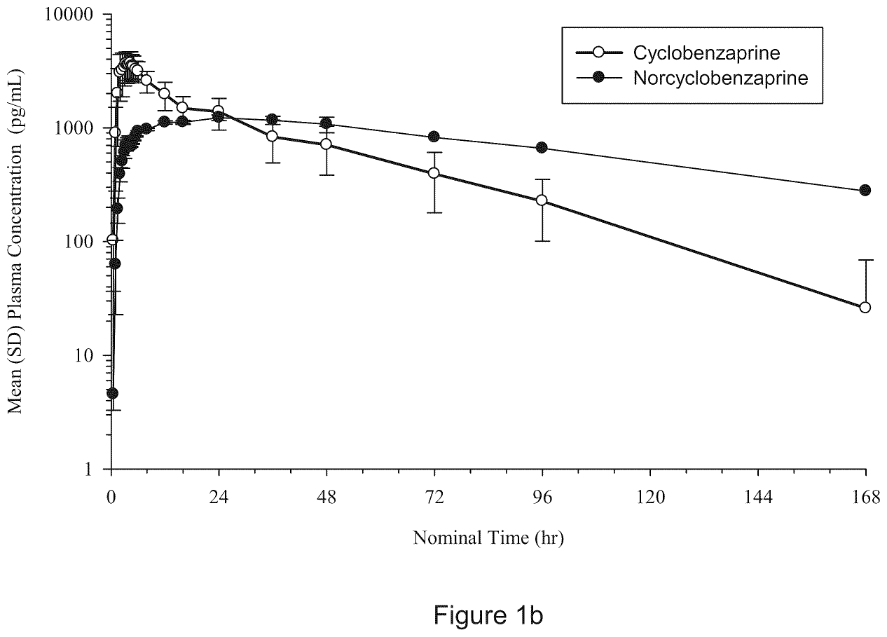

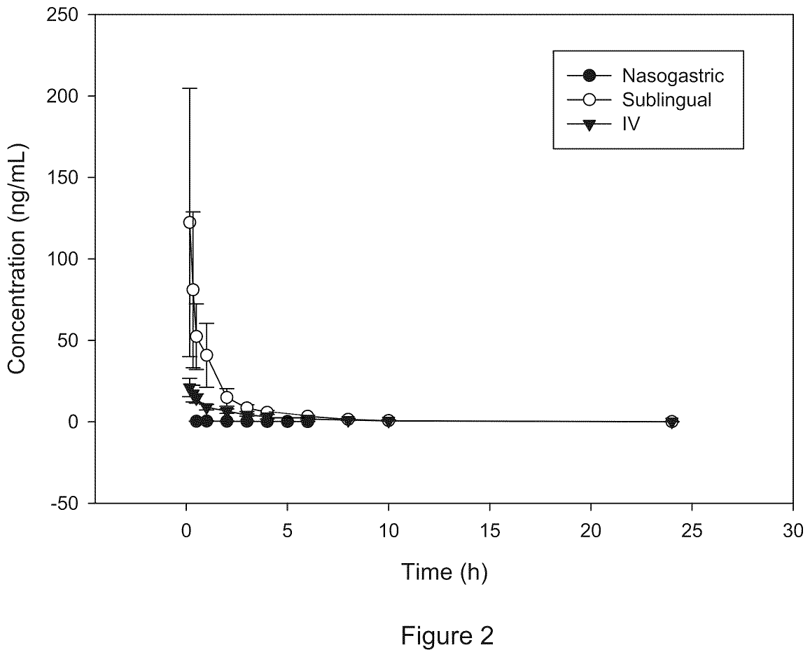

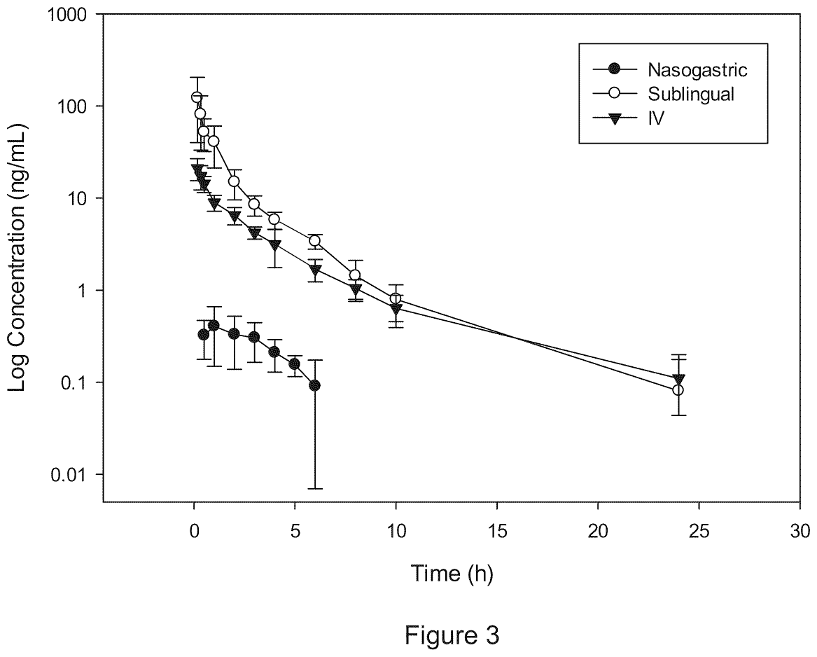

As a result of the higher Cₘₐₓ values achieved through sublingual administration, the area under the curve (AUC) for plasma concentration of a compound over time also is greater in comparison to the AUC afforded by an oral administration. AUC can be measured between two specific time points (e.g., AUC_{0-8 h}) or over an extrapolated period of time from 0 to infinity (AUC_{0-∞h}, AUC_{0-∞} or AUC_{inf}). AUC is typically given in units of ng hr mL⁻¹, so for example in the experiment on human subjects who received 5 mg immediate release cyclobenzaprine tablets in FIG. 1, the AUC_{0-∞h} was determined to be 103.1±35.8 ng hr mL⁻¹ and the AUC_{0-168 h} was 92.2±29.9 ng hr mL⁻¹. In another example, a dose of a 2.4 mg cyclobenzaprine sublingual tablet with basifying agent from 0 to 0.75 h resulted in an AUC_{0-0.75 h} of 135.6 ng hr mL⁻¹ in an experiment on Beagles. In the same experiment, for the sublingual tablet with basifying agent, the AUC_{0-∞h} was 179.0±50.2 ng hr mL⁻¹ and the AUC_{0-10 h} was 176.6±49.9 ng hr mL⁻¹. In the Beagle experiment, for the sublingual 2.4 mg tablet lacking the basifying agent, the AUC_{0-0.75 h} was 82.4 ng hr mL⁻¹, the AUC_{0-∞h} was 155.4±64.6 ng hr mL⁻¹ and the AUC_{0-10 h} was 151.6±64.0 ng hr mL⁻¹. In a Beagle experiment (FIGS. 2 and 3) for i.v. cyclobenzaprine at an average dose of 1.79 mg, the AUC_{0-∞h} was 44.9±4.15 ng hr mL⁻¹ and the AUC_{0-24 h} was 43.5±3.77 ng hr mL⁻¹. In a Beagle experiment (FIGS. 2 and 3) for sublingual solution cyclobenzaprine at an average dose of 1.79 mg, the AUC_{0-∞h} was 129.1±36.4 ng hr mL⁻¹ and the AUC_{0-24 h} was 126.9±37.1 ng hr mL⁻¹. By contrast, in an example from the literature, administering 2.5, 5.0 or 10 mg (10 mg in a 70 kg human being equivalent to 0.14 mg/kg in Beagles) cyclobenzaprine immediate release tablets to humans resulted in AUC_{0-8 h} of 11.1, 23.0, and 45.9 ng hr mL⁻¹, respectively and AUC_{0-∞h} of 44.2, 89.5, and 178.2 ng hr mL⁻¹, respectively (Winchell G. A. et al. “Cyclobenzaprine pharmacokinetics, including the effects of age, gender and hepatic insufficiency”, J. Clin. Pharmacol 2002 42:61). In some embodiments, for example, at a 2.4 mg dosing, the AUC₀₋₂₀min is about 0.04 ng hr mL⁻¹, AUC₀₋₃₀min is about 0.13 ng hr mL⁻¹, AUC₀₋₄₅min is about 0.33 ng hr mL⁻¹, AUC_{0-1 h} is about 0.61 ng hr mL⁻¹, AUC_{0-2 h} is about 2.10 ng hr mL⁻¹, AUC_{0-2.5 h} is about 2.95 ng hr mL⁻¹, AUC_{0-3 h} is about 3.93 ng hr mL⁻¹, AUC_{0-3.3 h} is about 4.66 ng hr mL⁻¹, AUC_{0-3.7 h} is about 5.46 ng hr mL⁻¹, AUC_{0-4 h} is about 6.27 ng hr mL⁻¹, AUC_{0-4.3 h} is about 7.12 ng hr mL⁻¹, AUC_{0-4.7 h} is about 7.99 ng hr mL⁻¹, AUC_{0-2.5 h} is about 8.81 ng hr mL⁻¹, AUC_{0-5.5 h} is about 10.06 ng hr mL⁻¹, AUC_{0-6 h} is about 11.27 ng hr mL⁻¹, AUC_{0-8 h} is about 15.11 ng hr mL⁻¹, AUC_{0-12 h} is about 50.30 ng hr mL⁻¹, AUC_{0-inf} is about 60.97 ng hr mL⁻¹. In some embodiments (with TNX-102 SL 2.8), the AUC₀₋₂₀min is about 0.04 ng hr mL⁻¹, AUC₀₋₃₀min is about 0.15 ng hr mL⁻¹, AUC₀₋₄₅min is about 0.39 ng hr mL⁻¹, AUC_{0-1 h} is about 0.72 ng hr mL⁻¹, AUC_{0-2 h} is about 2.45 ng hr mL⁻¹, AUC_{0-2.5 h} is about 3.45 ng hr mL⁻¹, AUC_{0-3 h} is about 4.59 ng hr mL⁻¹, AUC_{0-3.3 h} is about 5.44 ng hr mL⁻¹, AUC_{0-3.7 h} is about 6.37 ng hr mL⁻¹, AUC_{0-4 h} is about 7.32 ng hr mL⁻¹, AUC_{0-4.3 h} is about 8.30 ng hr mL⁻¹, AUC_{0-4.7 h} is about 9.32 ng hr mL⁻¹, AUC_{0-2.5 h} is about 10.27 ng hr mL⁻¹, AUC_{0-5.5 h} is about 11.74 ng hr mL⁻¹, AUC_{0-6 h} is about 13.14 ng hr mL⁻¹, AUC_{0-8 h} is about 17.63 ng hr mL⁻¹, AUC_{0-12 h} is about 58.68 ng hr mL⁻¹, AUC_{0-inf} is about 71.13 ng hr mL⁻¹. AUC also can be compared to the dose administered to generate an AUC to dose ratio which is sometimes referred to as dose normalized AUC, or dnAUC. The dose normalized dnAUC_{0-∞h} for the human data described above and in FIG. 1 is 20.6×10⁻⁶ hr mL⁻¹. The dose normalized AUC_{0-0.75 h} (dnAUC_{0-0.75 h}) for the Beagle data described above is dnAUC_{0-0.75 h}=135.6±6 ng hr mL⁻¹/2.4 mg=56.5×10⁻⁶ hr mL⁻¹. In a Beagle experiment (FIGS. 2 and 3) for IV cyclobenzaprine at an average dose of 1.79 mg, the dnAUC_{0-∞h} was 25.04×10⁻⁶ hr mL⁻¹ and the dnAUC_{0-24 h} was 24.2×10⁻⁶ hr mL⁻¹. In a Beagle experiment (FIGS. 2 and 3) for sublingual cyclobenzaprine in solution at an average dose of 1.79 mg, the dnAUC_{0-∞h} was 71.95×10⁻⁶ hr mL⁻¹ and the dnAUC_{0-24 h} was 70.72×10⁻⁶ hr mL⁻¹. The dose normalized AUC_{0-8 h} (dnAUC_{0-8 h}) for the human data described above (from Winchell et al) is 4.4×10⁻⁶ hr mL⁻¹, 4.6×10⁻⁶ hr mL⁻¹ and 4.6×10⁻⁶ hr mL⁻¹ for the 2.5, 5.0 and 10 mg cyclobenzaprine doses, respectively. The dose normalized dnAUC_{0-∞h} for the human data described above (from Winchell et al.) is 17.7×10⁻⁶ hr mL⁻¹, 17.9×10⁻⁶ hr mL⁻¹, and 17.8×10⁻⁶ hr mL⁻¹ for the 2.5, 5.0 and 10 mg cyclobenzaprine doses, respectively. In some embodiments, the dnAUC₀₋₂₀min is about 0.02±25%×10⁻⁶ hr mL⁻¹, the dnAUC₀₋₃₀min is about 0.05±25%×10⁻⁶ hr mL⁻¹, the dnAUC₀₋₄₅min is about 0.15±25%×10⁻⁶ hr mL⁻¹, the dnAUC_{0-1 h} is about 0.25±25%×10⁻⁶ hr mL⁻¹, the dnAUC_{0-2 h} is about 0.90±25%×10⁻⁶ hr mL⁻¹, the dnAUC_{0-2.5 h} is about 1.2±25%×10⁻⁶ hr mL⁻¹, the dnAUC_{0-3 h} is about 1.6±25%×10⁻⁶ hr mL⁻¹, the dnAUC_{3.3h} is about 1.8±25%×10⁻⁶ hr mL⁻¹, dnAUC_{0-3.7 h} is about 2.3±25%×10⁻⁶ hr mL⁻¹, the dnAUC_{0-4 h} is about 2.6±25%×10⁻⁶ hr mL⁻¹, the dnAUC_{0-4.3 h} is about 3.0±25%×10⁻⁶ hr mL⁻¹, the dnAUC_{0-4.7 h} is about 3.3±25%×10⁻⁶ hr mL⁻¹, the dnAUC_{0-5 h} is about 3.7±25%×10⁻⁶ hr mL⁻¹, the dnAUC_{0-5.5 h} is about 4.2±25%×10⁻⁶ hr mL⁻¹, the dnAUC_{0-6 h} is about 4.7±25%×10⁻⁶ hr mL⁻¹, the dnAUC_{0-8 h} is 6.3±25%×10⁻⁶ hr mL⁻¹, the dnAUC_{0-12 h} is about 20±25%×10⁻⁶ hr mL⁻¹, and the dnAUC_{0-∞h} is about 25±±25%×10⁻⁶ hr mL⁻¹. In some embodiments, a dnAUC_{0-8 h} is greater than or equal to 5±25%×10⁻⁶ hr mL⁻¹, greater than or equal to 6±25%×10⁻⁶ hr mL⁻¹, greater than or equal to 7±25%×10⁻⁶ hr mL⁻¹, greater than or equal to 8±25%×10⁻⁶ hr mL⁻¹, greater than or equal to 9±25%×10⁻⁶ hr mL⁻¹, greater than or equal to 10±25%×10⁻⁶ hr mL⁻¹, greater than or equal to 11±25%×10⁻⁶ hr mL⁻¹, greater than or equal to 12±25%×10⁻⁶ hr mL⁻¹, greater than or equal to 13±25%×10⁻⁶ hr mL⁻¹, greater than or equal to 14±25%×10⁻⁶ hr mL⁻¹, greater than or equal to 15±25%×10⁻⁶ hr mL⁻¹, greater than or equal to 16±25%×10⁻⁶ hr mL⁻¹, greater than or equal to 17±25%×10⁻⁶ hr mL⁻¹, greater than or equal to 18±25%×10⁻⁶ hr mL⁻¹, or greater than or equal to 19±25%×10⁻⁶ hr mL⁻¹. In some embodiments, a dnAUC_{0-8 h} is greater than or equal to 20±25%×10⁻⁶ hr mL⁻¹, greater than or equal to 22±25%×10⁻⁶ hr mL⁻¹, greater than or equal to 24±25%×10⁻⁶ hr mL⁻¹, greater than or equal to 26±25%×10⁻⁶ hr mL⁻¹, greater than or equal to 28±25%×10⁻⁶ hr mL⁻¹, or greater than or equal to 30±25%×10⁻⁶ hr mL⁻¹. In some embodiments, a dnAUC_{0-8 h} is greater than or equal to 40±25%×10⁻⁶ hr mL⁻¹, greater than or equal to 50±25%×10⁻⁶ hr mL⁻¹, greater than or equal to 60±25%×10⁻⁶ hr mL⁻¹, greater than or equal to 70±25%×10⁻⁶ hr mL⁻¹, greater than or equal to 80±25%×10⁻⁶ hr mL⁻¹, or greater than or equal to 90±±25%×10⁻⁶ hr mL⁻¹. In some embodiments, a dnAUC_{0-8 h} is greater than or equal to 100±25%×10⁻⁶ hr mL⁻¹, greater than or equal to 120±25%×10⁻⁶ hr mL⁻¹, greater than or equal to 140±25%×10⁻⁶ hr mL⁻¹, greater than or equal to 160±25%×10⁻⁶ hr mL⁻¹, greater than or equal to 180±25%×10⁻⁶ hr mL⁻¹, or greater than or equal to 200±25%×10⁻⁶ hr mL⁻¹. In some embodiments, a dnAUC_{0-10 h} is greater than or equal to 5±25%×10⁻⁶ hr mL⁻¹, greater than or equal to 6±25%×10⁻⁶ hr mL⁻¹, greater than or equal to 7±25%×10⁻⁶ hr mL⁻¹, greater than or equal to 8±25%×10⁻⁶ hr mL⁻¹, greater than or equal to 9±25%×10⁻⁶ hr mL⁻¹, greater than or equal to 10±25%×10⁻⁶ hr mL⁻¹, greater than or equal to 11±25%×10⁻⁶ hr mL⁻¹, greater than or equal to 12±25%×10⁻⁶ hr mL⁻¹, greater than or equal to 13±25%×10⁻⁶ hr mL⁻¹, greater than or equal to 14±25%×10⁻⁶ hr mL⁻¹, greater than or equal to 15±25%×10⁻⁶ hr mL⁻¹, greater than or equal to 16±25%×10⁻⁶ hr mL⁻¹, greater than or equal to 17±25%×10⁻⁶ hr mL⁻¹, greater than or equal to 18±25%×10⁻⁶ hr mL⁻¹, or greater than or equal to 19±25%×10⁻⁶ hr mL⁻¹. In some embodiments, a dnAUC_{0-10 h} is greater than or equal to 20±25%×10⁻⁶ hr mL⁻¹, greater than or equal to 22±25%×10⁻⁶ hr mL⁻¹, greater than or equal to 24±25%×10⁻⁶ hr mL⁻¹, greater than or equal to 26±25%×10⁻⁶ hr mL⁻¹, greater than or equal to 28±25%×10⁻⁶ hr mL⁻¹, or greater than or equal to 30±25%×10⁻⁶ hr mL⁻¹. In some embodiments, a dnAUC_{0-10 h} is greater than or equal to 40±25%×10⁻⁶ hr mL⁻¹, greater than or equal to 50±25%×10⁻⁶ hr mL⁻¹, greater than or equal to 60±25%×10⁻⁶ hr mL⁻¹, greater than or equal to 70±25%×10⁻⁶ hr mL⁻¹, greater than or equal to 80±25%×10⁻⁶ hr mL⁻¹, or greater than or equal to 90±25%×10⁻⁶ hr mL⁻¹. In some embodiments, a dnAUC_{0-10 h} is greater than or equal to 100±25%×10⁻⁶ hr mL⁻¹, greater than or equal to 120±25%×10⁻⁶ hr mL⁻¹, greater than or equal to 140±25%×10⁻⁶ hr mL⁻¹, greater than or equal to 160±25%×10⁻⁶ hr mL⁻¹, greater than or equal to 180±25%×10⁻⁶ hr mL⁻¹, or greater than or equal to 200±25%×10⁻⁶ hr mL⁻¹. In some embodiments, a dnAUC_{0-12 h} is greater than or equal to 20±25%×10⁻⁶ hr mL⁻¹, greater than or equal to 30±25%×10⁻⁶ hr mL⁻¹, greater than or equal to 40±25%×10⁻⁶ hr mL⁻¹, greater than or equal to 50±25%×10⁻⁶ hr mL⁻¹, greater than or equal to 60±25%×10⁻⁶ hr mL⁻¹, or greater than or equal to 70±25%×10⁻⁶ hr mL⁻¹. In some embodiments, a dnAUC_{0-12 h} is greater than or equal to 80±25%×10⁻⁶ hr mL⁻¹, greater than or equal to 90±25%×10⁻⁶ hr mL⁻¹, greater than or equal to 100±25%×10⁻⁶ hr mL⁻¹, greater than or equal to 120±25%×10⁻⁶ hr mL⁻¹, greater than or equal to 160±25%×10⁻⁶ hr mL⁻¹, or greater than or equal to 180±25%×10⁻⁶ hr mL⁻¹. In some embodiments, a dnAUC_{0-24 h} is greater than or equal to 24±25%×10⁻⁶ hr mL⁻¹, or greater than or equal to 25±25%×10⁻⁶ hr mL⁻¹, greater than or equal to 30±25%×10⁻⁶ hr mL⁻¹, greater than or equal to 35±25%×10⁻⁶ hr mL⁻¹, greater than or equal to 40±25%×10⁻⁶ hr mL⁻¹, greater than or equal to 50±25%×10⁻⁶ hr mL⁻¹, greater than or equal to 60±25%×10⁻⁶ hr mL⁻¹, greater than or equal to 70±25%×10⁻⁶ hr mL⁻¹, greater than or equal to 80±25%×10⁻⁶ hr mL⁻¹, or greater than or equal to 90±25%×10⁻⁶ hr mL⁻¹. In some embodiments, dnAUC_{0-24 h} is greater than or equal to 100±25%×10⁻⁶ hr mL⁻¹, greater than or equal to 110±25%×10⁻⁶ hr mL⁻¹, greater than or equal to 120±25%×10⁻⁶ hr mL⁻¹, greater than or equal to 130±25%×10⁻⁶ hr mL⁻¹, greater than or equal to 140±25%×10⁻⁶ hr mL⁻¹, or greater than or equal to 150±25%×10⁻⁶ hr mL⁻¹. In some embodiments, a dnAUC_{0-24 h} is greater than or equal to 160±25%×10⁻⁶ hr mL⁻¹, greater than or equal to 170±25%×10⁻⁶ hr mL⁻¹, greater than or equal to 180±25%×10⁻⁶ hr mL⁻¹, greater than or equal to 190±25%×10⁻⁶ hr mL⁻¹, greater than or equal to 200±25%×10⁻⁶ hr mL⁻¹, or greater than or equal to 210±25%×10⁻⁶ hr mL⁻¹. In some embodiments, a dnAUC_{0-24 h} is greater than or equal to 220±25%×10⁻⁶ hr mL⁻¹, greater than or equal to 240±25%×10⁻⁶ hr mL⁻¹, greater than or equal to 250±25%×10⁻⁶ hr mL⁻¹, greater than or equal to 260±25%×10⁻⁶ hr mL⁻¹, greater than or equal to 270±25%×10⁻⁶ hr mL⁻¹, or greater than or equal to 280±25%×10⁻⁶ hr mL⁻¹. In some embodiments, a dnAUC_{0-∞h} is greater than or equal to 24±25%×10⁻⁶ hr mL⁻¹, or greater than or equal to 25±25%×10⁻⁶ hr mL⁻¹, greater than or equal to 30±25%×10⁻⁶ hr mL⁻¹, greater than or equal to 35±25%×10⁻⁶ hr mL⁻¹, greater than or equal to 40±25%×10⁻⁶ hr mL⁻¹, greater than or equal to 50±25%×10⁻⁶ hr mL⁻¹, greater than or equal to 60±25%×10⁻⁶ hr mL⁻¹, greater than or equal to 70±25%×10⁻⁶ hr mL⁻¹, greater than or equal to 80±25%×10⁻⁶ hr mL⁻¹, or greater than or equal to 90±25%×10⁻⁶ hr mL⁻¹. In some embodiments, dnAUC_{0-∞h} is greater than or equal to 100±25%×10⁻⁶ hr mL⁻¹, greater than or equal to 110±25%×10⁻⁶ hr mL⁻¹, greater than or equal to 120±25%×10⁻⁶ hr mL⁻¹, greater than or equal to 130±25%×10⁻⁶ hr mL⁻¹, greater than or equal to 140±25%×10⁻⁶ hr mL⁻¹, or greater than or equal to 150±25%×10⁻⁶ hr mL⁻¹. In some embodiments, a dnAUC_{0-∞h} is greater than or equal to 160±25%×10⁻⁶ hr mL⁻¹, greater than or equal to 170±25%×10⁻⁶ hr mL⁻¹, greater than or equal to 180±25%×10⁻⁶ hr mL⁻¹, greater than or equal to 190±25%×10⁻⁶ hr mL⁻¹, greater than or equal to 200±25%×10⁻⁶ hr mL⁻¹, or greater than or equal to 210±25%×10⁻⁶ hr mL⁻¹. In some embodiments, a dnAUC_{0-∞h} is greater than or equal to 220±25%×10⁻⁶ hr mL⁻¹, greater than or equal to 240±25%×10⁻⁶ hr mL⁻¹, greater than or equal to 250±25%×10⁻⁶ hr mL⁻¹, greater than or equal to 260±25%×10⁻⁶ hr mL⁻¹, greater than or equal to 270±25%×10⁻⁶ hr mL⁻¹, or greater than or equal to 280±25%×10⁻⁶ hr mL⁻¹.

The product of AUC and body mass also can be compared to the dose administered to generate a ratio between the AUC times body mass product to dose which is herein referred to as dose- and body mass-normalized AUC, or dbmnAUC. The dose- and body mass-normalized dbmnAUC_{0-∞h} for the human data described above and in FIG. 1 is approximately, for 70 kg humans, 140.6×10⁻⁶ kg hr mL⁻¹. The dose- and body mass-normalized AUC_{0-0.75 h} (dbmnAUC_{0-0.75 h}) for the Beagle data described above (average body mass of Beagles was 12.5 kg) is dbmnAUC_{0.0.75 h}=12.5 kg×135.6 ng hr mL⁻¹/2.4 mg=708×10⁻⁶ kg hr mL⁻¹. In a Beagle experiment (FIGS. 2 and 3) for IV cyclobenzaprine at an average dose of 1.79 mg, the dbmnAUC_{0-∞h} was 12.5 kg×25.04×10⁻⁶ hr mL⁻¹ and the dbmnAUC_{0-24 h} was 314×10⁻⁶ kg hr mL⁻¹. In a Beagle experiment (FIGS. 2 and 3) for sublingual cyclobenzaprine in solution at an average dose of 1.79 mg, the dbmnAUC_{0-∞h} was 12.5 kg×71.95×10⁻⁶ hr mL⁻¹ and the dbmnAUC_{0-24 h} was 886×10⁻⁶ kg hr mL⁻¹. The dbmnAUC_{0-8 h} for the human data described above (from Winchell et al.), assuming 70 kg humans, is approximately 70 kg×4.4±25%×10⁻⁶ hr mL⁻¹=308±25%×10⁻⁶ kg hr mL⁻¹, 322±25%×10⁻⁶ kg hr mL⁻¹ and 322±25%×10⁻⁶ kg hr mL⁻¹ for the 2.5, 5.0 and 10 mg cyclobenzaprine doses, respectively. The dose normalized dbmnAUC_{0-∞h} for the human data described above (from Winchell et al.) is 70 kg×17.7±25%×10⁻⁶ hr mL⁻¹=1239±25%×10⁻⁶ kg hr mL⁻¹, 1253±25%×10⁻⁶ kg hr mL⁻¹, and 1246±25%×10⁻⁶ kg hr mL⁻¹ for the 2.5, 5.0 and 10 mg cyclobenzaprine doses, respectively. In some embodiments, the dbmnAUC₀₋₂₀min is about 1.1±25%×10⁻⁶ kg hr mL⁻¹, the dbmnAUC₀₋₃₀min is about 3.7±25%×10⁻⁶ kg hr mL⁻¹, the dbmnAUC₀₋₄₅min is about 9.7±25%×10⁻⁶ kg hr mL⁻¹, the dbmnAUC_{0-1 h} is about 18±25%×10⁻⁶ kg hr mL⁻¹, the dbmnAUC_{0-2 h} is about 62±25%×10⁻⁶ kg hr mL⁻¹, the dbmnAUC_{0-2.5 h} is about 86±25%×10⁻⁶ kg hr mL⁻¹, the dbmnAUC_{0-3 h} is about 115±25%×10⁻⁶ kg hr mL⁻¹, the dbmnAUC_{3.3h} is about 135±25%×10⁻⁶ kg hr mL⁻¹, dbmnAUC_{0-3.7 h} is about 160±25%×10⁻⁶ kg hr mL⁻¹, the dbmnAUC_{0-4 h} is about 180±25%×10⁻⁶ kg hr mL⁻¹, the dbmnAUC_{0-4.3 h} is about 210±25%×10⁻⁶ kg hr mL⁻¹, the dbmnAUC_{0-4.7 h} is about 230±25%×10⁻⁶ kg hr mL⁻¹, the dbmnAUC_{0-5 h} is about 260±25%×10⁻⁶ kg hr mL⁻¹, the dbmnAUC_{0-5.5 h} is about 290±25%×10⁻⁶ kg hr mL⁻¹, the dbmnAUC_{0-6 h} is about 330±25%×10⁻⁶ kg hr mL⁻¹, the dbmnAUC_{0-8 h} is 440±25%×10⁻⁶ kg hr mL⁻¹, the dbmnAUC_{0-12 h} is about 1500±25%×10⁻⁶ kg hr mL⁻¹, and the dbmnAUC_{0-inf} is about 1800±25%×10⁻⁶ kg hr mL⁻¹. In some embodiments, a dbmnAUC_{0-8 h} is greater than or equal to 350±25%×10⁻⁶ kg hr mL⁻¹. In some embodiments, a dbmnAUC_{0-8 h} is greater than or equal to 400±25%×10⁻⁶ hr mL⁻¹, greater than or equal to 500±25%×10⁻⁶ kg hr mL⁻¹, greater than or equal to 600±25%×10⁻⁶ kg hr mL⁻¹, greater than or equal to 700±25%×10⁻⁶ kg hr mL⁻¹, greater than or equal to 800±25%×10⁻⁶ kg hr mL⁻¹, or greater than or equal to 900±25%×10⁻⁶ kg hr mL⁻¹. In some embodiments, a dbmnAUC_{0-8 h} is greater than or equal to 1000±25%×10⁻⁶ kg hr mL⁻¹, greater than or equal to 1200±25%×10⁻⁶ kg hr mL⁻¹, greater than or equal to 1400±±25%×10⁻⁶ kg hr mL⁻¹, greater than or equal to 1600±25%×10⁻⁶ kg hr mL⁻¹, greater than or equal to 1800±25%×10⁻⁶ kg hr mL⁻¹, or greater than or equal to 2000±25%×10⁻⁶ kg hr mL⁻¹. In some embodiments, a dbmnAUC_{0-10 h} is greater than or equal to 400±25%×10⁻⁶ kg hr mL⁻¹, greater than or equal to 500±25%×10⁻⁶ kg hr mL⁻¹, greater than or equal to 600±25%×10⁻⁶ kg hr mL⁻¹, greater than or equal to 700±25%×10⁻⁶ kg hr mL⁻¹, greater than or equal to 800±25%×10⁻⁶ kg hr mL⁻¹, or greater than or equal to 900±25%×10⁻⁶ kg hr mL⁻¹. In some embodiments, a dbmnAUC_{0-10 h} is greater than or equal to 1000±25%×10⁻⁶ kg hr mL⁻¹, greater than or equal to 1200±25%×10⁻⁶ kg hr mL⁻¹, greater than or equal to 1400±25%×10⁻⁶ kg hr mL⁻¹, greater than or equal to 1600±25%×10⁻⁶ kg hr mL⁻¹, greater than or equal to 1800±25%×10⁻⁶ kg hr mL⁻¹, or greater than or equal to 2000±25%×10⁻⁶ kg hr mL⁻¹. In some embodiments, a dbmnAUC_{0-12 h} is greater than or equal to 500±25%×10⁻⁶ kg hr mL⁻¹, greater than or equal to 600±25%×10⁻⁶ kg hr mL⁻¹, greater than or equal to 700±25%×10⁻⁶ kg hr mL⁻¹, greater than or equal to 800±25%×10⁻⁶ kg hr mL⁻¹, or greater than or equal to 900±25%×10⁻⁶ kg hr mL⁻¹. In some embodiments, a dbmnAUC_{0-12 h} is greater than or equal to 1000±25%×10⁻⁶ kg hr mL⁻¹, greater than or equal to 1200±25%×10⁻⁶ kg hr mL⁻¹, greater than or equal to 1400±25%×10⁻⁶ kg hr mL⁻¹, greater than or equal to 1600±25%×10⁻⁶ kg hr mL⁻¹, greater than or equal to 1800±25%×10⁻⁶ kg hr mL⁻¹, or greater than or equal to 2000±25%×10⁻⁶ kg hr mL⁻¹. In some embodiments, a dbmnAUC_{0-24 h} is greater than or equal to 500±25%×10⁻⁶ kg hr mL⁻¹, greater than or equal to 600±25%×10⁻⁶ kg hr mL⁻¹, greater than or equal to 700±25%×10⁻⁶ kg hr mL⁻¹, greater than or equal to 800±25%×10⁻⁶ kg hr mL⁻¹, or greater than or equal to 900±25%×10⁻⁶ kg hr mL⁻¹. In some embodiments, dbmnAUC_{0-24 h} is greater than or equal to 1000±25%×10⁻⁶ kg mL⁻¹, greater than or equal to 1100±25%×10⁻⁶ hr mL⁻¹, greater than or equal to 1200±25%×10⁻⁶ kg hr mL⁻¹, greater than or equal to 1300±25%×10⁻⁶ kg hr mL⁻¹, greater than or equal to 1400±25%×10⁻⁶ kg hr mL⁻¹, or greater than or equal to 1500±25%×10⁻⁶ kg hr mL⁻¹. In some embodiments, a dbmnAUC₀₋₂₄ₕ is greater than or equal to 1600±25%×10⁻⁶ kg hr mL⁻¹, greater than or equal to 1700±25%×10⁻⁶ kg hr mL⁻¹, greater than or equal to 1800±25%×10⁻⁶ kg hr mL⁻¹, greater than or equal to 1900±25%×10⁻⁶ kg hr mL⁻¹, greater than or equal to 2000±25%×10⁻⁶ kg hr mL⁻¹, or greater than or equal to 2100±25%×10⁻⁶ kg hr mL⁻¹. In some embodiments, a dbmnAUC_{0-24 h} is greater than or equal to 2200±25%×10⁻⁶ kg hr mL⁻¹, greater than or equal to 2400±25%×10⁻⁶ kg hr mL⁻¹, greater than or equal to 2500±25%×10⁻⁶ kg hr mL⁻¹, greater than or equal to 2600±25%×10⁻⁶ kg hr mL⁻¹, greater than or equal to 2700±25%×10⁻⁶ kg hr mL⁻¹, or greater than or equal to 2800±25%×10⁻⁶ kg hr mL⁻¹. In some embodiments, a dnAUC_{0-∞h} is greater than or equal to 240±25%×10⁻⁶ kg hr mL⁻¹, or greater than or equal to 250±25%×10⁻⁶ kg hr mL⁻¹, greater than or equal to 300±25%×10⁻⁶ kg hr mL⁻¹, greater than or equal to 35±25%×10⁻⁶ kg hr mL⁻¹, greater than or equal to 400±25%×10⁻⁶ kg hr mL⁻¹, greater than or equal to 500±25%×10⁻⁶ kg hr mL⁻¹, greater than or equal to 600±25%×10⁻⁶ kg hr mL⁻¹, greater than or equal to 700±25%×10⁻⁶ kg hr mL⁻¹, greater than or equal to 800±±25%×10⁻⁶ kg hr mL⁻¹, or greater than or equal to 900±25%×10⁻⁶ kg hr mL⁻¹. In some embodiments, dbmnAUC_{0-∞h} is greater than or equal to 1000±25%×10⁻⁶ kg hr mL⁻¹, greater than or equal to 1100±25%×10⁻⁶ kg hr mL⁻¹, greater than or equal to 1200±25%×10⁻⁶ kg hr mL⁻¹, greater than or equal to 1300±25%×10⁻⁶ kg hr mL⁻¹, greater than or equal to 1400±25%×10⁻⁶ kg hr mL⁻¹, or greater than or equal to 1500±25%×10⁻⁶ kg hr mL⁻¹. In some embodiments, a dbmnAUC_{0-∞h} is greater than or equal to 1600±25%×10⁻⁶ kg hr mL⁻¹, greater than or equal to 1700±25%×10⁻⁶ kg hr mL⁻¹, greater than or equal to 1800±25%×10⁻⁶ kg hr mL⁻¹, greater than or equal to 1900±25%×10⁻⁶ kg hr mL⁻¹, greater than or equal to 2000±25%×10⁻⁶ kg hr mL⁻¹, or greater than or equal to 2100±25%×10⁻⁶ kg hr mL⁻¹. In some embodiments, a dbmnAUC_{0-∞h} is greater than or equal to 2200±25%×10⁻⁶ kg hr mL⁻¹, greater than or equal to 2400±25%×10⁻⁶ kg hr mL⁻¹, greater than or equal to 250±25%×10⁻⁶ kg hr mL⁻¹, greater than or equal to 2600±25%×10⁻⁶ kg hr mL⁻¹, greater than or equal to 2700±25%×10⁻⁶ hr mL⁻¹, or greater than or equal to 2800±25%×10⁻⁶ kg hr mL⁻¹. In some embodiments, a dbmnAUC_{0-∞h} is greater than or equal to 5000±25%×10⁻⁶ kg hr mL⁻¹, greater than or equal to 10000±25%×10⁻⁶ kg hr mL⁻¹, greater than or equal to 15000±25%×10⁻⁶ kg hr mL⁻¹, greater than or equal to 20000±25%×10⁻⁶ kg hr mL⁻¹, greater than or equal to 25000±25%×10⁻⁶ kg hr mL⁻¹, or greater than or equal to 30000±25%×10⁻⁶ kg hr mL⁻¹. In some embodiments, a dbmnAUC_{0-∞h} is greater than or equal to 35000±25%×10⁻⁶ kg hr mL⁻¹, greater than or equal to 40000±25%×10⁻⁶ kg hr mL⁻¹, greater than or equal to 45000±25%×10⁻⁶ kg hr mL⁻¹, greater than or equal to 50000±25%×10⁻⁶ kg hr mL⁻¹, greater than or equal to 55000±25%×10⁻⁶ hr mL⁻¹, or greater than or equal to 60000±25%×10⁻⁶ kg hr mL⁻¹.

In some embodiments, a composition of the invention is one that produces a bioequivalent effect to the compositions described herein. Bioequivalence may be determined by AUC, Cₘₐₓ, tₘₐₓ, mean absorption time, metabolite plasma concentration, mean residence time, rate constants, rate profiles, and Cₘₐₓ normalized to AUC. An exemplary test for bioequivalence is a confidence interval for Cₘₐₓ and/or AUC that is approximately 80%, 85%, 90%, 91%, 92%, 93%, 94%, 95%, 96%, 97%, 98%, 99%, 100%, 101%, 102%, 103%, 104%, 105%, 106%, 107%, 108%, 109%, 110%, 115%, 120%, or 125% of a given compound.

In some embodiments, a method of the invention is one that produces a bioequivalent effect to the compositions described herein. Bioequivalence may be determined by AUC, Cₘₐₓ, tₘₐₓ, mean absorption time, metabolite plasma concentration, mean residence time, rate constants, rate profiles, and Cₘₐₓ normalized to AUC. An exemplary test for bioequivalence is a confidence interval for Cₘₐₓ and/or AUC that is approximately 80%, 85%, 90%, 91%, 92%, 93%, 94%, 95%, 96%, 97%, 98%, 99%, 100%, 101%, 102%, 103%, 104%, 105%, 106%, 107%, 108%, 109%, 110%, 115%, 120%, or 125% of a given compound.

In some embodiments, the methods and compositions of the invention allow an administered compound including biologically active metabolites of the compound to be removed from the plasma more quickly than if the compound was administered orally. This is beneficial because the clearance of a compound can aid in the reduction of side effects. For example, if a subject takes a sublingual composition comprising cyclobenzaprine or amitriptyline before going to sleep, the cyclobenzaprine or amitriptyline may be rapidly absorbed but be substantially metabolized and excreted by the time the subject wakes up, minimizing fatigue, somnolence and grogginess felt upon waking. In some embodiments, a plasma level of a compound decreases by at least 50% of the Cₘₐₓ by 4 hours after administration, 5 hours after administration, 6 hours after administration, 7 hours after administration, 8 hours after administration, 9 hours after administration, 10 hours after administration, 11 hours after administration, 12 hours after administration, 13 hours after administration, 14 hours after administration, or 15 hours after administration. In some embodiments, a plasma level of a compound decreases by at least 50% of the Cₘₐₓ by 4 hours after tₘₐₓ, by at least 55% of the Cₘₐₓ by 4 hours after tₘₐₓ, by at least 60% of the Cₘₐₓ by 4 hours after tₘₐₓ, by at least 65% of the Cₘₐₓ by 4 hours after tₘₐₓ, by at least 70% of the Cₘₐₓ by 4 hours after tₘₐₓ, by at least 75% of the Cₘₐₓ by 4 hours after tₘₐₓ, by at least 80% of the Cₘₐₓ by 4 hours after tₘₐₓ, by at least 85% of the Cₘₐₓ by 4 hours after tₘₐₓ, by at least 90% of the Cₘₐₓ by 4 hours after tₘₐₓ, by at least 91% of the Cₘₐₓ by 4 hours after tₘₐₓ, by at least 92% of the Cₘₐₓ by 4 hours after tₘₐₓ, by at least 93% of the Cₘₐₓ by 4 hours after tₘₐₓ, by at least 94% of the Cₘₐₓ by 4 hours after tₘₐₓ, by at least 95% of the Cₘₐₓ by 4 hours after tₘₐₓ, by at least 96% of the Cₘₐₓ by 4 hours after tₘₐₓ, by at least 97% of the Cₘₐₓ by 4 hours after tₘₐₓ, by at least 98% of the Cₘₐₓ by 4 hours after tₘₐₓ, or by at least 99% of the Cₘₐₓ by 4 hours after tₘₐₓ. In some embodiments, a plasma level of a compound decreases by at least 50% of the Cₘₐₓ by 8 hours after administration, by at least 55% of the Cₘₐₓ by 8 hours after administration, by at least 60% of the Cₘₐₓ by 8 hours after administration, by at least 65% of the Cₘₐₓ by 8 hours after administration, by at least 70% of the Cₘₐₓ by 8 hours after administration, by at least 75% of the Cₘₐₓ by 8 hours after administration, by at least 80% of the Cₘₐₓ by 8 hours after administration, by at least 85% of the Cₘₐₓ by 8 hours after administration, by at least 90% of the Cₘₐₓ by 8 hours after administration, by at least 91% of the Cₘₐₓ by 8 hours after administration, by at least 92% of the Cₘₐₓ by 8 hours after administration, by at least 93% of the Cₘₐₓ by 8 hours after administration, by at least 94% of the Cₘₐₓ by 8 hours after administration, by at least 95% of the Cₘₐₓ by 8 hours after administration, by at least 96% of the Cₘₐₓ by 8 hours after administration, by at least 97% of the Cₘₐₓ by 8 hours after administration, by at least 98% of the Cₘₐₓ by 8 hours after administration, or by at least 99% of the Cₘₐₓ by 8 hours after administration. In some embodiments, a plasma level of a compound decreases by at least 50% of the Cₘₐₓ by 4 hours after administration, by at least 55% of the Cₘₐₓ by 4 hours after administration, by at least 60% of the Cₘₐₓ by 4 hours after administration, by at least 65% of the Cₘₐₓ by 4 hours after administration, by at least 70% of the Cₘₐₓ by 4 hours after administration, by at least 75% of the Cₘₐₓ by 4 hours after administration, by at least 80% of the Cₘₐₓ by 4 hours after administration, by at least 85% of the Cₘₐₓ by 4 hours after administration, by at least 90% of the Cₘₐₓ by 4 hours after administration, by at least 91% of the Cₘₐₓ by 4 hours after administration, by at least 92% of the Cₘₐₓ by 4 hours after administration, by at least 93% of the Cₘₐₓ by 4 hours after administration, by at least 94% of the Cₘₐₓ by 4 hours after administration, by at least 95% of the Cₘₐₓ by 4 hours after administration, by at least 96% of the Cₘₐₓ by 4 hours after administration, by at least 97% of the Cₘₐₓ by 4 hours after administration, by at least 98% of the Cₘₐₓ by 4 hours after administration, or by at least 99% of the Cₘₐₓ by 4 hours after administration.

In some embodiments, a composition or method of the invention affords an increased Cₘₐₓ and a decreased tₘₐₓ, in combination with increased clearance of cyclobenzaprine or amitriptyline. For example, a composition or method of the invention may afford a Cₘₐₓ from about 20 to about 200 ng/mL from about 0.05 to about 2.5 hours after administration, while also affording a minimum plasma concentration from about 1 to about 5 ng/mL from about 22 to about 26 hours after administration, wherein the composition is administered for four days or more of daily administration. In some embodiments, a composition is administered within two hours of sleep. In some embodiments, a method is for reducing the symptoms of fibromyalgia in a human patient.

In some embodiments, the methods and compositions of the invention allow a compound to be removed from the plasma more quickly than if the compound was administered orally. This is beneficial because the clearance of a compound can aid in the reduction of the accumulation of cyclobenzaprine or amitriptyline from the body when administered by nightly dosing and in a chronic dosing schedule. The minimal concentration or Cₘᵢₙ may be determined by measuring the plasma concentration of the compound being administered can be measured either at fixed time points or at a variable time point, e.g., at time points after the time point corresponding to Cₘₐₓ, for example 23 hours after Cₘₐₓ. The Cₘᵢₙ can be measured after a single dose or after repeated, multiple, or chronic dosing, for example in daily dosing. For example, Cₘᵢₙ can be measured 3 hours after administration, 4 hours after administration, 5 hours after administration, 6 hours after administration, 7 hours after administration, 8 hours after administration, 9 hours after administration, 10 hours after administration, 11 hours after administration, or 12 hours after administration. In some embodiments, Cₘᵢₙ can be measured 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, or 36 hours after administration. In some embodiments, a composition affords a Cₘᵢₙ of a compound less than or equal to 10 pg/mL, less than or equal to 11 pg/mL, less than or equal to 12 pg/mL, less than or equal to 13 pg/mL, less than or equal to 14 pg/mL, less than or equal to 15 pg/mL, less than or equal to 16 pg/mL, less than or equal to 17 pg/mL, less than or equal to 18 pg/mL, less than or equal to 19 pg/mL, less than or equal to 20 pg/mL, less than or equal to 21 pg/mL, less than or equal to 22 pg/mL, less than or equal to 23 pg/mL, less than or equal to 24 pg/mL, less than or equal to 25 pg/mL, less than or equal to 26 pg/mL, less than or equal to 27 pg/mL, less than or equal to 28 pg/mL, less than or equal to 29 pg/mL, less than or equal to 30 pg/mL, less than or equal to 31 pg/mL, less than or equal to 32 pg/mL, less than or equal to 33 pg/mL, less than or equal to 34 pg/mL, less than or equal to 35 pg/mL, less than or equal to 36 pg/mL, less than or equal to 37 pg/mL, less than or equal to 38 pg/mL, less than or equal to 39 pg/mL, less than or equal to 40 pg/mL, less than or equal to 50 pg/mL, less than or equal to 60 pg/mL, less than or equal to 70 pg/mL, less than or equal to 80 pg/mL, less than or equal to 90 pg/mL, less than or equal to 100 pg/mL, less than or equal to 120 pg/mL, less than or equal to 140 pg/mL, less than or equal to 160 pg/mL, less than or equal to 180 pg/mL, or less than or equal to 200 pg/mL. In some embodiments, a composition affords a Cₘᵢₙ of a compound less than or equal to 100 pg/mL, less than or equal to 110 pg/mL, less than or equal to 120 pg/mL, less than or equal to 130 pg/mL, less than or equal to 140 pg/mL, less than or equal to 150 pg/mL, less than or equal to 160 pg/mL, less than or equal to 170 pg/mL, less than or equal to 180 pg/mL, less than or equal to 190 pg/mL, less than or equal to 200 pg/mL, less than or equal to 210 pg/mL, less than or equal to 220 pg/mL, less than or equal to 230 pg/mL, less than or equal to 240 pg/mL, less than or equal to 250 pg/mL, less than or equal to 260 pg/mL, less than or equal to 270 pg/mL, less than or equal to 280 pg/mL, less than or equal to 290 pg/mL, less than or equal to 300 pg/mL, less than or equal to 310 pg/mL, less than or equal to 320 pg/mL, less than or equal to 330 pg/mL, less than or equal to 340 pg/mL, less than or equal to 350 pg/mL, less than or equal to 360 pg/mL, less than or equal to 370 pg/mL, less than or equal to 380 pg/mL, less than or equal to 390 pg/mL, less than or equal to 400 pg/mL, less than or equal to 500 pg/mL, less than or equal to 600 pg/mL, less than or equal to 700 pg/mL, less than or equal to 800 pg/mL, less than or equal to 900 pg/mL, less than or equal to 1.0 ng/mL, less than or equal to 1.20 ng/mL, less than or equal to 1.40 ng/mL, less than or equal to 1.60 ng/mL, less than or equal to 1.80 ng/mL, or less than or equal to 2.00 ng/mL. In some embodiments, a composition affords a Cₘᵢₙ of a compound less than or equal to 3.0 ng/mL, less than or equal to 4.0 ng/mL, less than or equal to 5.0 ng/mL, less than or equal to 6.0 ng/mL, less than or equal to 7.0 ng/mL, less than or equal to 8.0 ng/mL, or less than or equal to 10.0 ng/mL The minimal concentration at 24 hours or Cₘᵢₙ(24) may be determined by measuring the plasma concentration of the compound being administered approximately 24 hours after the last dose or immediately prior to the next dose. Cₘᵢₙ(24) is significant as a plasma value or by calculating the ratio of Cₘᵢₙ(24) and the dose administered, which is the dose-normalized minimum plasma concentration or dnCₘᵢₙ₍₂₄₎*. The dnCₘᵢₙ₍₂₄₎* is calculated by determining the ratio of plasma level to dose administered. For example, in a study (shown in FIG. 1) in which 5.0 mg of cyclobenzaprine in an immediate release tablet was administered PO, the mean Cₘᵢₙ(24) was 1.384 ng/mL at 24 hours and the dnCₘᵢₙ₍₂₄₎* was ((1.384 ng/mL)/(5.0 mg))=0.27680 ng/(mg mL), or 0.27680×10⁻⁶ ml⁻¹. In another example, in a study in which 2.4 mg of cyclobenzaprine in an sublingual tablet was administered, the mean Cₘᵢₙ₍₂₄₎ was 706.55 ng/mL at 24 hours and the dnCₘᵢₙ₍₂₄₎* was ((706.55 ng/mL)/(2.4 mg))=294.40 ng/(mg mL), or 0.29440×10⁻⁶ mL⁻¹. In some embodiments, a dnCₘᵢₙ₍₂₄₎* is less than or equal to 1.0±25%×10⁻⁶ mL⁻¹, less than or equal to 0.9±25%×10⁻⁶ mL⁻¹, less than or equal to 0.8±25%×10⁻⁶ mL⁻¹, less than or equal to 0.7±25%×10⁻⁶ mL⁻¹, less than or equal to 0.6±25%×10⁻⁶ mL⁻¹, less than or equal to 0.5±25%×10⁻⁶ mL⁻¹, less than or equal to 0.4±25%×10⁻⁶ mL⁻¹, or less than or equal to 0.3±25%×10⁻⁶ mL⁻¹. In some embodiments, a dnCₘᵢₙ₍₂₄₎* is less than or equal to 240±25%×10⁻⁹ mL⁻¹, less than or equal to 220±25%×10⁻⁹ mL⁻¹, less than or equal to 200±25%×10⁻⁹ mL⁻¹, less than or equal to 180±25%×10⁻⁹ mL⁻¹, less than or equal to 160±25%×10⁻⁹ mL⁻¹, less than or equal to 140±25%×10⁻⁹ mL⁻¹, less than or equal to 120±25%×10⁻⁶ mL⁻¹, less than or equal to 100±25%×10⁻⁶ mL⁻¹, less than or equal to 80±25%×10⁻⁹ mL⁻¹, less than or equal to 60±25%×10⁻⁹ mL⁻¹, less than or equal to 40±25%×10⁻⁹ mL⁻¹, less than or equal to 20±25%×10⁻⁹ mL⁻¹, or less than or equal to 10±25%×10⁻⁹ mL⁻¹. In some embodiments, an a dnCₘᵢₙ₍₂₄₎* is less than or equal to 9±25%×10⁻⁹ mL⁻¹, less than or equal to 9±25%×10⁻⁶ mL⁻¹, less than or equal to 7±25%×10⁻⁹ mL⁻¹, less than or equal to 6±25%×10⁻⁹ mL⁻¹, less than or equal to 5±25%×10⁻⁹ mL⁻¹, less than or equal to 4±25%×10⁻⁹ mL⁻¹, less than or equal to 3±25%×10⁻⁹ mL⁻¹, less than or equal to 2±25%×10⁻⁹ mL⁻¹, or less than or equal to 1±25%×10⁻⁹ mL⁻¹.

The dose- and body mass-normalized Cₘᵢₙ, or the dbmnCₘᵢₙ* is calculated by determining the ratio of the product of the plasma level and body mass to dose administered. The dbmnCₘᵢₙ* at 24 hours, or dbmnCₘᵢₙ₍₂₄₎*, may be determined by measuring the plasma concentration of the compound being administered approximately 24 hours after the last dose or immediately prior to the next dose in a daily dosing schedule, such as a bedtime dosing schedule. The dbmnCₘᵢₙ* may be determined by measuring the plasma concentration of the compound being administered can be measured either at fixed time points, for example 24 hours after administration (Cₘᵢₙ₍₂₄₎*), or at a variable time point, e.g., at time points after the time point corresponding to Cₘₐₓ, for example 23 hours after Cₘₐₓ. For example, in a study (shown in FIG. 1) in which 5.0 mg of cyclobenzaprine in an immediate release tablet was administered PO, the mean Cₘᵢₙ₍₂₄₎* was 1.384 ng/mL at 24 hours and, assuming a 70 kg human, the approximate dbmnCₘᵢₙ₍₂₄₎* was 70 kg×((1.384 ng/mL)/(5.0 mg))=19.4×10⁻⁶ kg mL⁻¹. For example, in a study in which 2.4 mg of cyclobenzaprine in an sublingual tablet was administered, the mean Cₘᵢₙ₍₂₄₎ was 706.55 ng/mL at 24 hours and the dnCₘᵢₙ₍₂₄₎* was ((706.55 ng/mL)/(2.4 mg))=294.40 ng/(mg mL), or 294.40×10⁻⁹ mL⁻¹ and, assuming a 70 kg human, the approximate dbmnCₘᵢₙ₍₂₄₎* was 70 kg×((706.55 ng/mL)/(2.4 mg))=20.608×10⁻⁶ kg mL⁻¹. In some embodiments, a dbmnCₘᵢₙ₍₂₄₎* is less than or equal to 1.0±25%×10⁻⁶ kg mL⁻¹, less than or equal to 0.9±25%×10⁻⁶ kg mL⁻¹, less than or equal to 0.8±25%×10⁻⁶ kg mL⁻¹, less than or equal to 0.7±25%×10⁻⁶ kg mL⁻¹, less than or equal to 0.6±25%×10⁻⁶ kg mL⁻¹, less than or equal to 0.5±25%×10⁻⁶ kg mL⁻¹, less than or equal to 0.4±25%×10⁻⁶ kg mL⁻¹, or less than or equal to 0.3±25%×10⁻⁶ kg mL⁻¹. In some embodiments, a dbmnCₘᵢₙ₍₂₄₎* is less than or equal to 240±25%×10⁻⁹ mL⁻¹, less than or equal to 220±25%×10⁻⁹ kg mL⁻¹, less than or equal to 200±25%×10⁻⁹ kg mL⁻¹, less than or equal to 180±25%×10⁻⁹ kg mL⁻¹, less than or equal to 160±25%×10⁻⁹ kg mL⁻¹, less than or equal to 140±25%×10⁻⁹ kg mL⁻¹, less than or equal to 120±25%×10⁻⁶ kg mL⁻¹, less than or equal to 100±25%×10⁻⁶ kg mL⁻¹, less than or equal to 80±25%×10⁻⁹ kg mL⁻¹, less than or equal to 60±25%×10⁻⁹ kg mL⁻¹, less than or equal to 40±25%×10⁻⁹ kg mL⁻¹, less than or equal to 20±25%×10⁻⁹ kg mL⁻¹, or less than or equal to 10±25%×10⁻⁹ kg mL⁻¹. In some embodiments, an a dbmnCₘᵢₙ₍₂₄₎* is less than or equal to 9±25%×10⁻⁹ kg mL⁻¹, less than or equal to 9±25%×10⁻⁶ mL⁻¹, less than or equal to 7±25%×10⁻⁹ kg mL⁻¹, less than or equal to 6±25%×10⁻⁹ kg mL⁻¹, less than or equal to 5±25%×10⁻⁹ kg mL⁻¹, less than or equal to 4±25%×10⁻⁹ kg mL⁻¹, less than or equal to 3±25%×10⁻⁹ kg mL⁻¹, less than or equal to 2±±25%×10⁻⁹ kg mL⁻¹, or less than or equal to 1±25%×10⁻⁹ kg mL⁻¹.

In some embodiments, the methods and compositions of the invention allow a compound to be absorbed into plasma without gut or hepatic metabolism which reduces the extent of demethylation by p450. This is beneficial because demethylation by p450 transforms cyclobenzaprine into the norcyclobenzaprine and amitriptyline into nortriptyline which have long half lives. Reducing the concentration of the secondary amine metabolites norcyclobenzaprine and nortriptyline is beneficial because the tertiary amines cyclobenzaprine and nortriptyline are cleared from the plasma and from the body more rapidly and the clearance of a compound can aid in the reduction of the accumulation of compounds (drug plus metabolites) that act on from the body when administered by nightly dosing and in a chronic dosing schedule. The ratio of the plasma concentration of a metabolite to the dose of the agent administered is the dose-normalized concentration of the metabolite or dnCₘₑₜ*. The ratio of the plasma concentration of norcyclobenzaprine to the dose of cyclobenzaprine administered is the dose-normalized concentration of norcyclobenzaprine or dnCₘₑₜ(norcycl)*. The ratio of the plasma concentration of nortriptyline to the dose of amitriptyline administered is the dose-normalized concentration of nortriptyline or dnCₘₑₜ(nortrip)*. The dnCₘₑₜ* may be measured at various times after administration of the compound either after a single dose or after multiple doses. The dnCₘₑₜ* can be measured either at fixed time points or at a variable time point, e.g., the time point corresponding to Cₘₐₓ. For example, dnCₘₑₜ* can be measured 5 minutes after administration, 10 minutes after administration, 15 minutes after administration, 30 minutes after administration, 45 minutes after administration, 1 hour after administration, 2 hours after administration, 3 hours after administration, 4 hours after administration, 5 hours after administration, 6 hours after administration, 7 hours after administration, 10 hours after administration, 11 hours after administration, or 12 hours after administration. In some embodiments, dnCₘₑₜ* can be measured 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, or 36 hours after administration. The dnCₘₑₜ* at 24 hours or dnCₘₑₜ₍₂₄₎* may be determined by measuring the plasma concentration of the compound being administered approximately 24 hours after the last dose or immediately prior to the next dose in a daily dosing schedule, such as a bedtime dosing schedule. For example, in a study in which 5.0 mg of cyclobenzaprine in an immediate release tablet was administered and the mean plasma concentration of norcyclobenzaprine was 1.227 ng/mL at 24 hours, the dnCₘₑₜ₍₂₄₎(norcycl) * is ((1.227 ng/mL)/(5.0 mg))=0.245 ng/(mL mg) or 0.245×10⁻⁶ mL⁻¹. The dnCₘₑₜ (24)* for multiple dosing of cyclobenzaprine or amitriptyline is expected to be higher. In some embodiments, a dnCₘₑₜ₍₂₄₎* is less than or equal to 1.0±25%×10⁻⁶ mL⁻¹, less than or equal to 0.9±25%×10⁻⁶ mL⁻¹, less than or equal to 0.8±25%×10⁻⁶ mL⁻¹, less than or equal to 0.7±25%×10⁻⁶ mL⁻¹, less than or equal to 0.6±25%×10⁻⁶ mL⁻¹, less than or equal to 0.5±25%×10⁻⁶ mL⁻¹, less than or equal to 0.4±25%×10⁻⁶ mL⁻¹, or less than or equal to 0.3±25%×10⁻⁶ mL⁻¹. In some embodiments, a dnCₘₑₜ₍₂₄₎* is less than or equal to 240±25%×10⁻⁹ mL⁻¹, less than or equal to 220±25%×10⁻⁹ mL⁻¹, less than or equal to 200±25%×10-9 mL⁻¹, less than or equal to 180±25%×10⁻⁹ mL⁻¹, less than or equal to 160±25%×10⁻⁹ mL⁻¹, less than or equal to 140±25%×10⁻⁹ mL⁻¹, less than or equal to 120±25%×10⁻⁶ mL⁻¹, less than or equal to 100±25%×10⁻⁶ mL⁻¹, less than or equal to 80±25%×10⁻⁹ mL⁻¹, less than or equal to 60±25%×10⁻⁹ mL⁻¹, less than or equal to 40±25%×10⁻⁹ mL⁻¹, less than or equal to 20±25%×10⁻⁹ mL⁻¹, or less than or equal to 10±25%×10⁻⁹ mL⁻¹. In some embodiments, an a dnCₘₑₜ₍₂₄₎* is less than or equal to 9±25%×10⁻⁹ mL⁻¹, less than or equal to 9±25%×10⁻⁶ mL⁻¹, less than or equal to 7±25%×10⁻⁹ mL⁻¹, less than or equal to 6±25%×10⁻⁹ mL⁻¹, less than or equal to 5±25%×10⁻⁹ mL⁻¹, less than or equal to 4±25%×10⁻⁹ mL⁻¹, less than or equal to 3±25%×10⁻⁹ mL⁻¹, less than or equal to 2±25%×10⁻⁹ mL⁻¹, or less than or equal to 1±25%×10⁻⁹ mL⁻¹.

The ratio of the product of the body mass and plasma concentration of norcyclobenzaprine to the dose of cyclobenzaprine administered is the dose- and body mass-normalized concentration of norcyclobenzaprine or dbmnCₘₑₜ(norcycl)*. The ratio of the product of the body mass and plasma concentration of nortriptyline to the dose of amitriptyline administered is the dose- and body mass-normalized concentration of nortriptyline or dbmnCₘₑₜ(nortrip)*. The dbmnCₘₑₜ* may be measured at various times after administration of the compound either after a single dose or after multiple doses. The dbmnCₘₑₜ* can be measured either at fixed time points or at a variable time point, e.g., the time point corresponding to Cₘₐₓ. For example, dbmnCₘₑₜ* can be measured 5 minutes after administration, 10 minutes after administration, 15 minutes after administration, 30 minutes after administration, 45 minutes after administration, 1 hour after administration, 2 hours after administration, 3 hours after administration, 4 hours after administration, 5 hours after administration, 6 hours after administration, 7 hours after administration, 10 hours after administration, 11 hours after administration, or 12 hours after administration. In some embodiments, dbmnCₘₑₜ* can be measured 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, or 36 hours after administration. The dbmnCₘₑₜ* at 24 hours or dbmnCₘₑₜ₍₂₄₎* may be determined by measuring the plasma concentration of the compound being administered approximately 24 hours after the last dose or immediately prior to the next dose in a daily dosing schedule, such as a bedtime dosing schedule. For example, in a study in which 5.0 mg of cyclobenzaprine in an immediate release tablet was administered and the mean plasma concentration of norcyclobenzaprine was 1.227 ng/mL at 24 hours, and the average body mass of the human is estimated to be 70 kg, the dbmnCₘₑₜ₍₂₄₎(norcycl) * is 70 kg×((1.227 ng/mL)/(5.0 mg))=17.2×10⁻⁶ kg mL⁻¹. The dbmnCₘₑₜ₍₂₄₎* for multiple dosing of cyclobenzaprine or amitriptyline is expected to be higher. In some embodiments, a dbmnCₘₑₜ₍₂₄₎* is less than or equal to 1.0±25%×10⁻⁶ kg mL⁻¹, less than or equal to 0.9±25%×10⁻⁶ kg mL⁻¹, less than or equal to 0.8±25%×10⁻⁶ kg mL⁻¹, less than or equal to 0.7±25%×10⁻⁶ kg mL⁻¹, less than or equal to 0.6±25%×10⁻⁶ kg mL⁻¹, less than or equal to 0.5±25%×10⁻⁶ kg mL⁻¹, less than or equal to 0.4±25%×10⁻⁶ kg mL⁻¹, or less than or equal to 0.3±25%×10⁻⁶ kg mL⁻¹. In some embodiments, a dbmnCₘₑₜ₍₂₄₎* is less than or equal to 240±25%×10⁻⁹ kg mL⁻¹, less than or equal to 220±25%×10⁻⁹ kg mL⁻¹, less than or equal to 200±25%×10⁻⁹ kg mL⁻¹, less than or equal to 180±25%×10⁻⁹ kg mL⁻¹, less than or equal to 160±25%×10⁻⁹ kg mL⁻¹, less than or equal to 140±25%×10⁻⁹ kg mL⁻¹, less than or equal to 120±25%×10⁻⁶ kg mL⁻¹, less than or equal to 100±25%×10⁻⁶ kg mL⁻¹, less than or equal to 80±25%×10⁻⁹ kg mL⁻¹, less than or equal to 60±25%×10⁻⁹ kg mL⁻¹, less than or equal to 40±25%×10⁻⁹ kg mL⁻¹, less than or equal to 20±25%×10⁻⁹ kg mL⁻¹, or less than or equal to 10±25%×10⁻⁹ kg mL⁻¹. In some embodiments, an a dbmnCₘₑₜ₍₂₄₎* is less than or equal to 9±25%×10-9 kg mL⁻¹, less than or equal to 9±25%×10⁻⁶ kg mL⁻¹, less than or equal to 7±25%×10⁻⁹ kg mL⁻¹, less than or equal to 6±25%×10⁻⁹ kg mL⁻¹, less than or equal to 5±25%×10⁻⁹ kg mL⁻¹, less than or equal to 4±25%×10⁻⁹ kg mL⁻¹, less than or equal to 3±25%×10⁻⁹ kg mL⁻¹, less than or equal to 2±25%×10⁻⁹ kg mL⁻¹, or less than or equal to 1±25%×10⁻⁹ kg mL⁻¹.

### Excipients

In some embodiments, a composition of the invention is useful as a medicament. In some embodiments, the invention provides for the use of a composition of the invention in the manufacture of a medicament. In some embodiments, it may be beneficial to include one or more excipients in the compositions of the invention. One of skill in the art would appreciate that the choice of any one excipient may influence the choice of any other excipient. For example, the choice of a particular excipient may preclude the use of one or more additional excipient because the combination of excipients would produce undesirable effects. One of skill in the art would be able to empirically determine which additional excipients, if any, to include in the formulations of the invention. For example, a compound of the invention can be combined with at least one pharmaceutically acceptable carrier such as a solvent, bulking agents, binder, humectant, disintegrating agent, solution retarder, disintegrant, glidant, absorption accelerator, wetting agent, solubilizing agent, lubricant, sweetening agent, or flavorant agent. A “pharmaceutically acceptable carrier” refers to any diluent or excipient that is compatible with the other ingredients of the formulation, and which is not deleterious to the recipient. A pharmaceutically acceptable carrier can be selected on the basis of the desired route of administration, in accordance with standard pharmaceutical practices.

### Bulking Agents

In some embodiments, it may be beneficial to include a bulking agent in the compositions of the invention. Bulking agents are commonly used in pharmaceutical compositions to provide added volume to the composition. Bulking agents are well known in the art. Accordingly, the bulking agents described herein are not intended to constitute an exhaustive list, but are provided merely as exemplary bulking agents that may be used in the compositions and methods of the invention.

Exemplary bulking agents may include carbohydrates, sugar alcohols, amino acids, and sugar acids. Bulking agents include, but are not limited to, mono-, di-, or poly-, carbohydrates, starches, aldoses, ketoses, amino sugars, glyceraldehyde, arabinose, lyxose, pentose, ribose, xylose, galactose, glucose, hexose, idose, mannose, talose, heptose, glucose, fructose, methyl α-D-glucopyranoside, maltose, lactone, sorbose, erythrose, threose, arabinose, allose, altrose, gulose, idose, talose, erythrulose, ribulose, xylulose, psicose, tagatose, glucosamine, galactosamine, arabinans, fructans, fucans, galactans, galacturonans, glucans, mannans, xylans, inulin, levan, fucoidan, carrageenan, galactocarolose, pectins, amylose, pullulan, glycogen, amylopectin, cellulose, microcrystalline cellulose, pustulan, chitin, agarose, keratin, chondroitin, dermatan, hyaluronic acid, xanthin gum, sucrose, trehalose, dextran, lactose, alditols, inositols, sorbitol, mannitol, glycine, aldonic acids, uronic acids, aldaric acids, gluconic acid, isoascorbic acid, ascorbic acid, glucaric acid, glucuronic acid, gluconic acid, glucaric acid, galacturonic acid, mannuronic acid, neuraminic acid, pectic acids, maize starch, and alginic acid.

### Disintegrants

In some embodiments, it may be beneficial to include a disintegrant in the compositions of the invention. Disintegrants aid in the breakup of solid compositions, facilitating delivery of an active pharmaceutical composition. Disintegrants are well known in the art. Some disintegrants have been referred to as superdisintegrants because they have fast properties, and may be used as disintegrants in the context of the invention. Accordingly, the disintegrants described herein are not intended to constitute an exhaustive list, but are provided merely as exemplary disintegrants that may be used in the compositions and methods of the invention. Exemplary disintegrants include crospovidone, microcrystalline cellulose, sodium carboxymethyl cellulose, methyl cellulose, sodium starch glycolate, calcium carboxymethyl croscarmellose sodium, polyvinylpyrrolidone, lower alkyl-substituted hydroxypropyl cellulose, Indion 414, starch, pre-gelatinized starch, calcium carbonate, gums, sodium alginate, and Pearlitol Flash®. Pearlitol Flash® (Roquette) is a mannitol-maize starch disintegrant that is specifically designed for orally dispersible tablets (ODT). Certain disintegrants have an efferverscent quality.

### Glidants

In some embodiments, it may be beneficial to include a glidant in the compositions of the invention. Glidants aid in the ability of a powder to flow freely. Glidants are well known in the art. Accordingly, the glidants described herein are not intended to constitute an exhaustive list, but are provided merely as exemplary glidants that may be used in the compositions and methods of the invention. Exemplary glidants include colloidal silica (silicon dioxide), magnesium stearate, starch, talc, glycerol behenate, DL-leucine, sodium lauryl sulfate, calcium stearate, and sodium stearate.

### Lubricants

In some embodiments, it may be beneficial to include a lubricant in the compositions of the invention. Lubricants help keep the components of a composition from clumping. Lubricants are well known in the art. Accordingly, the lubricants described herein are not intended to constitute an exhaustive list, but are provided merely as exemplary lubricants that may be used in the compositions and methods of the invention. Exemplary lubricants include calcium stearate, magnesium stearate, stearic acid, sodium stearyl fumarate, vegetable based fatty acids, talc, mineral oil, light mineral oil, hydrogenated vegetable oil (e.g., peanut oil, cottonseed oil, sunflower oil, sesame oil, olive oil, corn oil, safflower oil, canola oil, coconut oil and soybean oil), silica, zinc stearate, ethyl oleate, ethyl laurate.

### Sweeteners

In some embodiments, it may be beneficial to include a sweetener in the compositions of the invention. Sweeteners help improve the palatability of the composition by conferring a sweet taste to the composition. Sweeteners are well known in the art. Accordingly, the sweeteners described herein are not intended to constitute an exhaustive list, but are provided merely as exemplary sweeteners that may be used in the compositions and methods of the invention. Exemplary sweeteners include, without limitation, compounds selected from the saccharide family such as the mono-, di-, tri-, poly-, and oligosaccharides; sugars such as sucrose, glucose (corn syrup), dextrose, invert sugar, fructose, maltodextrin and polydextrose; saccharin and salts thereof such as sodium and calcium salts; cyclamic acid and salts thereof, dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide many also be used.

### Flavorants

In some embodiments, it may be beneficial to include a flavorant in the compositions of the invention. Flavorants help improve the palatability of the composition by conferring a more desirable taste to the composition. Flavorants are well known in the art. Accordingly, the flavorants described herein are not intended to constitute an exhaustive list, but are provided merely as exemplary flavorants that may be used in the compositions and methods of the invention. Exemplary flavorants include, without limitation, natural and/or synthetic (i.e., artificial) compounds such as peppermint, spearmint, wintergreen, menthol, cherry, strawberry, watermelon, grape, banana, peach, pineapple, apricot, pear, raspberry, lemon, grapefruit, orange, plum, apple, lime, fruit punch, passion fruit, pomegranate, chocolate (e.g., white, milk, dark), vanilla, caramel, coffee, hazelnut, cinnamon, combinations thereof, and the like.

### Coloring Agents

Coloring agents can be used to color code the composition, for example, to indicate the type and dosage of the therapeutic agent therein. Coloring Agents are well known in the art. Accordingly, the coloring agents described herein are not intended to constitute an exhaustive list, but are provided merely as exemplary coloring agents that may be used in the compositions and methods of the invention. Exemplary coloring agents include, without limitation, natural and/or artificial compounds such as FD & C coloring agents, natural juice concentrates, pigments such as titanium oxide, silicon dioxide, and zinc oxide, combinations thereof, and the like.

### Combination Therapy

As described above, the compositions and methods of the invention may be used to treat PTSD, depression, fibromyalgia, traumatic brain injury, sleep disorder, non-restorative sleep, chronic pain, and anxiety disorder. Any of the methods of treatment described also may be combined with a psychotherapeutic intervention to improve the outcome of the treatment. Exemplary psychotherapeutic interventions directed at either modifying traumatic memories or reducing emotional responses to traumatic memories, including psychological debriefing, cognitive behavior therapy and eye movement desensitization and reprocessing, systematic desensitization, relaxation training, biofeedback, cognitive processing therapy, stress inoculation training, assertiveness training, exposure therapy, combined stress inoculation training and exposure therapy, combined exposure therapy, and relaxation training and cognitive therapy. In each case, the goal of the intervention involves either modifying traumatic memories or reducing emotional responses to traumatic memories. The intended result is generally a improvement in the symptoms of PTSD or the reduction of occurrences of symptoms, as evidenced in terms of physiological responding, anxiety, depression, and feelings of alienation.

In some embodiments of the invention, a composition is combined with a drug which may further alleviate the symptoms of PTSD, depression, fibromyalgia, traumatic brain injury, sleep disorder, non-restorative sleep, chronic pain, or anxiety disorder. The drugs include an alpha-1-adrenergic receptor antagonist, a beta-adrenergic antagonist, an anticonvulsant, a selective serotonin reuptake inhibitor, a serotonin-norepinephrine reuptake inhibitor, and an analgesic. Exemplary anticonvulsants include carbamazepine, gabapentin, lamotrigine, oxcarbazepine, pregabalin, tiagabine, topiramate, and valproate. An exemplary alpha-1-adrenergic receptor antagonist is prazosin. Exemplary selective serotonin reuptake inhibitors or serotonin-norepinephrine reuptake inhibitors include, bupropion, citalopram, desvenlafaxine, duloxetine, escitalopram, fluoxetine, escitalopram, fluvoxamine, milnacipran, paroxetine, sertraline, trazodone, and venlafaxine. Exemplary analgesics include pregabalin, gabapentin, acetaminophen, tramadol, and non-steroidal anti-inflammatory drugs (e.g., ibuprofen and naproxen sodium). Additional drugs that can be used in combination with the compositions of the invention include sodium oxybate, zolpidem, pramipexole, modafinil, temazepam, zaleplon, and armodafinil.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention.

The following examples are set forth as being representative of the present invention. These examples are not to be construed as limiting the scope of the invention as these and other equivalent embodiments will be apparent in view of the present disclosure, figures, and accompanying claims.

### EXAMPLES

### Example 1

To study cyclobenzaprine metabolism, cyclobenzaprine HCl immediate release (Watson, bioequivalent to Flexeril 5 mg) was administered to 10 healthy human subjects in 5 mg tablets for oral administration and cyclobenzaprine and norcyclobenzaprine plasma concentrations were measured at 0.5 hours, 1 hour, 1.5 hours, 2 hours, 2.5 hours, 3 hours, 3 hours 20 minutes, 3 hours 40 minutes, 4 hours, 4 hours 20 minutes, 4 hours 40 minutes, 5 hours, 5.5 hours, 6 hours, 8 hours, 12 hours, 16 hours, 24 hours, 36 hours, 48 hours, 72 hours, 96 hours, and 168 hours (1 week) (Table 2, FIGS. 1a and 1b). A high performance liquid chromatographic method for the determination of cyclobenzaprine and norcyclobenzaprine in human EDTA K₃ plasma also was developed and validated. The conditions used were as follows:

Mobile Phase A (MPA) and Autosampler Rinsing Solution No. 1: Milli-Q Type Water/Methanol (40/60), Ammonium Formate 5 mM, Formic Acid 0.1% (0.1:100)

Mobile Phase B (MPB) and Autosampler Rinsing Solution No. 2: Methanol (100%)

Buffer Solution: Trizma® Base 500 mM, pH 11.0

Dissolving Solution: Milli-Q Type Water/Methanol (50/50)

Solvent delivery module: Hewlett Packard Series 1100, Agilent (Montreal, Canada)

Chromatographic mode: Reversed phase

Isocratic/gradient mode: Gradient

Time table program:

| Time (min.) | MPA (%) | MPB (%) |
|---|---|---|
| 0.00 | 100 | 0 |
| 0.50 | 100 | 0 |
| 0.51 | 0 | 100 |
| 1.50 | 0 | 100 |
| 1.51 | 100 | 0 |
| 3.50 | 100 | 0 |

Mobile phase A flow rate: 1.000 mL/min

Back-pressure: 130 bars (approximately)

CTC PAL (HTC-xt)* (stator wash station)

Injection macro:

Sample pickup

Needle dip in wash 1

Sample injection

Valve clean with solvent 2

Post clean with solvent 2

Valve clean with solvent 1

Post clean with solvent 1

(stator wash)

Autosampler method:

Air gap volume (mL): 3 Valve clean time solvent 2 (s): 2

Front volume (mL): 0 Post clean time solvent 2 (s): 2

Rear volume (mL): 0 Valve clean time solvent 1 (s): 3

Filling speed (L/s): 5 Post clean time solvent 1 (s): 3

Pullup delay (ms): 3000 Stator wash: 1

Inject to LC VLV1 Delay stator wash (s): 120

Injection speed (mL/s): 5 Stator wash time solvent 2 (s): 5

Pre inject delay (ms): 500 Stator wash time solvent 1 (s): 5

Post inject delay (ms): 500 Method syringe (μL): 100

Needle gap valve clean (mm): 3

Note: Solvent 1 corresponds to autosampler rinsing solution No. 1 and solvent 2 corresponds to autosampler rinsing solution No. 2. Therefore, the valve, post and stator washes are performed with methanol first and mobile phase A last.

Autosampler loop: 100 μL, stainless steel

Injection volume: 20 μL

Injection temperature: Room temperature

Pre-column filter: Supelco Filter 0.5 μm

Column: Supplier/Manufacturer ACE

Brand/Model ACE 3 C18

Length×width (mm) 30×4.6

Particle size (μm) 3

Column temperature: Room temperature

Retention times (Retention times may vary between runs. Retention times obtained without dual injection (ex: cohesive)):

Cyclobenzaprine: 1.05 minutes

Norcyclobenzaprine: 1.16 minutes

Internal standard A: 1.05 minutes

Internal standard B: 1.15 minutes

Autosampler run time: 3.50 minutes (TIME SAVER ON)

Acquisition time: 3.50 minutes

Detector Parameters

Source: TurboIonSpray

Split ratio: not applicable

Ionization mode: Positive

API 5000 (may be modified to optimize chromatography conditions, sensitivity, or reproducibility

Auxiliary gas pressure (GS2): 70 psi

Nebulizer gas pressure (GS1): 50 psi

Curtain gas pressure: 45 psi

CAD gas: 4

Interface heater (Ihe): ON

TurboIonSpray temperature: 450° C.

Ion Spray Voltage (ISV): 1800

State file parameters: DP=70; EP=10;

(cyclobenzaprine) CE=55; CXP=12

State file parameters: DP=65; EP=10;

(norcyclobenzaprine) CE=48; CXP=12

State file parameters: DP=70; EP=10;

(internal standard A) CE=55; CXP=12

State file parameters: DP=65; EP=10;

(internal standard B) CE=48; CXP=12

Mass acquisition parameters:

Analytes: MRM Dwell time=150 msec; Pause time=5 msec

IS: MRM Dwell time=90 msec; Pause time=5 msec

Cyclobenzaprine 276.4® 215.2 amu

Norcyclobenzaprine 262.4® 215.2 amu

Internal standard A 279.2® 215.1 amu

Internal standard B 265.4® 215.2 amu

Integration Parameters (may be changed to optimize peak integration)

| | Cyclo | Norcyclo | Int. Std. A | Int. Std. B |
|---|---|---|---|---|
| Noise Threshold: | 2 | 2 | 1 | 1 |
| Area Threshold: | 4 | 4 | 2 | 2 |
| RT Window (sec): | 30 | 30 | 30 | 30 |
| Bunching factor: | 1 | 2 | 2 | 2 |
| Num. Smooths: | 1 | 1 | 1 | 1 |
| Separation Width: | 0 | 0 | 0 | 0 |
| Separation Height: | 0 | 0 | 0 | 0 |

### Calibration Parameters

Peak Attribute: Area

Calibration equation: y=mx+b

Calibration regression: Linear

Weighting factor: 1/C2

Determination factor: r2

Cyclobenzaprine and norcyclobenzaprine were extracted from a 0.200 mL aliquot of human EDTA K₃ plasma using an automated liquid-liquid extraction procedure, and then injected into a liquid chromatograph equipped with a tandem mass spectrometry detector. Quantitation was based on peak area ratio of the analytes versus their stable labeled internal standards. A weighted (1/C2), linear regression was performed to determine the concentration of the analytes. All regressions and figures presented in this validation report were generated by MDS Sciex Analyst version 1.4.2 and Thermo Electron Corporation Watson LIMS software, version 7.0.0.01b. The results of method validation were acceptable, which demonstrate that the method is suitable for the determination of cyclobenzaprine and norcyclobenzaprine in human EDTA K₃ plasma over the range of 50 to 10000 pg/mL for cyclobenzaprine and 5 to 1000 pg/mL for norcyclobenzaprine. Cyclobenzaprine and norcyclobenzaprine were measured in plasma. Equilibrium receptor binding assays were performed on cell lines expressing recombinant human receptors expressed to determine intrinsic potency of cyclobenzaprine and norcyclobenzaprine on human serotonin 5-HT1a, 5-HT2a, 5-HT2b, 5-HT2c, 5-HT5a, and 5-HT6 receptors, adrenergic α-1A, adrenergic α-2 (A,B,C), histamine H1, and the muscarinic M1 and M2 receptors. Select receptors were analyzed in ligand-induced intracellular calcium mobilization.

The amount of norcyclobenzaprine in plasma was unexpectedly high and this discovery related to the improved methods that we employed relative to the methods in the literature which did not detect norcyclobenzaprine. In addition, the half-life of norcyclobenzaprine was unexpectedly long. The tₘₐₓ of cyclobenzaprine was calculated to be approximately 3.5 hours (AUC_{0-∞h}=103.1±35.8 ng hr mL⁻¹), while the tₘₐₓ of norcyclobenzaprine was calculated to be approximately 24.0 hours (AUC_{0-∞h}=169.5±94.3 ng hr mL⁻¹). Furthermore, the ratio of cyclobenzaprine to norcyclobenzaprine decreased more rapidly than anticipated, reaching a ratio of 1.1:1 within 24 hours and falling to 1:2 by 72 hours (Table 3). This is supported by the surprisingly long half life of norcyclobenzaprine, which was calculated to be approximately 73 hours (72.75±27.71 h), as compared to approximately 31 hours (30.95±7.18 h) for cyclobenzaprine. These data indicate that norcyclobenzaprine remains in a subject's system long after most of the cyclobenzaprine has been eliminated. In vitro, cyclobenzaprine and norcyclobenzaprine exhibited high affinity binding (Kᵢ) to receptors: 5-HT2a (5.2 and 13 nM) and 5-HT2c (5.2 and 43 nM), adrenergic α-1A (5.6 and 34 nM), α-2B (Kᵢ=21 and 150 nM) and α-2C (Kᵢ=21 and 48 nM,); H1 (1.3 nM and 5.6 nM); M1 (7.9 nM and 30 nM). Functionally, norcyclobenzaprine is an antagonist at 5-HT2a (IC₅₀=92 nM) by Ca+ mobilization. Cyclobenzaprine is a functional antagonist on 5-HT2B (IC₅₀=100 nM), which is consistent with a lack of association with any heart valve pathology. Cyclobenzaprine and norcyclobenzaprine are functional antagonists on 5-HT2c (IC₅₀=0.44 μM and 1.22 μM) and on α-2A (IC₅₀=4.3 μM and 6.4 μM). In contrast, both CBP and nCBP were shown to be functional agonists on 5-HT1a (EC₅₀=5.3 μM and 3.2 μM).

TABLE 2
Cyclobenzaprine and norcyclobenzaprine plasma concentrations

| Hours | 0 | 0.5 | 1 | 1.5 | 2 | 2.5 | 3 |
|---|---|---|---|---|---|---|---|
| Plasma | Below | 102 | 896 | 1999 | 3061 | 3206 | 3416 |
| cyclobenzaprine | limit of | | | | | | |
| concentration | detection | | | | | | |
| (pg/mL) | | | | | | | |
| Plasma | Below | 5 | 63 | 193 | 391 | 505 | 610 |
| norcyclobenzaprine | limit of | | | | | | |
| concentration | detection | | | | | | |
| (pg/mL) | | | | | | | |
| Hours | 3.33 | 3.67 | 4 | 4.33 | 4.67 | 5 | 5.5 |
| Plasma | 3634 | 3538 | 3574 | 3680 | 3480 | 3469 | 3245 |
| cyclobenzaprine | | | | | | | |
| concentration | | | | | | | |
| (pg/mL) | | | | | | | |
| Plasma | 703 | 730 | 753 | 681 | 722 | 773 | 839 |
| norcyclobenzaprine | | | | | | | |
| concentration | | | | | | | |
| (pg/mL) | | | | | | | |
| Hours | 6 | 8 | 12 | 16 | 24 | 36 | 48 |
| Plasma | 3142 | 2578 | 1962 | 1486 | 1384 | 831 | 707 |
| cyclobenzaprine | | | | | | | |
| concentration | | | | | | | |
| (pg/mL) | | | | | | | |
| Plasma | 928 | 967 | 1114 | 1118 | 1227 | 1166 | 1075 |
| norcyclobenzaprine | | | | | | | |
| concentration | | | | | | | |
| (pg/mL) | | | | | | | |

| Hours | 72 | 96 | 168 |
|---|---|---|---|
| Plasma | 394 | 227 | 26 |
| cyclobenzaprine | | | |
| concentration | | | |
| (pg/mL) | | | |
| Plasma | 820 | 659 | 278 |
| norcyclobenzaprine | | | |
| concentration | | | |
| (pg/mL) | | | |

TABLE 3
Cyclobenzaprine: norcyclobenzaprine ratio

| Hours | 0 | 0.5 | 1 | 1.5 | 2 | 2.5 | 3 |
|---|---|---|---|---|---|---|---|
| Ratio | N/A | 22.5 | 14.3 | 10.4 | 7.8 | 6.4 | 5.6 |
| Hours | 3.33 | 3.67 | 4 | 4.33 | 4.67 | 5 | 5.5 |
| Ratio | 5.2 | 4.8 | 4.7 | 5.4 | 4.8 | 4.5 | 3.9 |
| Hours | 6 | 8 | 12 | 16 | 24 | 36 | 48 |
| Ratio | 3.4 | 2.7 | 1.8 | 1.3 | 1.1 | 0.7 | 0.7 |

| Hours | 72 | 96 | 168 |
|---|---|---|---|
| Ratio | 0.5 | 0.3 | 0.1 |

### Example 2

To ensure the accuracy of data collected from in vivo studies, it was important to first develop in vitro analytical methods for assaying an active pharmaceutical ingredient that may be used in the compositions and methods of the invention. Accordingly, an LC-MS/MS analytical method for assaying cyclobenzaprine in beagle dogs was developed as described below.

Reagents were prepared as follows:

Methanol/UHQ water 20/80 v/v with 0.1% formic acid: 20 mL of methanol (VWR) was mixed with 80 mL of UHQ water (ADME) and 0.1 mL of formic acid (Merck).

Carbonate buffer (Na₂CO₃ 0.1 M/NaHCO₃ 0.1 M 50/50 v/v (pH 9.8): 2.65 g of Na₂CO₃ was diluted in 250 mL of UHQ water to make 0.1 M Na₂CO₃. 2.1 g of NaHCO₃ was diluted in 250 mL of UHQ water to make 0.1 M NaHCO₃. The final solution was prepared by mixing 250 mL of 0.1 M Na₂CO₃ and 250 mL of 0.1 M NaHCO₃.

Analyst® 1.5.1 (Applied Biosystems) software was used to capture and integrate the chromatographic peak area. Watson® 7.2.0.03 (Thermo Electro Corporation) software was used for concentration calculations, data analysis, to store concentration data and statistical calculation. Excel® (2003) (Microsoft) was used for the statistical calculation for carry over.

Labeled cyclobenzaprine ([¹³C, ²H₃]cyclobenzaprine HCl) (Alsachim) was used as an internal standard (IS) for the cyclobenzaprine HCl (Alsachim) reference sample. Cyclobenzaprine and IS standard solutions were prepared according to Tables 4 and 5 after dissolution of a definite amount in a dark flask in appropriate volume of solvent taking into account the respective corrective factors. The solutions were vortex mixed and sonicated until complete dissolution if necessary.

TABLE 4
Standard solutions

| | Cyclobenzaprine | Internal Standard |
|---|---|---|
| Stock solution (mg/mL) | 1 | 1 |
| Solvent | Methanol | Methanol |
| Container | Glass | Glass |

These solutions were aliquoted following their preparation, stored at −20° C.±5° C., and each aliquot was discarded after use.

TABLE 5
Diluted cyclobenzaprine solutions

| Spiking | | | | |
|---|---|---|---|---|
| solution (from | | | | |
| Table 4) | Used | | Final | Final |
| concentration | volume | | volume | concentration |
| (μg/mL) | (mL) | Solvent | (mL) | (μg/mL) |
| 1000 | 0.05 | Methanol | 5 | 10 |
| 10 | 0.25 | Methanol | 5 | 0.5 |
| 10 | 0.2 | Methanol | 5 | 0.4 |
| 10 | 0.15 | Methanol | 5 | 0.3 |
| 10 | 0.125 | Methanol | 5 | 0.25 |
| 10 | 0.05 | Methanol | 5 | 0.1 |
| 0.5 | 0.5 | Methanol | 5 | 0.05 |
| 0.5 | 0.25 | Methanol | 5 | 0.025 |
| 0.5 | 0.1 | Methanol | 5 | 0.01 |
| 0.1 | 0.15 | Methanol | 5 | 0.003 |
| 0.1 | 0.05 | Methanol | 5 | 0.001 |

TABLE 6
Diluted internal standard solutions

| Spiking | | | | |
|---|---|---|---|---|
| solution | Used | | Final | Final |
| concentration | volume | | volume | concentration |
| (μg/mL) | (mL) | Solvent | (mL) | (μg/mL) |
| 1000 | 0.025 | Methanol | 2.5 | 10 |
| 10 | 0.125 | Methanol | 5 | 0.25 |

In a polypropylene microcentrifuge tube, 20 μl of the appropriate diluted solution of cyclobenzaprine was added to 200 μl of Heparin lithium Beagle dog plasma (also referred to herein as blank dog plasma) previously centrifuged at 3500 rpm between 0 and 9° C. for approximately 5 min as follows:

TABLE 7
Preparation of calibration standards

| | | | | | | | | | | |
|---|---|---|---|---|---|---|---|---|---|---|
| Spiking | 0.001 | 0.003 | 0.01 | 0.025 | 0.05 | 0.1 | 0.25 | 0.3 | 0.4 | 0.5 |
| solution | | | | | | | | | | |
| conc. (μg/mL) | | | | | | | | | | |
| Volume | 20 | 20 | 20 | 20 | 20 | 20 | 20 | 20 | 20 | 20 |
| added (μl) | | | | | | | | | | |
| Volume of | 200 | 200 | 200 | 200 | 200 | 200 | 200 | 200 | 200 | 200 |
| blank dog | | | | | | | | | | |
| plasma (μl) | | | | | | | | | | |
| Conc. of | 0.1 | 0.3 | 1 | 2.5 | 5 | 10 | 25 | 30 | 40 | 50 |
| cyclobenzaprine | | | | | | | | | | |
| in | | | | | | | | | | |
| plasma | | | | | | | | | | |
| (ng/mL) | | | | | | | | | | |

TABLE 8
Preparation of blank and double blank

| Sample | S0 | S0SI |
|---|---|---|
| Vol. of methanol added | 20 | 20 |
| (μl) | | |
| Vol. of blank dog plasma | 200 | 200 |
| (μl) | | |

TABLE 9
Preparation of quality controls

| | | | | |
|---|---|---|---|---|
| Spiking | 0.001 | 0.003 | 0.25 | 0.4 |
| solution used | | | | |
| (μg/mL) | | | | |
| Vol. of solution | 20 | 20 | 20 | 20 |
| added (μl) | | | | |
| Vol. of blank | 200 | 200 | 200 | 200 |
| dog plasma (μl) | | | | |
| Conc. of | 0.1 | 0.3 | 25 | 40 |
| cyclobenzaprine | | | | |
| in plasma | | | | |
| (ng/ml) | | | | |

Calibration standards and quality control samples were prepare daily.

The extraction procedure was performed as follows: 20 μl of [¹³C, ²H₃]cyclobenzaprine at 0.25 μg/mL or 20 μl of methanol for S0 was added to 200 μl of blank dog plasma. To this solution, 200 μl of carbonate buffer was added and vortex mixed for 10 seconds. Subsequently, 1000 μl of hexane was added to this solution and mixed at 180 rpm for 10 minutes before centrifugation at 3500 rpm, between 0 and 9° C. for 5 minutes. The organic phase was transferred in a polypropylene tube and dried under a nitrogen stream at 40° C. 200 μl of methanol/water (20/80, v/v) with 0.1% formic acid was added to the dry residue, which was then sonicated for about 5 minutes and vortex mixed for 10 seconds. The mixture was then transferred into a polypropylene plate, centrifuged at 3500 rpm between 0 and 9° C. for 5 minutes, and stored between 0 and 9° C. before injection into the LC-MS/MS system. Chromatography was then performed using the parameters shown below.

TABLE 10
Chromatographic parameters

| | |
|---|---|
| HPLC Column | Onyx monolithic Phenomenex C 18 100 × 3.0 mm |
| Precolumn | — |
| Column temperature (° C.) | 40 |
| Type of column oven | CTO-20AC, Shimadzu |

TABLE 11
Automatic sampler parameters

| | |
|---|---|
| Type of automatic sampler | SIL-20AC, Shimadzu |
| Injection volume (μl) | 10 |
| Automatic sampler temperature (° C.) | 4 |
| Rinsing Volume (μl) | 300 |
| Rinsing solution | Acetonitrile/Water (70/30, v/v) |
| Rinse Mode | After aspiration |

TABLE 12
Pump parameters

| Type of pump | LC-20AD, Shimadzu | | |
|---|---|---|---|
| Flow rate (mL/min) | 1 | | |
| Mobile phase | Gradient: | | |
| | Time (min) | channel A | channel B |
| | 0 | 30 | 70 |
| | 1 | 90 | 10 |
| | 1.8 | 90 | 10 |
| | 1.9 | 30 | 70 |
| | 4 | 30 | 70 |

| | |
|---|---|
| | Channel A: Methanol + 0.1% formic acid |
| | Channel B: Water + 0.1% formic acid |
| Detection mode | LC-MS/MS |
| Type of detector | API4000 |

TABLE 13
Other parameters

| | |
|---|---|
| Run time (min) | 4 |
| Retention time | |
| Cyclobenzaprine | About 1.6 min |
| [¹³C, ²H₃] cyclobenzaprine | About 1.6 min |

TABLE 14
Comparison of cyclobenzaprine and [¹³C, ²H₃]
cyclobenzaprine in HPLC parameters

| | Parent | Daughter | Dwell | DP | CE | CXP | EP |
|---|---|---|---|---|---|---|---|
| Analyte | (m/z) | (m/z) | (msec) | (V) | (eV) | (V) | (V) |
| Cyclobenzaprine | 276.2 | 216.3 | 100 | 70 | 33 | 14 | 10 |
| [¹³C, ²H₃] | 280.3 | 216.3 | 100 | 70 | 33 | 14 | 10 |
| cyclobenzaprine | | | | | | | |

TABLE 15
Acquisition parameters
Ionisation mode: ESI+

TABLE 16
Tandem mass spectrometer tune conditions

| | |
|---|---|
| CAD (psi) (collision gas) | 4 |
| CUR (psi) (curtain gas) | 25 |
| GS1 (psi) (ion source gas 1) | 50 |
| GS2 (psi) (ion source gas 2) | 60 |
| IS (V) (ion spray voltage) | 5500 |
| TEM (° C.) (temperature) | 550 |

The data collected during HPLC methods development is described in the following tables. In particular, the data show that there was no carry over of cyclobenzaprine in consecutive runs when comparing cyclobenzaprine peak areas and blank samples (Table 17). The data also show that, when comparing [¹³C, ²H₃]cyclobenzaprine HCl area and internal standard area in blank samples, carry over is evident only in the first run. However, at 0.1% interference, the extent of carry over is negligible (Table 18).

TABLE 17
Comparison of cyclobenzaprine areas in lower limit of quantification
(LLOQ) sample and blank samples injected
after upper limit of quantification (ULOQ) sample

| | | Area of | |
|---|---|---|---|
| | | CYCLOBENZAPRINE | |
| | Area of | peak in LLOQ | |
| Run | CYCLOBENZAPRINE | calibration standard | % |
| Identification | peak in S0 | (LLOQ) | interference |
| 1 | No peak | 1268 | 0.0 |
| 2 | No peak | 1268 | 0.0 |
| 3 | No peak | 1268 | 0.0 |

TABLE 18
Comparison of [¹³C, ²H₃] Cyclobenzaprine HCl area in S0SI sample and
internal standard area in blank samples injected after ULOQ sample

| | Area of ¹³C, ²H₃ | Area of ¹³C, ²H₃ | |
|---|---|---|---|
| Run | CYCLOBENZAPRINE | CYCLOBENZAPRINE | % |
| Identification | peak in S0 | peak in S0SI | interference |
| 1 | 255 | 303814 | 0.1 |
| 2 | No peak | 303814 | 0.0 |
| 3 | No peak | 303814 | 0.0 |

TABLE 19
Back calculated dog plasma cyclobenzaprine concentrations (ng/mL) and
regression parameters

| Conc. | | | | | | | | | | | | | |
|---|---|---|---|---|---|---|---|---|---|---|---|---|---|
| (ng/mL) | 0.1000 | 0.3000 | 1.000 | 2.500 | 5.000 | 10.00 | 25.00 | 30.00 | 40.00 | 50.00 | Slope | Intercept | R² |
| Batch 1 | 0.09639 | 0.3322 | 1.009 | 2.495 | 4.89 | 10.19 | 25.38 | 28.78 | 39.74 | 47.85 | 0.03996 | 0.0005901 | 0.9978 |
| Batch 2 | 0.0940 | 0.3643 | 0.9103 | 2.302 | 5.066 | 10.16 | 26.4 | 29.81 | 40.61 | 45.98 | 0.03914 | 0.001496 | 0.9908 |
| n | 2 | 2 | 2 | 2 | 2 | 2 | 2 | 2 | 2 | 2 | 2 | 2 | 2 |
| Overall | 0.09521 | 0.3483 | 0.9597 | 2.399 | 4.978 | 10.18 | 25.89 | 29.3 | 40.18 | 46.92 | 0.03955 | 0.001043 | 0.9943 |
| Mean | | | | | | | | | | | | | |
| % | −4.79 | 16.1 | −4.03 | −4.04 | −0.44 | 1.8 | 3.56 | −2.33 | 0.45 | −6.16 | | | |
| Deviation | | | | | | | | | | | | | |

TABLE 20
Intra-run (repeatability), precision, and deviation of
a cyclobenzaprine assay in dog plasma

| | | D | E | F | G |
|---|---|---|---|---|---|
| | Curve | 0.1000 | 0.3000 | 25.00 | 40.00 |
| Measurement | No. | ng/ml | ng/ml | ng/ml | ng/ml |
| Individual Runs | 1 | 0.1079 | 0.3143 | 25.03 | 39.07 |
| | | 0.1172 | 0.3253 | 25.57 | 40.04 |
| | | 0.1131 | 0.2969 | 25.51 | 40.26 |
| | | 0.1181 | 0.3312 | 25.95 | 40.11 |
| | | 0.1161 | 0.3419 | 25.05 | 39.61 |
| | | 0.1298 | 0.3295 | 25.58 | 41.21 |
| Intrarun Mean | | 0.117 | 0.3232 | 25.45 | 40.05 |
| Intrarun SD | | 0.007265 | 0.01568 | 0.3525 | 0.7138 |
| Intrarun % CV | | 6.21 | 4.85 | 1.39 | 1.78 |
| Intrarun % Deviation | | 17 | 7.73 | 1.8 | 0.13 |
| N | | 6 | 6 | 6 | 6 |
| Individual Runs | 2 | 0.1013 | 0.31 | 25.9 | 43.98 |
| | | 0.1069 | 0.3208 | 26.7 | 42.82 |
| | | 0.07961 | 0.2957 | 26.34 | 43.4 |
| | | 0.1076 | 0.3177 | 26.01 | 42.63 |
| | | 0.0942 | 0.3312 | 26.49 | 43.95 |
| | | 0.09532 | 0.3486 | 27.41 | 43.44 |
| Intrarun Mean | | 0.09749 | 0.3207 | 26.48 | 43.37 |
| Intrarun SD | | 0.0104 | 0.0181 | 0.5459 | 0.5594 |
| Intrarun % CV | | 10.67 | 5.64 | 2.06 | 1.29 |
| Intrarun % Deviation | | −2.51 | 6.9 | 5.92 | 8.43 |
| N | | 6 | 6 | 6 | 6 |

TABLE 21
Inter-run (reproducibility), precision, and deviation of
a cyclobenzaprine assay in dog plasma

| | D | E | F | G |
|---|---|---|---|---|
| Curve | 0.1000 | 0.3000 | 25.00 | 40.00 |
| Number | ng/ml | ng/ml | ng/ml | ng/ml |
| 1 | 0.1079 | 0.3143 | 25.03 | 39.07 |
| | 0.1172 | 0.3253 | 25.57 | 40.04 |
| | 0.1131 | 0.2969 | 25.51 | 40.26 |
| | 0.1181 | 0.3312 | 25.95 | 40.11 |
| | 0.1161 | 0.3419 | 25.05 | 39.61 |
| | 0.1298 | 0.3295 | 25.58 | 41.21 |
| 2 | 0.1013 | 0.31 | 25.9 | 43.98 |
| | 0.1069 | 0.3208 | 26.7 | 42.82 |
| | 0.07961 | 0.2957 | 26.34 | 43.4 |
| | 0.1076 | 0.3177 | 26.01 | 42.63 |
| | 0.0942 | 0.3312 | 26.49 | 43.95 |
| | 0.09532 | 0.3486 | 27.41 | 43.44 |
| Mean Concentration Found (ng/ml) | 0.1073 | 0.3219 | 25.96 | 41.71 |
| Inter-run SD | 0.01332 | 0.01619 | 0.6924 | 1.838 |
| Inter-run % CV | 12.41 | 5.03 | 2.67 | 4.41 |
| Inter-run % Deviation | 7.3 | 7.3 | 3.84 | 4.28 |
| N | 12 | 12 | 12 | 12 |

### Example 3

The LC-MS/MS analytical method described above was validated using acceptance criteria of ±25% (except for the lower limit of quantification (LLOQ), which was set at an acceptance criterion of ±30%) with respect to the concentration response relationship and intra- and inter-run precision of deviation. Cyclobenzaprine HCl and the internal standard (IS) [¹³C, ²H₃]cyclobenzaprine HCl were used, as described above.

For validation of the LC-MS/MS method, the target LLOQ was set to 0.1 ng/mL. The upper limit of quantification (ULOQ) therefore corresponds to a maximum of 500-fold times the LLOQ, or 50 ng/mL. The method required a lithium heparinized plasma sample volume of 200 μl, and the extraction was a liquid liquid extraction with hexane. The extracts were injected using a HPLC system and an AP14000® (Applied Biosystems) for the MS/MS detection, with an “Onyx monolithic Phenomenex C18 100×3.0 mm” column. No data were excluded from the calculations. The difference between the mean concentration observed and the nominal concentration were used to estimate the accuracy of the method. The coefficient of variation was used to estimate the precision of the method.

Blank dog plasma was spiked with cyclobenzaprine HCl at four concentrations: LLOQ, 3×LLOQ, 0.5×ULOQ, and 0.8×ULOQ. The concentration-response relationship was determined from the calibration standards at concentrations ranging from the LLOQ up to the ULOQ on 2 different runs. At least eight calibration points different from zero, prepared the same day of the analysis, were used for each calibration curve. One blank dog plasma samples not spiked (S0) and two spiked only with the internal standard (S0SI) were analyzed with each calibration curve. The weighting factor was determined during the qualification according to the results of calibration curve regression fits. The simplest model that adequately described the concentration-response relationship was used.

Target acceptance criteria included deviation for 75% of the calibration standards that ranged between ±30.00% of the nominal value for the LLOQ concentration level and between ±25.00% of the nominal values for the other concentration levels. Deviation % was measured as ((Cmeas−Cn)/Cn)×100, wherein Cmeas is the measured or back-calculated concentration and Cn is the nominal concentration. The lowest and highest levels were included in the calibration curve, and calibration standards were excluded (except LLOQ and ULOQ) from the final calibration curve only if the back calculated deviation was not comprised within ±25.00% of the nominal values. Using this method, for each S0 and S0SI sample, interferences at the cyclobenzaprine retention time in the multiple reaction monitoring (MRM) traces specific for cyclobenzaprine should be lower than 30.00% of the cyclobenzaprine peak area in the LLOQ calibration standard, and for each S0 sample, interferences at internal standard retention time in the MRM trace specific for the internal standard should be lower than 2.00% of the internal standard peak area in S0SI. If these criteria are not fulfilled, a documented investigation should be performed on 3×LLOQ fresh QC samples in order to evaluate a possible impact on cyclobenzaprine measured concentrations to draw a conclusion on the analytical run qualification. A summary of the criteria to include an analytical batch or run is given in Table 22, below.

TABLE 22
Acceptance criteria

| | QC samples acceptance | |
|---|---|---|
| Series description | criteria | Calibration curve acceptance criteria |
| Series with 24 | At least 67% (16 out of 24) | At least 75% of calibration standards |
| fresh QC samples | of QC samples should have | (including LLOQ and ULOQ) should be |
| (4 concentration | a deviation between | taken into account for calculation of |
| levels in sextuplet) | ±25.00% (or ±30.00% for | calibration curve regression parameters |
| | LLOQ concentration level) | Possible interference peak area at |
| | with at least 2 QC per level | cyclobenzaprine retention time in S0SI and |
| | inside the acceptance criteria | S0 samples should be <30.00% of |
| | | cyclobenzaprine peak area in LLOQ |
| | | calibration standard |
| | | Possible interference peak area at internal |
| | | standard retention time in S0 samples |
| | | should be <2.00% of internal standard |
| | | peak area in S0SI sample |

The intra-run and inter-run precision and deviation of the assay method were tested in dog plasma for each concentration level on two different runs. For each concentration, QC samples were extracted in sextuplet within the same run. Precision % was measured as (standard deviation/Cmean)×100. All QC results were used for precision and mean deviation calculations except if any were rejected for a defined analytical problem. In order to meet the acceptance criteria, precision was calculated for each concentration and should be <25.00% except for LLOQ where <30.00% is acceptable (calculated with n=6 for each repeatability test and n=12 for the reproducibility test). The mean deviation also was calculated and should be within ±30.00% at LLOQ and within ±25.00% for the other concentrations accepted (calculated with n=6 for each repeatability test and n=12 for the reproducibility test).

One S0 sample was injected three times immediately following the last higher calibration standard (ULOQ) of at least one calibration curve. The MRM chromatograms were examined for the presence of peaks at the retention times of cyclobenzaprine and the IS, and the area of all peaks were measured. In order to meet the acceptance criteria, the peak area obtained at the retention time of the cyclobenzaprine in the MRM trace specific for cyclobenzaprine in each S0 should be less than 30.00% of the peak area obtained for cyclobenzaprine in the first calibration standard (LLOQ). The peak area obtained at the retention time of the internal standard in the MRM trace specific for the internal standard in each S0 should be less than 2.00% of the peak area obtained for internal standard in the S0SI.

Cyclobenzaprine concentrations were calculated using Watson® 7.2.0.03 directly from the peak area obtained after automatic integration of chromatograms by Analyst® 1.5.1. Concentrations of the QC samples were calculated by interpolation with the weighted calibration curves prepared in the same conditions and assessed daily after automatic integration. The results of the concentrations, once accepted, were expressed in “ng/mL”. Statistics (mean, SD, precision and deviation) were calculated with Watson® 7.2.0.03, except for carry over, which was calculated with Excel®.

### Example 4

In order to study the effects of the compositions and methods of the invention, a protocol was developed to estimate the levels of unchanged cyclobenzaprine plasma concentrations after a single oral, sublingual or intravenous administration of cyclobenzaprine hydrochloride to a female beagle dog. The protocol was designed as outlined in Table 23.

The in vivo protocol uses the analytical method described above to test the cyclobenzaprine HCl, with a calibration range from 0.1 to 50 ng/mL. The method requires a lithium heparinized plasma sample volume of 200 μl. The extraction is a liquid liquid extraction with hexane, and the extracts are injected using an HPLC system and an AP14000® (Applied Biosystems) for the MS/MS detection, with a “Onyx monolithic Phenomenex C18 100×3.0 mm” column.

TABLE 23
In vivo study design

| | |
|---|---|
| Animals | 5 non naive female beagle dogs |
| Compound | Cyclobenzaprine hydrochloride |
| Administration routes | Oral by nasogastric tube (NG) |
| | Sublingual (SL) |
| | Intravenous (IV) |
| Administered dose | 0.14 mg/kg (corresponding to a 10 mg dose |
| | in a 70 kg human) |
| Formulation concentration | NG: 0.028 mg/mL |
| | Sublingual: 2.5 mg/mL |
| | IV: 0.14 mg/mL |
| Vehicle | Aqueous solution of monopotassium |
| | phosphate (KH₂PO₄) at pH 7.4 |
| Administration volume | NG: 5 mL/kg |
| | Sublingual: 0.056 mL/kg |
| | IV: 1 mL/kg |

To check the quality of the method during the biological sample assays, fresh QC samples are prepared at 3×LLOQ, 0.5×ULOQ and 0.8×ULOQ concentration levels, in duplicate. Each assay series consists of one sample of blank lithium heparinized beagle dog plasma not spiked (S0), two samples of blank lithium heparinized beagle dog plasma spiked only with the internal standard (S0SI), eight calibration standards, a minimum of six quality control (QC) samples covering three different concentrations of cyclobenzaprine in duplicate, distributed throughout and at the end of the series, and the biological samples being assayed.

The criteria described hereafter are used to validate calibration curves: deviation for 75% of calibration standards should be ±25.00% of the nominal value and ±30.00% for the LLOQ; the lowest and the highest levels have to be included in the calibration curve; and the calibration standard should be excluded from the final calibration line if the back-calculated concentration was not ±25.00% of the nominal value. The criteria described hereafter are used to perform an additional investigation if necessary: in each S0SI, possible interferences at retention time of cyclobenzaprine should be lower than 30.00% of the cyclobenzaprine peak area in the LLOQ calibration standard. If these criteria are not met, an investigation should be performed on pre-dose and 3×LLOQ QC samples in order to evaluate a possible impact on measured cyclobenzaprine concentrations and, therefore, on the analytical run validation.

A series is considered validated if at least 67% of QC samples have a deviation range of ±25.00% of the nominal concentrations. Additionally, any rejected QC samples should not correspond to the last analyzed QC samples of the series. Therefore, only concentrations measured between validated QC samples are considered validated. If dilutions are necessary, diluted QCs will be added in order to validate the dilution procedure. Diluted concentrations will be validated if at least 1 QC out of 2 has a deviation range of ±25.00% of the nominal concentration.

Concentrations of the samples are calculated using Watson® 7.2.0.03 directly from chromatograms after automatic integration by Analyst® 1.5.1 and expressed as ng/mL. Mean plasma concentrations will be calculated (when calculable, i.e. n>2) using individual concentrations and will be expressed with the corresponding standard deviation value and variation coefficients (when calculable, i.e. n>3) (with CV(%)=(SD/mean)×100). The individual plasma concentrations are tabulated for each dog and scheduled sampling time. Concentrations below the LLOQ are designated as “BLQ.” All BLQ concentrations are substituted by zero for calculation of the descriptive statistics of the concentrations.

Pharmacokinetic analysis is carried out using KINETICA® (Version 4.3 (Thermo Electron Corporation)). An independent model (non compartmental analysis) is used. All BLQ values in the absorption phase are substituted by zero, except for BLQ values between evaluable concentrations, which are treated as missing values, before the calculation of the pharmacokinetic variables. The terminal BLQ values are ignored. The following pharmacokinetic parameters are calculated:

Cₘₐₓ (ng/mL): observed maximal plasma concentration (for oral and sublingual administration)

Tₘₐₓ (h): time since administration at which maximum plasma concentration is found (for oral and sublingual administration).

AUCₜ(ng/(mL×h)): area under the plasma concentration time curve from administration to the last observed concentration at time t measured by trapezoidal rules.

AUC_{inf} (ng/mL×h): area under the plasma concentration time curve from administration up to infinity with extrapolation of the terminal phase. This can also be represented as AUC_{0-∞}.

% AUCₑₓₜᵣₐ: percentage of extrapolated AUC, calculated as (AUC_{inf}−AUCₜ/AUC_{inf})×100

T_{1/2}* (h): half-life of elimination, calculated as ln 2/Kₑₗ

Kₑₗ* (l/h): estimated by the linear regression of the logarithm of the terminal concentration as a function of time using Kinetica® software.

Cl and Cl/F* (L/h): apparent plasma clearance, calculated as dose/AUC_{inf}

V_{d} and V_{d}/F* (L): apparent volume of distribution, calculated as Cl/Kₑₗ

$$Absolute bioavailability F \% ( \% ) = ( ( AUC by extravascular administration ) / AUC by IV administration ) \times 100$$

*These parameters are calculated only if % AUCₑₓₜᵣₐ is lower than 20% and if the elimination phase contains three time points.

### Example 5

To test the effects of the methods and compositions of the invention described herein, a protocol was designed for the administration of cyclobenzaprine HCl in beagle dogs. The protocol is described below.

5 non-naive female dogs are used, each dog receiving the test substance by oral administration via nasogastric tube to the stomach (NG) administration, sublingual administration (SL), and by intravenous (IV) administration. There is a “wash-out” period of at least two weeks between each type of administration. Blood samples are drawn as follows:

### Session 1: Oral Administration

A single NG dose of 0.14 mg/kg (under a volume of 5 ml/kg and a solution concentration of 0.028 mg/mL) is administered. Blood samples are taken pre-dosing, and then at 30 min, 1, 2, 3, 4, 5, 6, 8, 10, 12 and 24 hours after administration (for a total of 12 blood samples per animal).

### Session 2: Sublingual Administration

After a wash-out period of at least 2 weeks, dogs are sedated using propofol (6.5 mg/kg IV). They will be then given a single sublingual dose of 0.14 mg/kg (under a volume of 0.056 mL/kg and a solution concentration of 2.5 mg/mL). Blood samples are taken pre-dosing, and then at 10 min, 20 min, 30 min, 1, 2, 3, 4, 6, 8, 10 and 24 hours after administration (for a total of 12 blood samples per animal). After administration, animals do not have any access to water for 30 minutes.

### Session 3: Intravenous Administration

After a wash-out period of at least 2 weeks, dogs are given a single IV dose of 0.14 mg/kg (under a volume of 1 mL/kg, bolus of approximately 30 sec, and a solution concentration of 0.14 mg/mL). Blood samples are taken pre-dosing, and then at 10 min, 20 min, 30 min, 1, 2, 3, 4, 6, 8, 10 and 24 hours after administration (for a total of 12 blood samples per animal).

Blood sampling was designed to minimize animal suffering and to ensure the quality of the biological samples, and was adapted from basic procedures commonly used in studies performed in dogs. Serial blood samples (1 tube of approximately 5 mL) are collected from a jugular vein using vacuum tubes containing lithium heparin. After sealing each tube, the blood samples are manually agitated and stored on ice until centrifugation (within 30 minutes of sampling). The samples are centrifuged at 1500 g, at 4° C., for 10 minutes. The entire resultant plasma obtained from each tube is immediately transferred to suitably labeled polypropylene tubes (3 aliquots of plasma of at least 500 μl each), which are stored upright at approximately −80° C. and protected from light until bioanalysis.

The dogs are fasted overnight before each administration, and food is given to the dogs 4 hours after each treatment. The cyclobenzaprine HCl is dosed at 0.14 mg/kg for each of the three routes of administration (PO, sublingual, and IV). In each of the three routes, a potassium phosphate buffer (pH 7.4) is used as the vehicle.

Female beagle dogs, weighing 12-18 kg, obtained from HARLAN or CEDS are used in these trials. The dogs are housed in groups in a kennel with free access to food and water, under natural lighting and in a controlled ambient temperature of 18±3° C. During the pharmacokinetic phase, dogs are housed singly in a floor area of approximately 1 or 2 m². During this phase, the animal house is maintained under artificial lighting (12 hours) between 7:00 and 19:00 in a controlled ambient temperature of 18±3° C.

### Example 6

The solubility of cyclobenzaprine HCl was tested both in purified water (Table 24) and in an aqueous solution containing monobasic potassium phosphate (KH₂PO₄) with NaOH to adjust the solution to pH 7.4. The (KH₂PO₄) solution was prepared according to the current USP (USP 34) (Table 25). Briefly, 50 mL of 0.2 M monobasic potassium phosphate (KH₂PO₄) was mixed with 39.1 mL of 0.2 M NaOH, and water was added to bring the solution to a final volume of 200 mL. During the test, the change in the pH of the solution also was measured for each addition. The test was performed on a 100 mL sample, adding 5 g of cyclobenzaprine HCl each time to the initial aliquot of 10 g. Each pH value was measured only after complete dissolution of the added quantity of cyclobenzaprine HCl. The measured values are reported in the tables below. The solubility data for cyclobenzaprine HCl reported in the literature (30 g for 100 g of water) was consistent with use of the aqueous KH₂PO₄ solution (pH 7.4) as a solvent.

TABLE 24
Dissolution in purified water (volume = 100 mL)

| Grams of | | |
|---|---|---|
| cyclobenzaprine | pH | |
| HC1 added | value | Molarity |
| 0 | 5.80 | 0 |
| 10 | 3.31 | 0.32 |
| 15 | 3.24 | 0.48 |
| 20 | 3.16 | 0.64 |
| 25 | 3.11 | 0.80 |
| 30 | 3.07 | 0.96 |

TABLE 25
Dissolution in Phosphate Buffer Solution pH 7.4
(volume = 100 mL)

| Grams of | | |
|---|---|---|
| cyclobenzaprine | pH | |
| HC1 added | value | Molarity |
| 0 | 7.37 | 0 |
| 10 | 6.43 | 0.32 |
| 15 | 6.29 | 0.48 |
| 20 | 6.21 | 0.64 |
| 25 | 6.15 | 0.80 |
| 30 | 6.10 | 0.96 |

According to the protocol relative to the pre-clinical study described above, the administered solution for a sublingual route had to be at a concentration of 2.5 mg/mL (solvent: aqueous KH₂PO₄ solution, pH 7.4, as described above). The volume of the cyclobenzaprine HCl solution administered was 0.056 mL/kg. Looking to the available literature, and in anticipation of gathering data after the setup of the preliminary formulation, we hypothesized an average weight of 10 kg for each dog. The quantity of cyclobenzaprine HCl solution administered under the tongue was therefore 0.56 mL, equivalent to 1.4 mg of cyclobenzaprine HCl.

In order to simulate the ambient pH under the animal tongue, artificial saliva was prepared taking into account the standard procedure in the available literature (see, for example, UNI EN 12868:2002), with some modifications. Briefly, 4.2 g of sodium bicarbonate, 500 mg of sodium chloride, 200 g of potassium carbonate and 30 mg of sodium nitrite were dissolved into 900 mL of purified water under agitation with a magnetic stirrer. The final pH of the solution was between about pH 8 and pH 9, which was acidified to approximately pH 5.5 using lactic acid.

In order to consider different possible volumes of saliva under the tongues of the animals, the 0.56 mL of cyclobenzaprine HCl solution (solvent: H₂O; basifying agent: aqueous KH₂PO₄ solution, pH 7.4, as described above) corresponding to the dose administered to the dogs, was added to 1 mL, 3 mL and 5 mL of saliva, and the pH values were measured as described in Table 26. The same pH measurements were performed adding the formulation without the basifying agent to the saliva instead of the cyclobenzaprine HCl alone (Table 24).

TABLE 26
pH measurements of a cyclobenzaprine
HCl solution in artificial saliva

| | 1 mL | 3 mL | 5 mL |
|---|---|---|---|
| | artificial | artificial | artificial |
| | saliva | saliva | saliva |
| Solution | pH = 7.19 | pH = 7.04 | pH = 6.73 |
| corresponding | | | |
| to the dose | | | |
| (0.56 mL of | | | |
| aqueous solution | | | |
| containing | | | |
| KH₂PO₄ | | | |
| with 2.5 | | | |
| mg/ml of | | | |
| cyclobenzaprine | | | |
| HC1) | | | |

TABLE 27
pH measurements of a cyclobenzaprine HCl tablet in
artificial saliva (control: lacking a basifying agent)

| | 1 mL | 3 mL | 5 mL |
|---|---|---|---|
| | artificial | artificial | artificial |
| | saliva | saliva | saliva |
| 1 tablet | pH = 5.40 | pH = 5.47 | pH = 5.51 |
| (corresponding | | | |
| to 2.4 mg of | | | |
| cyclobenzaprine | | | |
| HC1) | | | |

In order to increase the pH under the tongue after the administration of the tablet to a pH value as similar as possible to the aqueous KH₂PO₄ solution, pH 7.4, as described above, K₂HPO₄ was added to the tablet formulation. The quantity had been determined by pH trials on solutions obtained by dissolving the tablets of the formulation lacking the K₂HPO₄ basifying agent in the artificial saliva. By adding 1.05 mg of K₂HPO₄ to the control formulation (lacking the K₂HPO₄ basifying agent), the results depicted in Table 28 were obtained.

TABLE 28
pH measurements of a cyclobenzaprine HCl tablet in
artificial saliva (basifying-agent-containing)

| | 1 mL | 3 mL | 5 mL |
|---|---|---|---|
| | artificial | artificial | artificial |
| | saliva | saliva | saliva |
| 1 tablet | pH = 7.11 | pH = 6.83 | pH = 6.56 |
| (corresponding | | | |
| to 2.4 mg of | | | |
| cyclobenzaprine | | | |
| HC1) | | | |

### Example 7

An exemplary composition formulated for sublingual administration is a sublingual tablet designed to quickly disintegrate under the tongue. To develop this type of composition, the galenic properties of cyclobenzaprine HCl were studied. A solubility test confirmed that slightly basic media (e.g., aqueous KH₂PO₄ solution, pH 7.4, described above) is a possible solvent for cyclobenzaprine HCl.

The specifications for sublingual dosage forms are not defined by pharmacopoeias, so a preliminary pool of excipients was selected in order to obtain a tablet with a disintegration time in compliance with USP specifications referred to an oral dispersible forms (disintegration in less than 30 seconds). This specification is one target for formulations, but is not a mandatory specification. On the basis of this characteristic, a disintegrant (Crospovidone) and a highly palatable disintegrant (Pearlitol Flash) had been selected. On the basis of the characteristics of the aqueous KH₂PO₄ solution, pH 7.4, described above, a stoichiometric ratio of K₂HPO₄ was introduced in one of the preliminary formulations as basifying agent. In addition, a formulation lacking a basifying agent was produced. For each formulation approach, batches of cyclobenzaprine HCl from two different suppliers were tested with the aim of selecting the best performing batch for further testing. The final mixing was obtained in both cases by dry mixing, adding a lubricant only in the last mixing step. Due to the low concentration of the active ingredient, a progressive dilution method was applied.

The tabletting phase was performed on a GP1 tabletting machine equipped with a 4 mm punch. The punch selection took into account the method of administration, as it affects both the diameter and the shape of the tablet in order to comply with the sublingual route and transmucosal absorption. Final mixing and the corresponding tablets were tested for all the standard parameters as reported here below. Two formulations containing the K₂HPO₄ as a basifying agent were manufactured as described in Table 35. These formulations differed only in that they use cyclobenzaprine HCl from different suppliers. The analytical results of each formulation is summarized in Table 36.

TABLE 35
Basifying-agent-containing cyclobenzaprine
HCl formulations

| | | | | Proto- | Proto- |
|---|---|---|---|---|---|
| | | | | type | type |
| | Com- | | | A | B |
| Compo- | mercial | | | (mg/ | (mg/ |
| nent | Name | Function | Supplier | tab) | tab) |
| Cycloben- | Cycloben- | Active | Dipharma | 2.40 | — |
| zaprine | zaprine | | | | |
| HCl | HCl | Ingredient | | | |
| Cycloben- | Cycloben- | Active | Sifavitor | — | 2.40 |
| zaprine | zaprine | Ingredient | | | |
| HCl | HC1 | | | | |
| Mannitol | Pearlitol | Disinte- | Roquette | 31.55 | 31.55 |
| and | Flash | grant | | | |
| Maize | | | | | |
| starch | | | | | |
| Crospovi- | Kollidon | Disinte- | BASF | 2.00 | 2.00 |
| done | CL | grant | | | |
| Colloidal | Aerosil | Glidant | Evonik | 0.50 | 0.50 |
| Silica | 200 | | | | |
| Sodium | Lubrisanaq | Lubricant | Pharma | 0.50 | 0.50 |
| Stearyl | | | Trans | | |
| Fumarate | | | Sanaq | | |
| Dipotas- | Dipotas- | Basifying | VWR | 1.05 | 1.05 |
| sium | sium | agent | | | |
| hydrogen | hydrogen | | | | |
| phosphate | phosphate | | | | |

| | | |
|---|---|---|
| Total Weight | 38.00 | 38.00 |

TABLE 36
Analytical results from basifying-agent-containing
cyclobenzaprine HCl formulations

| | | | |
|---|---|---|---|
| | Specifica- | Prototype | Prototype |
| Analysis | tions | A | B |

Powder

| | | | |
|---|---|---|---|
| Distribution | TBD | Assay = | Assay = |
| | | 96.2% | 99.9% |
| | | SD = | SD = |
| | | 3.5 | 6.5 |
| | | RSD = | RSD = |
| | | 3.7% | 6.5% |
| Bulk Density | / | 0.53 | 0.50 |
| | | g/mL | g/mL |
| Tapped Density | / | 0.60 | 0.59 |
| (300 strokes) | | g/mL | g/mL |
| Carr's Index | / | 12.28% | 15.00% |
| Hausner Ratio | / | 1.14 | 1.18 |

Tablets

| | | | |
|---|---|---|---|
| Uniformity of | AV | 13.1 | 13.7 |
| Content | <15 | | |
| Assay | 95-105% | 99.4% | 103.8% |
| Purity : | TBD | | |
| Impurity 1 | | Absent | Absent |
| Impurity 2 | | Absent | Absent |
| Impurity 3 | | Absent | Absent |
| Impurity 4 | | 0.097 | 0.129 |
| Total unknown | | Absent | Absent |
| Each other | | 0.097 | 0.129 |
| impurity | | | |
| Disintegration | TBD | 54″ | 40″ |
| test | | | |
| Hardness | To be | 32.40N | 20.20N |
| | Defined | | |
| Friability Test | <1.0% | 0.3% | 0.3% |
| KF | To be | 1.50% | 1.36% |
| | Defined | | |

* Preliminary analytical conditions were applied to these preliminary trials;
TBD: to be determined

Formulations lacking the K₂HPO₄ basifying agents were manufactured as described below in Table 37. The analytical results from these formulations are described in Table 38.

TABLE 37
Cyclobenzaprine HCl formulations lacking a basifying agent

| | | | | Proto- | Proto- |
|---|---|---|---|---|---|
| | | | | type | type |
| | Com- | | | C | D |
| Compo- | mercial | | | (mg/ | (mg/ |
| nent | Name | Function | Supplier | tab) | tab) |
| Cycloben- | Cycloben- | Active | Dipharma | 2.40 | — |
| zaprine | zaprine | Ingre- | | | |
| HC1 | HCl | dient | | | |
| Cycloben- | Cycloben- | Active | Sifavitor | — | 2.40 |
| zaprine | zaprine | Ingre- | | | |
| HCl | HC1 | dient | | | |
| Mannitol | Pearlitol | Disinte- | Roquette | 32.60 | 32.60 |
| and | Flash | grant | | | |
| Maize | | | | | |
| starch | | | | | |
| Crospovi- | Kollidon | Disinte- | BASF | 2.00 | 2.00 |
| done | CL | grant | | | |
| Colloidal | Aerosil | Glidant | Evonik | 0.50 | 0.50 |
| Silica | 200 | | | | |
| Sodium | Lubrisanaq | Lubri- | Pharma | 0.50 | 0.50 |
| Stearyl | | cant | Trans | | |
| Fumarate | | | Sanaq | | |

| | | |
|---|---|---|
| Total weight | 38.00 | 38.00 |

TABLE 38
Analytical results from basifying-agent-containing
cyclobenzaprine HCl formulations

| | | | |
|---|---|---|---|
| Analyses | Specifica- | Prototype | Prototype |
| | tions | C | D |

Powder

| | | | |
|---|---|---|---|
| Distribution | TBD | Assay = | Assay = |
| | | 102.8% | 102.2% |
| | | SD = | SD = |
| | | 2.3 | 2.6 |
| | | RSD = | RSD = |
| | | 2.2% | 2.5% |
| Bulk | / | 0.52 | 0.51 |
| Density | | g/mL | g/mL |
| Tapped Density | / | 0.59 | 0.58 |
| (300 strokes) | | g/mL | g/mL |
| Carr's Index | / | 12.07% | 11.86% |
| Hausner Ratio | / | 1.14 | 1.14 |

Tablets

| | | | |
|---|---|---|---|
| Uniformity of | AV | 4.7 | 7.5 |
| Content | <15 | | |
| Assay | 95-105% | 102.2% | 98.8% |
| Purity : | TBD | | |
| Impurity 1 | | Absent | Absent |
| Impurity 2 | | Absent | Absent |
| Impurity 3 | | Absent | Absent |
| Impurity 4 | | 0.085 | 0.115 |
| Each other | | Absent | Absent |
| impurity | | | |
| Total impurities | | 0.085 | 0.115 |
| Disintegration | TBD | 1′ 5″ | 42″ |
| test | | | |
| Hardness | To be | 31.4N | 20.80N |
| | Defined | | |
| Friability Test | <1.0% | 0.2% | 0.3% |
| KF | To be | 1.41% | 1.31% |
| | Defined | | |

*Preliminary analytical conditions were applied to these preliminary trials;
TBD: to be determined

On the basis of the distribution, assays and uniformity of content results, both formulations were identified as suitable.

### Example 8

In order to evaluate the stability of the formulations described above, prototype A, B, C, D were maintained under the 50° C. stress condition for 30 days. The results are described below in Tables 39 and 40, respectively. Cyclobenzaprine HCl, without excipients, also was tested from each manufacturer over the course of 15 days, as shown in Table 41. In sum, the formulations were stable, especially considering the high-stress storage conditions. Differences between the formulation containing the K₂HPO₄ basifying agents and the formulation lacking the K₂HPO₄ basifying agents were minimal.

TABLE 39
Basifying-agent-containing cyclobenzaprine HCl
formulations at 50° C., for 30 days

| Purity | Basifying-agent- | | Basifying-agent- | |
|---|---|---|---|---|
| Profile of | containing | | containing | |
| Prototypes | prototype A | | prototype B | |
| (%) | T0* | T30days | T0* | T30days |
| Impurity 1 | Absent | Absent | Absent | Absent |
| (RT = about 56′) | | | | |
| Impurity 2 | Absent | 0.006% | Absent | 0.008% |
| (RT = about 6′) | | | | |
| Impurity 4 | Absent | Absent | Absent | Absent |
| (RT = about 15′) | | | | |
| Each Unknown | | | | |
| Impurity | | | | |
| Unknown 3 | Absent | 0.014% | Absent | 0.010% |
| (RT = about 7.5′) | | | | |
| Unknown 5 | 0.003% | 0.018% | 0.003% | 0.019% |
| (RT = about 11.1′) | | | | |
| Unknown 1 | 0.034% | 0.002% | 0.028% | 0.011% |
| (RT = about 16′) | | | | |
| Unknown 2 | Absent | 0.115% | Absent | 0.107% |
| (RT = about 17.5′) | | | | |
| Unknown 4 | Absent | Absent | Absent | Absent |
| (RT = about 26′) | | | | |
| Total Impurities | 0.037% | 0.155% | 0.031% | 0.147% |

*Data obtained on samples stored at room temperature

TABLE 40
Cyclobenzaprine HCl formulations without
basifying agent at 50° C., for 30 days

| Purity | | | | |
|---|---|---|---|---|
| Profile of | | | | |
| Prototypes | Prototype C | | Prototype D | |
| (%) | T0* | T30days | T0* | T30days |
| Impurity 1 | Absent | Absent | Absent | Absent |
| (RT = about 56′) | | | | |
| Impurity 2 | 0.004% | 0.006% | 0.005% | 0.008% |
| (RT = about 6′) | | | | |
| Impurity 4 | Absent | Absent | Absent | Absent |
| (RT = about 15′) | | | | |
| Each Unknown | | | | |
| Impurity | | | | |
| Unknown 3 | 0.003% | 0.011% | Absent | 0.009% |
| (RT = about 7.5′) | | | | |
| Unknown 5 | 0.002% | 0.017% | 0.004% | 0.018% |
| (RT = about 11.1′) | | | | |
| Unknown 1 | Absent | Absent | 0.018% | 0.011% |
| (RT = about 16′) | | 0.040% | | |
| Unknown 2 | 0.037% | Absent | 0.019% | 0.163% |
| (RT = about 17.5′) | | % | | |
| Unknown 4 | 0.011% | | 0.004% | Absent |
| (RT = about 26′) | | | | % |
| Total Impurities | 0.053% | 0.068% | 0.045% | 0.068% |

*Data obtained on samples stored at room temperature

TABLE 41
Cyclobenzaprine HCl active ingredient from
two manufacturers

| Purity | Cyclobenzaprine | | Cyclobenzaprine | |
|---|---|---|---|---|
| Profile | HCl | | HCl | |
| of APIs | (Sifavitor) | | (Dipharma) | |
| (%) | T0* | T15days | T0* | T15days |
| Impurity 1 | Absent | Absent | Absent | Absent |
| (RT = about 56′) | | | | |
| Impurity 2 | Absent | Absent | Absent | Absent |
| (RT = about 6′) | | | | |
| Impurity 4 | Absent | Absent | Absent | Absent |
| (RT = about 15′) | | | | |
| Each Unknown | | | | |
| Impurity | | | | |
| Unknown 3 | Absent | Absent | 0.028% | 0.008% |
| (RT = about 7.5′) | | | | |
| Unknown 1 | 0.020% | Absent | 0.005% | Absent |
| (RT = about 16′) | | | | |
| Unknown 2 | Absent | 0.019% | Absent | 0.003% |
| (RT = about 17.5′) | | | | |
| Total Impurities | 0.020% | 0.019% | 0.005% | 0.003% |

*Data obtained on samples stored at room temperature

As described above, the micronized cyclobenzaprine HCl supplied by Dipharma and the un-micronized cyclobenzaprine HCl supplied by Sifavitor are equivalent in terms of their galenic activities and properties. In order to verify this conclusion, a further trial was designed using un-micronized cyclobenzaprine HCl supplied by Dipharma in both the formulations (with and without the K₂HPO₄ basifying agent). This formulation was created to compare the same characteristics of the un-micronized cyclobenzaprine HCl supplied by two different manufacturers (Table 42). The formulation then was analyzed and compared with the previous formulations as shown in Table 43.

TABLE 42
Un-micronized cyclobenzaprine HCl formulation
(basifying-agent-containing)

| | | | | Cycloben- |
|---|---|---|---|---|
| | | | | prine |
| | Com- | | | za HCl (not |
| | mercial | | | micronized) |
| Component | Name | Function | Supplier | (mg/tab) |
| Cyclobenza- | Cyclobenza- | Active | Dipharma | 2.40 |
| prine HCl | prine HC1 | Ingredient | | |
| Mannitol | Pearlitol | Disinte- | Roquette | 31.55 |
| and Maize | Flash | grant | | |
| starch | | | | |
| Crospovi- | Kollidon | Disinte- | BASF | 2.00 |
| done | CL | grant | | |
| Colloidal | Aerosil 200 | Glidant | Evonik | 0.50 |
| Silica | | | | |
| Sodium | Lubrisanaq | Lubricant | Pharma | 0.50 |
| Stearyl | | | Trans | |
| Fumarate | | | Sanaq | |
| K₂HPO₄ | K₂HPO₄ | Basifying | VWR | 1.05 |
| | | agent | | |

| | |
|---|---|
| Total weight | 38.00 |

TABLE 43
Analysis of un-micronized cyclobenzaprine HCl
formulation (containing K₂HPO₄ basifying agent)

| | | Cycloben- | | Cycloben- |
|---|---|---|---|---|
| | | zaprine | Cycloben- | zaprine |
| | | HCl | zaprine | HCl |
| | | formulation | HCl | formulation |
| | | (Dipharma, | formulation | (Sifavitor, |
| | Specifi- | not | (Dipharma, | not |
| Analyses | cations | micronized) | micronized) | micronized) |
| Distribution | TBD | Assay = | Assay = | Assay = |
| | | 97.5% | 96.2% | 99.9% |
| | | SD = | SD = | SD = |
| | | 4.5 | 3.5 | 6.5 |
| | | RSD = | RSD = | RSD = |
| | | 4.6% | 3.7% | 6.5% |

Tablets

| | | | | |
|---|---|---|---|---|
| Assay | 95-105% | 101.5% | 99.4% | 103.8% |
| Disintegration | <30″ | 17″ | 54″ | 40″ |
| test | | | | |
| Hardness | TBD | 27.20N | 32.40N | 20.20N |

The control cyclobenzaprine HCl formulations (lacking a basifying agent) described above were duplicated, substituting un-micronized cyclobenzaprine HCl for micronized cyclobenzaprine HCl (Table 44). The formulation then was analyzed and compared with the previous formulations lacking the K₂HPO₄ basifying agent as shown in Table 45.

TABLE 44
Un-micronized cyclobenzaprine HCl formulation
(lacking the basifying agent)

| | | | | Cycloben- |
|---|---|---|---|---|
| | | | | zaprine |
| | Com- | | | HCl (not |
| | mercial | | | micronized) |
| Component | Name | Function | Supplier | (mg/tab) |
| Cycloben- | Cycloben- | Active | Dipharma | 2.40 |
| zaprine | zaprine | | | |
| HCl | HCl | Ingredient | | |
| Mannitol | Pearlitol | Disinte- | Roquette | 32.60 |
| and | Flash | grant | | |
| Maize | | | | |
| starch | | | | |
| Crospovi- | Kollidon | Disinte- | BASF | 2.00 |
| done | CL | grant | | |
| Colloidal | Aerosil | Glidant | Evonik | 0.50 |
| Silica | 200 | | | |
| Sodium | Lubrisanaq | Lubricant | Pharma | 0.50 |
| Stearyl | | | Trans | |
| Fumarate | | | Sanaq | |

| | |
|---|---|
| Total weight | 38.00 |

TABLE 45
Analysis of un-micronized cyclobenzaprine HCl formulation (control:
lacking a basifying agent)

| | | | | |
|---|---|---|---|---|
| | | Cyclobenzaprine | Cyclobenzaprine | Cyclobenzaprine |
| | | HCl formulation | HCl formulation | HCl formulation |
| | | (Dipharma, not | (Dipharma, | (Sifavitor, not |
| Analyses | Specifications | micronized) | micronized) | micronized) |

Powder

| | | | | |
|---|---|---|---|---|
| Distribution | TBD | Assay = 101.3% | Assay = 102.8% | Assay = 102.2% |
| | | SD = 2.69 | SD = 2.3 | SD = 2.6 |
| | | RSD = 2.65% | RSD =2.2% | RSD = 2.5% |

Tablets

| | | | | |
|---|---|---|---|---|
| Assay | 95-105% | 106.9% | 102.2% | 98.8% |
| Disintegration | <30″ | 20″ | 65″ | 42″ |
| test | | | | |
| Hardness | TBD | 28.40 N | 31.40 N | 20.80 N |

In summary, the Dipharma batch of un-micronized cyclobenzaprine HCl demonstrated, for the tested formulations, good distribution and performance (e.g., it had a particularly low disintegration time in tablet form). The Dipharma batch of micronized cyclobenzaprine HCl demonstrated, for the tested formulations, good distribution and performance, with the exception of some electrostatic phenomena typical of a micronized powder. The Sifavitor batch of not-micronized cyclobenzaprine HCl demonstrated, for the tested formulations, good distribution and performance.

All the analytical results reported in Example 8 and referred to the formulations reported above had been performed according to the following analytical conditions:

Assay

ANALYTICAL EQUIPMENT: HPLC JASCO equipped with Auto-Sampler AS-1555, Pump PU-1580 and detector UV-2075 Plus

ANALYTICAL COLUMN: ALLTIMA C₁₈ 5 μm 150×4.6 mm or equivalent

MOBILE PHASE:
49.8% Water
25% Methanol
25% Acetonitrile
0.2% Methanesulphonic acid
Mobile phase corrected to pH 3.60±0.10 with diethylamine

FLOW RATE: 1.5 ml/min

WAVELENGTH: 240 nm

INJECTED VOLUME: 10 μl

TEMPERATURE: 25° C.

ACQUISITION LENGTH: 8 minutes

SOLVENT: 50% Methanol

50% Phosphate buffer: (dissolve 6.80 g/l Potassium dihydrogen phosphate and 1.57 g/l Sodium hydroxide in water; correct to pH 7.40±0.10 with Sodium hydroxide 1N if necessary)

CYCLOBENZAPRINE RETENTION TIME: About 5.0 minutes

PRODUCT STANDARD PREPARATION: Weigh about 20 mg of cyclobenzaprine HCl reference (or working) standard in a 50 mL volumetric flask and add 40 ml of solvent; sonicate for 5 minutes and dilute to volume with solvent. Transfer 2.5 mL of this solution in a 10 ml volumetric flask and dilute to volume with solvent (Concentration of cyclobenzaprine HCl=about 100 g/mL).

SAMPLE PREPARATION (powder): Weigh about 160 mg of cyclobenzaprine HCl powder in a 100 mL volumetric flask, add 70 mL of solvent, agitate with magnetic stirrer for 10 minutes and sonicate for 5 minutes; dilute to volume with solvent. Filter with Syringe filter with Hydrophilic 0.45 μm PVDF membrane before injection. The concentration of cyclobenzaprine HCl is 100 μg/mL].

SAMPLE PREPARATION (tablets): Weigh 10 tablets of Cyclobenzaprine HCl in a 100 mL volumetric flask, add 80 mL of solvent, agitate with magnetic stirrer for 10 minutes and sonicate for 5 minutes; dilute to volume with solvent. Transfer 4 mL in a 10 mL volumetric flask and dilute to volume with solvent. Filter with syringe filter with Hydrophilic 0.45 μm PVDF membrane before injection. [The concentration of cyclobenzaprine HCl is 100 μg/mL].

### Purity

ANALYTICAL EQUIPMENT: HPLC JASCO equipped with Auto-Sampler AS-1555, Pump PU-1580, and detector UV-2075 Plus

ANALYTICAL COLUMN: ALLTIMA C₁₈ 5 μm 150×4.6 mm or equivalent

MOBILE PHASE:
59.8% Water
20% Methanol
20% Acetonitrile
0.2% Methanesulphonic acid
Mobile phase corrected to pH 3.60±0.10 with diethylamine

FLOW RATE: 2.0 ml/min

WAVELENGTH: 240 nm

INJECTED VOLUME: 20 μl

TEMPERATURE: 25° C.

ACQUISITION LENGTH: 60 minutes

SOLVENT: Methanol

RETENTION TIME: About 12.0 minutes

RELATED SUBSTANCE RETENTION TIME: Dibenzosuberenone (Impu 1): about 56.0 minutes

Carbinole (Impu 2): about 6.0 minutes

Amitriptyline (Impu 4): about 15.0 minutes

RELATED SUBSTANCES STANDARD PREPARATION (for Known and Unknown impurities): Weigh about 10 mg of cyclobenzaprine HCl reference (or working) standard and about 10 mg of cyclobenzaprine HCl Impurity 1, 2, 4 reference (or working) standard in a 100 mL volumetric flask, add 80 ml of Methanol and sonicate for 10 minutes; dilute to volume with Methanol.

Transfer 1.0 mL of this solution in a 100 ml volumetric flask and dilute to volume with solvent. The concentration of impurity is 1 μg/mL, corresponding to 0.1%.

SAMPLE PREPARATION: Weigh exactly 4 cyclobenzaprine HCl tablets in a 10 mL volumetric flask, add 5 mL of solvent and sonicate for 10 minutes; dilute to volume with solvent. Filter with syringe filter with hydrophilic 0.45 μm PVDF membrane before injection. The concentration of Cyclobenzaprine HCl is about 1000 μg/mL.

Starting from the all the data collected in the formulation setups, further batches of formulations both with and without basifying agent were manufactured. As the final step of the galenic development, the big laboratory batches were manufactured according to the formulations reported in Table 46 and 47:

TABLE 46
Basifying-agent-containing cyclobenzaprine HCI formulations

| | Commercial | | | |
|---|---|---|---|---|
| Component | Name | Function | Supplier | (mg/tab) |
| Cyclobenzaprine | Cyclobenzaprine | Active | Dipharma | 2.40 |
| HCl* | HCl | Ingredient | | |
| Mannitol and | Pearlitol Flash | Disintegrant | Roquette | 31.55 |
| Maize starch | | | | |
| Crospovidone | Kollidon CL | Disintegrant | BASF | 2.00 |
| | | agent | | |
| Colloidal Silica | Aerosil 200 | Glidant | Evonik | 0.50 |
| | | agent | | |
| Sodium Stearyl | Lubrisanaq | Lubricant | Pharma | 0.50 |
| Fumarate | | | Trans | |
| | | | Sanaq | |
| Potassium | Potassium | Basifying | VWR | 1.05 |
| Phosphate | Phosphate | Agent | | |
| Dibasic | Dibasic | | | |

| | |
|---|---|
| Total Weight | 38.00 |

TABLE 47
Cyclobenzaprine HCl formulations lacking a basifying agent

| | Commercial | | | |
|---|---|---|---|---|
| Component | Name | Function | Supplier | (mg/tab) |
| Cyclobenzaprine | Cyclobenzaprine | Active | Dipharma | 2.40 |
| HCl* | HCl | Ingredient | | |
| Mannitol and | Pearlitol Flash | Disintegrant | Roquette | 32.60 |
| Maize starch | | | | |
| Crospovidone | Kollidon CL | Disintegrant | BASF | 2.00 |
| | | agent | | |
| Colloidal Silica | Aerosil 200 | Glidant | Evonik | 0.50 |
| | | agent | | |
| Sodium Stearyl | Lubrisanaq | Lubricant | Pharma | 0.50 |
| Fumarate | | | Trans | |
| | | | Sanaq | |

| | |
|---|---|
| Total Weight | 38.00 |

An additional formulation was manufactured starting from the basifying-agent-containing cyclobenzaprine HCl formulation. This coated version was obtained using a a coating mixture also containing the basifying agent as described in Table 48.

TABLE 48
Basifying-agent-containing cyclobenzaprine HCl formulations
(coated formulation)

| | Commercial | | | |
|---|---|---|---|---|
| Component | Name | Function | Supplier | (mg/tab) |
| Cyclobenzaprine | Cyclobenzaprine | Active | Dipharma | 2.40 |
| HCl* | HCl | Ingredient | | |
| Mannitol and | Pearlitol Flash | Diluter | Roquette | 31.55 |
| Maize starch | | | | |
| Crospovidone | Kollidon CL | Disintegrant | BASF | 2.00 |
| | | agent | | |
| Colloidal Silica | Aerosil 200 | Glidant | Evonik | 0.50 |
| | | agent | | |
| Sodium Stearyl | Lubrisanaq | Lubricant | Pharma | 0.50 |
| Fumarate | | | Trans | |
| | | | Sanaq | |
| Potassium | Potassium | Basifying | VWR | 1.05 |
| Phosphate | Phosphate | Agent | | |
| Dibasic | Dibasic | | | |
| Coating Mixture | Water | Coating | APR | 0.30 |
| (water, | Opadry Clear | Layer | Colorcon | |
| Opadry Clear, | Potassium | | VWR | |
| Potassium | phosphate | | | |
| phosphate | dibasic | | | |

| | | |
|---|---|---|
| dibasic) | Total Weight | 38.30 |

Time zero data from a stability study on the large laboratory batches in different packaging materials is are reported in the Table 49.

TABLE 49
Stability study, time zero data

| | Batch with | Batch lacking | Batch with |
|---|---|---|---|
| | basifying agent | basifying agent | basifying agent |
| Parameter | (core) | (core) | (coated) |
| Appearance | White to off- | White to off- | White to off- |
| | white, | white, | white, |
| | biconvex round | biconvex round | biconvex round |
| | tablet | tablet | tablet |
| Cyclobenzaprine | 93.1 | 99.7 | 107.1 |
| HCl Assay | | | |
| (%) | | | |
| Impurity A (%) | Absent | Absent | Absent |
| ImpurityB (%) | <LOQ | <LOQ | <LOQ |
| ImpurityC (%) | Absent | Absent | Absent |
| Each Unk | 0.01 | <LOQ | <LOQ |
| impurity (%)- | | | 0.02 |
| Unk1 | | | |
| (RRT = 0.83) | | | |
| Total impurities | 0.01 | <LOQ | |
| (%) | | | |
| pH in water | 7.23 | 5.47 | 7.25 |
| Hardness (N) | 28.80 | 37.40 | 43.20 |
| Disintegration | 26 | 22 | 64 |
| Time (sec) | (16; 23; 40; 21; | (18; 40; 16; 23; | (76; 76; 57; 59; |
| | 27; 31) | 20; 16) | 60; 57) |
| KF (%) | 2.02 | 2.06 | 2.59 |

To study stressed stability in the batch lacking the basifying agent, the samples are stored at 40° C. and 50° C. and are analyzed after 1, 2, 3, and 4 weeks. Data from the 1 week timepoint are reported in Table 50.

TABLE 50
Stability study, 1 week, no basifying agent

| | Tentative | 1 week, | 1 week, |
|---|---|---|---|
| Parameter | Specifications | 50° C. | 40° C. |
| Impurity A (%) | 0.2 | <LOQ | Absent |
| ImpurityB (%) | 0.2 | <LOQ | <LOQ |
| ImpurityC (%) | 0.2 | Absent | Absent |
| Each Unk impurity | 0.5 | 0.02 | <LOQ |
| (%)- | | | |
| Unk 1 (RRt = 0.83) | | | |
| Total impurities | 2.0 | 0.02 | <LOQ |
| (Area) | | | |

All of the analysis perfumed starting from the large laboratory batches were performed according to the following optimized and validated conditions:

### Purity

Analytical equipment: HPLC JASCO equipped with Auto-Sampler AS-2055, PU-2080, and detector Diode Array MD 2010 Plus

Analytical column: SYMMETRY C18 5 μm 250×4.6 mm or equivalent

Mobile phase: 25% methanol, 25% acetonitrile, 50% Butylamine buffer/CH₃COOH (In 950 ml of water add 10 ml of 99.5% Butylamine, correct to pH 6.20±0.10 with glacial acetic acid and dilute to volume of 1 L with water)

Flow rate: 1.5 mL/min

Wavelength: 239 nm

Injected volume: 10 μl

Temperature: 28° C.

Acquisition length: 35 minutes

Solvent: Mobile phase

Retention time: Impurity A (Carbinole) about 6′, Impurity B (Amitriptyline) about 14′, Impurity C (Dibenzosuberenone) about 29′, Cyclobenzaprine HCl about 11′

For the related substances standard preparation (for Known and Unknown impurities): weigh about 10 mg of cyclobenzaprine HCl reference (or working) standard and about 10 mg of cyclobenzaprine HCl Impurity A, B and C reference (or working) standard in a 100 mL volumetric flask, add 5 mL of Acetonitrile and then 75 mL of solvent; sonicate for 5 minutes and dilute to volume with solvent.

Transfer 1.0 mL of this solution in a 50 ml volumetric flask and dilute to volume with solvent for a final concentration of impurity of 2 μg/mL.

LOQ level: Transfer 1.0 mL of the last solution in a 10 ml volumetric flask and dilute to volume with solvent for a concentration of impurity of 0.2 μg/mL.

For the sample preparation: place 10 cyclobenzaprine HCl tablets exactly weighed in a 25 mL volumetric flask (or 20 cyclobenzaprine HCl tablets in a 50 mL volumetric flask), add 20 mL (or 40 mL) of solvent and sonicate for 5 minutes; dilute to volume with solvent. Filter with a syringe filter with hydrophilic 0.45p m PVDF membrane before injection. The concentration of Cyclobenzaprine HCl is about 1000 μg/mL.

### Assay

ANALYTICAL EQUIPMENT: HPLC JASCO equipped with Auto-Sampler AS-1555, Pump PU-1580, and detector UV-2075 Plus

ANALYTICAL COLUMN: ALLTIMA C18 5 μm 150×4.6 mm or equivalent

MOBILE PHASE:
49.8% Water
25% Methanol
25% Acetonitrile
0.2% Methanesulphonic acid
Mobile phase corrected to pH 3.60±0.10 with diethylamine

FLOW RATE: 1.5 ml/min

WAVELENGTH: 240 nm

INJECTED VOLUME: 10 μl

TEMPERATURE: 25° C.

ACQUISITION LENGTH: 8 minutes

SOLVENT:
50% Methanol
50% Phosphate buffer (dissolve 6.80 g/l Potassium dihydrogen phosphate and 1.57 g/l Sodium hydroxide in water; correct to pH 7.40±0.10 with Sodium hydroxide 1N if necessary)

CYCLOBENZAPRINE RETENTION TIME: about 5.0 minutes

PRODUCT STANDARD PREPARATION: Weigh about 20 mg of cyclobenzaprine HCl reference (or working) standard in a 50 mL volumetric flask and add 40 ml of solvent; sonicate for 5 minutes and dilute to volume with solvent. Transfer 2.5 mL of this solution in a 10 ml volumetric flask and dilute to volume with solvent, for a concentration of cyclobenzaprine HCl equaling about 100 g/mL.

SAMPLE PREPARATION (powder): Weigh about 160 mg of cyclobenzaprine HCl powder in a 100 mL volumetric flask, add 70 mL of solvent, agitate with magnetic stirrer for 10 minutes and sonicate for 5 minutes; dilute to volume with solvent. Filter with a syringe filter with hydrophilic 0.45p m PVDF membrane before injection, for a cyclobenzaprine HCl concentration of 100 μg/mL.

SAMPLE PREPARATION (tablets): Weigh 10 tablets of cyclobenzaprine HCl in a 100 mL volumetric flask, add 80 mL of solvent, agitate with magnetic stirrer for 10 minutes and sonicate for 5 minutes; dilute to volume with solvent. Transfer 4 mL in a 10 mL volumetric flask and dilute to volume with solvent. Filter with a syringe filter with hydrophilic 0.45 μm PVDF membrane before injection, for a cyclobenzaprine HCl concentration of 100 μg/mL.

### Example 9

As referred to in Example 4, a study was designed in order to assess the effects of the compositions and methods of the invention. The study utilized beagle dogs, which were administered cyclobenzaprine HCl orally, sublingually, or intravenously. The pharmacokinetic parameters were then calculated. A summary of the study design is as follows:

Test substance: cyclobenzaprine hydrochloride

Administration routes: oral (PO), sublingual, and intravenous (IV)

Species: beagle dog

Gender: female

Matrix: plasma

Vehicle: aqueous KH₂PO₄ solution adjusted with NaOH to pH 7.4

Dose: 0.14 mg/kg (approximately corresponding to 10 mg dose in 70 kg human)

Formulation concentrations: PO: 0.028 mg/mL; sublingual: 2.5 mg/mL; IV: 0.14 mg/mL

Administration volumes: PO: 5 mL/kg; sublingual: 0.056 mL/kg; IV: 1 mL/kg

Blood was collected in a fasted condition for all three routes of administration. For PO administration, blood was collected at 0 h (pre-dose) and at 0.5 h, 1 h, 2 h, 3 h, 4 h, 5 h, 6 h, 8 h, 10 h, 12 h and 24 h post-administration. For sublingual and IV administration, blood was collected after the predose and at 10 min, 20 min, 0.5 h, 1 h, 2 h, 3 h, 4 h, 6 h, 8 h, 10 h, and 24 h post-administration. Analysis of the cyclobenzaprine was performed substantially as described above. Briefly, the method involved liquid-liquid extraction with hexane. The extracts were injected using an HPLC system and an API4000® (Applied Biosystems) for the MS/MS detection, with an “Onyx monolithic Phenomenex C18 100×3.0 mm” column. The method was linear from 0.1 to 50 ng/mL for cyclobenzaprine in dog plasma. Quality control samples were prepared at 0.3, 25 and 40 ng/mL for cyclobenzaprine in dog plasma. Results obtained from the analysis of quality control samples were within the limits of acceptance defined in the protocol, therefore validating the concentrations measured in plasma samples.

Intravenous (IV) administration of cyclobenzaprine HCl in a solution containing a basifying agent resulted in surprisingly dynamic range in the plasma concentration-time profile and a surprisingly low bioavailability of cyclobenzaprine administered by nasogastric tube. In previous Beagle experiments, 2 mg/kg of cyclobenzaprine was administered to Beagles (6.7-8.2 kg) with a radioactive label (Hucker, 1978 Drug Metabolism and Disposition 6(6):659). Doses were expressed as free base, and so should be adjusted by MW Free Base 275 g/mole/MW HCl salt 312 g/mole. Thus, 1.76 mg/kg of cyclobenzaprine HCl was administered. Cyclobenzaprine was administered by in saline solution for intravenous (IV) dosing or in gelatin capsules for oral (PO) dosing. In the Hucker study, the plasma levels of cyclobenzaprine were measured by recovery of radioactive equivalents of a ⁴C labeled cyclobenzaprine, so the plasma levels were “equivalent” to certain values (in μg/ml) of cyclobenzaprine. By this method, Beagles who received 1.76 mg/kg cyclobenzaprine HCl by mouth had plasma levels of 0.72 μg/ml [equivalent](at 0.5 h), 1.14 μg/ml [equivalent](1 hr), 1.46 μg/ml [equivalent](2 hr), 0.92 μg/ml [equivalent](4 hr), 0.58 μg/ml [equivalent](6 hr), 0.10 μg/ml [equivalent](24 hr) and dogs who received 2 mg/kg cyclobenzaprine by intravenous injection in saline had plasma levels of 0.43 μg/ml [equivalent](0.5 h), 0.53 μg/ml [equivalent](1 hr), 0.60 μg/ml [equivalent](2 hr), 0.55 μg/ml [equivalent](4 hr), 0.45 μg/ml [equivalent](6 hr), 0.12 μg/ml [equivalent](24 hr). In our Beagle studies, the dose was 0.14 mg/kg, which is approximately 1/12.6 the dose of the Hucker studies. Solely for the purposes of comparing the pharmacokinetic profiles of Hucker and our studies, assuming dose proportionality, the data from Hucker were adjusted to be equivalent to dose of 0.14 mg/kg cyclobenzaprine HCl and are shown in Table 51.

TABLE 51
Comparison of pharmacokinetics

| Hucker | | | | | |
|---|---|---|---|---|---|
| Dose Adjusted | | | | | |
| for 0.14 mg/kg | | | Current study | | |
| t | PO | IV | NG | IV | SL |
| 0.167 | | | | 21.07 | 122.3 |
| 0.33 | | | | 17.33 | 81.03 |
| 0.5 | 57.1 | 34.1 | 0.32 | 14.3 | 52.3 |
| 1 | 90.5 | 42.1 | 0.41 | 9.0 | 40.8 |
| 2 | 115.9 | 47.6 | 0.33 | 6.5 | 15.0 |
| 4 | 73.0 | 43.7 | 0.21 | 3.2 | 5.8 |
| 6 | 46.0 | 35.7 | 0.15 | 1.7 | 3.4 |
| 24 | 7.9 | 9.5 | — | 0.1 | 0.1 |

all plasma levels in ng/ml

Comparison revealed that adjusted for dose and dose-proportionality to our study, Hucker's PO bioavailability from a gelatin capsule was much higher (peak at 115.9 ng/ml at 2 hours) than the bioavailability of cyclobenzaprine oral solution via a nasogastric tube (NG) (peak at 0.41 ng/ml at 1 h). The profile of IV administered cyclobenzaprine in Hucker was relatively flate from 0.5 h to 6 h, adjusted for dose and dose-proportionality to our study, varying from 34.1 ng/ml at 0.5 h to 47.6 ng/ml (at 2 hours) to 35.7 ng/ml whereas our profile was dynamic in that period of time and decreased from 52.3 to 3.4 ng/ml. Hucker did not measure plasma levels at 0.167 h or 0.33 h which in our study were the highest plasma levels and and shows an even more dynamic range. Hucker did not study sublingual administration.

Sublingual administration of cyclobenzaprine HCl resulted in surprisingly improved pharmacokinetic properties and bioavailability as compared to PO administration (Table 52, FIGS. 2 and 3; individual PO, sublingual (SL), and IV data: Tables 53-56). In particular, the Cₘₐₓ for cyclobenzaprine was significantly higher when administered sublingually, from approximately 0.48 ng/mL (PO) to approximately 137 ng/mL (SL). The Tₘₐₓ also diminished from one hour to 10 minutes, and the bioavailability increased from approximately 3.8% to 292%. As is standard practice, bioavailability for IV administration was considered to be 100%. We believe that a possible explanation for the sublingual bioavailability may be that the IV measurement was taken after the true IV Cₘₐₓ value should have been recorded. Regardless, in comparing the bioavailability of PO and sublingual administration, there is a nearly 77-fold increase.

TABLE 52
Mean ± SD plasma pharmacokinetic parameters

| | Cmax | Tmax * | AUCₜ | AUC_{inf} | % | Kel | t_{1/2} | Cl** | Vd** | Bioavailability |
|---|---|---|---|---|---|---|---|---|---|---|
| Route | (ng/mL) | (h) | (ng/mL*h) | (ng/mL*h) | AUCextra | (1/h) | (h) | (L/h/kg) | (L/kg) | F % |
| PO | 0.4782 ± | 1.00 | 1.591 ± | 3.626 ± 3.339 | 36.68 ± 28.68 | 0.4782 | 1.449 | 72.34 | 151.3 | 3.778 ± 2.294 |
| | 0.2616 | | 0.8142 | | | | | | | |
| Sub- | 136.6 ± | 0.17 | 126.9 ± | 129.1 ± 36.45 | 1.84 ± 1.81 | 0.2964 ± | 2.852 ± | 1.158 ± | 5.260 ± 3.987 | 292.4 ± 84.97 |
| lingual | 69.94 | | 37.05 | | | 0.1442 | 1.437 | 0.3288 | | |
| IV | NA | NA | 43.51 ± | 44.94 ± 4.149 | 3.13 ± 1.77 | 0.1677 ± | 4.723 ± | 3.138 ± | 21.27 ± 7.173 | — |
| | | | 3.766 | | | 0.07679 | 1.685 | 0.3068 | | |

* Median
**Cl and Vd are Cl/F and Vd/F for PO and sublingual routes
NA: Not Applicable

TABLE 53
Cyclobenzaprine plasma concentrations (ng/mL) measured after oral
administration of cyclobenzaprine HCl

| Time | | | | | | | | | | | |
|---|---|---|---|---|---|---|---|---|---|---|---|
| Hours | Dog 1 | Dog 2 | Dog 3 | Dog 4 | Dog 5 | Low | High | Mean | S.D. | % CV | n |
| 0 | BLQ | BLQ | BLQ | BLQ | BLQ | BLQ | BLQ | BLQ | NC | NC | 5 |
| 0.5 | 0.2324 | 0.2011 | 0.2843 | 0.3300 | 0.5700 | 0.2011 | 0.5700 | 0.3236 | 0.1463 | 45.21 | 5 |
| 1 | 0.3215 | 0.2671 | 0.2033 | 0.3860 | 0.8486 | 0.2033 | 0.8486 | 0.4053 | 0.2568 | 63.36 | 5 |
| 2 | 0.242 | 0.2345 | 0.1859 | 0.6610 | 0.3261 | 0.1859 | 0.6610 | 0.3299 | 0.1918 | 58.14 | 5 |
| 3 | 0.2465 | 0.2756 | 0.206 | 0.2414 | 0.5494 | 0.2060 | 0.5494 | 0.3038 | 0.1395 | 45.92 | 5 |
| 4 | 0.1589 | 0.2057 | 0.1264 | 0.2193 | 0.3384 | 0.1264 | 0.3384 | 0.2097 | 0.08092 | 38.59 | 5 |
| 5 | 0.1570 | 0.1623 | 0.1035 | 0.1386 | 0.2119 | 0.1035 | 0.2119 | 0.1547 | 0.03942 | 25.48 | 5 |
| 6 | 0.1530 | 0.1326 | BLQ | BLQ | 0.1671 | BLQ | 0.1671 | 0.09054 | 0.08356 | 92.29 | 5 |
| 8 | BLQ | BLQ | BLQ | BLQ | 0.1255 | BLQ | 0.1255 | BLQ | NC | NC | 5 |
| 10 | BLQ | BLQ | BLQ | BLQ | 0.1121 | BLQ | 0.1121 | BLQ | NC | NC | 5 |
| 12 | BLQ | BLQ | BLQ | BLQ | BLQ | BLQ | BLQ | BLQ | NC | NC | 5 |
| 24 | BLQ | BLQ | BLQ | BLQ | BLQ | BLQ | BLQ | BLQ | NC | NC | 5 |

TABLE 54
Cyclobenzaprine plasma concentrations (ng/mL) measured after
sublingual administration of cyclobenzaprine HCl

| Time | | | | | | | | | | | |
|---|---|---|---|---|---|---|---|---|---|---|---|
| Hours | Dog 1 | Dog 2 | Dog 3 | Dog 4 | Dog 5 | Low | High | Mean | S.D. | % CV | n |
| 0 | BLQ | BLQ | BLQ | BLQ | BLQ | BLQ | BLQ | BLQ | NC | NC | 5 |
| 0.16667 | 38.94 | 86.95 | 164.9 | 244.6 | 76.21 | 38.94 | 244.6 | 122.3 | 82.30 | 67.28 | 5 |
| 0.33333 | 38.39 | 41.03 | 64.52 | 147.2 | 114.0 | 38.39 | 147.2 | 81.03 | 47.84 | 59.05 | 5 |
| 0.5 | 72.36 | 32.33 | 28.82 | 66.00 | 61.78 | 28.82 | 72.36 | 52.26 | 20.19 | 38.63 | 5 |
| 1 | 22.62 | 23.49 | 34.74 | 60.88 | 62.42 | 22.62 | 62.42 | 40.83 | 19.61 | 48.02 | 5 |
| 2 | 11.50 | 8.706 | 14.25 | 17.76 | 22.55 | 8.706 | 22.55 | 14.95 | 5.409 | 36.18 | 5 |
| 3 | 10.91 | 6.663 | 9.565 | 5.866 | 9.348 | 5.866 | 10.91 | 8.470 | 2.120 | 25.03 | 5 |
| 4 | 5.323 | 3.946 | 6.611 | 7.016 | 6.027 | 3.946 | 7.016 | 5.785 | 1.210 | 20.92 | 5 |
| 6 | 3.882 | 2.361 | 3.682 | 3.687 | 3.369 | 2.361 | 3.882 | 3.396 | 0.6072 | 17.88 | 5 |
| 8 | 2.573 | 1.430 | 1.085 | 0.7974 | 1.287 | 0.7974 | 2.573 | 1.434 | 0.6793 | 47.37 | 5 |
| 10 | 1.364 | 0.8100 | 0.5219 | 0.5297 | 0.7659 | 0.5219 | 1.364 | 0.7983 | 0.3427 | 42.93 | 5 |
| 24 | BLQ | 0.1430 | 0.2610 | BLQ | BLQ | BLQ | 0.2610 | 0.0808 | 0.1182 | 146.29 | 5 |

BLQ: Below the Limit of Quantification (0.1 ng/mL)
italic: out of range (50 ng/mL): value given for indication

TABLE 55
Cyclobenzaprine plasma concentrations (ng/mL) measured after IV
administration of cyclobenzaprine HCl

| Time | | | | | | | | | | | |
|---|---|---|---|---|---|---|---|---|---|---|---|
| Hours | Dog 1 | Dog 2 | Dog 3 | Dog 4 | Dog 5 | Low | High | Mean | S.D. | % CV | n |
| 0 | BLQ | BLQ | BLQ | BLQ | BLQ | BLQ | BLQ | BLQ | NC | NC | 5 |
| 0.16667 | 15.41 | 18.56 | 17.38 | 25.48 | 28.52 | 15.41 | 28.52 | 21.07 | 5.633 | 26.73 | 5 |
| 0.33333 | 13.44 | 13.74 | 13.70 | 22.02 | 23.74 | 13.44 | 23.74 | 17.33 | 5.106 | 29.46 | 5 |
| 0.5 | 10.73 | 12.23 | 14.55 | 17.49 | 16.61 | 10.73 | 17.49 | 14.32 | 2.855 | 19.94 | 5 |
| 1 | 11.08 | 8.868 | 7.459 | 10.31 | 7.071 | 7.071 | 11.08 | 8.958 | 1.743 | 19.46 | 5 |
| 2 | 8.553 | 4.797 | 6.823 | 5.831 | 6.528 | 4.797 | 8.553 | 6.506 | 1.385 | 21.29 | 5 |
| 3 | 4.427 | 4.248 | 5.060 | 4.047 | 3.282 | 3.282 | 5.060 | 4.213 | 0.6440 | 15.29 | 5 |
| 4 | 5.139 | 1.881 | 3.879 | 2.989 | 1.866 | 1.866 | 5.139 | 3.151 | 1.394 | 44.24 | 5 |
| 6 | 1.682 | 1.963 | 2.193 | 1.663 | 0.9617 | 0.9617 | 2.193 | 1.693 | 0.4633 | 27.37 | 5 |
| 8 | 1.413 | 1.000 | 1.036 | 1.089 | 0.7013 | 0.7013 | 1.413 | 1.048 | 0.2537 | 24.21 | 5 |
| 10 | 0.8358 | 0.8134 | 0.6166 | 0.6831 | 0.2325 | 0.2325 | 0.8358 | 0.6363 | 0.2433 | 38.24 | 5 |
| 24 | BLQ | 0.1328 | 0.1787 | 0.1164 | 0.1216 | BLQ | 0.1787 | 0.1099 | 0.06617 | 60.21 | 5 |

BLQ: Below the Limit of Quantification (0.1 ng/mL)

TABLE 56
Cyclobenzaprine pharmacokinetic parameters after administration of
cyclobenzaprine HCl

| | | Cmax | Tmax | AUCₜ | AUC_{inf} | % | Kel | t_{1/2} | Cl* | Vd* | Bioavailability |
|---|---|---|---|---|---|---|---|---|---|---|---|
| Route | Animal | (ng/mL) | (h) | (ng/mL*h) | (ng/mL*h) | AUCextra | (1/h) | (h) | (L/h/kg) | (L/kg) | F % |
| PO | dog 1 | 0.3215 | 1.00 | 1.238 | 9.326 | 86.72 | NC | NC | NC | NC | 2.79 |
| | dog 2 | 0.2756 | 3.00 | 1.245 | 1.849 | 32.66 | NC | NC | NC | NC | 3.01 |
| | dog 3 | 0.2843 | 0.50 | 0.8647 | 1.165 | 25.81 | NC | NC | NC | NC | 1.88 |
| | dog 4 | 0.6610 | 2.00 | 1.646 | 1.935 | 14.97 | 0.4782 | 1.449 | 72.34 | 151.3 | 3.46 |
| | dog 5 | 0.8486 | 1.00 | 2.961 | 3.856 | 23.22 | NC | NC | NC | NC | 7.75 |
| | N | 5 | 5 | 5 | 5 | 5.00 | 1 | 1 | 1 | 1 | 5 |
| | Mean | 0.4782 | 1.50 | 1.591 | 3.626 | 36.68 | 0.4782 | 1.449 | 72.34 | 151.3 | 3.78 |
| | Stdev | 0.2616 | 1.00 | 0.8142 | 3.339 | 28.68 | NC | NC | NC | NC | 2.29 |
| | % CV | 54.71 | 66.67 | 51.17 | 92.09 | 78.21 | NC | NC | NC | NC | 60.74 |
| | SEM | 0.1170 | 0.45 | 0.3641 | 1.493 | 12.83 | NC | NC | NC | NC | 1.03 |
| | Harmean | 0.3862 | 1.03 | 1.347 | 2.191 | 26.21 | NC | NC | NC | NC | 3.05 |
| | GeoMean | 0.4266 | 1.25 | 1.454 | 2.724 | 30.27 | NC | NC | NC | NC | 3.35 |
| | Median | 0.3215 | 1.00 | 1.245 | 1.935 | 25.81 | NC | NC | NC | NC | 3.01 |
| | Min | 0.2756 | 0.50 | 0.8647 | 1.165 | 14.97 | NC | NC | NC | NC | 1.88 |
| | Max | 0.8486 | 3.00 | 2.961 | 9.326 | 86.72 | NC | NC | NC | NC | 7.75 |
| Sublingual | dog 1 | 72.36 | 0.50 | 98.64 | 103.9 | 5.02 | 0.2615 | 2.651 | 1.348 | 5.155 | 222.11 |
| | dog 2 | 86.95 | 0.17 | 86.08 | 87.12 | 1.20 | 0.1365 | 5.077 | 1.607 | 11.77 | 208.22 |
| | dog 3 | 164.9 | 0.17 | 123.2 | 124.4 | 0.99 | 0.2126 | 3.260 | 1.125 | 5.294 | 268.00 |
| | dog 4 | 244.6 | 0.17 | 176.6 | 177.6 | 0.59 | 0.5084 | 1.364 | 0.7881 | 1.550 | 370.85 |
| | dog 5 | 114.0 | 0.33 | 150.1 | 152.2 | 1.39 | 0.3630 | 1.909 | 0.9196 | 2.533 | 392.93 |
| | N | 5 | 5 | 5 | 5 | 5.00 | 5 | 5 | 5 | 5 | 5 |
| | Mean | 136.6 | 0.27 | 126.9 | 129.1 | 1.84 | 0.2964 | 2.852 | 1.158 | 5.260 | 292.42 |
| | Stdev | 69.94 | 0.15 | 37.05 | 36.45 | 1.81 | 0.1442 | 1.437 | 0.3288 | 3.987 | 84.97 |
| | % CV | 51.22 | 55.90 | 29.19 | 28.24 | 98.30 | 48.65 | 50.39 | 28.40 | 75.79 | 29.06 |
| | SEM | 31.28 | 0.07 | 16.57 | 16.30 | 0.81 | 0.06448 | 0.6427 | 0.1470 | 1.783 | 38.00 |
| | Harmean | 113.0 | 0.22 | 118.5 | 120.9 | 1.12 | 0.2430 | 2.339 | 1.085 | 3.316 | 273.56 |
| | GeoMean | 123.7 | 0.24 | 122.6 | 124.9 | 1.37 | 0.2687 | 2.580 | 1.121 | 4.170 | 282.72 |
| | Median | 114.0 | 0.17 | 123.2 | 124.4 | 1.20 | 0.2615 | 2.651 | 1.125 | 5.155 | 268.00 |
| | Min | 72.36 | 0.17 | 86.08 | 87.12 | 0.59 | 0.1365 | 1.364 | 0.7881 | 1.550 | 208.22 |
| | Max | 244.6 | 0.50 | 176.6 | 177.6 | 5.02 | 0.5084 | 5.077 | 1.607 | 11.77 | 392.93 |
| IV | dog 1 | NA | NA | 44.41 | 47.23 | 5.98 | 0.2960 | 2.342 | 2.964 | 10.01 | — |
| | dog 2 | NA | NA | 41.34 | 42.38 | 2.46 | 0.1274 | 5.441 | 3.303 | 25.93 | — |
| | dog 3 | NA | NA | 45.97 | 47.72 | 3.67 | 0.1020 | 6.798 | 2.934 | 28.77 | — |
| | dog 4 | NA | NA | 47.62 | 48.49 | 1.78 | 0.1348 | 5.141 | 2.887 | 21.41 | — |
| | dog 5 | NA | NA | 38.20 | 38.88 | 1.76 | 0.1781 | 3.891 | 3.601 | 20.21 | — |
| | N | 0 | 0 | 5 | 5 | 5.00 | 5 | 5 | 5 | 5 | — |
| | Mean | NA | NA | 43.51 | 44.94 | 3.13 | 0.1677 | 4.723 | 3.138 | 21.27 | — |
| | Stdev | NA | NA | 3.766 | 4.149 | 1.77 | 0.07679 | 1.685 | 0.3068 | 7.173 | — |
| | % CV | NA | NA | 8.655 | 9.232 | 56.64 | 45.80 | 35.68 | 9.776 | 33.73 | — |
| | SEM | NA | NA | 1.684 | 1.855 | 0.79 | 0.03434 | 0.7536 | 0.1372 | 3.208 | — |
| | Harmean | NA | NA | 43.24 | 44.62 | 2.53 | 0.1468 | 4.134 | 3.115 | 18.56 | — |
| | Geo Mean | NA | NA | 43.37 | 44.78 | 2.79 | 0.1560 | 4.444 | 3.126 | 20.04 | — |
| | Median | NA | NA | 44.41 | 47.23 | 2.46 | 0.1348 | 5.141 | 2.964 | 21.41 | — |
| | Min | NA | NA | 38.20 | 38.88 | 1.76 | 0.1020 | 2.342 | 2.887 | 10.01 | — |
| | Max | NA | NA | 47.62 | 48.49 | 5.98 | 0.2960 | 6.798 | 3.601 | 28.77 | — |

*Cl and Vd are Cl/F and Vd/F for PO and sublingual routes
italic: % AUCextra >20%: value given for indication
NC: Not Calculated
NA: Not Applicable

To further investigate the cause of the sublingual bioavailability measured at greater than 100%, several additional hypotheses were proposed. These included an analytical interaction between Propofol (used as an anaesthetic in the sublingual route) and cyclobenzaprine, binding of cyclobenzaprine to the device used for administrations, and in vivo enzymatic competition in the liver between Propofol and cyclobenzaprine.

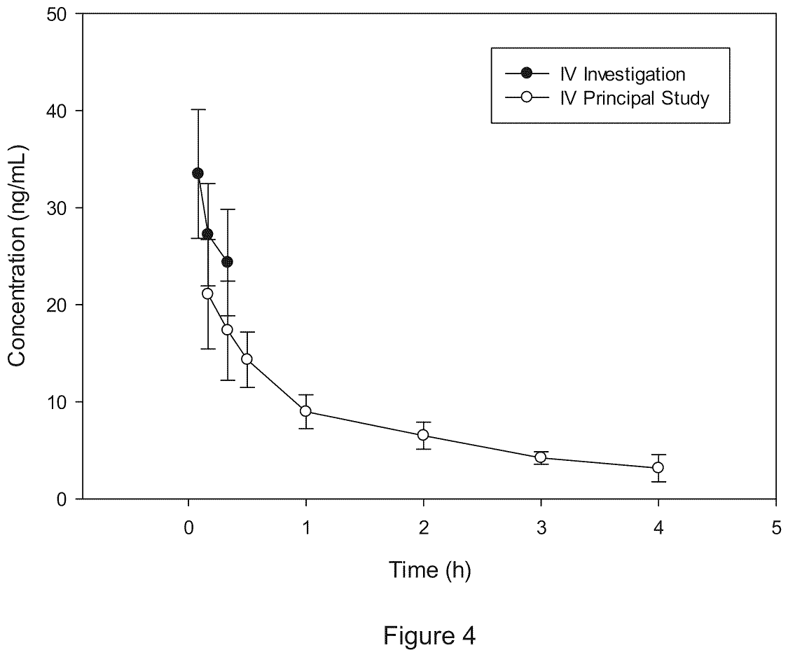

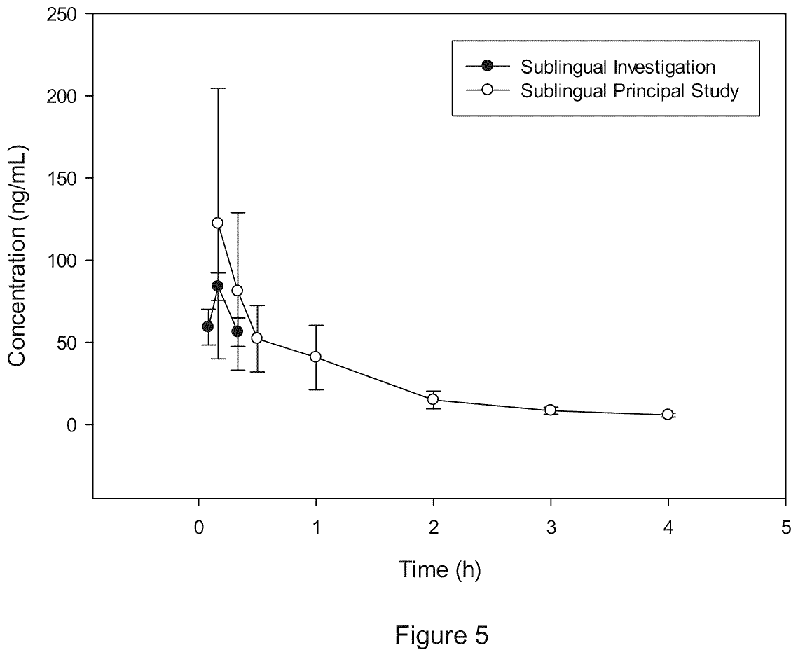

To address the hypothesis that there was in vivo enzymatic competition in the liver between Propofol and cyclobenzaprine, four dogs were treated either sublingually or by IV, with or without Propofol as follows: one dog was treated by IV without Propofol pre-anesthesia; one dog was treated by IV with Propofol pre-anesthesia; one dog was treated sublingually without Propofol pre-anesthesia; and one dog was treated sublingually with Propofol pre-anesthesia. Each dog was sampled at pre-dose before and after Propofol (when possible) and at 5, 10 and 20 minutes post-dose. FIGS. 4 and 5 depict the mean cyclobenzaprine concentration time profiles after IV and sublingual administration, respectively, of cyclobenzaprine HCl as compared to the principal study described above. Table 57 shows the plasma pharmacokinetic parameters for the dogs. The bioavailability calculated in the same condition is about 200%. Concentrations obtained in dogs pre-treated with Propofol or not are similar. Therefore, enzymatic liver competition does not seem to be responsible for the high bioavailability. Dog 4 (treated by sublingual route without Propofol) had concentrations slightly lower than Dog 3 (with Propofol), but the animal swallowed and slobbered, which may explain the difference.

TABLE 57
Plasma pharmacokinetic parameters for dogs treated with or without Propofol

| | | | | | | Bioavailability F % IV |
|---|---|---|---|---|---|---|
| | | Cmax | Tmax | AUCₜ | Bioavailability | without propofol vs |
| Route | Animal | (ng/mL) | (h) | (ng/mL*h) | F % | sublingual with propofol |
| IV | Dog 1 with propofol | 26.84 | 0.0833 | 7.666 | — | — |
| | Dog 2 without propofol | 40.10 | 0.0833 | 11.56 | — | — |
| Sublingual | Dog 3 with propofol | 92.23 | 0.1667 | 22.78 | 297.1 | 197.0 |
| | Dog 4 without propofol | 75.57 | 0.1667 | 17.45 | 150.9 | — |

Dog 4 swallowed and slobbered

To address the hypothesis that cyclobenzaprine bound to the device used for administrations, a binding test was performed with the formulations containing or not containing cyclobenzaprine. The results are depicted in Table 58, which shows that binding of cyclobenzaprine to the administration device was about 5% for the sublingual material and 10% for the intravenous material. Therefore, the binding hypothesis does not explain the bioavailability.

TABLE 58
Binding test results

| | | | | % from initial |
|---|---|---|---|---|
| Formulation | Mean | SD | % CV | formulation |
| Formulation IV | 31.62 | 0.67 | 2.11 | — |
| Mock-dose IV | 28.53 | 0.36 | 1.26 | 90.22 |
| Formulation sublingual | 17.68 | 0.42 | 2.35 | — |
| Mock-dose sublingual | 16.96 | 0.30 | 1.74 | 95.93 |

To determine if propofol may be interfering with the analysis of cyclobenzaprine in either a pure solution or in plasma, a study was performed to combine propofol and cyclobenzaprine in vitro and determine if the measurement of cyclobenzaprine was affected. The results of the test are shown in Table 59. Pre-doses before and after propofol are below the level of quantification (i.e., below 0.1 ng/mL), with no interfering chromatographic peak and no analytical contribution of Propofol observed. Therefore, there does not appear to be an interaction between Propofol and cyclobenzaprine.

TABLE 59
Analytical contribution of Propofol to cyclobenzaprine

| | Measured | % from |
|---|---|---|
| | concentration | Cyclobenzaprine at |
| | (ng/ml) | 0.5 × ULOQ |
| Pure solution of Cyclobenzaprine | 24.14 | — |
| at 0.5 × ULOQ | | |
| Pure solution of Cyclobenzaprine | 26.23 | 108.6 |
| at 0.5 × ULOQ + propofol at | | |
| 10 000 ng/ml | | |
| Plasma sample spiked with | 25.18 | — |
| Cylobenzaprine at 0.5 × ULOQ | | |
| Plasma sample spiked with | 24.18 | 96.0 |
| Cylobenzaprine at 0.5 × ULOQ + | | |
| propofol at 10 000 ng/ml | | |

Based on the above tests, it seems that our initial hypothesis that the bioavailability phenomenon could be explained by the delay between administration and blood sampling (which would be slightly different for intravenous and sublingual routes) may be the most likely. This delay could result in metabolism occurring for a longer period of time after intravenous administration than by the sublingual route. Regardless, we conclude that the bioavailability of cyclobenzaprine administered sublingually in Beagle dogs is very high, and likely close to 100%.

### Example 10

To study the levels of cyclobenzaprine plasma concentrations after a single sublingual administration of cyclobenzaprine HCl in female beagle dogs, two sublingual tablet formulations were compared: a control tablet without a basifying agent and a tablet with a basifying agent (K₂HPO₄). Pharmacokinetic parameters were then calculated and compared.

Each dog received a sublingual cyclobenzaprine tablet, first in a formulation with basifying agent, and then in a formulation without basifying agent, with a washout period of two weeks between each administration. For sublingual dosing, dogs were given propofol and were put in a lying position (ventral decubitis). The mouth was gently opened and the tablet was placed under the tongue. The tongue was then immediately replaced in its initial position and the mouth was closed. The dog was left in the same position until waking. Dogs treated with cyclobenzaprine in the formulation with basifying agent (K₂HPO₄) were sedated using 6.5 mg/kg of propofol intravenously. They were then given a single sublingual dose of 1 tablet equivalent to 2.4 mg of cyclobenzaprine HCl (i.e., one tablet per dog). After the washout period of two weeks, dogs were sedated in the same way and given a single sublingual dose of the formulation without basifying agent. Blood samples after both types of treatment were taken pre-dose and at 5, 10, 20, 30, and 45 minutes, and at 1, 2, 3, 4, 6, 8, and 10 hours after dose administration. Blood was taken from the jugular vein. In all cases, after administration, animals did not have access to water for 30 minutes.

Cyclobenzaprine analysis was performed according to the analytical method described above. The method involved liquid-liquid extraction with hexane. The extracts were injected using an HPLC system and an API4000® (Applied Biosystems) for MS/MS detection, with an “Onyx monolithic Phenomenex C18 100×3.0 mm” column. The method was linear from 0.1 to 50 ng/mL for cyclobenzaprine in dog plasma. Quality control samples were prepared at 0.3, 25, and 40 ng/mL for cyclobenzaprine in dog plasma. Results obtained from the analysis of quality control samples were within the limits of acceptance defined in the protocol, therefore verifying the concentrations measured in plasma samples.

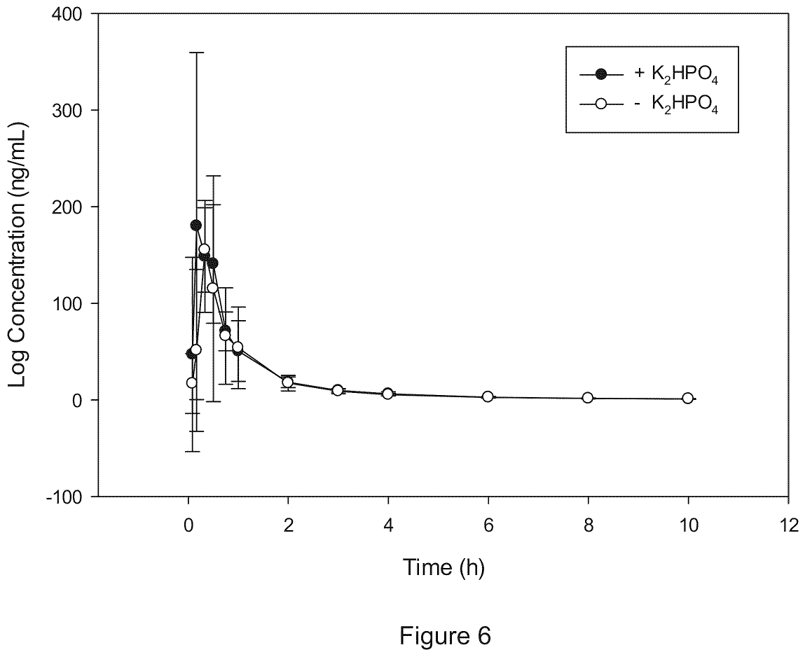

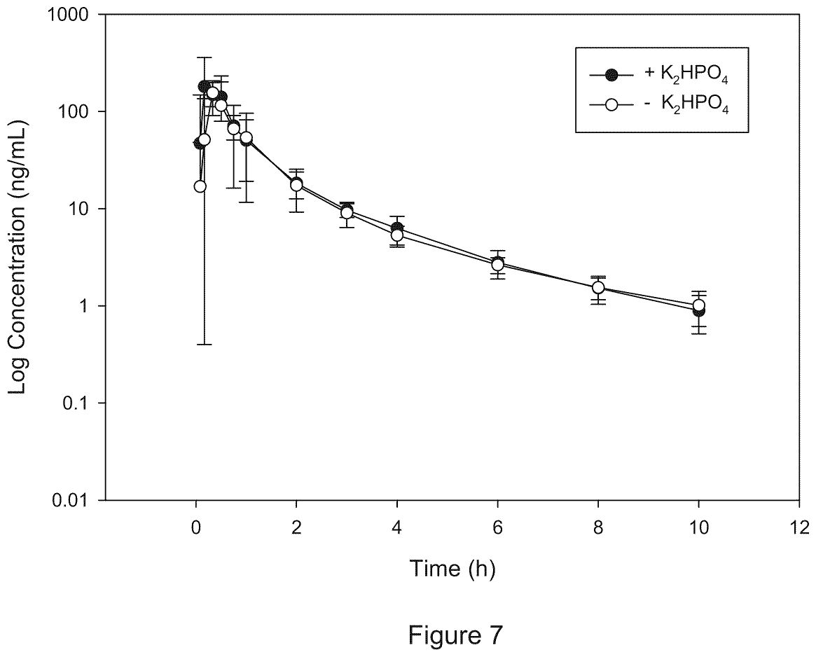

Sublingual administration was performed by placing the control tablet or tablet with K₂HPO₄ under the tongue when female beagles were slightly anesthetized with propofol. The results obtained showed that cyclobenzaprine in the tablet with K₂HPO₄ seemed to be about 25% more bioavailable than cyclobenzaprine when administered in the tablet without K₂HPO₄ (FIGS. 6 and 7; Tables 60-64). In addition, the AUC values calculated from 0 to 0.75 hours in beagles treated with cyclobenzaprine with K₂HPO₄ (AUC=135.6 ng h/mL) versus control cyclobenzaprine (AUC=82.4 ng h/mL) are quite different. Thus, the pharmacokinetic parameters associated with cyclobenzaprine in sublingual formulations appear to be improved with the addition of a basifying agent such as K₂HPO₄.

TABLE 60
Mean ± SD plasma PK parameters

| | Cmax | Tmax* | AUCt | AUCinf | % | Kel | t_{1/2} | Cl/F | Vd/F |
|---|---|---|---|---|---|---|---|---|---|
| Formulation | (ng/mL) | (h) | (ng/mL*h) | (ng/mL*h) | AUCextra | (1/h) | (h) | (L/h) | (L) |
| Buffered | 256.3 ± 108.5 | 0.33 | 176.6 ± 49.91 | 179.0 ± 50.23 | 1.390 ± 0.5573 | 0.3665 ± 0.05934 | 1.926 ± 0.2747 | 14.13 ± 3.297 | 40.06 ± 12.75 |
| tablet | | | | | | | | | |
| Unbuffered | 192.4 ± 76.69 | 0.33 | 151.6 ± 63.95 | 155.4 ± 64.57 | 2.489 ± 1.342 | 0.3001 ± 0.07501 | 2.425 ± 0.5932 | 17.18 ± 5.349 | 59.39 ± 20.12 |
| tablet | | | | | | | | | |

*Median

TABLE 61
Individual cyclobenzaprine plasma concentrations, with K₂HPO₄

| Time | | | | | | | | | | | |
|---|---|---|---|---|---|---|---|---|---|---|---|
| Hours | Dog 1 | Dog 2 | Dog 3 | Dog 4 | Dog 5 | Low | High | Mean | S.D. | % CV | n |
| 0 | BLQ | BLQ | BLQ | BLQ | BLQ | BLQ | BLQ | BLQ | NA | NA | 5 |
| 0.08333 | 5.801 | 226.6 | 1.212 | BLQ | 1.689 | 0.000 | 226.6 | 47.06 | 100.4 | 213.34 | 5 |
| 0.16667 | 381.2 | 366.6 | 81.84 | 4.318 | 65.44 | 4.318 | 381.2 | 179.9 | 179.5 | 99.78 | 5 |
| 0.33333 | 101.7 | 242.6 | 165.3 | 120.3 | 112.2 | 101.7 | 242.6 | 148.4 | 57.97 | 39.06 | 5 |
| 0.5 | 45.39 | 171.9 | 117.6 | 165.7 | 202.8 | 45.39 | 202.8 | 140.7 | 61.38 | 43.62 | 5 |
| 0.75 | 56.10 | 81.05 | 49.61 | 68.50 | 99.84 | 49.61 | 99.84 | 71.02 | 20.12 | 28.33 | 5 |
| 1 | 25.39 | 63.98 | 29.21 | 99.64 | 34.43 | 25.39 | 99.64 | 50.53 | 31.38 | 62.10 | 5 |
| 2 | 12.61 | 26.63 | 14.55 | 16.46 | 21.07 | 12.61 | 26.63 | 18.26 | 5.632 | 30.84 | 5 |
| 3 | 8.422 | 12.27 | 8.636 | 9.107 | 9.878 | 8.422 | 12.27 | 9.663 | 1.561 | 16.15 | 5 |
| 4 | 3.454 | 6.701 | 8.924 | 5.240 | 7.067 | 3.454 | 8.924 | 6.277 | 2.053 | 32.71 | 5 |
| 6 | 1.880 | 3.258 | 2.515 | 2.188 | 4.122 | 1.880 | 4.122 | 2.793 | 0.9027 | 32.32 | 5 |
| 8 | 1.059 | 1.190 | 2.151 | 1.275 | 1.939 | 1.059 | 2.151 | 1.523 | 0.4887 | 32.09 | 5 |
| 10 | 0.4612 | 1.350 | 0.9891 | 0.5514 | 1.117 | 0.4612 | 1.350 | 0.8937 | 0.3780 | 42.30 | 5 |

BLQ: Below the Limit of Quantification (0.1 ng/mL)
BLQ: Below the Limit of Quantification (1 ng/mL) (diluted plasma sample according to the protocol)
NA: Not Applicable

TABLE 62
Individual cyclobenzaprine plasma concentrations, without K₂HPO₄

| Time | | | | | | | | | | | |
|---|---|---|---|---|---|---|---|---|---|---|---|
| Hours | Dog 1 | Dog 2 | Dog 3 | Dog 4 | Dog 5 | Low | High | Mean | S.D. | % CV | n |
| 0 | BLQ | BLQ | BLQ | BLQ | BLQ | BLQ | BLQ | BLQ | NA | NA | 5 |
| 0.08333 | 1.448 | 4.871 | BLQ | 72.10 | 6.170 | 0.000 | 72.10 | 16.92 | 30.95 | 182.92 | 5 |
| 0.16667 | 13.27 | 6.661 | 199.7 | 4.470 | 31.79 | 4.470 | 199.7 | 51.18 | 83.72 | 163.58 | 5 |
| 0.33333 | 137.6 | 98.65 | 178.0 | 214.6 | 147.1 | 98.65 | 214.6 | 155.2 | 43.65 | 28.13 | 5 |
| 0.5 | 46.23 | 71.56 | 57.51 | 322.8 | 76.76 | 46.23 | 322.8 | 115.0 | 116.8 | 101.57 | 5 |
| 0.75 | 41.83 | 154.8 | 51.25 | 38.07 | 44.52 | 38.07 | 154.8 | 66.09 | 49.82 | 75.38 | 5 |
| 1 | 42.66 | 46.86 | 22.13 | 127.3 | 30.35 | 22.13 | 127.3 | 53.86 | 42.21 | 78.37 | 5 |
| 2 | 14.85 | 19.42 | 10.73 | 30.58 | 11.29 | 10.73 | 30.58 | 17.37 | 8.156 | 46.95 | 5 |
| 3 | 8.077 | 11.78 | 6.866 | 11.82 | 6.506 | 6.506 | 11.82 | 9.010 | 2.613 | 29.00 | 5 |
| 4 | 5.598 | 6.742 | 3.844 | 6.271 | 4.108 | 3.844 | 6.742 | 5.313 | 1.289 | 24.26 | 5 |
| 6 | 2.186 | 3.386 | 2.565 | 2.809 | 2.222 | 2.186 | 3.386 | 2.634 | 0.4928 | 18.71 | 5 |
| 8 | 1.290 | 2.205 | 1.365 | 1.559 | 1.286 | 1.286 | 2.205 | 1.541 | 0.3874 | 25.14 | 5 |
| 10 | 0.4694 | 1.459 | 1.180 | 1.209 | 0.7364 | 0.4694 | 1.459 | 1.011 | 0.3991 | 39.48 | 5 |

BLQ: Below the Limit of Quantification (0.1 ng/mL)
BLQ: Below the Limit of Quantification (1 ng/mL) (diluted plasma sample according to the protocol)
NA: Not Applicable

TABLE 63
Pharmacokinetic parameters, individual beagle dogs

| | | Cmax | Tmax | AUCt | AUCinf | % | Kel | t1/2 | CI/F | Vd/F |
|---|---|---|---|---|---|---|---|---|---|---|
| Formulation | Animal | (ng/mL) | (h) | (ng/mL*h) | (ng/mL*h) | AUCextra | (1/h) | (h) | (L/h) | (L) |
| With | Dog 1 | 381.2 | 0.167 | 136.5 | 137.9 | 1.012 | 0.3307 | 2.096 | 17.41 | 52.64 |
| K₂HPO₄ | Dog 2 | 366.6 | 0.167 | 260.1 | 263.0 | 1.101 | 0.4663 | 1.486 | 9.124 | 19.57 |
| | Dog 3 | 165.3 | 0.33 | 140.0 | 142.8 | 1.942 | 0.3568 | 1.943 | 16.81 | 47.12 |
| | Dog 4 | 165.7 | 0.50 | 175.7 | 177.2 | 0.8532 | 0.3647 | 1.900 | 13.55 | 37.14 |
| | Dog 5 | 202.8 | 0.50 | 170.8 | 174.3 | 2.040 | 0.3141 | 2.207 | 13.77 | 43.84 |
| | N | 5 | 5 | 5 | 5 | 5 | 5 | 5 | 5 | 5 |
| | Mean | 256.3 | 0.3328 | 176.6 | 179.0 | 1.390 | 0.3665 | 1.926 | 14.13 | 40.06 |
| | Stdev | 108.5 | 0.1665 | 49.91 | 50.23 | 0.5573 | 0.05934 | 0.2747 | 3.297 | 12.75 |
| | % CV | 42.34 | 50.03 | 28.26 | 28.06 | 40.11 | 16.19 | 14.26 | 23.33 | 31.84 |
| | SEM | 48.54 | 0.07446 | 22.32 | 22.46 | 0.2492 | 0.02654 | 0.1229 | 1.474 | 5.704 |
| | Harmean | 223.5 | 0.2631 | 167.4 | 169.8 | 1.227 | 0.3598 | 1.891 | 13.41 | 35.44 |
| | GeoMean | 238.8 | 0.2967 | 171.7 | 174.1 | 1.304 | 0.3630 | 1.909 | 13.79 | 37.98 |
| | Median | 202.8 | 0.33 | 170.8 | 174.3 | 1.101 | 0.3568 | 1.943 | 13.77 | 43.84 |
| | Min | 165.3 | 0.167 | 136.5 | 137.9 | 0.8532 | 0.3141 | 1.486 | 9.124 | 19.57 |
| | Max | 381.2 | 0.50 | 260.1 | 263.0 | 2.040 | 0.4663 | 2.207 | 17.41 | 52.64 |
| Without | Dog 1 | 137.6 | 0.33 | 110.2 | 111.4 | 1.020 | 0.4132 | 1.678 | 21.55 | 52.15 |
| K₂HPO₄ | Dog 2 | 154.8 | 0.75 | 154.6 | 161.6 | 4.291 | 0.2105 | 3.293 | 14.86 | 70.58 |
| | Dog 3 | 199.7 | 0.167 | 125.4 | 129.7 | 3.287 | 0.2768 | 2.504 | 18.51 | 66.86 |
| | Dog 4 | 322.8 | 0.50 | 260.9 | 264.7 | 1.410 | 0.3240 | 2.139 | 9.068 | 27.99 |
| | Dog 5 | 147.1 | 0.33 | 106.9 | 109.5 | 2.435 | 0.2761 | 2.511 | 21.91 | 79.36 |
| | N | 5 | 5 | 5 | 5 | 5 | 5 | 5 | 5 | 5 |
| | Mean | 192.4 | 0.4154 | 151.6 | 155.4 | 2.489 | 0.3001 | 2.425 | 17.18 | 59.39 |
| | Stdev | 76.69 | 0.2210 | 63.95 | 64.57 | 1.342 | 0.07501 | 0.5932 | 5.349 | 20.12 |
| | % CV | 39.86 | 53.21 | 42.18 | 41.56 | 53.94 | 24.99 | 24.46 | 31.14 | 33.87 |
| | SEM | 34.30 | 0.09884 | 28.60 | 28.88 | 0.6004 | 0.03355 | 0.2653 | 2.392 | 8.996 |
| | Harmean | 174.6 | 0.3251 | 136.2 | 139.7 | 1.896 | 0.2858 | 2.310 | 15.45 | 51.74 |
| | GeoMean | 182.4 | 0.3688 | 142.9 | 146.6 | 2.181 | 0.2928 | 2.367 | 16.37 | 55.92 |
| | Median | 154.8 | 0.33 | 125.4 | 129.7 | 2.435 | 0.2768 | 2.504 | 18.51 | 66.86 |
| | Min | 137.6 | 0.167 | 106.9 | 109.5 | 1.020 | 0.2105 | 1.678 | 9.068 | 27.99 |
| | Max | 322.8 | 0.75 | 260.9 | 264.7 | 4.291 | 0.4132 | 3.293 | 21.91 | 79.36 |

TABLE 64
Relative bioavailability,
individual beagle dogs

| | F (%) = |
|---|---|
| | AUCinf (with K₂HPO₄)/ |
| | AUCinf (without |
| Animal | K₂HPO₄) * 100 |
| Dog 1 | 123.79 |
| Dog 2 | 162.75 |
| Dog 3 | 110.10 |
| Dog 4 | 66.94 |
| Dog 5 | 159.18 |
| N | 5 |
| Mean | 124.55 |
| Stdev | 39.33 |
| % CV | 31.57 |
| SEM | 17.59 |
| Harmean | 112.29 |
| GeoMean | 118.77 |
| Median | 123.79 |
| Min | 66.94 |
| Max | 162.75 |

### Example 11

To study whether sublingual cyclobenzaprine is efficiently absorbed in humans, a solution for sublingual administration containing 2.4 mg of cyclobenzaprine HCl (2.4 mg/mL) formulation in an aqueous solution containing K₂HPO₄ at pH 7.0-7.4 may be used in lieu of sublingual tablets that would introduce a disintegration factor. Sublingual administration of cyclobenzaprine in the context of the invention may take place through, inter alia, sublingual tablets or a liquid solution. As described above, with a sublingual formulation, cyclobenzaprine is expected to be more bioavailable and to provide more predictable absorption of cyclobenzaprine than oral tablets, such as those currently available. As a result, patients may be less likely to receive too little drug to receive therapeutic effect, and yet may also be less likely to be overdosed, reducing the potential for side-effects, i.e., next-day drowsiness and/or noncompliance due to intolerance. Sublingual administration also is expected to bypass the first-pass hepatic metabolism that results in, among other metabolites, demethylation of cyclobenzaprine to norcyclobenzaprine.

The extent to which the pH of sublingual oral solutions affects the rate or efficiency of sublingual absorption is not completely understood. The results described in Example 9 found rapid absorption at pH 7.4. To more completely characterize the extent to which absorption is affected by the pH of oral solution formulations, the cyclobenzaprine HCl formulation 2.4 mg sublingual solution was tested at a pH of 7.4 and a pH of 3.5. To establish a baseline for absolute bioavailability, cyclobenzaprine 2.4 mg also was administered as an IV solution (0.6 mg/mL) at pH 7.4.

The single-center, randomized, open-label, single-dose, comparative, parallel-design pharmacokinetic study described below was appropriately designed to compare the rate and extent of absorption of cyclobenzaprine HCl formulation 2.4 mg sublingual solution (2.4 mg/mL) at pH 7.4 versus cyclobenzaprine HCl formulation 2.4 mg sublingual solution (2.4 mg/mL) at pH 3.5, oral cyclobenzaprine 5 mg immediate-release tablets, and intravenous cyclobenzaprine at a dose of 2.4 mg (0.6 mg/mL) in an aqueous solution containing K₂HPO₄ at pH 7.4. The safety and tolerability of the cyclobenzaprine HCl formulation 2.4 mg sublingual solution at pH 3.5 and 7.4 also was assessed and compared to the safety and tolerability of cyclobenzaprine 5 mg tablets and cyclobenzaprine 2.4 mg intravenous solution.

Potential subjects were screened by medical and psychiatric history and laboratory and physical examinations 2 to 30 days prior to dose administration. Baseline evaluations were conducted and subjects are randomly assigned to a formulation/treatment 1 day prior to drug administration (Day −1). The next morning, after all pre-dose assessments were completed and eligible subjects agreed to continue, subjects were randomly assigned to study medication and received the assigned dose of test or reference drug. Subjects were required to have fasted for at least 10 hours prior to dosing and for 4 hours thereafter.

Subjects were confined from at least 10 hours before dosing until after the 72-hour discharge procedures. Subjects were required to remain seated or semi-reclined and to avoid lying down or sleeping, unless medically necessary or procedurally required, for up to 4 hours after administration of the assigned study drug. Due to the long confinement period, supervised outings were permitted during the period of confinement. No outing was allowed on the day of dosing (Day 1).

Tests for thyroid-stimulating hormone (TSH), human immunodeficiency virus types 1 and 2 (HIV1 and HIV2), hepatitis B (HBsAg), and hepatitis C (HCAb) are conducted at screening only, and urine drug screens, alcohol breath tests, and urine cotinine tests were conducted at screening and admission. Physical examinations, laboratory tests, vital signs, 12-lead electrocardiograms (ECGs), weight/body mass index (BMI), and pregnancy tests were performed at specified intervals. Monitoring for adverse events (AEs) and concomitant medications were conducted continuously. Blood and urine samples were collected at specified intervals for the measurement of levels of cyclobenzaprine and norcyclobenzaprine in plasma and urine. A post-study follow-up telephone call was scheduled 10±3 days after administration of the assigned study medication. For subjects who discontinued prematurely, every effort was made to complete the discharge assessments and the follow-up telephone call.

The objective of comparing the rate and extent of absorption of sublingual cyclobenzaprine HCl solution 2.4 mg (2.4 mg/mL) in an aqueous solution containing K₂HPO₄ at pH 3.5 versus sublingual cyclobenzaprine HCl solution 2.4 mg (2.4 mg/mL) in aqueous solution containing K₂HPO₄ at pH 7.4 was met through analysis of multiple plasma and urine samples collected from the subjects and by comparing those associated with the two formulations. The objective of comparing the rate and extent of absorption of sublingual cyclobenzaprine HCl solution 2.4 mg (2.4 mg/mL) at pH 3.5 and pH 7.4 (test formulations) versus oral cyclobenzaprine 5 mg tablets, and intravenous cyclobenzaprine 2.4 mg (0.6 mg/mL) in an aqueous solution containing K₂HPO₄ (reference formulations) was met through analysis of multiple plasma and urine samples collected from the subjects and comparing those associated with these formulations or treatments. The objective of assessing the safety and tolerability of cyclobenzaprine HCl 2.4 mg (2.4 mg/mL) sublingual solution formulations at pH 3.5 and 7.4 was addressed by monitoring AEs, clinical laboratory values, vital signs, 12-lead ECGs, weight/BMI, concomitant medications, and overall well-being prior to, during, and at the close of the 4-day in-house dosing period.

Before undergoing any study-related screening procedures, each potential subject provided signed informed consent. The investigator determined the potential subject's suitability for the study by interviewing the subject and by performing per-protocol screening assessments. Subjects were administered a single-dose treatment according to a block randomization scheme. Six subjects were randomly assigned to each of the four groups, for a total enrollment of 24 subjects.

The four treatments were as follows:

Treatment A: 1 dose of 2.4 mg cyclobenzaprine HCl sublingual solution (2.4 mg/mL) in aqueous solution containing K₂HPO₄ at pH 3.5, administered as 1 mL held under the tongue for 90 seconds without swallowing

Treatment B: 1 dose of 2.4 mg cyclobenzaprine HCl sublingual solution (2.4 mg/mL) in aqueous solution containing K₂HPO₄ at pH 7.4, administered as 1 mL held under the tongue for 90 seconds without swallowing

Treatment C: 1 dose of 5 mg cyclobenzaprine immediate-release tablets, swallowed with 240 mL of room-temperature water

Treatment D: 1 dose of 2.4 mg cyclobenzaprine USP in aqueous solution containing K₂HPO₄ at pH 7.4 (0.6 mg/mL), administered intravenously as a 4 mL bolus injection over 30 seconds

Each subject participated for up to approximately 43 days, including an up to 30-day screening period, a 4-day in-house dosing period, and a follow-up telephone call 10±3 days after study drug administration.

The low-dose cyclobenzaprine HCl sublingual solution 2.4 mg (2.4 mg/mL) was administered sublingually via Becton Dickinson 1 mL needle-less syringe. This sublingual solution consisted of cyclobenzaprine USP dissolved in aqueous solution containing K₂HPO₄ at a concentration of 2.4 mg/mL. The solution was manufactured as two formulations that were identical except that one is provided at pH 7.4 and the other is provided at pH 3.5. The two cyclobenzaprine HCl 2.4 mg sublingual formulations were filled in single-use 3 mL vials (1.5 mL per 3 mL vial), labeled, packaged, and provided for use in the study.

Cyclobenzaprine intravenous solution containing 0.6 mg/mL of cyclobenzaprine USP was used as a reference comparator in this trial. This solution was identical to the cyclobenzaprine HCl 2.4 mg sublingual solution except that it was formulated to a concentration of 0.6 mg/mL, with a pH adjusted to 7.4. This cyclobenzaprine 2.4 mg intravenous solution was filled in sterile 10 mL single-use vials (5 mL per 10 mL vial), labeled, packaged, and provided for use in the study.

Blood and urine samples for pharmacokinetic analysis were collected and vital signs recorded pre-dose and at specified intervals after dosing. Laboratory tests and 12-lead ECGs were conducted on Day −1 and prior to discharge on the morning of Day 4. A serum 3-HCG pregnancy test was conducted for all female subjects on Day −1. A urine 3-HCG pregnancy test was conducted for all female subjects prior to discharge on the morning of Day 4. Monitoring for adverse events and concomitant medications was conducted continuously.

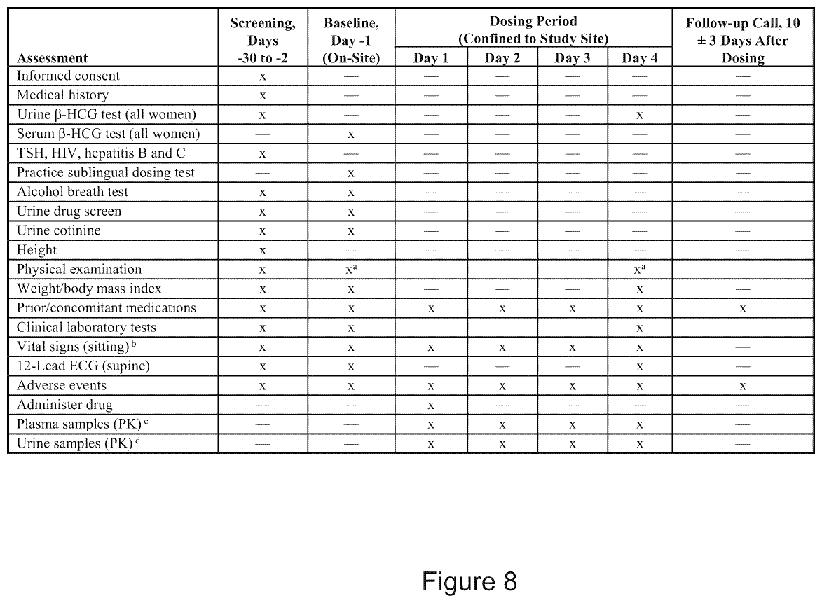

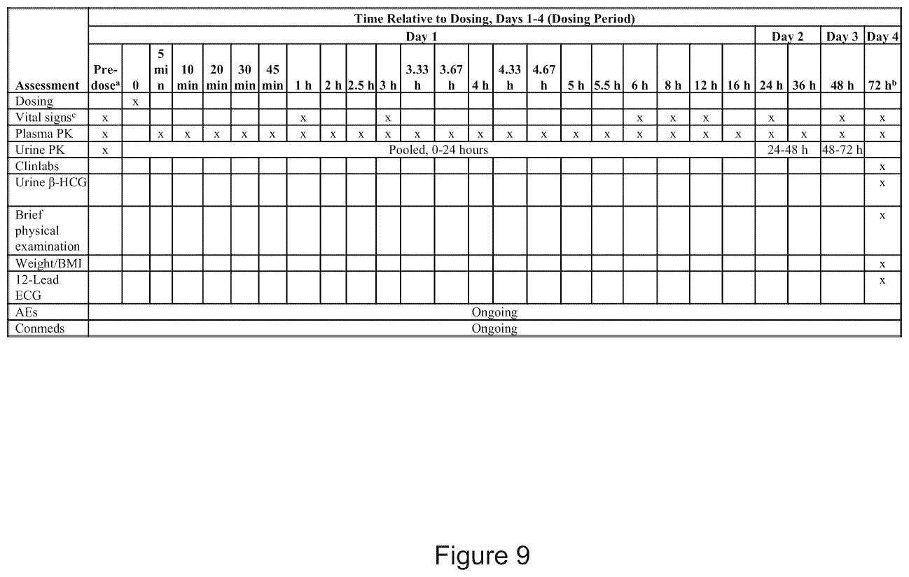

For pharmacokinetic analysis, a total of 25 blood samples (6 mL per sample) are taken: within 30 minutes pre-dose and 5, 10, 20, 30, and 45 minutes and 1, 2, 2.5, 3, 3.33, 3.67, 4, 4.33, 4.67, 5, 5.5, 6, 8, 12, 16, 24, 36, 48, and 72 hours post-dose. Actual sampling times were used for statistical analyses. Unless otherwise specified or for subject safety, when blood draws and other procedures coincide, blood draws had precedence. A dead-volume intravenous catheter was used for blood collection to avoid multiple skin punctures, when appropriate. Otherwise, blood samples were collected by direct venipuncture. Charts of daily and hourly assessments are shown in FIGS. 8 and 9, respectively.

Also for pharmacokinetic analysis, a single urine sample was collected within 30 minutes pre-dose (one sample), after which urine was pooled for the duration of the dosing period, from 0 to 24, 24 to 48, and 48 to 72 hours post-dose. If a subject could not void his or her bladder within 30 minutes before dosing, a sample from earlier that morning may have been used as the pre-dose sample. Urine voided by subjects within 10 minutes of the end of the interval were included in the earlier sample. Subjects were asked to void their bladders within 5 minutes before the end of each collection interval, so that each new interval would begin with an empty bladder. Any urine voided by subjects but not collected was documented. Pharmacokinetic parameters (plasma) included AUC₀₋ₜ, AUC_{0-inf}, Cₘₐₓ, Residual area, Tₘₐₓ, T_{1/2 el}, Kₑₗ, and F. F was calculated for the sublingual and oral formulations of cyclobenzaprine only. Pharmacokinetic parameters (urine) included Ae₀₋ₜ, Rₘₐₓ, and Tₘₐₓ.

For blood samples, ANOVA was performed on Tₘₐₓ, Kₑₗ, and T_{1/2 el} and on AUC₀₋ₜ, AUC_{0-inf}, and Cₘₐₓ at the alpha level of 0.05. The ratio of means (treatments A/B, A/C, A/D, B/C, and B/D) and 90% geometric confidence interval (CI) for the ratio of means, based on least-squares means from the ANOVA of the In-transformed data, was calculated for AUC₀₋ₜ, AUC_{0-inf}, and Cₘₐₓ. For all analytes, the ratio of means (treatments A/B, A/C, A/D, B/C, and B/D) and 90% geometric CI for the ratio of means, based on least-squares means from the ANOVA of the In-transformed data, were calculated for Ae₀₋ₜ and Rₘₐₓ.

Discharge from the study unit occurred following completion of the scheduled discharge assessments 72±1.5 hours following study drug administration (morning of Day 4). Barring safety concerns, the subjects were discharged from the study unit after these examinations (i.e., 4 days after dosing), at the discretion of the investigator.

A follow-up call was made to each subject by a study staff member 3-9 days subsequent to discharge from the study unit (that is, 10±3 days after dosing). The subject was asked to report any adverse events he or she may have experienced since discharge from the study unit. The subject was considered to have completed the study after the follow-up call.

This experiment was designed to be a stringent test of transmucosal absorption after sublingual administration of the oral cyclobenzaprine solution, so subjects were instructed to hold the solution under their tongues for 90 seconds, spit out the contents of their mouths, rinse their mouths with 60 mL of water, and then drink 240 mL of water. Unlike the anesthetized beagles, some of the alert humans were expected to swallow part of the sublingual solutions as part of an involuntary reflex. Table 65 shows the results of the study.

TABLE 65
Mean plasma cyclobenzaprine pharmacokinetics

| | | AUC₀₋ₜ | AUC_{0-inf} | Residual | Cₘₐₓ | Tₘₐₓ | T_{1/2 el} | Kₑₗ |
|---|---|---|---|---|---|---|---|---|
| Treatment | N | (pg · hr/mL) | (pg · hr/mL) | Area (%) | (pg/mL) | (hr) | (hr) | (1/hr) |
| TNX-102 2.4 | 4 ª | 16,065 | 20,304 | 20.5 | 742.7 | 5.208 | 35.1 | 0.020275 |
| mg SL | | | | | | | | |
| solution at pH | | | | | | | | |
| 7.1 | | | | | | | | |
| TNX-102 2.4 | 4 ^{b} | 2,083 | 3,142 | 33.5 | 180.6 | 5.208 | 13.5 | 0.0833 |
| mg SL | | | | | | | | |
| solution at pH | | | | | | | | |
| 3.5 | | | | | | | | |
| Cyclobenzaprine | 6 | 65,751 | 78,151 | 15.9 | 10,652.2 | 0.041 | 31.5 | 0.0224 |
| 2.4 mg IV | | | | | | | | |
| solution | | | | | | | | |
| Cyclobenzaprine | 6 | 57,840 | 69,696 | 14.8 | 3,019.6 | 5.470 | 28.4 | 0.0256 |
| 5 mg oral | | | | | | | | |
| tablets ^{c} | | | | | | | | |

^{a} The means for 2.4 mg sublingual solution at pH 7.1 exclude Subject 7 (outlier) and Subject 10, who appeared to have swallowed the sublingual solution medication.
^{b} The means for 2.4 mg sublingual solution at pH 3.5 exclude Subject 4, who appeared to have swallowed the sublingual solution medication.
^{c} Each subject received a single cyclobenzaprine 5 mg oral tablet.

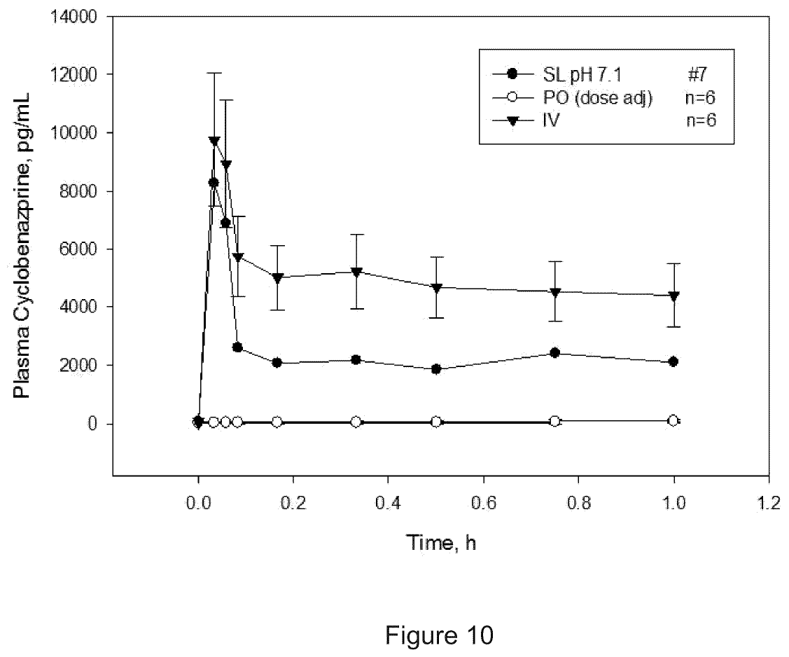

As expected, several subjects swallowed their sublingual doses. Subject 7, who was excluded from the analysis summarized in Table 65 because her absorption of a sublingual dose was so much more rapid than that of others given one of the sublingual treatments, seems likely to have been the most fully compliant with the intended dosing procedure and to best represent the potential of sublingual dosing. Analysis of the data show that Subject 7 in the cohort receiving 2.4 mg sublingual solution at pH 7.1 had a pharmacokinetic profile strikingly similar to the mean of the entire cohort receiving 2.4 mg IV solution during the 0- to 1-hour time frame, while the cohort receiving cyclobenzaprine 5 mg tablets showed almost no absorption during this time. The efficiency of absorption in Subject 7 was approximately half the absorption in the cohort receiving 2.4 mg IV solution. The concentration-time profiles of the cohort receiving 2.4 mg IV solution and of Subject 7 in the cohort receiving 2.4 mg sublingual solution at pH 7.1 had two distinct phases in the first hour, with the first phase showing a rapid increase and clearance from plasma before 5 min (0.83 h) and a second phase showing relatively flat plasma concentrations from 10 min (0.167 hour) to 60 min (1 hour). The plasma levels for oral dosing shown in FIG. 10 were decreased by a factor of 2.4/5.0 to facilitate comparison of data for the 2.4 mg sublingual (SL) solution to data for the cyclobenzaprine 5 mg oral (PO) tablet group, assuming dose proportionality. During the first hour, the plasma samples were obtained at 0 min, 2 min (0.033 h), 3 min (0.058 h), 5 min (0.083 hr), 10 min (0.167 h), 20 min (0.33 h), 30 min (0.5 h), 45 min (0.75 h) and 60 min (1 h) (FIG. 10).

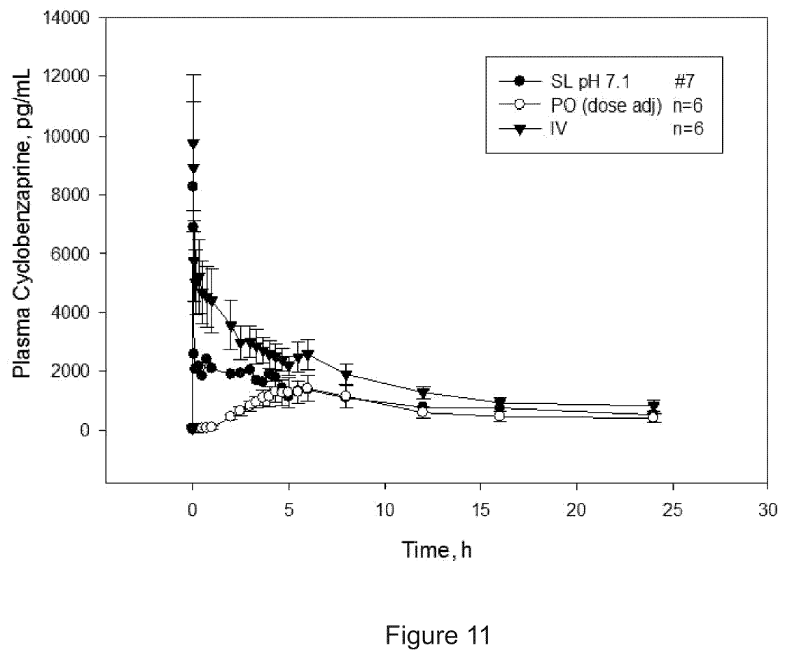

Preliminary analysis of pharmacokinetic data over 24 hours showed that Subject 7, in the cohort receiving 2.4 mg SL solution at pH 7.1, continued to show a concentration-time profile over 24 hours similar to that of the entire cohort receiving 2.4 mg IV solution. Analysis of the pharmacokinetic data over 24 hours also showed that the plasma levels of the cohort receiving 2.4 mg IV solution fell rapidly between hours 1 and 5, while the plasma levels of the cohort receiving cyclobenzaprine 5 mg tablets increased to Tₘₐₓ at approximately 5 hours. The plasma levels of the cohort receiving 2.4 mg IV solution showed a small increase after 5 hours consistent with some of the initial dose entering the bile and subsequently being taken up by the portal circulation (enterohepatic recirculation). The plasma levels for oral dosing shown were decreased by a factor of 2.4/5.0 to facilitate comparison of data for the 2.4 mg SL solution to data for the cyclobenzaprine 5 mg oral tablet group, assuming dose proportionality (FIG. 11).

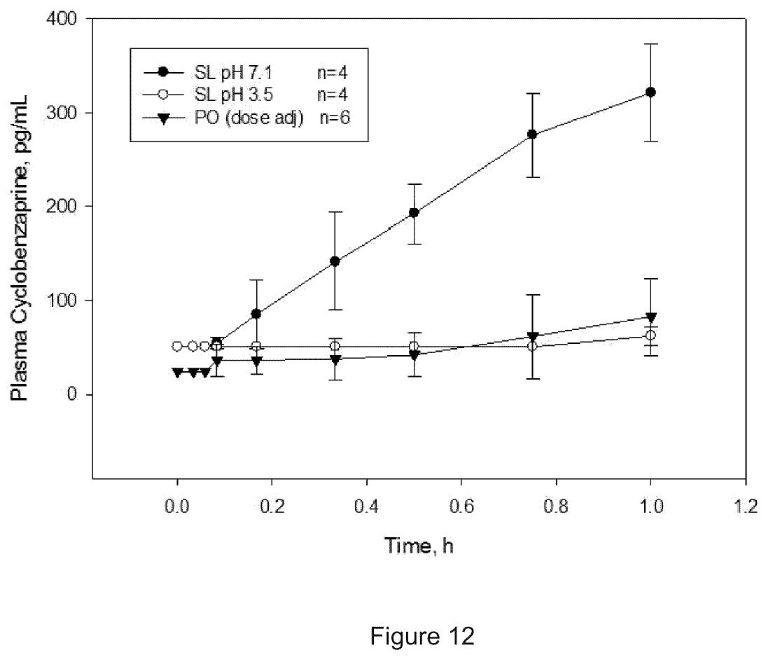

Because Subject 7 was an outlier, we also compared the mean pharmacokinetics of four of the other subjects from the cohort receiving 2.4 mg SL solution at pH 7.1 against subjects receiving 2.4 mg SL solution at pH 3.5 and subjects receiving cyclobenzaprine 5 mg oral tablets. The group receiving 2.4 mg SL solution at pH 7.1 showed a rapid rise in plasma cyclobenzaprine over the first hour (FIG. 12). In contrast, very low levels of cyclobenzaprine were observed over this time period in either the cohort receiving 2.4 mg SL solution at pH 3.5 or the cyclobenzaprine 5 mg oral tablet cohort. The group mean for 2.4 mg SL solution at pH 7.1 excludes Subject 7 (outlier) and Subject 10 (who appeared to have swallowed the SL solution medication), and the group mean for 2.4 mg SL solution at pH 3.5 excludes Subject 4 (who appeared to have swallowed the SL solution medication). The plasma levels shown for cyclobenzaprine 5 mg oral tablets were decreased by a factor of 2.4/5.0 to facilitate comparison of data for the 2.4 mg SL solution groups to data for the cyclobenzaprine 5 mg oral tablet group, assuming dose proportionality. These findings indicated that the phosphate in the 2.4 mg SL solution at pH 7.1 was associated with increased transmucosal absorption of cyclobenzaprine after sublingual administration.

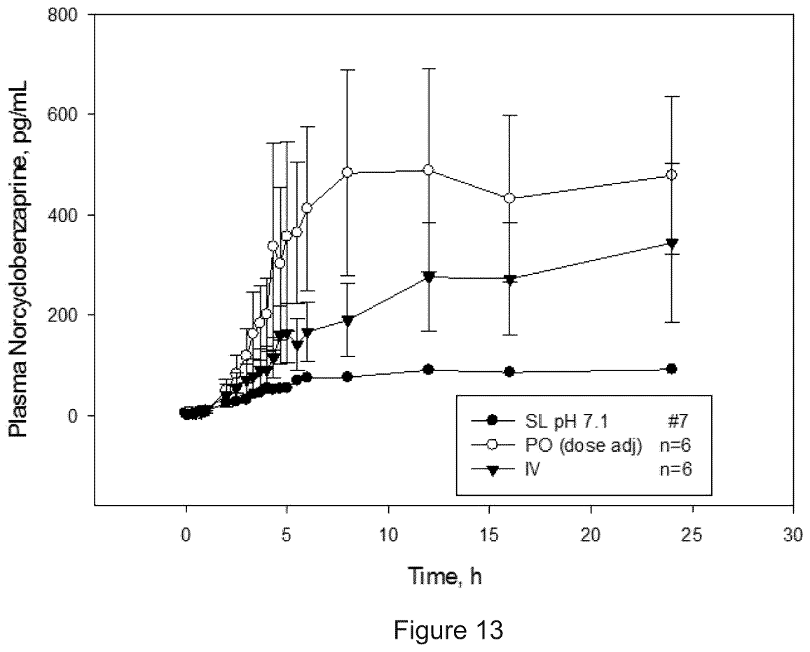

In order to determine whether sublingual administration of 2.4 mg SL solution (cyclobenzaprine HCl) had an effect on the formation of the long-lived metabolite, norcyclobenzaprine, plasma levels of this metabolite were determined. As shown in FIG. 13, levels of norcyclobenzaprine from the IV cohort and from Subject 7 trended lower than the group mean observed for the cyclobenzaprine 5 mg oral tablet cohort, which is consistent with a reduction in first-pass metabolism by sublingual or IV administration.

The 2.4 mg SL solution at pH 7.1 had a benign safety profile. All treatment emergent adverse events (TEAEs) in this cohort were mild, and all but one (lipase increased) had resolved by the time of discharge from the study site (the lipase elevation was detected in a sample obtained at discharge, and the subject could not be reached for follow-up). TEAEs in all four cohorts were mild or moderate and generally compatible with the labeling for marketed cyclobenzaprine tablets.

An additional pharmacokinetic analysis of the data was performed by calculating partial AUCs from time 0 hr to each sampling time point up to 8 hours for each individual subject. The partial AUC data in group C was then dose-normalized to the same dose as in group B (5 mg to 2.4 mg), assuming linear pharmacokinetics. Geometric means were calculated for the partial AUCs in the two groups. Geometric means were preferred compared to arithmetic means due to the usually log-normal distribution of pharmacokinetic parameters such as AUC. Finally, the partial AUCs among the group were statistically compared. In line with the using geometric means, the partial AUCs were log-transformed prior to performing a t-test for unpaired samples. The results indicated that AUC(0-0.5 hr), AUC(0-0.75 hr), AUC(0-1 hr), AUC(0-2 hr) and AUC(0-2.5 hr) were significantly higher after the administration of the phosphate-containing solution as compared to the tablet (Table 66). This means that at any time for the first 2.5 hr after dosing, the phosphate-containing solution achieved a higher systemic exposure as compared to the tablet.

TABLE 66
Partial AUC for Groups B and C

| Group | AUC_{0-0.5 hr} | AUC_{0-0.75 hr} | AUC_{0-1 hr} | AUC_{0-2 hr} | AUC_{0-2.5 hr} |
|---|---|---|---|---|---|
| B | 73 | 152 | 246 | 828 | 1210 |
| C | 1.0 | 5.4 | 22 | 296 | 564 |
| p-value | 0.0068 | 0.0094 | 0.0037 | 0.0870 | 0.1419 |

### Example 12

A single-dose, open-label, randomized, parallel-design study of the comparative pharmacokinetics and safety of sublingual cyclobenzaprine tablets was performed. The study compared sublingual cyclobenzaprine tablets (with phosphate) at 2.4 mg and 4.8 mg doses, sublingual cyclobenzaprine tablets (without phosphate) at 2.4 mg, and cyclobenzaprine 5 mg oral tablets. The study compares 1) the rate and extent of absorption of 2.4 mg sublingual cyclobenzaprine HCl tablets with and without phosphate; and 2) the rate and extent of absorption of 2.4 mg sublingual cyclobenzaprine HCl tablets (with phosphate) administered at doses of 2.4 mg and 4.8 mg vs. 2.4 mg sublingual cyclobenzaprine HCl tablets (without phosphate) at a 2.4 mg dose vs. cyclobenzaprine 5 mg oral tablets; and 3) assesses the safety and tolerability of 2.4 mg sublingual cyclobenzaprine HCl tablets (with phosphate) at doses of 2.4 mg and 4.8 mg vs. 2.4 mg sublingual cyclobenzaprine HCl tablets (without phosphate) at a 2.4 mg dose vs. cyclobenzaprine 5 mg oral tablets.

Patients were selected substantially as described in Example 11. Cyclobenzaprine was administered as follows:

Treatment A: A single dose of one TNX-102 2.4 mg SL tablet (with phosphate). Subjects were asked to keep the tablet under the tongue until dissolved and not to crush or chew it. Subjects were asked not to drink any water until at least 1 hour after dosing.

Treatment B: A single dose of one TNX-102-A 2.4 mg SL tablet (without phosphate). Subjects were asked to keep the tablet under the tongue until dissolved and not to crush or chew it. Subjects were asked not to drink any water until at least 1 hour after dosing.

Treatment C: A single dose of two TNX-102 2.4 mg SL tablets (with phosphate). Subjects will be asked to keep the tablets under the tongue until dissolved and not to crush or chew them. Subjects were asked not to drink any water until at least 1 hour after dosing.

Treatment D: A single dose of one cyclobenzaprine 5 mg oral immediate-release tablet (Watson Pharmaceuticals), to be swallowed with 240 mL of room-temperature water. Subjects were asked to swallow the administered tablet whole and not to crush or chew it.

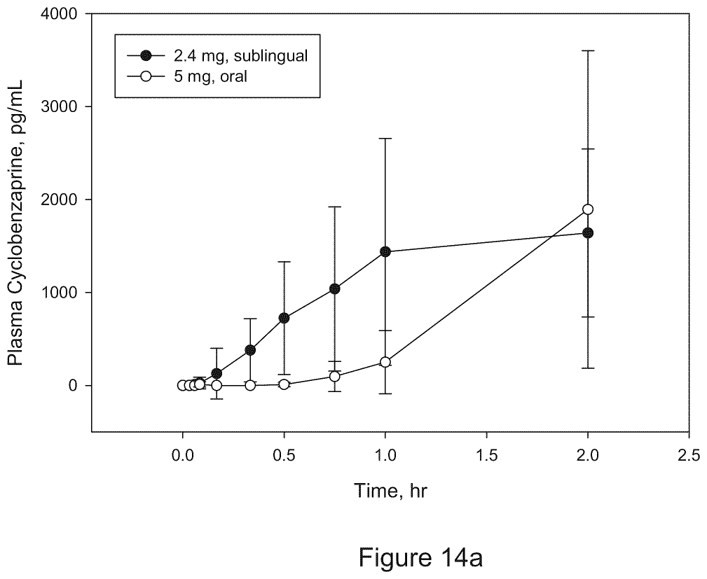

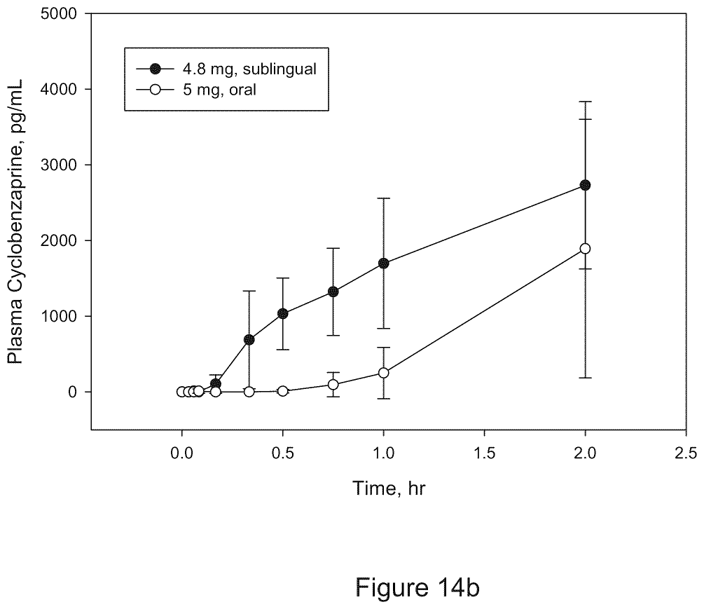

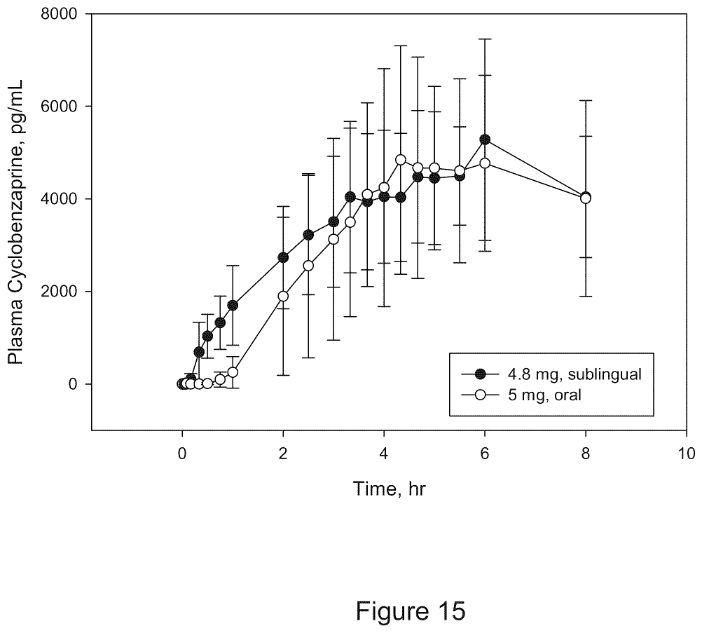

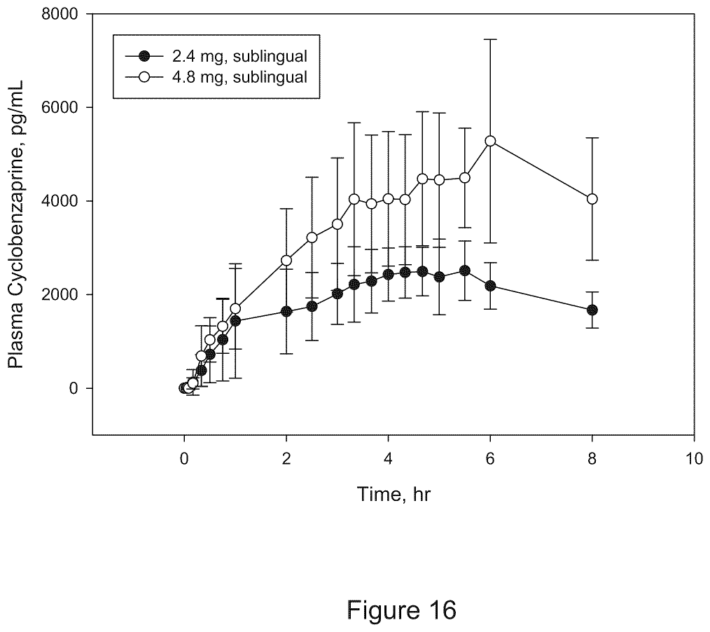

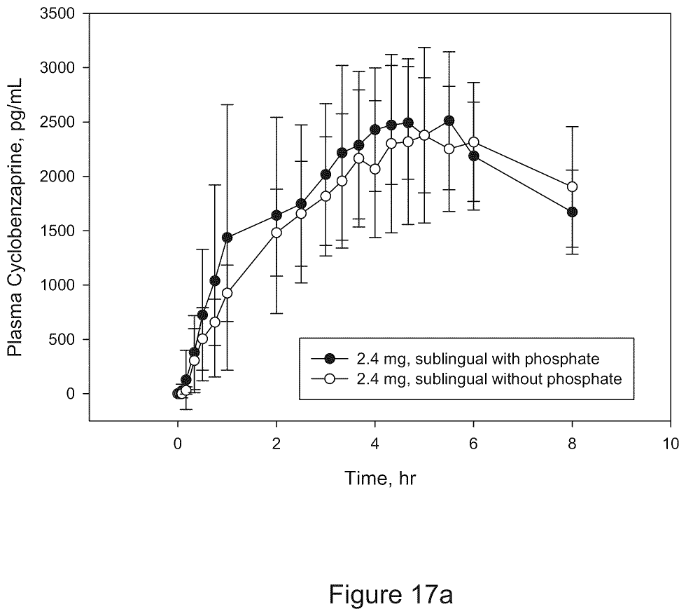

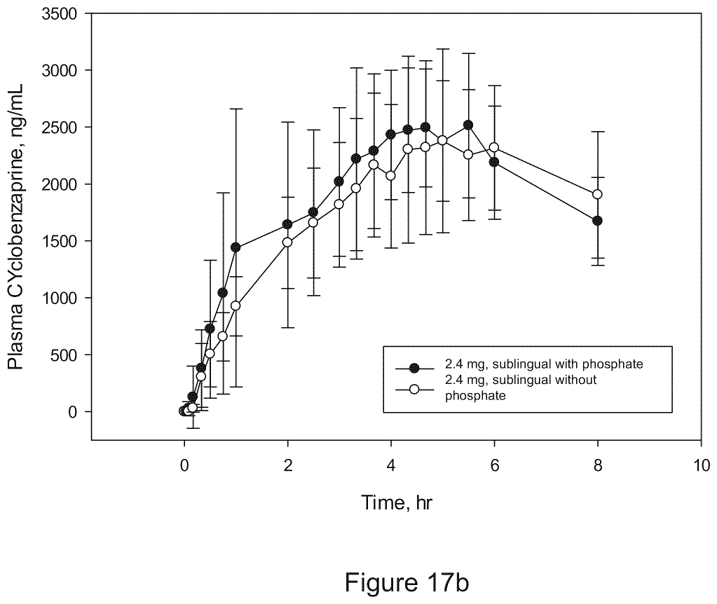

The sublingual cyclobenzaprine HCl tablets were well tolerated, with no serious adverse affects, although some subjects experienced numbness of the tongue. Compared to 5 mg of oral cyclobenzaprine, 4.8 mg of sublingual cyclobenzaprine HCl significantly increased the rate of absorption in the first two hours after administration (FIGS. 14 and 15). Indeed, at some time points, the sublingual cyclobenzaprine produced approximately 20 fold higher mean dose-adjusted plasma levels of cyclobenzaprine as compared to oral administration. The sublingually administered cyclobenzaprine also resulted in a higher AUC than the orally administered cyclobenzaprine. Sublingual cyclobenzaprine HCl also resulted in approximate dose proportionality between the 2.4 mg and the 4.8 mg doses (FIG. 16). Additionally, the sublingual tablets with phosphate showed a trend towards faster cyclobenzaprine absorption than the tablets without phosphate (FIG. 17). Table 67 shows the statistical significance of partial AUC analysis using a one-way ANOVA test with Bonferroni one-way comparisons between the different treatment groups.

Partial AUC at different times were calculated to examine the effect of sublingual administration on absorption. Statistical comparison for In-transformed AUC data was analyzed by One-Way ANOVA with Bonferroni one-way comparisons (95% confidence level). Comparing the partial AUC for cyclobenzaprine HCl SL 2.4 mg (1 tablet) versus an AUC for cyclobenzaprine 5 mg IR tablets (dose-normalized to 2.4 mg) revealed a statistical significant increase in absorption for the SL 2.4 mg 1 tablet administration versus cyclobenzaprine 5 mg IR: AUC₀₋₂₀min, 37 ng hr L⁻¹ vs. 0 ng hr L⁻¹, p<0.05; AUC₀₋₃₀min, 128 ng hr L⁻¹ vs. 1 ng hr L⁻¹, p<0.05; AUC₀₋₄₅min, 333 ng hr L⁻¹ vs. 2 ng hr L⁻¹, p<0.05; AUC₀₋₁ₕ, 614 ng hr L⁻¹ vs. 5 ng hr L⁻¹, p<0.05; AUC₀₋₂ₕ, 2098 ng hr L⁻¹ vs. 386 ng hr L⁻¹, p<0.05; AUC_{0-2.5 h}, 2955 ng hr L⁻¹ vs. 791 ng hr L⁻¹, p<0.05; AUC_{0-3 h}, 3931 ng hr L⁻¹ vs. 1355 ng hr L⁻¹, p<0.05.

Partial AUC at different times were analyzed to show the effect of cyclobenzaprine HCl SL administration on absorption. Comparing the mean partial AUC for 2 tablets of 2.4 mg cyclobenzaprine HCl versus a mean partial AUC for cyclobenzaprine 5 mg IR tablets (dose-normalized to 2.4 mg) revealed a statistically significant increase in absorption: AUC₀₋₂₀min, 23 ng hr L⁻¹ vs. 0 ng hr L⁻¹, p<0.05; AUC₀₋₃₀min, 86 ng hr L⁻¹ vs. 1 ng hr L⁻¹, p<0.05; AUC₀₋₄₅min, 223 ng hr L⁻¹ vs. 2 ng hr L⁻¹, p<0.05; AUC_{0-1 h}, 405 ng hr L⁻¹ vs. 5 ng hr L⁻¹, p<0.05; AUC_{0-2 h}, 1478 ng hr L⁻¹ vs. 386 ng hr L⁻¹, p<0.05; AUC_{0-2.5 h}, 2167 ng hr L⁻¹ vs. 791 ng hr L⁻¹, p<0.05.

TABLE 67
One-Way ANOVA w/Bonferroni one-way comparisons (95% confidence level)

| Comparison/ | | | | | | | | |
|---|---|---|---|---|---|---|---|---|
| p-value | AUC₀₋₂₀ₘᵢₙ | AUC₀₋₃₀ₘᵢₙ | AUC₀₋₄₅ₘᵢₙ | AUC₀₋₁ₕᵣ | AUC₀₋₂ₕᵣ | AUC_{0-2.5hr} | AUC₀₋₃ₕᵣ | AUC_{0-3.33hr} |
| A vs. D | <0.05 | <0.05 | <0.05 | <0.05 | <0.05 | <0.05 | <0.05 | — |
| B vs. D | <0.05 | <0.05 | <0.05 | <0.05 | <0.05 | <0.05 | — | — |
| C vs. D | <0.05 | <0.05 | <0.05 | <0.05 | <0.05 | <0.05 | — | — |

### Example 13

Although cyclobenzaprine has been shown to interact with both the serotonergic and noradrenergic receptor systems, the functional interactions of cyclobenzaprine with isolated receptors are not fully characterized and those of norcyclobenzaprine are unknown. Therefore, plasma norcyclobenzaprine was measured in healthy subjects after oral administration of cyclobenzaprine and the binding and functional activity of cyclobenzaprine and norcyclobenzaprine was studied on a set of CNS targets with potential relevance to cyclobenzaprine actions.

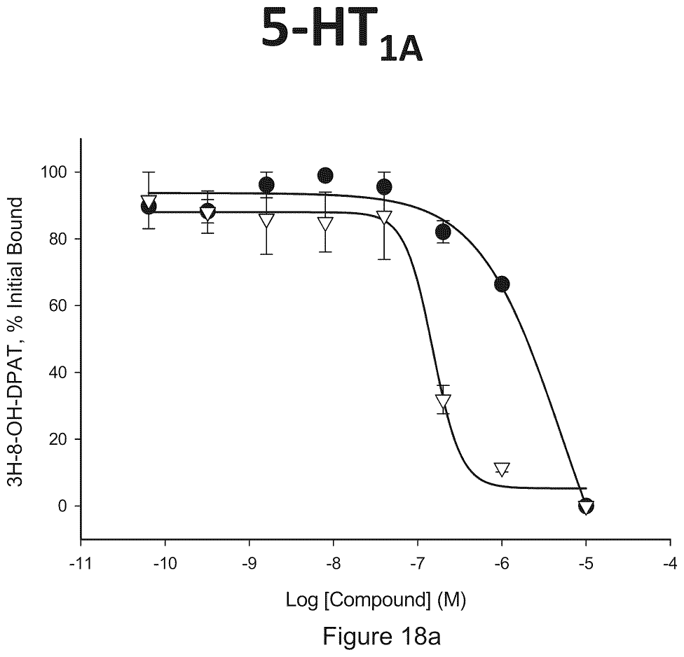

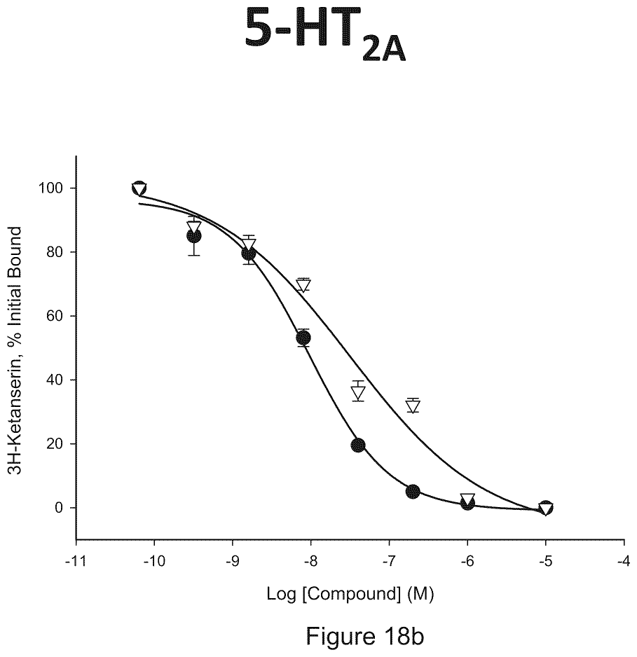

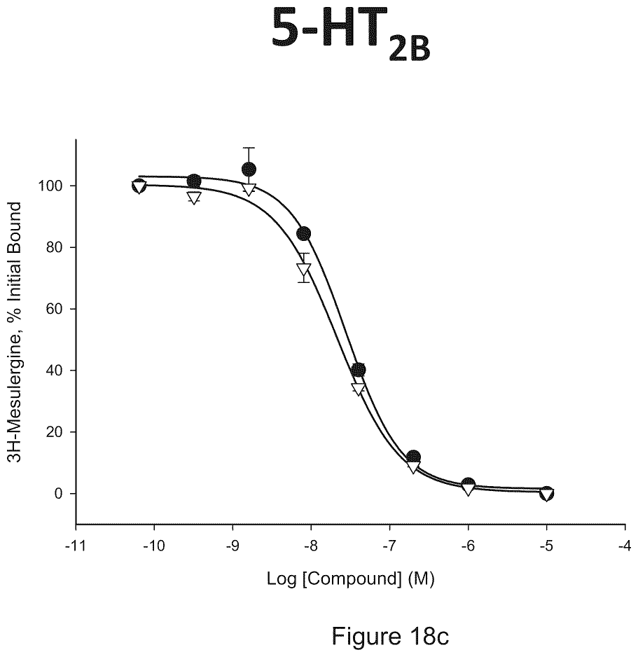

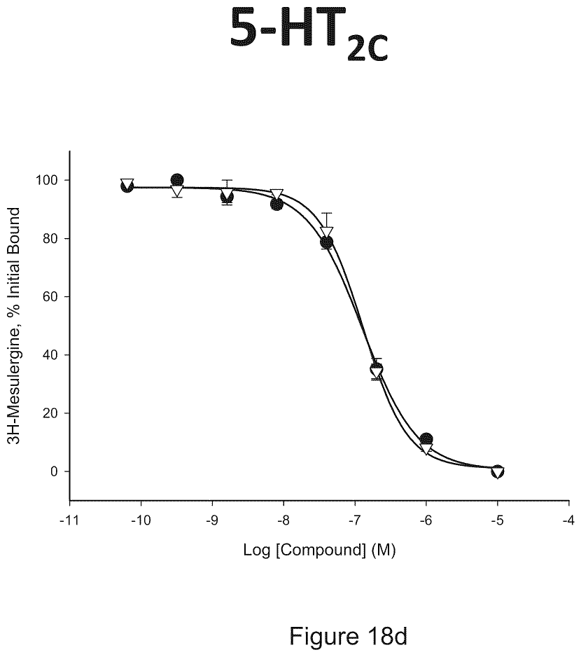

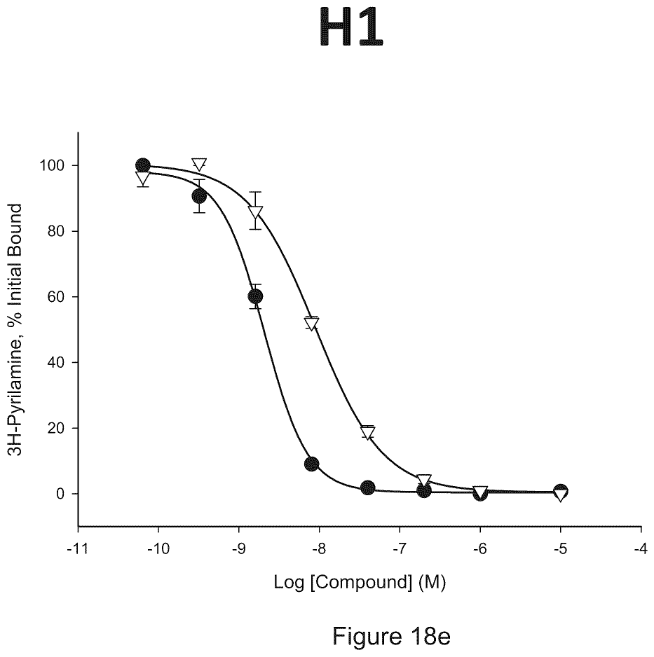

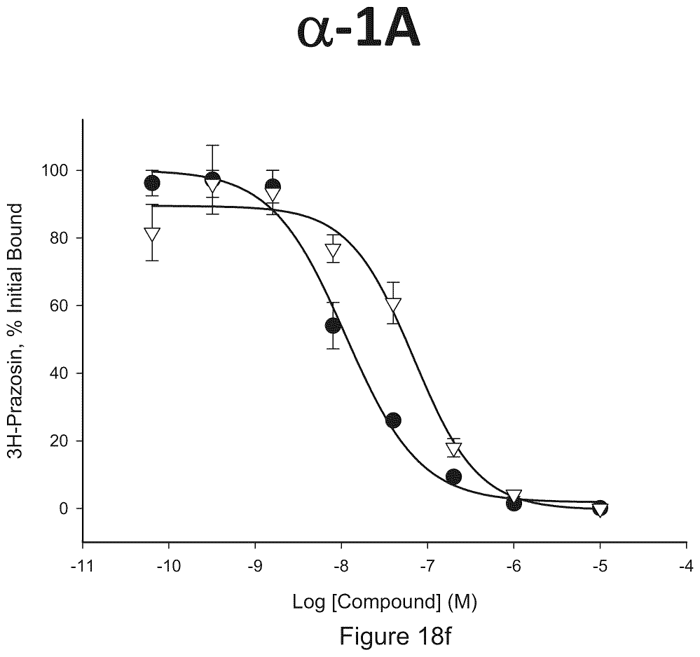

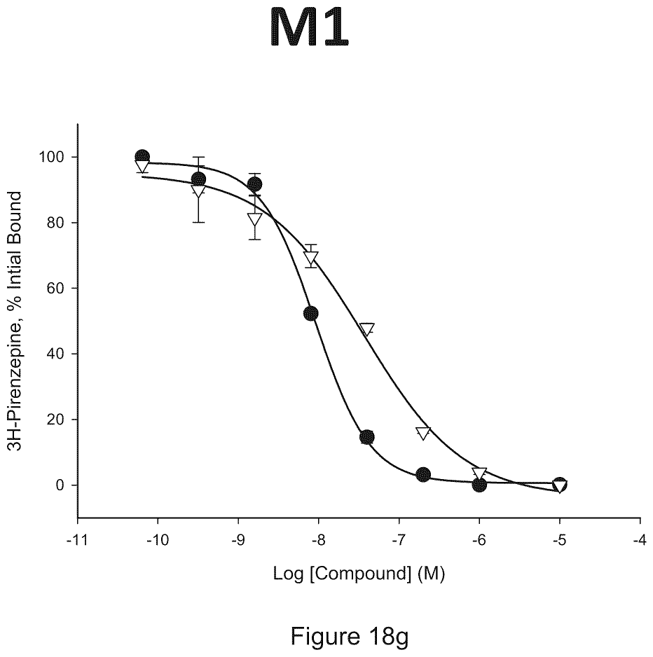

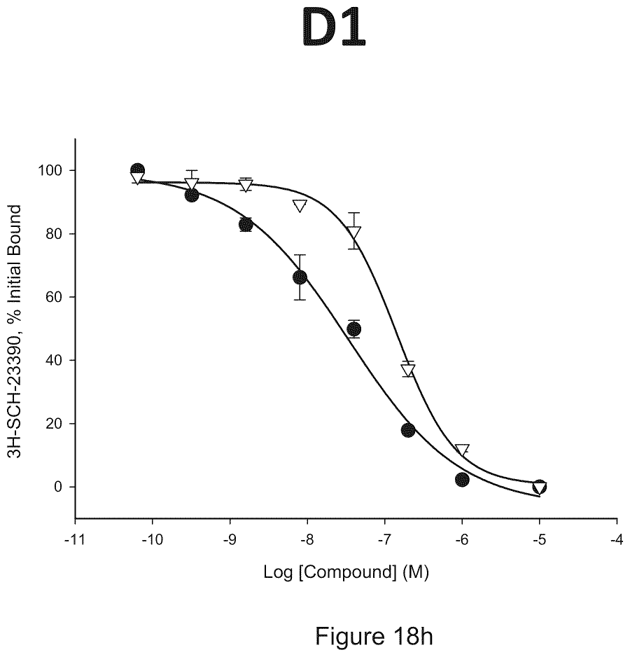

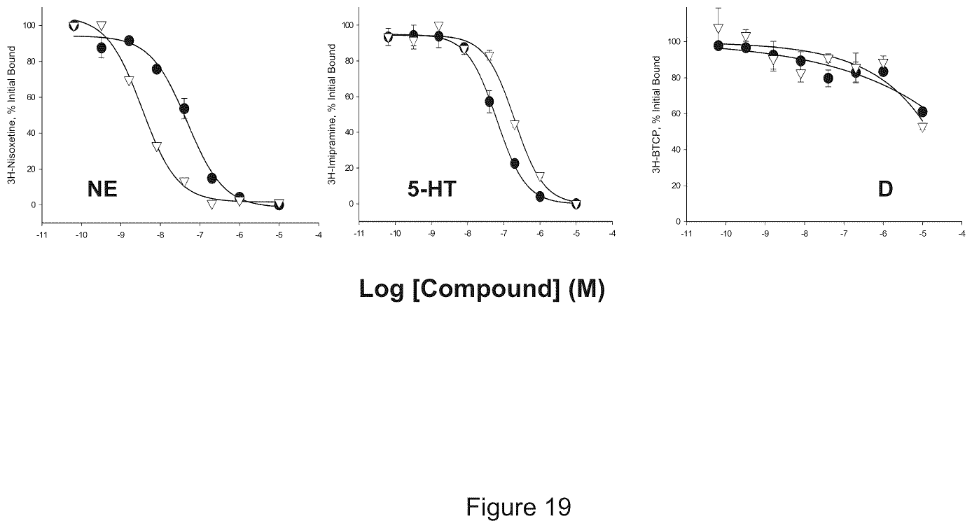

Cyclobenzaprine and norcyclobenzaprine were screened on a broad panel of receptors, channels, enzymes and transporters. Equilibrium receptor binding assays were performed on cell lines expressing select recombinant human serotonin, adrenergic, histamine, and muscarinic receptors. Select receptors also were analyzed for functional antagonism in ligand-induced intracellular calcium mobilization and β-arrestin signaling. FIGS. 18a-h depict the equilibrium binding of cyclobenzaprine (circles) and norcyclobenzaprine (triangles) to cells expressing various recombinant human receptors. In particular, FIG. 18b depicts binding to the 5-HT_{2A} receptor from a Kᵢ about approximately 10^{-6.8} to 10^{-8.8}, which may be consistent with a dynamic effect of bedtime dosing on the central nervous system despite baseline levels of cyclobenzaprine in the blood or central nervous system. Table 68 also shows the Hill plot (also called the Hill slope or the slope factor) ratios depicting the slopes of the curves for the receptors of FIGS. 18a-h. A wide dynamic range is indicated by a slope ratio of less than 1, while a narrow dynamic range is indicated by a slope ratio of greater than 1. A narrow dynamic range means that over a narrow range of concentrations of cyclobenzaprine, if the cyclobenzaprine concentration is increasing, then the population of receptors (e.g., the H-1 receptors) will go from unoccupied to occupied. Likewise, if the concentration of cyclobenzaprine is decreasing, then the population of receptors will go from occupied to unoccupied. A broad dynamic range means that over a wide range of concentrations of cyclobenzaprine, if the cyclobenzaprine concentration is increasing, then the population of receptors (e.g., 5HT2A receptors) will go from unoccupied to occupied. Likewise, if the concentration of cyclobenzaprine is decreasing, then the receptors will go from occupied to unoccupied. Because Kᵢ is the point of inflection (50:50 bound:unbound) in the curve, at a concentration of cyclobenzaprine of 10 times the Kᵢ, a “narrow dynamic range” receptor would be expected to have Henderson-Hasselbach-like characteristics and the receptor would be expected to be 95:5 bound:unbound and in a range beyond the linear range where more ligand would bind a diminishingly small amount of receptor. However, for a “broad dynamic range” receptor, it would be expected that the receptor would be less than 95% bound and still in a linear range where more ligand would bind more receptor. These considerations are important because the Kᵢ values for the 5HT2A and H-1 receptors are close to the therapeutic levels of cyclobenzaprine, where 2.75 ng/ml of cyclobenzaprine (10 nM) is in the plasma. Therapeutically, bedtime doses of cyclobenzaprine should change the occupancy of the 5HT2A receptors and the H-1 receptors, but 5HT2A receptors should be affected to a greater degree because the 5HT2A receptor has a broad dynamic range. FIG. 19 depicts similar binding studies for transporters expressed in the central nervous system. Table 69 shows the binding affinities and functional potency of cyclobenzaprine and norcyclobenzaprine on various central nervous system proteins.

TABLE 68
Hill plot ratios (or Hill slope)

| Receptor | Cyclobenzaprine | Norcyclobenzaprine |
|---|---|---|
| 5-HT1A | 0.87 | 4.07 |
| 5-HT2A | 0.86 | 0.50 |
| 5-HT2B | 1.24 | 1.10 |
| 5-HT2C | 1.13 | 1.40 |
| H1 | 1.61 | 1.04 |
| α-1A | 1.01 | 1.16 |
| M1 | 1.28 | 0.70 |
| D1 | 0.61 | 1.15 |

TABLE 69
Binding and functional potency of cyclobenzaprine and
norcyclobenzaprine on targets expressed in the central nervous system

| | | | IC₅₀ (nM) | | EC₅₀ (nM) | |
|---|---|---|---|---|---|---|
| | Kᵢ (nM) | | (Antagonist) | | (Agonist) | |
| | CBP | nCBP | CBP | nCBP | CBP | nCBP |
| 5-HT_{1A} | 1100 | 76 | | 5300 | 3200 | |
| 5-HT_{2A} | 5.2 | 13 | 230 | 140 | | |
| 5-HT_{2A} | | | 210 (IP) | 450 (IP) | | |
| 5-HT_{2A} | | | 99 (β-arrestin) | 181 (β-arrestin) | | |
| 5-HT_{2B} | 15 | 12 | 100 | 580 | | |
| 5-HT_{2B} | | | 760 (IP) | 1400 (IP) | | |
| 5-HT_{2C} | 43 | 43 | 444 | 1220 | | |
| 5-HT_{2C} | | | 770 (IP) | 2000 (IP) | | |
| 5-HT_{5A} | 730 | 1600 | ND | ND | | |
| 5-HT₆ | 480 | 1400 | 2000 | 2800 | | |
| 5-HT₇ | 67 | 140 | ND | ND | | |
| H₁ | 1.3 | 5.9 | 5.2 | 16 | | |
| H₁ | | | 2.7 (β-arrestin) | 6.1 (β-arrestin) | | |
| α_{1A} | 5.6 | 34 | 4.9 | 16 | | |
| α_{1B} | 9.1 | 11 | 530 | 790 | | |
| α_{1B} | | | 144 (β-arrestin) | 173 (β-arrestin) | | |
| α_{2A} | 360 | 1800 | 4300 | 6400 | | |
| α_{2B} | 21 | 150 | 9800 | 41000 (CAMP) | | |
| α_{2C} | 25 | 48 | NA | NA (CAMP) | | |
| M₁ | 7.9 | 30 | 0.71 | 8.7 | | |
| M₁ | | | 81 (β-arrestin) | 266 (β-arrestin) | | |
| M₂ | 250 | 76 | 3.3 | 33 | | |
| D₁ | 12 | 57 | 65 | 300 | | |
| D_{2S} | 120 | 410 | ND | ND | | |
| D₃ | 34 | 98 | ND | ND | | |
| D_{4.4} | 180 | 250 | ND | ND | | |
| D₅ | 60 | 280 | ND | ND | | |
| Dopamine TP | >10,000 | >10,000 | ND | ND | | |
| Norepinephrine TP | 35 | 2.6 | ND | ND | | |
| Serotonin TP | | 29 | 91 | ND | ND | |
| Sigma 1 | | 120 | 790 | ND | ND | |
| Sigma 2 | | 480 | 2000 | ND | ND | |

IP: inositol triphosphate;
cAMP: 3′-5′-adenosine monophosphate;
TP: transporter;
NA: No Activity;
ND: Not Done

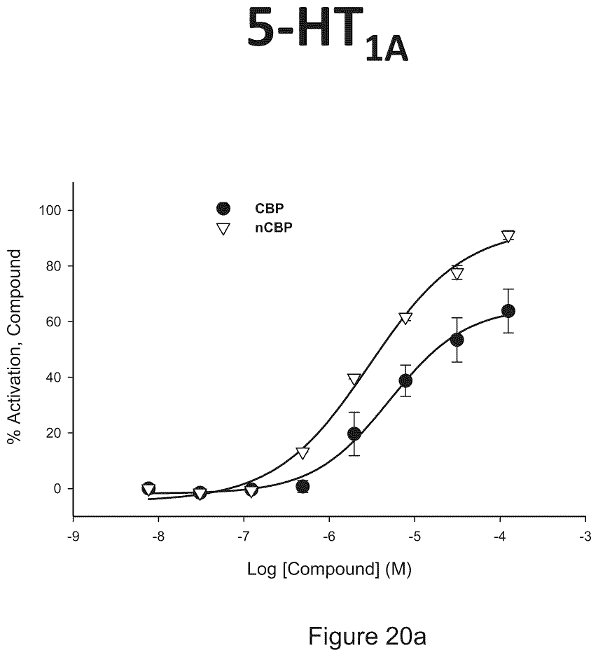

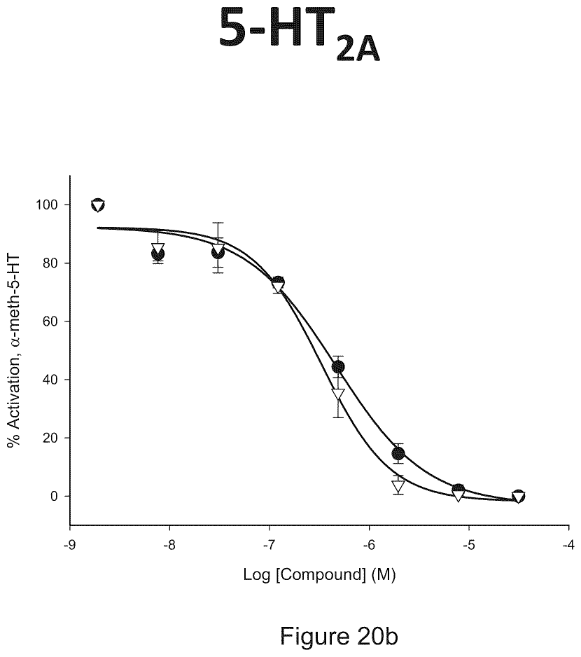

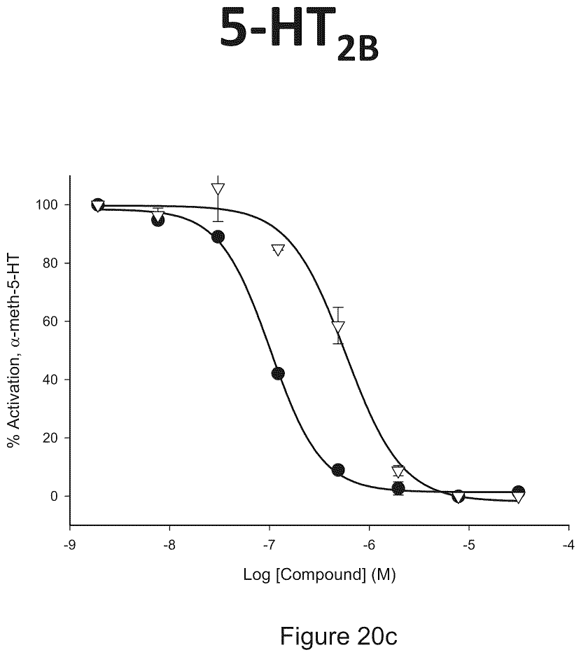

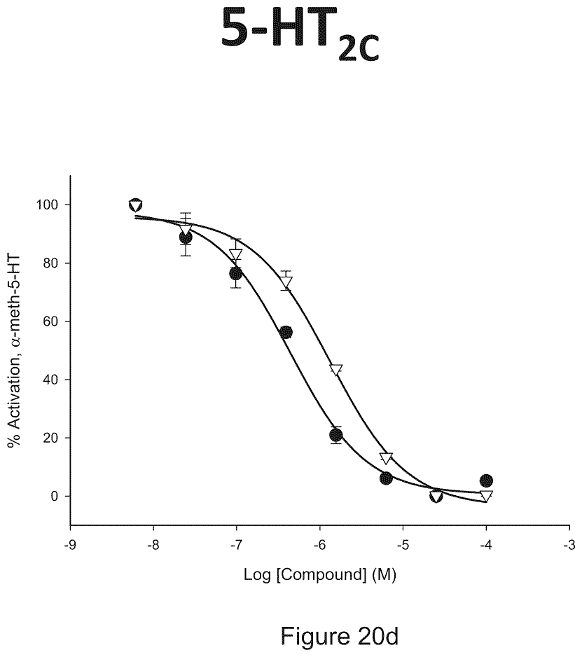

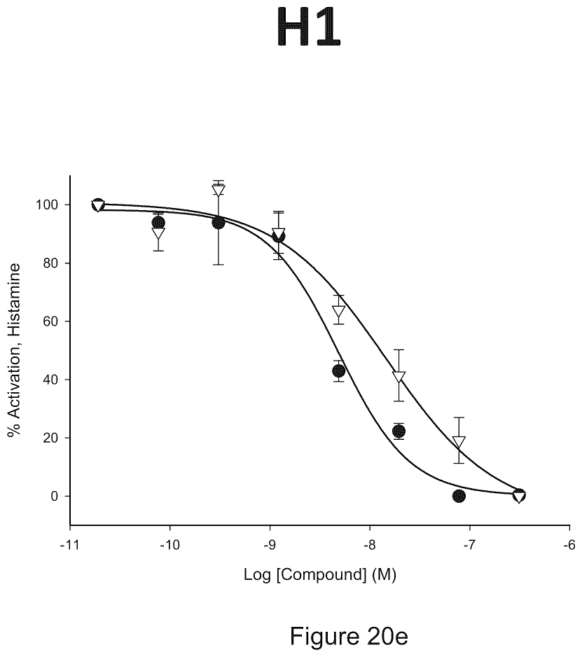

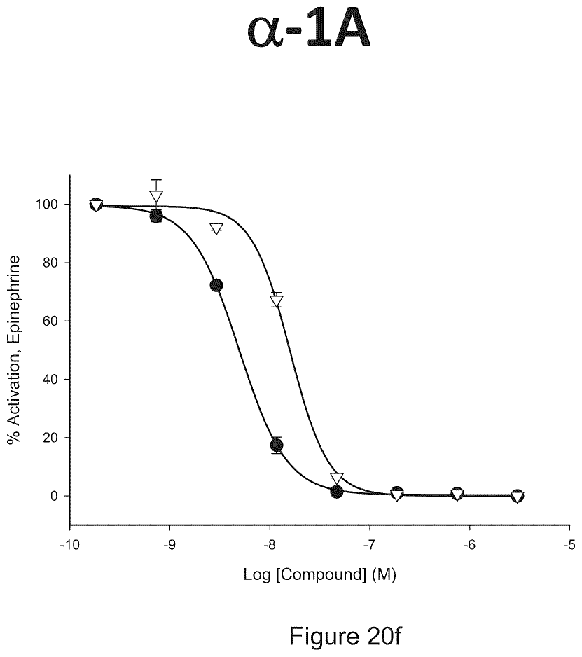

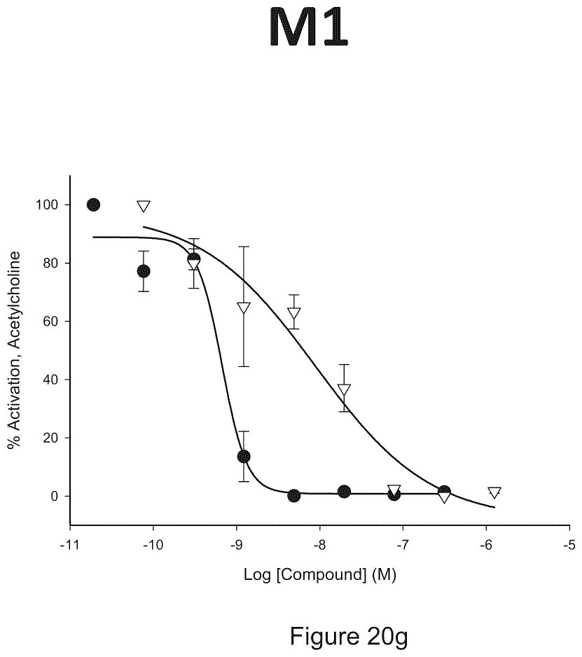

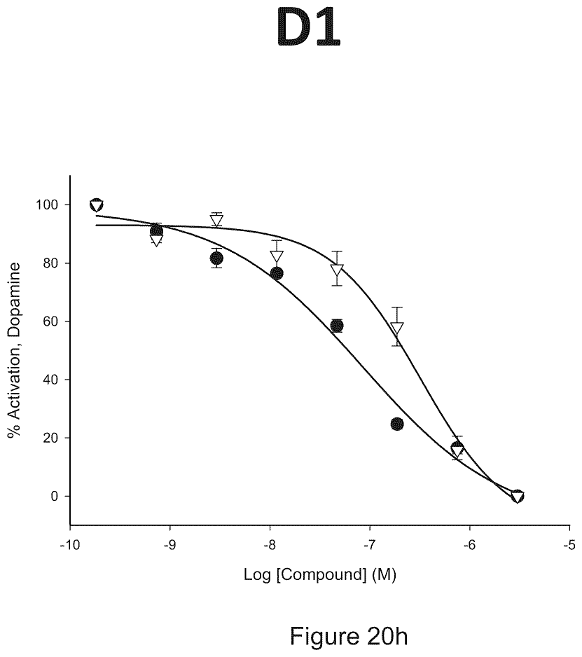

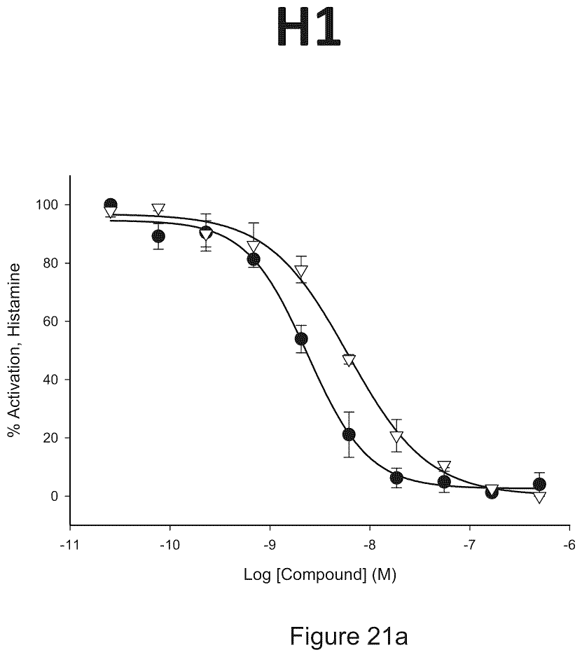

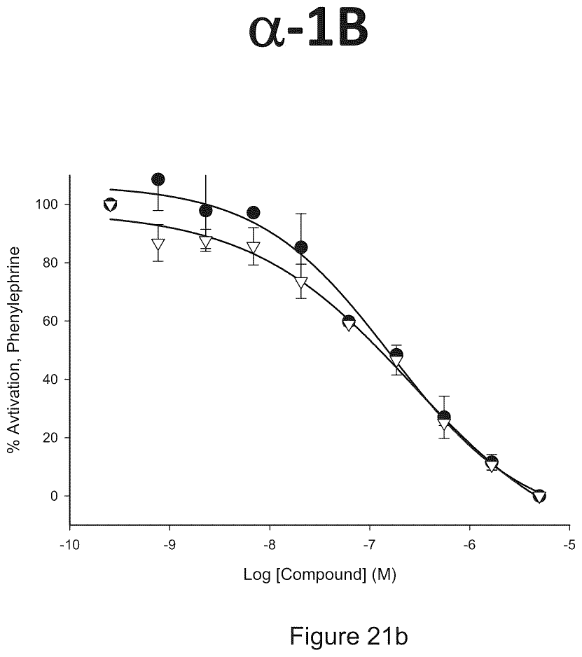

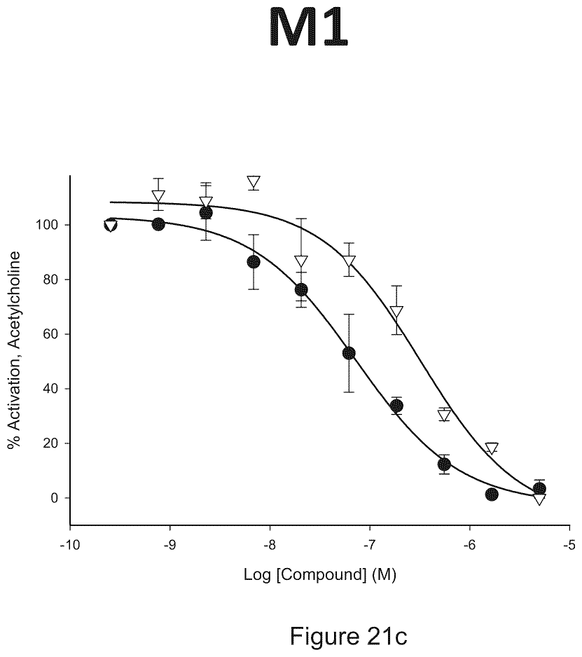

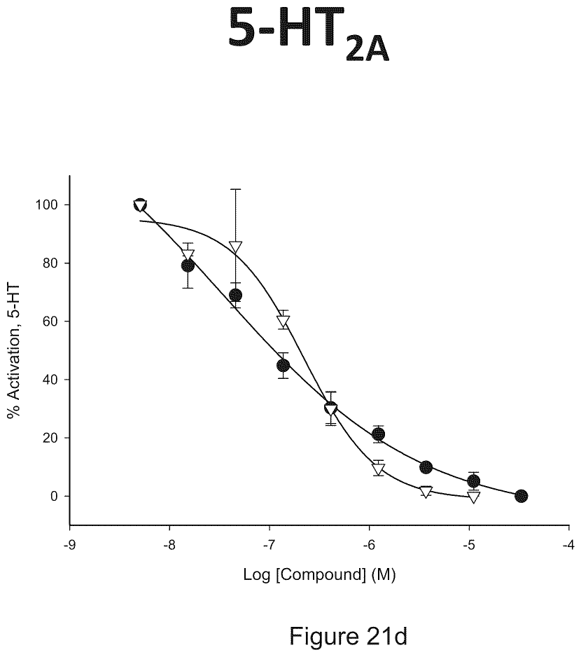

After performing the binding studies described above, functional studies of cyclobenzaprine and norcyclobenzaprine on central nervous system receptors were performed. These assays tested both G-protein-dependent signal transduction by intracellular Ca²⁺ mobilization (FIGS. 20a-h, Table 70), cAMP production or turnover of inositol triphosphate, and G-protein-independent signal transduction by β-arrestin signaling (FIGS. 21a-d, Table 71). Cyclobenzaprine and norcyclobenzaprine exhibited high affinity binding (Kᵢ) to receptors: 5-HT_{2A} (Kᵢ=5.2 and 13 nM, respectively), 5-HT_{2B} (15 and 12 nM), and 5-HT_{2C} (43 and 43 nM), adrenergic α_{1A} (5.6 and 34 nM), α_{1B} (9.1 and 11 nM), α_{2B} (21 and 150 nM) and α_{2C} (25 and 48 nM,); H₁ (1.3 and 5.9 nM); and M₁ (7.9 and 30 nM). Cyclobenzaprine and norcyclobenzaprine were functional antagonists of 5-HT_{2A} (IC₅₀=230 and 140 nM), 5-HT_{2B} (100 and 580 nM), H₁ (5.2 and 16 nM), α_{1A} (4.9 and 16 nM), M₁ (0.71 and 8.7) and M₂ (3.3 and 33 nM) via Ca²⁺ mobilization. By contrast, both cyclobenzaprine and norcyclobenzaprine were functional agonists of 5-HT_{1A} (EC₅₀=5.3 and 3.2 μM). Cyclobenzaprine and norcyclobenzaprine also were functional antagonists of 5-HT_{2A} (IC₅₀=99 and 181 nM), H₁ (2.7 and 6.1 nM), α_{1B} (144 and 173 nM), and M₁ (81 and 266 nM) via β-arrestin signaling.

TABLE 70
Four parameter Hill slope (or fit
slope) ratios; Ca²⁺ signaling

| Receptor | Cyclobenzaprine | Norcyclobenzaprine |
|---|---|---|
| 5-HT1A | −0.97 | −0.79 |
| 5-HT2A | 0.96 | 1.27 |
| 5-HT2B | 1.71 | 1.54 |
| 5-HT2C | 0.96 | 0.94 |
| H1 | 1.22 | 0.79 |
| α-1A | 1.83 | 2.27 |
| M1 | 2.94 | 0.62 |
| D1 | 0.62 | 0.98 |

TABLE 71
Four parameter Hill slope (or fit
slope) ratios; β-arrestin signaling

| Receptor | Cyclobenzaprine | Norcyclobenzaprine |
|---|---|---|
| H1 | 1.69 | 1.09 |
| α-1B | 0.73 | 0.70 |
| M1 | 0.97 | 1.05 |
| 5-HT2A | 0.99 | 1.11 |

### Example 14

An amitriptyline formulation (Formulation (a)) not containing the basifying agent was prepared by blending 0.049 g of amitriptyline hydrochloride with 0.052 g of sodium starch glycolate, 0.399 g of spray-dried lactose, and 0.200 g of microcrystalline cellulose. This powder was then blended with 0.024 g of magnesium stearate, and the entire mixture was compressed into a tablet. When placed in 15 mL of water, the tablet disintegrated in less than 30 seconds. The pH of the resulting slurry was measured to be 4.92.

An amitriptyline formulation (Formulation (b)) containing the basifying agent was prepared by first blending 0.051 g of amitriptyline hydrochloride 0.105 g of sodium bicarbonate. After mixing, this powder was blended with 0.052 g of sodium starch glycolate, 0.356 g of spray-dried lactose, and 0.205 g of microcrystalline cellulose. Finally, the resulting powder was blended with 0.025 g of magnesium stearate, and the entire mixture was compressed into a tablet. When placed in 15 mL of water, the tablet disintegrated in less than 30 seconds. The pH of the resulting slurry was measured to be 7.49.

Additional studies of formulations containing the basifying agent were performed following the procedure described above. After compression, tablets of each formulation were noted to disintegrate in less than 30 seconds when placed in 15 mL of water. The compositions of these formulations, and the pH of the resulting slurries, are summarized in Table 72.

TABLE 72
Amitriptyline formulations and pHs

| | | | | | Jul. 3, 2012 |
|---|---|---|---|---|---|
| | Formulation | Formulation | Formulation | Formulation | Formulation |
| Ingredient | (c) | (d) | (e) | (f) | (g) |
| Amitriptyline | 0.051 g | 0.050 g | 0.050 g | 0.049 g | 0.048 g |
| hydrochloride | | | | | |
| Basifying agent | KH₂PO₄: | Na₂CO₃: | CaCO₃: | TRIS: | Na citrate: |
| | 0.099 g | 0.100 g | 0.098 g | 0.101 g | 0.100 g |
| sodium starch | 0.050 g | 0.051 g | 0.050 g | 0.052 g | 0.051 g |
| glycolate | | | | | |
| spray-dried | 0.349 g | 0.351 g | 0.356 g | 0.351 g | 0.354 g |
| lactose | | | | | |
| microcrystalline | 0.200 g | 0.199 g | 0.201 g | 0.201 g | 0.204 g |
| cellulose | | | | | |
| magnesium | 0.025 g | 0.025 g | 0.026 g | 0.025 g | 0.024 g |
| stearate | | | | | |
| pH of slurry | 5.83 | 10.33 | 7.10 | 8.86 | 7.28 |
| after | | | | | |
| disintegration | | | | | |
| of tablet in 15 | | | | | |
| mL of water | | | | | |

1.-149. (canceled)

150. A method for treating fibromyalgia or reducing one or more associated symptoms of fibromyalgia in a subject in need thereof comprising administering a pharmaceutical composition comprising cyclobenzaprine hydrochloride, a basifying agent, and a pharmaceutically acceptable carrier,
wherein, on administration of the pharmaceutical composition, a combination of the saliva in the oral cavity and the basifying agent converts at least a portion of the cyclobenzaprine hydrochloride to cyclobenzaprine, and
wherein the cyclobenzaprine is transmucosally absorbed in the oral cavity.

151. The method of claim 150, wherein the basifying agent is selected from the group consisting of potassium dihydrogen phosphate, dipotassium hydrogen phosphate, tripotassium phosphate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

152. The method of claim 151, wherein the basifying agent is dipotassium hydrogen phosphate.

153. The method of claim 150, wherein the saliva in the oral cavity has a pH of about 7.1 to 7.4 after administration of the pharmaceutical composition.

154. The method of claim 150, wherein the transmucosal absorption is sublingual absorption.

155. The method of claim 154, wherein the pharmaceutical composition is in a form selected from the group consisting of a liquid, a sublingual tablet, a sublingual film, a sublingual powder, and a sublingual spray solution.

156. The method of claim 151, wherein the cyclobenzaprine hydrochloride is present in the pharmaceutical composition in an amount of less than about 10 mg.

157. The method of claim 156, wherein the cyclobenzaprine hydrochloride is present in the pharmaceutical composition in an amount of less than about 5 mg.

158. The method of claim 157, wherein the cyclobenzaprine hydrochloride is present in the pharmaceutical composition in an amount of 2.8 mg.

159. The method of claim 150, wherein the administration of the pharmaceutical composition is chronic dosing.

160. The method of claim 150, wherein the pharmaceutical composition is administered at bedtime.

161. The method according to claim 150, wherein the cyclobenzaprine on transmucosal absorption affords a cyclobenzaprine dnAUC₀₋₂₀min of greater than or equal to 0.02±25%×10⁻⁶ hr mL⁻¹, a dnAUC₀₋₃₀min of greater than or equal to 0.05±25%×10⁻⁶ hr mL⁻¹, a dnAUC₀₋₄₅min of greater than or equal to 0.14±25%×10⁻⁶ hr mL⁻¹, a dnAUC_{0-1 h} of greater than or equal to 0.26±25%×10⁻⁶ hr mL⁻¹, a dnAUC_{0-2 h} of greater than or equal to 0.87±25%×10⁻⁶ hr mL⁻¹, or a dnAUC_{0-2.5 h} of greater than or equal to 1.23±25%×10⁻⁶ hr mL⁻¹.

162. The method of claim 150, wherein the cyclobenzaprine on transmucosal absorption affords a cyclobenzaprine dnAUC_{0-∞h} of greater than or equal to 20 hr mL⁻¹.

163. The method of claim 150, wherein the cyclobenzaprine on transmucosal absorption affords a cyclobenzaprine dnCₘₐₓ* of greater than or equal to 1.0±25%×10⁻⁶ mL⁻¹.

164. The method of claim 150, wherein the cyclobenzaprine on transmucosal absorption affords a cyclobenzaprine plasma concentration selected from the group consisting of: 50% or less of the Cₘₐₓ 4 hours after administration; 50% or less of the Cₘₐₓ 6 hours after administration; 50% or less of the Cₘₐₓ 8 hours after administration; or 50% or less of the Cₘₐₓ 12 hours after administration.

165. The method of claim 150, wherein the cyclobenzaprine on transmucosal absorption affords a Cₘₐₓ of cyclobenzaprine selected from the group consisting of: greater than or equal to 10 ng/mL; greater than or equal to 15 ng/mL; greater than or equal to 20 ng/mL; greater than or equal to 25 ng/mL; greater than or equal to 30 ng/mL; greater than or equal to 10 ng/mL above the baseline level of cyclobenzaprine in the individual immediately prior to administration; greater than or equal to 15 ng/mL above the baseline level of cyclobenzaprine in the individual immediately prior to administration; greater than or equal to 20 ng/mL above the baseline level of cyclobenzaprine in the individual immediately prior to administration; greater than or equal to 25 ng/mL above the baseline level of cyclobenzaprine in the individual immediately prior to administration; and greater than or equal to 30 ng/mL above the baseline level of cyclobenzaprine in the individual immediately prior to administration.

166. The method of claim 150, wherein the cyclobenzaprine on transmucosal absorption affords a tₘₐₓ of cyclobenzaprine selected from the group consisting of: less than 4.7 hours, less than 4 hours; less than 3 hours; less than 2 hours; less than 1 hour; less than 45 minutes; less than 30 minutes; or less than 15 minutes.

167. The method of claim 150, wherein the cyclobenzaprine on transmucosal absorption affords a Cₘₐₓ of cyclobenzaprine greater than or equal to 2 ng/mL.
